(19) United States
(12) Patent Application Publication
(10) Pub. No.: US 2006/0216722 A1
(43) Pub. Date: Sep. 28, 2006

(54) Glomerular expression profiling

(76) Inventors: Christer Betsholtz, Vastra Frolunda (SE); Karl Tryggvason, Stockholm (SE); Minoru Takemoto, Akita-shi (JP); Liqun He, Stockholm (SE); Jaakko Patrakkas, Stockholm (SE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/090,997

(22) Filed: Mar. 25, 2005

(51) Int. Cl. C12Q 1/68; C07H 21/04

(52) U.S. Cl. 435/6; 536/24.3

(57) ABSTRACT

The present invention provides compositions comprising various glomerular probe sets and methods for their use.

A compact disc submission containing a Sequence Listing is hereby expressly incorporated by reference. The submission includes two compact discs (“COPY 1” and “COPY 2”), which are identical in content. Each disc contains the file entitled “04-1059 SeqList.txt,” 9.8 MB in size, created Mar. 23, 2005.

A compact disc submission containing Table 14A and Table 14B is hereby expressly incorporated by reference. The submission includes two compact discs (“COPY 1” and “COPY 2”), which are identical in content. Each disc contains the file entitled “Table 14A.csv,” 604 KB in size, created Mar. 24, 2005, and the file entitled “Table 14B.csv,” 687 KB in size, created Mar. 24, 2005.

### BACKGROUND

The kidney glomerulus is a highly specialized filtration unit, capable of filtering large volumes of plasma into primary urine, which allows for excretion of low molecular weight waste products, while restricting passage of plasma proteins of the size of albumin and larger (1). The filter constitutes three layers of the glomerular capillary wall: a fenestrated endothelium, glomerular basement membrane (GBM), and a slit diaphragm located between interdigitating foot processes of epithelial podocytes. The ability of the glomerular filter to exclude plasma proteins from the filtrate is essential for life. Leakage of plasma proteins can result in nephrotoxic proteinuria leading to a pathologic chain reaction with end-stage renal disease (ESRD) as a final outcome. For ESRD patients, life-long dialysis or renal replacement constitute the only available treatment options. About two-thirds of ESRD cases are the result of a primary glomerular insult. Glomeruli are affected in systemic diseases, such as diabetes, hypertension, lupus and infections, as well as in drug-induced toxicity, but the molecular pathomechanisms of these disorders are not understood. The central role of the glomerulus in renal pathology makes it reasonable to assume that efficient prevention and treatment of some of the major progressive renal disorders require new therapies targeting specific pathogenic processes in the glomerulus. Although we currently lack knowledge about the molecular pathogenesis of the common glomerular disorders, recent insight into the genetic basis of certain rare hereditary glomerular diseases has identified specific components of the glomerular filter, as well as the podocytes as major targets of glomerular pathogenic pathways (2-11).

The GBM, which is synthesized by both endothelial cells and podocytes, contains specific proteins, such as type IV collagen, laminin, proteoglycans and nidogen (12). The composition of the GBM switches during glomerular development from fetal collagen IV (α1:α1:α2), laminin-1 (α1:β1:γ1), laminin-8 (α4:β1:γ1) and laminin-10 (α5:β1:γ1) to adult collagen IV (α3:α4:α5) and laminin-11 (α5:β2:γ1) (12, 13). Podocyte differentiation is crucial for this GBM switch. Mutations in adult type IV collagen lead to distortion of the GBM, hematuria and Alport syndrome (2, 7, 12), and defects in the laminin β2 chain of laminin-11 cause Pierson congenital nephrotic syndrome (8), which emphasizes the role of the GBM in the glomerular filter.

Podocytes are highly specialized epithelial cells, which enclose the glomerular capillaries by interdigitating foot processes bridged by a slit diaphragm (14). Although podocytes account for only about 15% of the total number of glomerular cells, they play a major role in glomerular biology and particularly in glomerular disease. Based on electron microscopy, it has been proposed that the slit diaphragm is a structured, zipper-like filter with pores smaller than albumin (15), thus constituting a size-selective molecular sieve. This structure was recently confirmed by electron tomography, and the transmembrane protein nephrin was demonstrated to be a structural component of the slit diaphragm zipper (16). The slit diaphragm has been shown to have a central role in the pathomechanisms of many severe glomerular diseases. Malfunction or absence of nephrin leads to lethal congenital nephrotic syndrome of the Finnish type (3) characterized by massive proteinuria and loss of the slit diaphragm filter structure (16). Additional proteins, such as podocin (4), CD2 associated protein (CD2AP) (17), ZO-1 (18), FAT-1 (19), Neph1 (20-22) and P-cadherin (23), which have been localized to the slit diaphragm region are potential components of a slit diaphragm protein complex. The podocin gene NPHS2 is mutated in human steroid-resistant nephrotic syndrome (4), as well as in late-onset familial focal segmental glomerulosclerosis (FSGS) (24). CD2AP mutations have also been associated with sporadic cases of FSGS (25). In animal models, the loss of nephrin (26), Neph1 (27), FAT-1 (28), or CD2AP (17, 25) disrupts the slit diaphragm, thereby causing proteinuria. CD2AP binds nephrin, podocin and actin, hence potentially forming a structural bridge between the slit diaphragm and the podocyte cytoskeleton (29). Interestingly, mutations in the ACTN4 gene, which encodes alpha-actinin 4 (a component of the actin cytoskeleton), leads to familial FSGS (30). In mice, both loss- and gain-of-function mutations of alpha-actinin 4 lead to glomerular disease and proteinuria (31, 32).

Podocytes also play a pivotal role in glomerular development by secreting vascular endothelial growth factor (VEGF) (33), which attracts endothelial cells into the developing glomerular tuft. VEGF may also have a late role in establishing the fenestrations in the glomerular capillary endothelium. The role of VEGF in the glomerulus is highly dosage sensitive. Systemic inhibition of VEGF causes proteinuria (34, 35), and genetic reduction in podocyte VEGF expression leads to glomerular abnormalities, including loss of capillary fenestrations. VEGF overexpression in podocytes, on the other hand, leads to collapsing glomerulopathy similar to HIV-associated nephropathy (36). In concert with VEGF, podocytes also secrete the growth factors angiopoietin I and TGF-β1, which may play important roles in glomerular microvascular assembly (37, 38). Podocyte associated transcription factors, such as LMX1B and WT1, which are important for podocyte differentiation, have also been associated with the glomerular disorders Nail-Patella, Denys-Drash and Frasier syndromes (6, 9, 10).

The recent recognition of specific GBM- and podocyte-associated proteins as central players in rare glomerular diseases emphasizes the need for more comprehensive studies on glomerulus biology, as the results may provide a new understanding of the pathomechanisms of the common and complex glomerular diseases that currently constitute the main challenge of clinical nephrology. Such studies should also involve analyses of the glomerular mesangial and endothelial cells, the role of which in glomerular disease is largely unknown. A number of studies have recently described the mapping of the transcriptome of different parts of the kidney, including subportions of the nephron (39-44), but none of these studies was specifically focused on the glomerulus. In two studies, isolated glomeruli were included in the analysis, but the transcription data obtained were incomplete, as shown by the lack of information about many of the known podocyte-specific transcripts (42, 44). Most likely, the difficulties associated with molecular profiling of glomeruli reflect the fact that glomeruli constitute less than 10% of the kidney tissue, and moreover, that the podocyte is the least abundant cell type in the glomerulus, contributing to only about 1% of the entire kidney tissue. Therefore, low abundance podocyte transcripts, like the nephrin mRNA, are difficult to detect unless glomeruli or podocytes are enriched before the analysis.

### SUMMARY OF THE INVENTION

In one aspect, the present invention provides compositions comprising a plurality of isolated probes that in total selectively binds to at least 2 of the glomerular markers disclosed herein in Table 6 or Table 7, complements thereof, or their expression products, wherein at least 10% of the probes in total are selective for glomerular markers, and methods and kits for the use of such compositions.

In a further aspect, the present invention provides compositions comprising a plurality of isolated probes that in total selectively bind to at least 51 of the glomerular markers disclosed herein in Table 9, complements thereof, or their expression products, wherein at least 10% of the probes in total are selective for glomerular markers, and methods and kits for the use of such compositions.

In a further aspect, the present invention provides compositions comprising a plurality of isolated probes that in total selectively bind to at least 12 of the podocyte markers disclosed herein in Table 3, complements thereof, or their expression products, wherein at least 1.5% of the probes in total are selective for podocyte markers, and methods and kits for the use of such compositions.

In a further aspect, the present invention provides compositions comprising a plurality of isolated probes that in total selectively bind to at least 7 of the non-podocyte glomerular markers disclosed herein in Table 4, complements thereof, or their expression products, wherein at least 8.5% of the probes in total are selective for non-podocyte glomerular markers, and methods and kits for the use of such compositions.

The present invention also provides an isolated nucleic acid sequence comprising or consisting of a nucleotide sequence according to SEQ ID NO:2043, expression vectors comprising the nucleotide sequence, and host cells transfected with the expression vector.

The present invention further provides novel dendrin nucleic acids and polypeptides comprising or consisting of the amino acid sequence of SEQ ED NOS:2041-2042.

### BRIEF DESCRIPTION OF THE FIGURES

FIG. 1 Evaluation of normalization and subtraction procedure.

A: The four glomerular cDNA libraries, the number of sequenced clones from each library, and the corresponding numbers of different annotated genes and non-annotated ESTs are listed in the left panel. To the right; schematic illustrations of the theoretical distribution of cDNA relative to the original transcript abundance in the standard (St) normalized, and super-normalized libraries.

B: The relative abundance of different housekeeping genes in the adult standard (blue bars), adult normalized (red bars), and adult super-normalized (green bars) libraries. Eafa1, elongation factor 1 alpha 1; B2m, β2 microglobulin; Gapd, Glyceraldehyde 3-phosphate dehydrogenase; Ftl1, Ferritin light chain 1; Oaz1, Ornithine decarboxylase antizyme; Rps8, 40S ribosomal protein S8.

FIG. 2 GlomChip design and performance

A: GlomChip was printed with 16704 GlomBase EST clones, 1344 other mouse cDNA clones and 10 different Arabidopsis Thaliana (A. Thaliana) PCR-products. Mouse housekeeping gene cDNAs and/or A. Thaliana cDNAs were put in every two corners of 34×34 spots square in order to control for serial contamination during printing, and to facilitate spot segmentation during analysis.

B: TA typical two-target hybridization result. Background hybridization was deduced from the A. Thaliana spots. Note that the weak horizontal band of hybridizing clones on each 34×34 spot quadrant represent the clones derived from normalized libraries, i.e. clones that on average represent mRNAs of lower abundance than the clones from the standard libraries seen at the top and bottom of each quadrant.

C and D: Identification of genes with glomerulus-restrictively expression pattern. GlomChip was hybridized against labeled targets from different tissues; isolated glomeruli, rest of kidney, brain capillary fragments, GFP positive glomerular cells, and GFP negative glomerular cells. Step-wise comparisons between pairs of tissues provided lists of significantly upregulated genes in each tissue category, or not significantly different (n.s.) (Gene category (GC) 1-8). GlomBase cDNAs and IMAGE clones are categorized separately (C and D, respectively). The threshold for differential expression was set to 2-fold difference at statistical significance (p<0.05).

FIG. 3 Isolation of podocytes from Podocin-Cre x Z/EG mice.

A: Postnatal day 1 kidneys from Podocin-Cre x Z/EG mice examined by fluorescence microscopy. Note the crescent of GFP-positive podocytes in each glomerulus. B: Dynabead-isolated glomeruli from Podocin-Cre x Z/EG mice. C&D: Single cell suspensions were prepared from isolated glomeruli and evaluated under the microscope with or without fluorescent. E: Glomerular cells sorted by GFP fluorescence (quadrangle).

FIG. 4 Glomerular expression determined by non-radioactive in situ hybridization.

Results from E18.5 kidneys are shown. A: Podocyte-expressed genes. Nphs2 (podocin), Podx1 (podocalyxin), Sem2 (semaphorin sem2), Pi15 (protease inhibitor 15). B: Mesangial, juxtaglomerular and endothelial cell-expresed genes. Sfrp2 (secreted frizzled-related protein 2), Igfbp5 (insulin-like growth factor binding protein 5), Akr1b7 (Aldo-keto reductase family 1, member B7). Lmo7 (lim domain only protein 7).

FIG. 5 Temporal expression of glomerular markers during nephron development.

Expression of known and novel markers for glomerular cells through the different stages of nephron and glomerular development. Note that only Sfrs2 is expressed at the earliest stage of nephron development, whereas all other markers appear in the podocyte and mesangial/juxtaglomerular apparatus cells at their first appearance during S-shaped (podocytes) and capillary loop (mesangial cells) stages. For abbreviations, see legend to FIG. 4.

FIG. 6 Expression of dendrin mRNA and protein in the glomerulus and localization of the dendrin protein to podocyte foot processes.

A: Dark-field image of radioactive in situ hybridization of dendrin to E18.5 mouse kidney. Inset shows silver grains distributed over the podocyte crescent in a capillary loop stage glomerulus. B: Immunohistochemistry localizes the dendrin protein to glomeruli. Inset shows strong staining of the podocytes. C: Dendrin immuno-electron microscopy of podocyte foot processes. Note the localization of gold labeling to the inner leaflet of the foot process plasma membrane in regions where these appose to form slit diaphragms (arrows). D: Western blot analysis demonstrates an 80 kDa dendrin protein species in Dynabead-isolated glomeruli (lane2) but not in the rest of kidney (lane 1).

FIG. 7 Comparison of results using GlomChip, Stanford cDNA chip and SAGE nephron expression approaches.

GlomChip contains 13368 cDNA clones corresponding to 6053 different genes. The Stanford cDNA chip used by Higgins et al (44) contains 41,859 probes. The SAGE study (42) analyzed more than 90,000 different tags. Using GlomChip, 356 different ENSEMBL mouse genes were identified to be significantly upregulated in the mouse glomerulus compared with rest of kidney tissue. By the Stanford cDNA chip analysis, the 139 genes predominantly expressed in human glomerulus corresponds to 118 different ENSEMBL mouse homolog genes. From the SAGE analysis, 229 Tags were identified to be enriched in human glomerulus, corresponding to 143 ENSEMBL mouse homolog genes. The overlap between the three studies is illustrated. Genes/proteins previously published to be expressed in the glomerulus (Table 8) are listed in the respective area, together with their expression ratios (glomerulus/rest of kidney) and statistical P value.

### TABLE LEGENDS

Table 1: Distribution of Sequenced Clones Among Different Mouse Glomerulus Libraries.

Distribution of sequenced clones among different mouse glomerulus libraries. After removing vector sequence, sequences shorter than 100 nucleotides were excluded for further analysis. St, standard; n1, normalized; n2, super normalized.

Table 2: Comparison of GlomBase Content to that of 11 Kidney EST Libraries.

For comparison we selected a set of known podocyte markers. Numbers represent the total number of ESTs in each library. For Glombase, the numbers show the total representation in the four libraries, as well as representation in the standard libraries only (in parenthesis). For example, a total of 10 nephrin ESTs were found in GlomBase, of which 8 derived from the standard libraries. The following kidney libraries were compared with GlomBase: Library 1: Stratagene mouse kidney library, library 2: GuayWoodford mouse kidney day 0 library, library 3: GuayWoodford mouse kidney day 7 library, library 4: C57BL/6J kidney library, library 5: RIKEN 0 day neonate kidney library, library 6: RIKEN adult male kidney library, library 7: RIKEN kidney library, library 8: RIKEN El 6 kidney library, library 9: RIKEN E17 kidney library, library 10: Sugano mouse kidney library, library 11: NCI CGAP Kid14 library

Table 3: List of category 6 genes in FIG. 2 C-D.

Table 4: List of category 7 genes in FIG. 2 C-D.

Table 5: List of category 8 genes in FIG. 2 C-D.

Table 6: List of novel mouse glomerular markers.

Table 7: List of novel human glomerular markers.

Table 8. Result of literature search for glomerulus gene and protein expression demonstrated with cellular resolution by in situ hybridization or immunohistochemistry. The Table provides the following information (in columns from left to right): 1) gene name or acronym. 2) ENSEMBL ID number. 3) Literature reference. 4) PubMed ID for reference. 5) Presence in Glombase (Y/N). 6) Number of ESTs in GlomBase. 7) Species from which information was derived. 8) Selected in GlomChip analysis (Y/N), 9) Selected in SAGE study by Chabardes-Garonne et al., 2003. 10) Selected in array study by Higgins et al., 2004.

Table 9. List of category 1 genes in FIG. 2 C,D.

Table 10. List of category 2 genes in FIG. 2 C,D.

Table 11. List of mouse category 3 genes in FIG. 2 C,D.

Table 11A. List of corresponding human category 3 genes.

Table 12. List of category 4 genes in FIG. 2 C,D.

Table 13. List of category 5 genes in FIG. 2 C,D.

Table 14A-B. List of mouse glomerular markers in the mouse GlomBase™ (14A) and list of human glomerular markers (14B) in the human GlomBase™. This Table is provided on CD only.

Table 15. List of non-novel mouse Category 3 glomerular markers.

Table 16. List of non-novel human Category 3 glomerular markers.

Table 17. List of 942 mouse glomerular expressed EST sequences that did not match ENSEMBL annotated genes, but matched the mouse genome.

### DETAILED DESCRIPTION OF THE INVENTION

All publications, GenBank and ENSEMBL Accession references, patents and patent applications cited herein are hereby expressly incorporated by reference for all purposes.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

In a first aspect, the present invention provides compositions comprising a plurality of isolated probes that in total selectively binds to at least 2 of the glomerular markers disclosed herein in Table 6 or Table 7, complements thereof, or their expression products, wherein at least 10% of the probes in total are selective for glomerular markers. Table 6 lists those mouse glomerular genes that have been identified herein as glomerular markers and which were not previously known to be expressed in the glomerulus. Table 7 lists the human genes corresponding to the mouse genes listed in Table 6 (“homologues”), and comprise novel human glomerular markers. Tables 6 and 7 include database accession information for each of the listed glomerular markers, while the relevant nucleotide and amino acid sequences are provided in the sequence listing (and the corresponding SEQ ID NOS. are provided in Tables 6 and 7). The human homologue for a specific mouse gene listed in Table 6 can be determined by comparing the “Gene name” as listed in each of Tables 6 and 7. The human homologues (the human gene corresponding directly to the mouse gene) were identified through genome-wide scans for homologs and then using certain criteria discrimination between orthologs and so called paralogs. Paralogs are homologous genes in the same genome that arose through gene duplication. The human orthologs were defined in the ENSEMBL database, and their definition has been used herein to assign the human homologues.

As demonstrated below, expression products from polynucleotides comprising the nucleic acid sequence disclosed herein in Table 6 (mouse 280 novel glomerular markers) or Table 7 (human 264 novel glomerular markers) have been identified as novel glomerular markers (i.e.: not previously known to be expressed in the glomerulus). The number of novel glomerular markers in Table 6 is 280 (see number in left-hand column), while over 400 nucleic acid and amino acid sequences corresponding to the 280 novel glomerular markers are disclosed in Table 6 (see columns with SEQ ID NOS.) Where a given glomerular marker is correlated with multiple nucleic acid SEQ ID NOS. in Table 6 or 7, this reflects the presence of alternatively spliced nucleic acids (and their resulting encoded amino acid sequences) from the same gene.

The compositions according to each aspect and embodiment of the invention described below can be used to profile a glomerular tissue sample to identify glomerular expression profiles of interest. Such “glomerular expression profiling” can be used, for example, to establish expression profiles and specific biomarkers for various patient populations with renal disease-related indications, including but not limited to nephropathy, proteinuria, nephrotoxicity, end stage renal disease, diabetes, hypertension, infections, nephrotic syndromes, and glomerulosclerosis. Such glomerular expression profiles can be used, for example, to establish pathogenic pathways for different renal diseases, which will improve on renal histopathology as a means to measure renal disease conditions. Such methods are also useful, for example, to define glomerular profiles and biomarkers in various types of renal disease patient populations that correlate with a positive response to a particular therapeutic strategy and/or particular drug candidate; such profiles and biomarkers can then be used to screen patients to identify those patients that are suitable candidates for treatment with the drug. The methods of the invention can also be used, for example, to identify profiles and biomarkers associated with renal toxicity, wherein pre-clinical drug candidates can then be screened for such renal toxicity-associated profiles and biomarkers to weed out at an early stage of development those drug candidates that induce renal toxicity.

As used herein according to each aspect and embodiment of the invention, the term “glomerular marker . . . or their expression products” (also referred to simply as “glomerular marker”) means a nucleic acid or protein product expressed in the glomerulus. In various embodiments, the glomerular marker comprises DNA (including but not limited to cDNA), RNA (including but not limited to mRNA), or polypeptides (including but not limited to full length proteins or fragments thereof). In a preferred embodiment, the glomerular marker comprises RNA. The definition of “glomerular marker” used herein does not require that the glomerular marker be expressed only in the glomerulus.

As used herein according to each aspect and embodiment of the invention, the term “probe” refers to any compound or compounds that can be used to selectively bind to a glomerular marker of interest. In various non-limiting examples, the probe can comprise DNA (including but not limited to polynucleotide probes), RNA (including but not limited to polynucleotide probes), and polypeptides (including but not limited to antibodies). In a preferred embodiment, the probes comprise DNA. As used herein a “probe” does not include compounds used as negative controls that do not selectively bind to a marker of interest (including but not limited to randomized or scrambled sequence compounds, and competitor nucleic acids and proteins used to minimize non-specific binding), but does include control probes that selectively bind to non-glomerular markers. The compositions of the various aspects of the invention, and embodiments thereof, may contain multiple probes for a single glomerular marker; for example, a composition according to each aspect of the invention may comprise a single polynucleotide probe for a 100 nucleotide region of each of two different glomerular markers, or it may comprise a polynucleotide probe for each of three different 100 nucleotide region of each of each of ten different glomerular markers. Those of skill in the art will understand that many such permutations are possible based on the teachings herein.

As used herein according to each aspect and embodiment of the invention, the term “selectively binds to” means that the probe preferentially binds to the glomerular marker of interest, and minimally or not at all to other markers, under standard conditions. For example, where the probes comprise polynucleotides, specific hybridization conditions used will depend on the length of the polynucleotide probes employed, their GC content, as well as various other factors as is well known to those of skill in the art. (See, for example, Tijssen (1993) Laboratory Techniques in Biochemistry and Molecular Biology—Hybridization with Nucleic Acid Probes part I, chapt 2, “Overview of principles of hybridization and the strategy of nucleic acid probe assays,” Elsevier, N.Y. (“Tijssen”)). In one embodiment, stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. High stringency conditions are selected to be equal to the Tm for a particular probe. An example of stringent conditions are those that permit selective hybridization of the isolated polynucleotides to the genomic or other target nucleic acid to form hybridization complexes in 0.2×SSC at 65° C. for a desired period of time, and wash conditions of 0.2×SSC at 65° C. for 15 minutes. It is understood that these conditions may be duplicated using a variety of buffers and temperatures. SSC (see, e.g., Sambrook, Fritsch, and Maniatis, in: Molecular Cloning, A Laboratory Manual, Cold Spring Harbor Laboratory Press, 1989) is well known to those of skill in the art, as are other suitable hybridization buffers.

As will be apparent to those of skill in the art, the compositions of the various aspects and embodiments of the invention can further comprise other components that may be of use in assays for glomerular expression profiles, including but not limited to buffer solutions, hybridization solutions, and reagents for storing the compositions.

In this first aspect, at least 10% of the probes of the composition are selective for glomerular markers, such as those disclosed herein in Tables 3, 4, 5, 6, 7, 9, and 11-17, as well as other glomerular probes not disclosed herein.

The compositions of the invention may contain probes that are not glomerular specific (for example, for use as control sequences to verify the glomerular-specific nature of an assay in which the compositions are used), so long as such probes do not make up more than 90% of the probes of the composition. In various preferred embodiments, at least 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for glomerular markers, such as those disclosed herein in Tables 3, 4, 5, 6, 7, 9, and 11-17, as well as other glomerular probes not disclosed herein.

In one preferred embodiment or each of the aspects and embodiments of the present invention, the plurality of probes comprises polynucleotide probes. The term “polynucleotide” as used herein with respect to each aspect and embodiment of the invention refers to DNA or RNA, preferably DNA, and more preferably cDNA or oligonucleotide probes derived from expressed portions of the glomerular marker gene, in either single- or double-stranded form, of any length. In a preferred embodiment, polynucleotide probes of the invention are at least 10 nucleotides in length, more preferably at least 15 nucleotides in length, and even more preferably at least 25 nucleotides in length. It includes the recited sequences as well as their complementary sequences, which will be clearly understood by those of skill in the art. Such polynucleotide probes preferably comprise oligonucleotides for hybridization analyses; alternatively primer pairs of probes are preferred when polymerase chain reaction detection techniques are to be employed. Those of skill in the art are well aware of how to design appropriate primer pairs for a given target polynucleotide.

The term “polynucleotide” encompasses nucleic acids containing known analogues of natural nucleotides which have similar or improved binding properties, for the purposes desired, as the disclosed polynucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs), methylphosphonate linkages or alternating methylphosphonate and phosphodiester linkages (Strauss-Soukup (1997) Biochemistry 36:8692-8698), and benzylphosphonate linkages, as discussed in U.S. Pat. No. 6,664,057.

The polynucleotide probes according to the different aspects and embodiments of the invention are “isolated”, which means that the polynucleotides are free of sequences which naturally flank the polynucleotide in the genomic DNA of the organism from which the nucleic acid is derived, except as specifically described herein. It is preferred that the isolated polynucleotide probes are substantially free of other cellular material, gel materials, culture medium, and contaminating polypeptides or nucleic acids (such as from nucleic acid libraries or expression products therefrom), except as described herein, when produced by recombinant techniques. The polynucleotides of the invention may be isolated from a variety of sources, such as by PCR amplification from genomic DNA, mRNA, or cDNA libraries derived from mRNA, using standard techniques; or they may be synthesized in vitro, by methods well known to those of skill in the art, as discussed in U.S. Pat. No. 6,664,057 and references disclosed therein. Synthetic polynucleotides can be prepared by a variety of solution or solid phase methods. Detailed descriptions of the procedures for solid phase synthesis of polynucleotide by phosphite-triester, phosphotriester, and H-phosphonate chemistries are widely available. (See, for example, U.S. Pat. No. 6,664,057 and references disclosed therein). Methods to purify polynucleotides include native acrylamide gel electrophoresis, and anion-exchange HPLC, as described in Pearson (1983) J. Chrom. 255:137-149. The sequence of the synthetic polynucleotides can be verified using standard methods.

In another preferred embodiment or each of the aspects and embodiments of the present invention, the plurality of probes comprises polypeptide probes. This embodiment is particularly preferred where the probes are selective for polypeptide expression products of the glomerular markers. In one example, such polypeptides comprise antibodies, such as polyclonal and monoclonal, antibodies. The term antibody as used herein is intended to include antibody fragments thereof which are selectively reactive with the glomerular marker polypeptides, or fragments thereof. Antibodies can be fragmented using conventional techniques, and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. Antibodies can be made by well-known methods, such as described in Harlow and Lane, Antibodies; A Laboratory Manual, Cold Spring Harbor Laboratory, Cold Spring Harbor, N.Y., (1988). For example, preimmune serum is collected prior to the first immunization. A polypeptide of interest, or antigenic fragment thereof, together with an appropriate adjuvant, is injected into an animal in an amount and at intervals sufficient to elicit an immune response. Animals are bled at regular intervals, preferably weekly, to determine antibody titer. The animals may or may not receive booster injections following the initial immunization. At about 7 days after each booster immunization, or about weekly after a single immunization, the animals are bled, the serum collected, and aliquots are stored at about −20° C. Polyclonal antibodies can then be purified directly by standard techniques. Monoclonal antibodies can be produced by obtaining spleen cells from the animal. (See Kohler and Milstein, Nature 256, 495-497 (1975)). In one example, monoclonal antibodies (mAb) of interest are prepared by immunizing inbred mice with a polypeptide of interest, or an antigenic fragment thereof. The mice are immunized by the IP or SC route in an amount and at intervals sufficient to elicit an immune response. The mice receive an initial immunization on day 0 and are rested for about 3 to about 30 weeks. Immunized mice are given one or more booster immunizations of by the intravenous (IV) route. Lymphocytes, from antibody positive mice are obtained by removing spleens from immunized mice by standard procedures known in the art. Hybridoma cells are produced by mixing the splenic lymphocytes with an appropriate fusion partner under conditions which will allow the formation of stable hybridomas. The antibody producing cells and fusion partner cells are fused in polyethylene glycol at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells and are screened for antibody production by an immunoassay such as solid phase immunoradioassay. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973.

In various preferred embodiments of this first aspect of the invention, the composition comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, or 280 probes that selectively bind to between 3 and 281 of the glomerular markers, disclosed herein in Table 6 (mouse), complements thereof, or their expression products.

In various further preferred embodiments, the of this first aspect of the invention, the composition comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, or 264 probes that selectively bind to between 3 and 265 of the glomerular markers disclosed herein in Table 7 (human), complements thereof, or their expression products.

In further preferred embodiments of the first aspect of the invention, the composition comprises probes that selectively bind to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, or 544 of the glomerular markers, disclosed herein in Tables 6 (mouse) and Table 7 (human), complements thereof, or their expression products.

In various further preferred embodiments of this first aspect and of the invention and its other embodiments, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, or 356 of the glomerular markers disclosed herein in Table 9, complements thereof, human homologues thereof, or their expression products. Table 9 lists those glomerular markers shown with more than a two-fold increase in glomerular expression compared to non-glomerular renal tissue (“Category 1 genes”).

In various further preferred embodiments of this first aspect of the invention and its other embodiments, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, or 142 of the glomerular markers disclosed herein in Table 11, complements thereof, human homologues thereof (See Table 11A), or their expression products. Table 11 lists those glomerular markers shown with more than a two-fold increase in expression in glomeruli compared to non-glomerular tissue after removing those showed at least a two-fold increase in expression levels in brain capillary compared to non-podocyte glomerular tissue (“Category 3 genes”).

In various further preferred embodiments of this first aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 of the glomerular markers disclosed herein in Table 3, complements thereof, human homologues thereof, or their expression products. Table 3 lists those Category 3 glomerular markers with more than a two-fold increase in expression in podocytes compared to non-podocyte glomerular tissue (“Category 6 genes”).

In various further preferred embodiments of this first aspect of the invention, the plurality of probes in total selectively binds to at least 2, 3, 4, 5, 6, 7, 8, 9, 10, 11, 12, 13, 14, 15, 16, 17, or 18 of the glomerular markers disclosed herein in Table 4, complements thereof, human homologues thereof, or their expression products. Table 4 lists those Category 3 glomerular markers shown with more than a two-fold increase in expression in glomerular mesangial and endothelial cells compared to podocytes (“Category 7 genes”).

In various further preferred embodiments of this first aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41,42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of the glomerular markers disclosed herein in Table 5, complements thereof, human homologues thereof, or their expression products. Table 5 lists those Category 3 glomerular markers that did not show differential expression between podocytes and non-podocyte glomerular tissue (“Category 8 genes”).

In various further preferred embodiments of this first aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85,.86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 of the glomerular markers disclosed herein in Table 13, complements thereof, human homologues thereof, or their expression products. Table 13 lists those glomerular markers from Table 9 with less than a two-fold increase in expression in glomeruli relative to brain capillary(“Category 5 genes”).

In various further preferred embodiments of this first aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 of the glomerular markers disclosed herein in Table 12, complements thereof, human homologues thereof, or their expression products. Table 12 lists those glomerular markers from Table 9 shown with less than a two-fold increase in expression in glomeruli relative to brain capillary (“Category 4 genes”).

In a second aspect, the present invention provides a composition comprising a plurality of isolated probes that in total selectively bind to at least 51 of the glomerular markers disclosed herein in Table 9 (Category 1 genes), complements thereof, or their expression products, wherein at least 10% of the probes in total are selective for glomerular markers.

In this second aspect at least 10% of the probes of the composition are selective for glomerular markers, such as those disclosed herein in Tables 3, 4, 5, 6, 7, 9, and 11-17, as well as other glomerular probes not disclosed herein.

The compositions of the invention may contain probes that are not glomerular specific (for example, for use as control sequences to verify the glomerular-specific nature of an assay in which the compositions are used), so long as such probes do not make up more than 90% of the probes of the composition. In various preferred embodiments, at least 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for glomerular markers, such as those disclosed herein in Tables 3, 4, 5, 6, 7, 9, and 11-17, as well as other glomerular probes not disclosed herein.

In various preferred embodiments of this second aspect of the invention, the plurality of probes in total selectively bind to at least 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, or 358 of the glomerular markers disclosed herein in Table 9, complements thereof, human homologues thereof, or their expression products. Table 9 lists those Category 1 glomerular markers as discussed above.

In one preferred embodiment of this second aspect of the invention, the plurality of probes in total selectively binds to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, or 544 of the glomerular markers, disclosed herein in Tables 6 (mouse) and Table 7 (human), complements thereof, or their expression products.

In various further preferred embodiments of this second aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, or 142, of the glomerular markers disclosed herein in Table 11, complements thereof, human homologues thereof (see Table 11A), or their expression products.

In various further preferred embodiments of this second aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 of the glomerular markers disclosed herein in Table 3, complements thereof, human homologues thereof, or their expression products.

In various further preferred embodiments of this second aspect of the invention, the plurality of probes in total selectively binds to at least 2, 3, 4, 5, 6, 7, 8, 9, 10, 11, 12, 13, 14, 15, 16, 17, or 18 of the glomerular markers disclosed herein in Table 4, complements thereof, human homologues thereof, or their expression products.

In various further preferred embodiments of this second aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 of the glomerular markers disclosed herein in Table 13, complements thereof, human homologues thereof, or their expression products.

In various further preferred embodiments of this second aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 of the glomerular markers disclosed herein in Table 12, complements thereof, human homologues thereof, or their expression products.

In various further preferred embodiments of this second aspect of the invention, the plurality of probes in total selectively binds to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76 of the glomerular markers disclosed herein in Table 5, complements thereof, human homologues thereof, or their expression products.

In a third aspect, the present invention provides a composition comprising a plurality of isolated probes that in total selectively bind to at least 12 of the podocyte markers disclosed herein in Table 3, complements thereof, human homologues thereof, or their expression products, wherein at least 1.5% of the probes in total are selective for podocyte markers. In various preferred embodiments of this third aspect of the invention, the plurality of probes it total selectively binds to at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 of the glomerular markers disclosed herein in Table 3, complements thereof, human homologues thereof, or their expression products.

As used herein, the term “podocyte marker” means glomerular markers that are up-regulated two-fold or more in glomerular podocytes relative to non-podocyte glomerular tissue. The compositions of this third aspect of the invention are particularly useful for profiling of podocyte-specific gene expression.

In this third aspect, at least 1.5% of the probes of the composition are selective for podocyte markers, such as those disclosed herein in Table 3, as well as other podocyte probes not disclosed herein.

The compositions of the invention may contain probes that are not podocyte-specific (for example, for use as control sequences to verify the podocyte-specific nature of an assay in which the compositions are used), so long as such probes do not make up more than 98.5% of the probes of the composition. In various preferred embodiments, at least 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for podocyte markers, such as those disclosed herein in Table 3, as well as other podocyte probes not disclosed herein.

As will be apparent to those of skill in the art, the compositions of this third aspect of the invention can also comprise fuirther probes as disclosed in the various preferred embodiments of the first and second aspect of the invention described above.

In an especially preferred embodiment of this third aspect, at least one or more of the isolated probes in the composition is a novel glomerular marker selected from those disclosed in Table 6 or Table 7.

In a fourth aspect, the present invention provides a composition comprising a plurality of isolated probes that in total selectively bind to at least 2 of the podocyte markers disclosed herein in both Table 3 and in Table 6, complements thereof, human homologues thereof, or their expression products, wherein at least 1.5% of the probes in total are selective for podocyte markers. Examples of such podocyte markers that are disclosed in both Table 3 and Table 6 include those numbered as follows in Table 3: 5, 7-11, 13, 15-18, 20-21, 23-32, 34-42, and 44-48. These podocyte markers were not known as glomerular markers prior to the present study, and thus were not known as glomerular podocyte markers.

In this fourth aspect, at least 1.5% of the probes of the composition are selective for podocyte markers, such as those disclosed herein in Table 3, as well as other podocyte probes not disclosed herein.

The compositions of the invention may contain probes that are not podocyte-specific (for example, for use as control sequences to verify the podocyte-specific nature of an assay in which the compositions are used), so long as such probes do not make up more than 98.5% of the probes of the composition. In various preferred embodiments, at least 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for podocyte markers, such as those disclosed herein in Table 3, as well as other podocyte probes not disclosed herein.

In various preferred embodiments of this fourth aspect of the invention, the plurality of probes it total selectively binds to at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 of the glomerular markers disclosed herein in both Table 3 and Table 6, complements thereof, human homologues thereof (such as in Table 7), or their expression products.

As will be apparent to those of skill in the art, the compositions of this fourth aspect of the invention can also comprise further probes as disclosed in the various preferred embodiments of the first and second aspect of the invention described above.

In a fifth aspect, the present invention provides a composition comprising a plurality of isolated probes that in total selectively bind to at least 7 of the non-podocyte glomerular markers disclosed herein in Table 4, complements thereof, human homologues thereof, or their expression products, wherein at least 8.5% of the probes in total are selective for non-podocyte glomerular markers. In various preferred embodiments of this third aspect of the invention, the plurality of probes it total selectively binds to at least 8, 9, 10, 11, 12, 13, 14, 15, 16, 17, or 18 of the glomerular markers disclosed herein in Table 4, complements thereof, human homologues thereof, or their expression products.

As used herein, the term “non-podocyte glomerular markers” means glomerular markers that are up-regulated two-fold or more in glomerular mesangial and/or endothelial cells relative to podocytes. The compositions of this third aspect of the invention are particularly useful for profiling of up-regulated glomerular mesangial and/or endothelial cell markers.

In this fifth aspect, at least 8.5% of the probes of the composition are selective for non-podocyte glomerular markers, such as those disclosed herein in Table 4, as well as other non-podocyte glomerular markers not disclosed herein.

The compositions of the invention may contain probes that are not non-podocyte glomerular-specific (for example, for use as control sequences to verify the non-podocyte glomerular-specific nature of an assay in which the compositions are used), so long as such probes do not make up more than 91.5% of the probes of the composition. In various preferred embodiments, at least 9%, 9.5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for non-podocyte glomerular markers, such as those disclosed herein in Table 4, as well as other non-podocyte glomerular probes not disclosed herein.

As will be apparent to those of skill in the art, the compositions of this fifth aspect of the invention can also comprise further probes as disclosed in the various preferred embodiments of the first and second aspect of the invention described above.

In an especially preferred embodiment of this third aspect, at least one or more of the isolated probes in the composition is a novel glomerular marker selected from those disclosed in Table 6 or Table 7.

In a sixth aspect, the present invention provides a composition comprising a plurality of isolated probes that in total selectively bind to at least 2 of the non-podocyte glomerular markers disclosed herein in both Table 4 and Table 6, complements thereof, human homologues thereof, or their expression products, wherein at least 8.5% of the probes in total are selective for non-podocyte glomerular markers. Examples of such non-podocyte glomerular markers that are disclosed in both Table 4 and Table 6 include those numbered as follows in Table 4: 6, 9-10, and 12-16. These non-podocyte glomerular markers were not known as glomerular markers prior to the present study, and thus were not known as non-podocyte glomerular markers. In various preferred embodiments of this sixth aspect of the invention, the plurality of probes in total selectively binds to at least 3, 4, 5, 6, 7, or 8 of the glomerular markers disclosed herein in both Table 4 and Table 6, complements thereof, human homologues thereof (such as in Table 7), or their expression products

In this sixth aspect at least 8.5% of the probes of the composition are selective for non-podocyte glomerular markers, such as those disclosed herein in Table 4, as well as other non-podocyte glomerular markers not disclosed herein.

The compositions of the invention may contain probes that are not non-podocyte glomerular-specific (for example, for use as control sequences to verify the non-podocyte glomerular-specific nature of an assay in which the compositions are used), so long as such probes do not make up more than 91.5% of the probes of the composition. In various preferred embodiments, at least 9%, 9.5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for non-podocyte glomerular markers, such as those disclosed herein in Table 4, as well as other non-podocyte glomerular probes not disclosed herein

As will be apparent to those of skill in the art, the compositions of this sixth aspect of the invention can also comprise further probes as disclosed in the various preferred embodiments of the first and second aspect of the invention described above.

In a seventh aspect, the present invention provides a composition comprising a plurality of isolated probes that in total selectively bind to at least 2 of the glomerular markers disclosed herein in both Table 11 and Table 6, complements thereof, human homologues thereof, or their expression products, wherein at least 5% of the probes in total are selective for the up-regulated glomerular markers. Examples of such up-regulated glomerular markers that are disclosed in both Table 11 and Table 6 include those numbered as follows in Table S4: 6-8, 10, 12-14, 17, 19, 21-22, 24, 27, 29-32, 35, 38-41, 43-62, 64-65, 67-69, 71-74, 76-78, 80-82, 84-87, 89-91, 93-94, 96-100, 102-103, 105-109, 111-112, 114-126, 129-142. These up-regulated glomerular markers were not known as glomerular markers prior to the present study, and thus further not known as up-regulated glomerular markers. In various preferred embodiments of this sixth aspect of the invention, the plurality of probes in total selectively binds to at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, or 107 of the up-regulated glomerular markers disclosed herein in both Table 11 and Table 6, complements thereof, human homologues thereof, or their expression products.

In this seventh aspects at least 5% of the probes of the composition are selective for up-regulated glomerular markers, such as those disclosed herein in both Tables 11 and 6, as well as other non-podocyte glomerular markers not disclosed herein.

The compositions of the invention may contain probes that are not specific for up-regulated glomerular markers (for example, for use as controls), so long as such probes do not make up more than 95% of the probes of the composition. In various preferred embodiments, at least 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for up-regulated glomerular markers, such as those disclosed herein in both Tables 11 and 6, as well as other up-regulated glomerular markers not disclosed herein.

As will be apparent to those of skill in the art, the compositions of this seventh aspect of the invention can also comprise further probes as disclosed in the various preferred embodiments of the first and second aspect of the invention described above.

In a further aspect, the present invention provides a composition comprising a plurality of isolated probes that in total selectively bind to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, or 143 of the glomerular markers disclosed herein in Table 11, complements thereof, human homologues thereof, or their expression products, wherein at least 5% of the probes in total are selective for the up-regulated glomerular markers. These up-regulated glomerular markers are expected to be extremely sensitive to changes in glomerular function caused by disease, therapeutic intervention, or other causes, and thus probes selective for them will be of great value in glomerular profiling.

In this aspect at least 5% of the probes of the composition are selective for up-regulated glomerular markers, such as those disclosed herein in Table 11, as well as other non-podocyte glomerular markers not disclosed herein.

The compositions of the invention may contain probes that are not specific for up-regulated glomerular markers (for example, for use as controls), so long as such probes do not make up more than 95% of the probes of the composition. In various preferred embodiments, at least 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the probes of the composition are selective for up-regulated glomerular markers, such as those disclosed herein in Table 11, as well as other up-regulated glomerular markers not disclosed herein.

As will be apparent to those of skill in the art, the compositions of this aspect of the invention can also comprise further probes as disclosed in the various preferred embodiments of the first and second aspect of the invention described above.

In a further embodiment of each of the above aspects and embodiments of the compositions of the invention, the compositions further comprise isolated probes selective for at least 2 of the glomerular markers listed in Tables 15 or 16. These genes were previously known to be expressed in the glomerulus, and thus their addition to the compositions of the invention provides for additional ability to characterize glomerular expression profiles as described herein. In various further preferred embodiments, the compositions further comprise isolated probes selective for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, or 133 of the glomerular markers listed in Tables 15 or 16.

In a further embodiment of each of the above aspects and embodiments of the compositions of the invention, the compositions further comprise isolated probes selective for at least 10 of the mouse glomerular markers listed in Table 14, the human glomerular markers listed in Table 14, or a combination thereof. In various preferred embodiments, the compositions further comprise probes selective for at least 50, 100, 150, 200, 250, 300, 350, 400, 450, 500, 550, 600, 650, 700, 750, 80, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500 6600, 6700, 6800, 6900, or 7000 of the mouse glomerular markers listed in Table 14, the human glomerular markers listed in Table 14, or a combination thereof.

In further preferred embodiments of each of the above aspects and embodiments of compositions according to the invention, the composition comprises probes that selectively bind to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 502, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 573, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 929, 929, 930, 931, 932, 933, 934, 935, 936, 937, 938, 939, 940, 941, or 942 of the glomerular markers, disclosed herein in Table 17, complements thereof, or their expression products. Table 17 discloses expressed sequence tags (ESTs) that have been identified herein as expressed in the glomerulus; thus, these markers are useful for glomerular profiling according to the methods of the invention.

The compositions of the various aspects and embodiments of the invention can be in lyophilized form, or may comprise a solution containing the probes, including but not limited to buffer solutions, hybridization solutions, and solutions for keeping the compositions in storage. Such a solution can be made as such, or the composition can be prepared at the time of use, by combining the probes. The probes can be labeled with a detectable label. In a preferred embodiment, the detectable labels on probes for different glomerular markers are distinguishable, to facilitate differential detection. Such probe labeling can be carried out using standard methods in the art. Useful detectable labels include but are not limited to radioactive labels such as ³²p, ³H, and ¹⁴C; fluorescent dyes such as fluorescein isothiocyanate (FITC), rhodamine, lanthanide phosphors, Texas red, and ALEXIS™ (Abbott Labs), CY™ dyes (Amersham); electron-dense reagents such as gold; enzymes such as horseradish peroxidase, beta-galactosidase, luciferase, and alkaline phosphatase; calorimetric labels such as colloidal gold; magnetic labels such as those sold under the mark DYNABEADS™; biotin; dioxigenin; or haptens and proteins for which antisera or monoclonal antibodies are available. The label can be directly incorporated into the probe, or it can be attached to a molecule that hybridizes or binds to the probe. The labels may be coupled to the probes by any means known to those of skill in the art. In various embodiments where the probes comprise polynucleotides, the polynucleotides are labeled using nick translation, PCR, or random primer extension (see, e.g., Sambrook et al. supra). Methods for detecting the label include, but are not limited to spectroscopic, photochemical, biochemical, immunochemical, physical or chemical techniques.

Alternatively, the compositions can be placed on a solid support, such as in a microarray, bead, or microplate format. The term “microarray” as used herein refers to a plurality of probe sets immobilized on a solid surface to which sample nucleic acids or proteins are contacted for binding assays (such as glomerular mRNA, derived cDNA, or protein isolated from a patient with a renal disorder).

Thus, in an eighth aspect, the present invention provides an arrayed composition comprising a support structure on which are arrayed compositions of the invention, as disclosed above. In this aspect, the plurality of probes are generally present at defined locations on the support structure. Such arrays can comprise one or more of the compositions of the invention. Such arrays can thus comprise, for example, polynucleotide arrays or polypeptide (such as antibody) arrays. A given support structure can have single or multiple probes for a given glomerular marker, as discussed above, and can also have various control markers, as discussed above.

In this aspect, the probes are immobilized on a microarray solid surface using standard methods in the art and as disclosed below. A wide variety of materials can be used for the solid surface. Examples of such solid surface materials include, but are not limited to, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose and polyacrylamides.

A variety of different materials may be used to prepare the microarray solid surface to obtain various properties. For example, proteins (e.g., bovine serum albumin) or mixtures of macromolecules (e.g., Denhardt's solution) can be used to minimize non-specific binding, simplify covalent conjugation, and/or enhance signal detection, particularly when using polynucleotide arrays. If covalent bonding between a compound and the surface is desired, the surface will usually be functionalized or capable of being functionalized. Functional groups which may be present on the surface and used for linking include, but are not limited to, carboxylic acids, aldehydes, amino groups, cyano groups, ethylenic groups, hydroxyl groups, and mercapto groups. Methods for linking a wide variety of compounds to various support structures are well known to those of skill in the art.

In a preferred embodiment of this eighth aspect, the locations on an array containing probes of the present invention range in size between 1 μm and 1 cm in diameter, more preferably between 1 μm and 5 mm in diameter, and even more preferably between 5 μm and 1 mm in diameter. The probes may be arranged on the support structures at different densities, depending on factors such as the nature of the label, the support structure, and the size of the probe. One of skill will recognize that each location on the microarray may comprise a mixture of probes of different size and sequences for a given glomerular marker. The size and complexity of the probes fixed onto the locations can be adjusted to provide optimum binding and signal production for a given detection procedure, and to provide the required resolution.

The invention also provides methods of making a glomerular array, comprising arraying one or more of the compositions of the present invention on a solid support, as disclosed above.

In a ninth aspect, the present invention provides methods to profile a glomerular expression pattern from a subject, comprising

a) providing one of more compositions of the invention;

b) contacting the one or more compositions with glomerular polynucleotides and/or polypeptides under conditions to promote selective binding of the probes to their glomerular marker target; and

c) detecting presence of the glomerular marker targets by binding of the probes to their glomerular marker target., wherein the glomerular marker targets detected comprise a glomerular expression pattern.

Samples containing glomerular polynucleotides and/or polypeptides (hereinafter “glomerular sample”) are preferably derived from a subject of interest, such as a subject suffering from a renal disease-related indication, including but not limited to nephropathy, proteinuria, nephrotoxicity, end stage renal disease, diabetes, hypertension, infections, nephrotic syndromes, and glomerulosclerosis. Samples containing such glomerular samples can be obtained by means known to those of skill in the art and as described herein, and can be subjected to various steps to make them more suitable for the assays disclosed herein, such as partial of substantial purification of the polynucleotides or polypeptides, using standard methods in the art.

In a preferred embodiment, the methods further comprises removing unbound glomerular polynucleotides and/or polypeptides prior to detection, using standard techniques such as washing with buffer solutions or various chromatographic techniques.

If the methods of the invention are conducted in solution, then either the probes in the compositions or the glomerular sample are preferably labeled to facilitate detection of their glomerular marker target upon binding. In a preferred embodiment, the compositions are present on a support structure, and the glomerular polynucleotides and/or polypeptides are labeled to facilitate detection. Any method for signal detection can be used with the methods of the invention, including but not limited to polymerase chain reaction, spectroscopic, photochemical, biochemical, immunochemical, physical or chemical techniques. In a preferred embodiment, the compositions are arrayed on a solid support and the glomerular polynucleotides or polypeptides are labeled (using labels as described above), so that their binding to the array can be detected using various types of signal detection techniques.

The methods of the invention can be used to profile a glomerular sample of interest to determine expression pattern of glomerular markers of interest. Such “glomerular expression profiling” can be used, for example, to establish expression profiles and specific biomarkers for various patient populations with renal disease-related indications, including but not limited to nephropathy, proteinuria, nephrotoxicity, end stage renal disease, diabetes, hypertension, infections, nephrotic syndromes, and glomerulosclerosis. Such glomerular expression profiles can be used, for example, to establish pathogenic pathways for different renal diseases, which will improve on renal histopathology as a means to measure renal disease conditions. Such methods are also useful, for example, to define glomerular profiles and biomarkers in various types of renal disease patient populations that correlate with a positive response to a particular therapeutic strategy and/or particular drug candidate; such profiles and biomarkers can then be used to screen patients to identify those patients that are suitable candidates for treatment with the drug. The methods of the invention can also be used, for example, to identify profiles and biomarkers associated with renal toxicity, wherein pre-clinical drug candidates can then be screened for such renal toxicity-associated profiles and biomarkers to weed out at an early stage of development those drug candidates that induce renal toxicity.

In a preferred embodiment of this ninth aspect of the invention, the method comprises monitoring up-regulated glomerular genes, wherein the composition is one according to the second, third, fourth, fifth, sixth, or seventh aspect of the invention. These compositions comprise genes known to be up-regulated in the glomerulus relative to elsewhere in the kidney, and thus are expected to be much more sensitive to changes in glomerular function. As a result, such compositions are ideal for use in the methods of the invention described herein.

In further preferred embodiments of this ninth aspect of the invention, the composition(s) is/are selected from the group consisting of:

a) probes selective for between 2 and 359 glomerular specific markers listed in Table 9;

b) probes selective for between 2 and 142 glomerular specific markers listed in Table 11;

c) probes selective for between 2 and 48 podocyte up-regulated markers listed in Table 3;

d) probes selective for between 2 and 18 non-podocyte up-regulated glomerular markers listed in Table 4; and

e) probes selective for between 2 and 78 glomerular up-regulated glomerular markers listed in Table 5;

or combinations thereof. As will be apparent to those of skill in the art, any number of the recited probes or combinations can be used in this embodiment, as disclosed in the various aspects and embodiments above. Probes listed in (a)-(e) comprise genes known to be up-regulated in the glomerulus relative to elsewhere in the kidney, and thus are expected to be much more sensitive to changes in glomerular function. As a result, such probes are ideal for use in the methods of the invention described herein

In a tenth aspect, the present invention also provides an isolated polynucleotide comprising or consisting of a nucleotide sequence according to SEQ ID NO:2043 (also listed as MTG_{—}602467023 in Table 3) expression vectors comprising the polynucleotide, and host cells transfected with the expression vector. This sequence is referred to herein as “GeneX”, and was identified as a glomerular specific marker herein. Thus, probes for Gene X, such as the nucleic acid itself or probes derived therefrom, have utility in assays for glomerular profiling as disclosed herein. The present invention further comprises an isolated polynucleotide comprising or consisting of a nucleotide sequence as disclosed in Table 17 (SEQ ID NOS: 2044-2986), expression vectors comprising the polynucleotide, and host cells transfected with the expression vector. Table 17 discloses expressed sequence tags (ESTs) that have been identified herein as expressed in the glomerulus; thus, these markers are useful for glomerular profiling according to the methods of the invention.

In an eleventh aspect, the present invention further provides novel dendrin nucleic acids and polypeptides comprising or consisting of the nucleic acid sequence of SEQ ID NO:2041 or the amino acid sequence of SEQ ID NO:2042 (also recited herein as MTG 602468169; ENSMUSG0000059213 in Table 3). This sequence differs from the previously reported mouse dendrin sequence (ENSEMBL mouse release 26.33b.1, 2004-09-03). As disclosed herein, probes for dendrin have utility in assays for glomerular profiling. This aspect of the invention further comprises expression vectors comprising the polynucleotide, host cells transfected with the expression vector, and antibodies selective for one or more epitopes within the amino acid sequence according to SEQ ID NO:2042. The making of polynucleotides and antibodies are described above. Polypeptides according to this aspect of the invention can be purified by standard techniques, as described below.

The expression vectors of the tenth and eleventh aspects of the invention comprise the isolated polynucleotide operatively linked to a promoter. A promoter and the isolated polynucleotide are “operatively linked” when the promoter is capable of driving expression of the polynucleotide expression product.

As used herein, the term “vector” refers to a nucleic acid molecule capable of transporting the polypeptide to which it has been linked. One type of vector is a “plasmid”, which refers to a circular double stranded DNA into which additional DNA segments may be cloned. Another type of vector is a viral vector, wherein additional DNA segments may be cloned into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors), are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. The vector may also contain additional sequences, such as a polylinker for subcloning of additional nucleic acid sequences and a polyadenylation signal to effect proper polyadenylation of the transcript. The nature of the polyadenylation signal is not believed to be crucial to the successful practice of the invention, and any such sequence may be employed, including but not limited to the SV40 and bovine growth hormone poly-A sites. Also contemplated as an element of the vector is a termination sequence, which can serve to enhance message levels and minimize read through from the construct into other sequences. Finally, expression vectors typically have selectable markers, often in the form of antibiotic resistance genes that permit selection of cells that carry these vectors.

In a further embodiment of the tenth and eleventh aspects, the present invention provides recombinant host cells in which the expression vectors disclosed herein have been introduced. As used herein, the term “host cell” is intended to refer to a cell into which a nucleic acid of the invention, such as a recombinant expression vector of the invention, has been introduced. Such cells may be prokaryotic, which can be used, for example, to rapidly produce a large amount of the expression vectors of the invention, or may be eukaryotic, for functional studies. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The host cells can be transiently or stably transfected with one or more of the expression vectors of the invention. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. Alternatively, the host cells can be infected with a recombinant viral vector of the invention. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.).

In a twelfth aspect, the present invention provides methods for identifying glomerular marker polynucleotides, comprising

a) perfusing a target kidney in an organism with a solution containing magnetic beads, wherein the magnetic bead diameter is approximately equivalent to the capillary diameter of glomerular capillaries;

b) removing glomerular-containing kidney tissue from the organism;

c) digesting the glomerular-containing kidney tissue to separate glomeruli from associated kidney tissue;

d) magnetically isolating glomeruli from the digested glomerular-containing kidney tissue;

e) isolating mRNA from the isolated glomeruli

f) normalizing the mRNA to at least partially suppress high copy number mRNA transcripts;

g) identifying mRNA that are expressed in the glomerulus, wherein such mRNA are glomerular marker polynucleotides.

Methods for isolation of target cells using magnetic beads have been previously described (WO 03/093458, incorporated by reference herein in its entirety). Examples of magnetic beads that can be used according to this ninth aspect of the invention include, but are not limited to, spherical DYNABEADS™ (Dynal). Such beads are made of materials (such as iron) providing magnetic properties when placed within a magnetic field. The diameter of bead chosen necessarily varies depending on the application. The diameter chosen corresponds to the diameter of the glomerular capillary that will be selectively embolized with magnetic beads, facilitating isolation with a magnet. 4.5 μm diameter beads are the appropriate size to specifically embolize murine glomerular capillaries and to minimize cell damage.

Digesting the glomerular-containing kidney tissue can be carried out using standard methods in the art. For example, the digesting can be performed using collagenase. The method can further comprise filtering the digested selected tissue or region prior to the magnetic isolation step. mRNA isolation can be accomplished by standard techniques in the art, including but not limited to the methods described below.

Normalization of high copy number mRNA transcripts is utilized to provide a better representation of the different glomerular-specific polynucleotides, and can be carried out using methods known in the art, including but not limited to the method disclosed in Diatchenko et al., Proc. Natl. Acad. Sci. USA 93:6025-6030 (1996).

Identifying mRNA that are expressed in the glomerulus can be accomplished by any means known in the art, including but not limited to in situ hybridization, immunohistochemistry (for protein expression products) or the methods disclosed below.

In one preferred embodiment, the methods of this twelfth aspect of the invention further comprise identifying podocyte-specific glomerular polynucleotides, wherein such identifying comprises identifying those glomerular marker polynucleotides that are expressed in glomerular podocytes. Any method for detecting expression of the glomerular marker polynucleotides in podocytes can be used, including in situ hybridization, immunohistochemistry, or the methods disclosed below.

In a further preferred embodiment, the methods of this twelfth aspect of the invention further comprise identifying non-podocyte-specific glomerular polynucleotides, wherein such identifying comprises identifying those glomerular marker polynucleotides that are expressed in glomerular endothelial and/or mesangial cells. Any method for detecting expression of the glomerular marker polynucleotides in glomerular endothelial and/or mesangial cells can be used, including in situ hybridization, immunohistochemistry, or the methods disclosed below.

In a thirteenth aspect, the present invention provides glomerular specific nucleic acid libraries, comprising predominately glomerular-specific genes as disclosed herein. Embodiments of this aspect of the invention include, but are not limited to, glomerular-specific nucleic acid libraries comprising the glomerular specific genes of one or more of: Tables 3, 4, 5, 6, 7, 9, 10, 11, 12, 13, and 14. Methods for making nucleic acid libraries, including expression libraries, is standard in the art; exemplary methods for making and using such libraries are as described below.

### EXAMPLES

Glomerular disease is a major health care problem, but knowledge about the developmental and molecular biology of the renal filtration unit and its diseases is still limited, although new insight into disease mechanisms has emerged from studies on rare hereditary disorders. In the present study, we report on the assembly and use of a transcription-profiling platform dedicated to the study of mouse renal glomeruli. By using a novel method for glomerulus isolation (45), we constructed a series of high complexity EST libraries from glomeruli at different stages of development. From these libraries, a total of 15,627 EST clones were sequenced, and by annotation against ENSEMBL found to map to 6,053 different genes, estimated to cover 85% of the glomerular transcriptome. Microarray analysis of isolated glomeruli, non-glomerular kidney tissue, isolated extra-renal microvessel fragments, and FACS-sorted podocytes identified most known glomerular and podocyte-specific transcripts. To identify novel podocyte-specific transcripts, the EST clones were arrayed and hybridized against labeled targets from isolated glomeruli, non-glomerular kidney tissue, FACS-sorted podocytes and brain capillary fragments. This revealed the existence of over 300 novel glomerular cell-enriched transcripts, the expression of many of which was further localized to podocytes, mesangial cells, and juxtaglomerular cells by in situ hybridization. For one of the podocyte-restricted transcripts, dendrin, previously regarded to be brain-specific, we expressed the protein, generated antibodies, and used them to localize dendrin to the podocyte foot processes. Our results provide quantitative expression data for known podocyte genes, some of which are mutated in hereditary nephrotic syndromes, and they also identify novel transcripts and proteins specific to podocytes and mesangial cells, thereby pinpointing candidate genes and proteins involved in the pathogenesis or susceptibility to glomerular diseases.

Materials and Methods

Mice

RNA for cDNA library construction and microarray hybridization was isolated from C57BL/6 and 129/sv strains of mice or hybrids between the two strains. Podocyte isolation experiments were done using podocin-Cre, Z/EG double transgenic mice, which also contained ICR background. Genotyping of littermates was done as described (82). Mice were housed at the Department of Experimental Biomedicine at Göteborg University and the animal facility of the Department of Medical Biochemistry and Biophysics at Karolinska Institutet in accordance with Swedish animal research regulations. Animal experiments were approved by a local committee for ethics in animal research.

RNA Preparation and cDNA Library Construction.

Glomeruli were isolated from newborn and adult mice using Dynabead perfusion (45). Using RNeasy mini kits (Qiagen Inc., Valencia, Calif.), 400 μg of glomerular total RNA was isolated from about two million glomeruli obtained from 100 “adult” mice of ages ranging between 3 weeks and 6 months. An additional 350 μg of glomerular total RNA was isolated from approximately 200,000 glomeruli obtained from 400 newborn mice of ages 1 to 5 days. The RNA was used to produce standard oligo dT-primed cDNA libraries (custom synthesis by Incyte Inc., Palo Alto, Calif.) (83) one each from adult and newborn glomerular RNAs, respectively. In addition, two normalized libraries were generated from the adult standard library, using Incyte proprietary technology, in which high abundance transcripts were suppressed to different degrees.

Sequencing and Annotation of cDNA Clones.

10,944 cDNA clones picked at random from the three adult glomerulus libraries and 5,000 clones from the newborn glomerulus library were sequenced from the 5′ ends in single reads of 500-800 bp length (custom sequencing by Incyte and Genome Vision (Genome Vision Inc., Waltham, Mass.). After vector clipping, sequences shorter than 100 nucleotides were discarded. Remaining sequences were annotated by blast searches against the ENSEMBL mouse gene predictions (http://www.ensembl.org/). Hits with E-values <1e-30 and alignment identity >85% were considered significant and the annotations linked to the best hit were assigned to the clones. Blast searches were also performed against NCBI EST databases and the mouse Unigene cluster database (http://www.ncbi.nlm.nih.gov/UniGene/).

Construction of a Mouse Glomerulus cDNA Chip, GlomChip.

Amplification of the clones was done by PCR using the primers: M13F 5′ TGC AAG GCG ATT AAG TTG 3′ and M13R 5′ AAT TTC ACA CAG GAA ACA GC 3′. The reactions were set up in 384 well PCR-plates (Cycleplate-384 DW, Robbins Scientific, West Midlands, UK) using a Hamilton Microlab 4200 robot (Robbins Scientific). All amplifications were performed in GeneAmp PCR system 9700 (Applied Biosystem, Foster City, Calif.) using the following PCR conditions: 95° C. for 15 min, followed by 40 cycles of 94° C. for 30 s, 52.5° C. for 45 s and 72° C. for 3 min with a final holding step at 72° C. for 7 min. The PCR reactions (20 μl) contained 1× Hot Star PCR Buffer (Qiagen), 0.5 mM MgCl₂, 0.25 mM dNTPs (Invitrogen, San Diego, Calif.), 0.9 μM of each primer, 1 unit Hot Star Taq polymerase (Qiagen), 1× Master Amp Betaine Enhancer (Eppicentre, Madison, Wis.) and 1 μl of DNA template. PCR products were purified using Multiscreen 384-well filter plates (Millipore, Billerica, Mass.), transferred to polystyrene low-profile conical bottom GENETIX plates (Genetix Limited, Hampshire, UK), vacuum-dried, resuspended in 50% DMSO (Sigma-Aldrich, St. Louis, Mo.) and printed using a Microgrid II robot (Genomic Solution Ltd., Cambridgeshire, UK) on gamma-amino-propyl-silane-coated UltraGAPS slides (Corning Inc., London, UK). The slides were printed with an array of 16,704 mouse glomerulus cDNAs, including the 15,944 sequenced clones and 760 clones for which the sequencing reaction had failed, 1344 randomly selected sequence-verified mouse EST clones (obtained from Invitrogen, San Diego, Calif.) and control DNAs including 10 different Arabidopsis Thaliana PCR-products (Stratagene, Amsterdam, Netherlands). The printing was done with a pitch of 0.130 mm between the spots and the whole array was printed in triplicates on the slides.

Tissue and Podocyte Isolation

Mouse glomeruli and brain capillary fragments were prepared as described (45, 54, 84). Podocytes were separated from isolated glomeruli from 8-day-old Podocin-Cre, Z/EG double transgenic mice as follows: Isolated glomeruli were incubated with trypsin solution containing 0.2% trypsin-EDTA (Sigma-Aldrich), 100 ug/ml Heparin and 100 U/ml DNase I in PBS for 25 min at 37° C., with mixing by pipetting every 5 min. The trypsin was inactivated with soybeans trypsin inhibitor (Sigma-Aldrich) and the cell suspension sieved through a 30 um pore size filter (BD bioscience, Franklin Lakes, N.J.). Cells were collected by centrifugation at 200×g for 5 min at 4° C. and resuspended in 1 ml PBS supplemented with 0.1% BSA. To separate GFP-expressing (GFP+) and GFP-negative (GFP−) cells, glomerular cell were sorted using a FACSVantage SE (BD, San Jose, Calif., USA) operating at a sheath pressure of 22 psi. Autofluorescent cells were excluded by analyzing the emission of orange light (585 nm).

Target Preparation and GlomChip Hybridization.

mRNA was amplified using T7 RNA amplification as described (85). Five μg of amplified RNA was primed with 5 μg of random hexamers (Promega UK Ltd., Southampton, UK) and labeled in a reverse transcription reaction with Cy3-dUTP (Amersham Pharmacia Biotech AB, Uppsala, Sweden). To allow for standardization of results, all hybridizations were done in competition with Cy5-dUTP labeled common reference samples. The common reference was made as a mixture of amplified RNA from 13 different sources including mouse brain, heart, thymus, lung, liver, spleen, aorta, kidney, skeletal muscle, testis, adult mouse glomeruli, post-natal-day 5 glomeruli and streptozotocin-induced diabetic mouse glomeruli. RNA samples were amplified separately, pooled and aliquoted in small tubes and kept at −80° C. until use. The differently labeled targets were combined, mixed with 10 μg of yeast tRNA and 10 μg of poly A+ RNA, vacuum-dried and resuspended in 128 μl of DIGeasy hybridization buffer (Roche Diagnostics GmbH, Mannheim, Germany) containing 1% BSA. The hybridization mix was incubated at 100° C. for 2 min followed by 37° C. for 30 min and then added to the chip. Before hybridization, the glasses were rehydrated over a bath of hot double-distilled water and baked at 80° C. for 4 hours followed by prehybridization with DIGeasy hybridization buffer containing 1% BSA for 1 hour at 42° C. The slides were then inserted into a GeneTAC Hybridization Station (Genomic Solution) and hybridized according to the following protocol: Adding the hybridization mix at 50° C., followed by hybridization with labeled target at 44° C. for 3 h, 42° C. for 3 h and 40° C. for 12 h with agitation. After the hybridization, all washing steps were performed at 24° C. in the same robot in the following order: 2×SSC, 0.1% SDS for 5 times, 1×SSC for 5 times and finally held in 0.1×SSC. The slides were air-dried and scanned using a GenePix 4000B scanner (Axon instruments Inc., Union City, Calif.). Image segmentation and spot quantification was performed with ImaGene software (Biodiscovery, Marina Del Rey, Calif.).

Microarray Data Processing.

Local background median was subtracted from each spot. The log2-transformed ratios (Cy3 intensity/Cy5 intensity) were plotted versus the mean of the log2 intensities. The ratios were normalized using the limma package (86). For comparison between two samples, t-test was used to exam the differential expression at the 5% individual significance level. Multiple test correction was done using the false discovery rate method (87).

In situ Hybridization,

Non-radioactive and radioactive in situ hybridization were done as previously described (26, 88).

Production of Antibodies and Western Blotting

The two GlomBase dendrin clones were both predicted in the 3′ UTR region. We followed the prediction of the rat dendrin cDNA sequence (89) to generate a pair of PCR primers (5′-TCC AAG CTG TTG GTG ATT GA-3′ and 5′-CAG TGG CAG AGT TGG AAT TG-3′) that were used to amplify a full length mouse dendrin cDNA sequence from a kidney cDNA library template (Clonetech). The amplified fragment was cloned into a TOPO TA cloning vector (Invitrogen) and sequence verified in multiple clones.

For dendrin antigen and antibody production, we chose to express amino acid residues 55-384 from pET-28a(+) expression vector (Novagen) transfected into BL21 (DE3) cells. Production of a 6 His-tag fusion protein was induced by IPTG. Protein purification involved the following steps: 1) cell lysis with lysozyme and sonication, 2) pelleting of inclusion bodies (10,000×g for 30 min), 3) solubilization in 8 M urea, and 4) purification on sequential S-sepharose ion exchange and sephadex S-200 gel filtration columns. The purified recombinant protein was used to raise polyclonal antibodies in two NZW rabbits using standard protocols (SVA, Uppsala, Sweden).

For western blotting, tissue samples (kidney, brain, liver, heart, spleen, lung, and skeletal muscle) were collected from adult mice. From kidney, glomeruli were isolated using Dynabeads (45). Tissue samples were homogenized on ice with a manual grinder in homogenization buffer (100 mM NaCl, 10 mM Tris, ph 7.5, 1 mM EDTA, 1 mM PMSF with proteinase inhibitors), and solubilized in RIPA buffer (1% Triton X-100, 1% sodium deoxycholate, 0.1% SDS, 158 mM NaCl, 10 mM Tris, ph 7.5, 1 mM EGTA, 1 mM PMSF with proteinase inhibitors). Also, COS-7-cells transiently expressing full-length dendrin or intracellular part of nephrin (used as a control) were prepared for Western analysis. Ten micrograms of total protein were separated on 10% polyacrylamide gel, and transferred into polyvinyl difluoride membrane. After 1 h incubation at room temperature (RT) in blocking solution (5% dry milk powder, 3% casein enzymatic hydrolysate, 1% BSA, 0.1% tween-PBS), the membrane was blotted either with the anti-dendrin antiserum (diluted 1:2000 in blocking solution) or pre-immune serum for overnight (+4° C.). Then, the membrane was washed in 0.1% tween-PBS for 30min (RT) followed by incubation in HRP-conjugated goat anti-rabbit-IgG (Dako). Peroxidase activity was detected with Western Chemiluminence reagent (PerkinElmer Life Sciences).

Immunohistochemistry and Immunoelectron Microscopy Analyses

For immunohistochemical analysis, kidney tissue samples from 3-week old mice were placed in cryoprotectant medium (Tissue Tek, Sakura), and frozen in liquid nitrogen. Cryosections (10 μm) were fixed in acetone for 20 min (−20° C.), and blocked in 5% normal goat serum for 30 min (RT). Then, the sections were incubated either in anti-dendrin antiserum (1:1000 in 0.5% normal goat serum) or preimmune serum for 1 h (RT) followed by 30 min incubation (RT) in FITC-labeled anti-rabbit IgG (Jackson ImmunoResearch Laboratories). Each incubation was followed by three 5-minute washes in PBS. In addition, COS-7 cells transfected transiently with full-length dendrin were prepared for immunofluorescence studies. Microscopic observations were performed with a standard Leica DM RX light microscope.

Immuno-electron microscopy using rabbit polyclonal antiserum against dendrin antiserum was done carried out essentially as described (90).

Construction and Large-Scale Sequencing of High-Complexity Mouse Glomerulus cDNA Libraries.

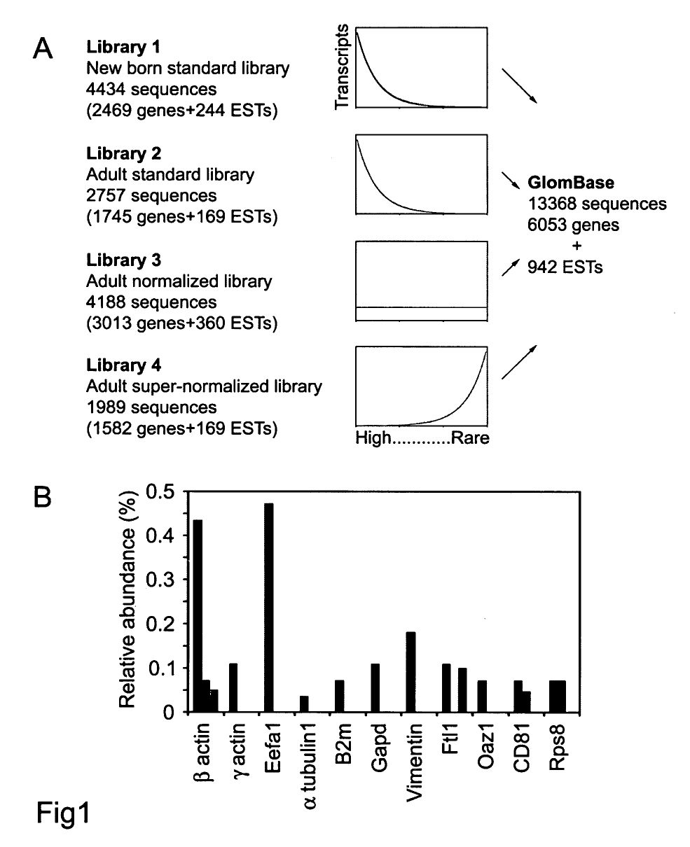

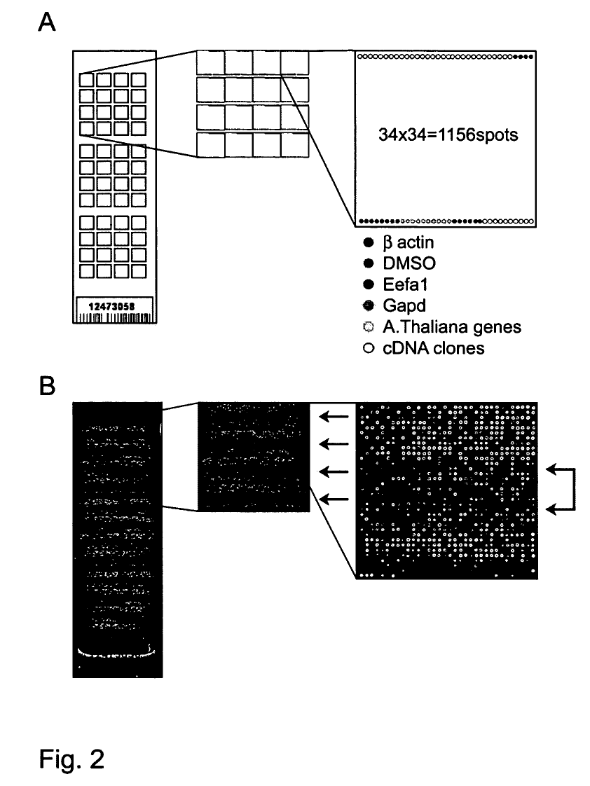

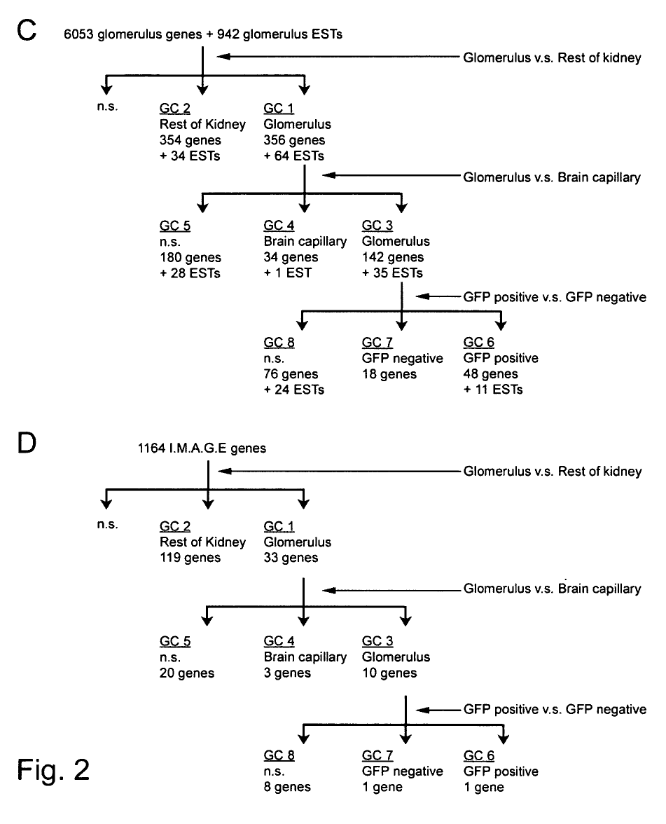

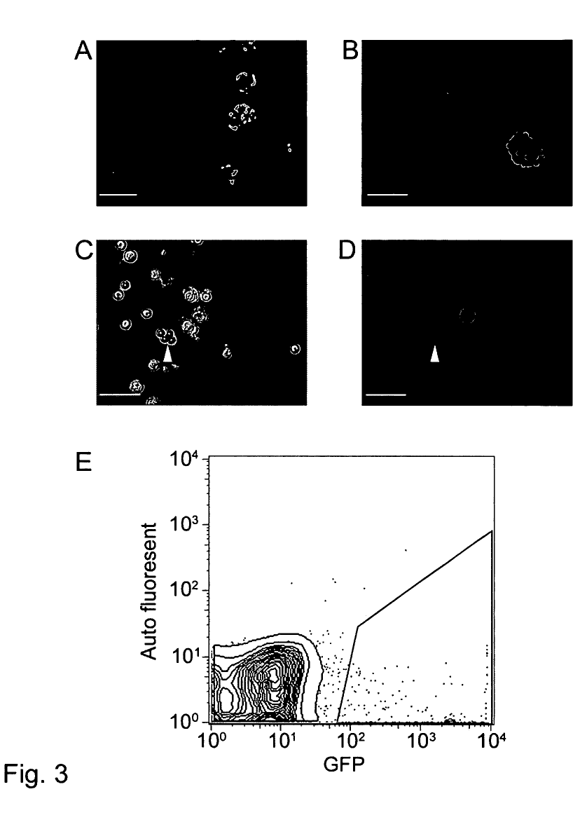

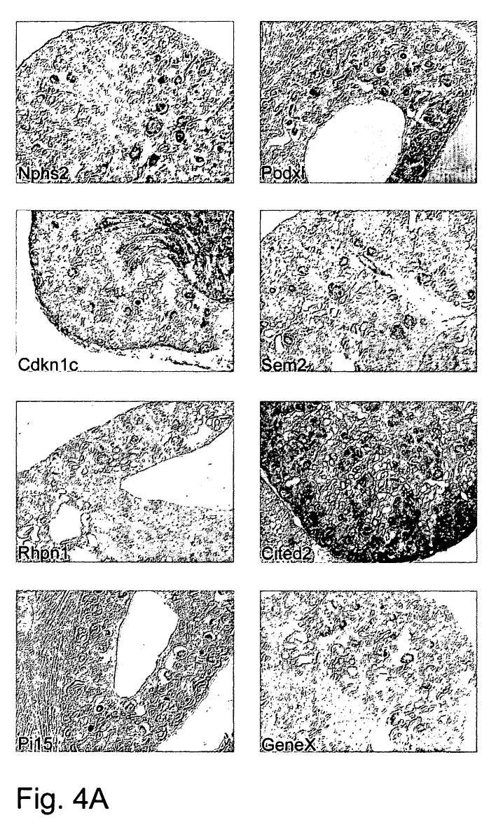

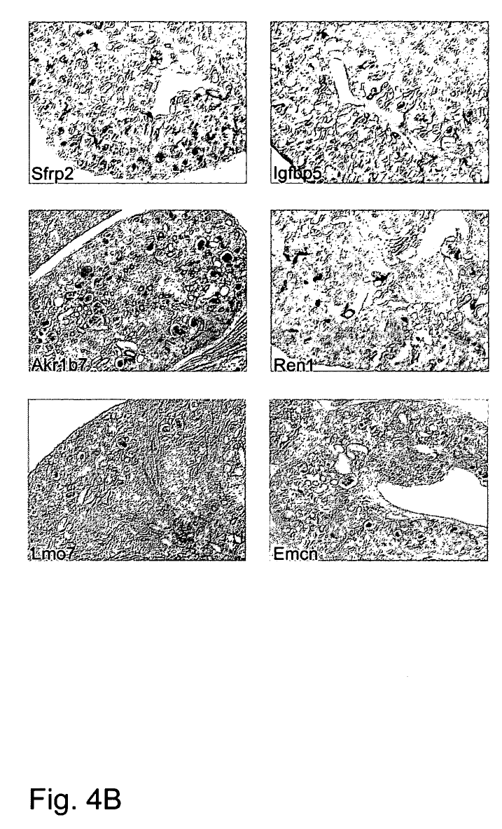

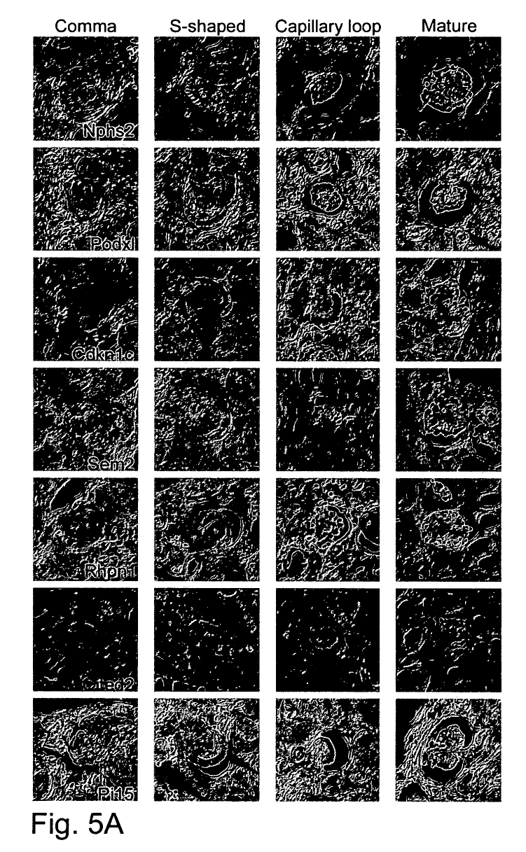

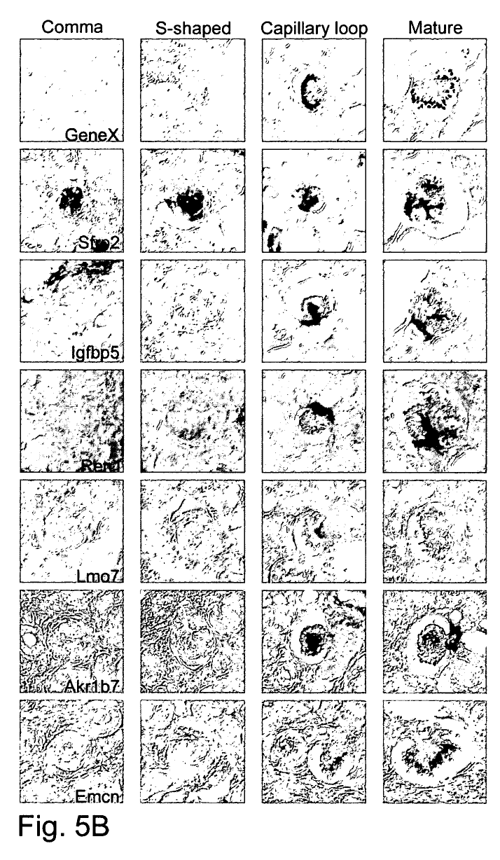

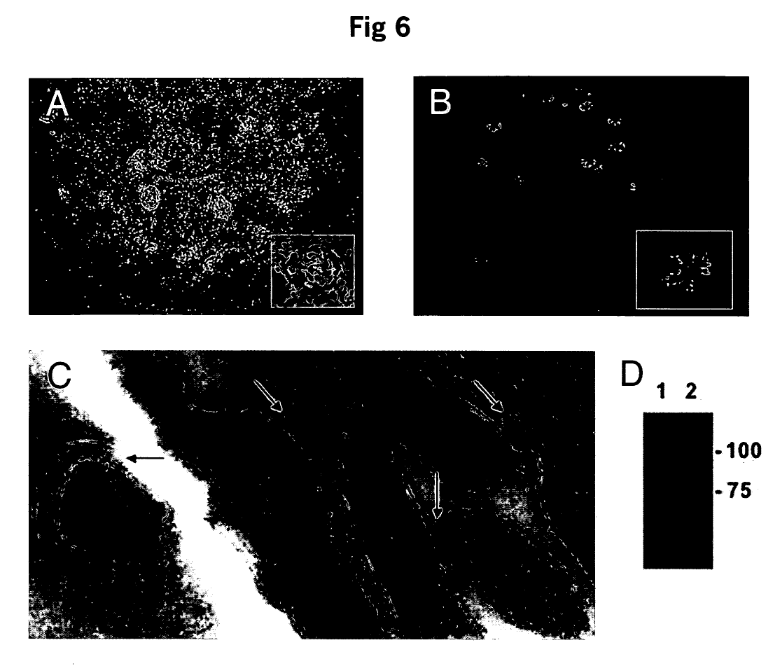

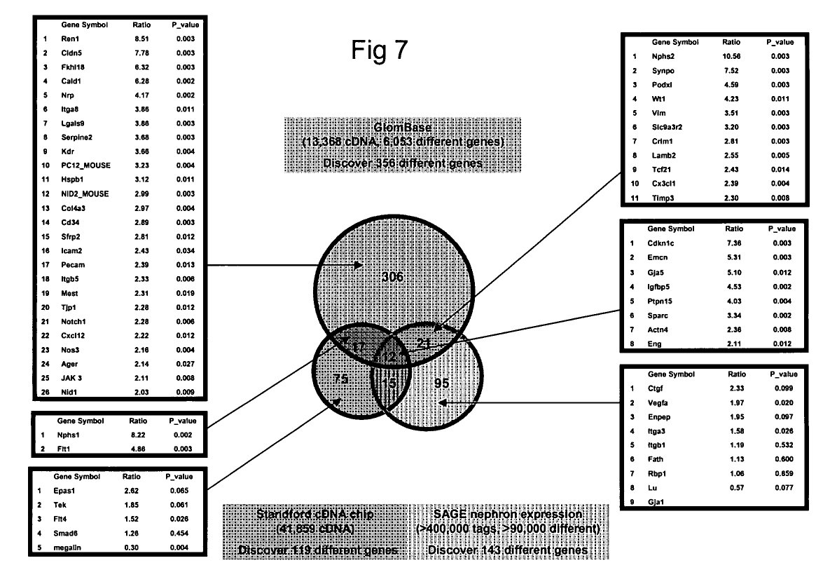

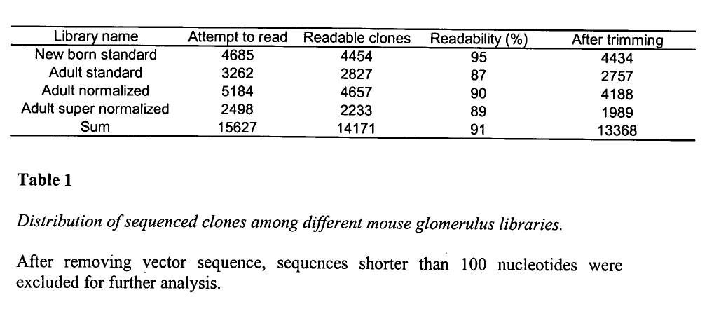

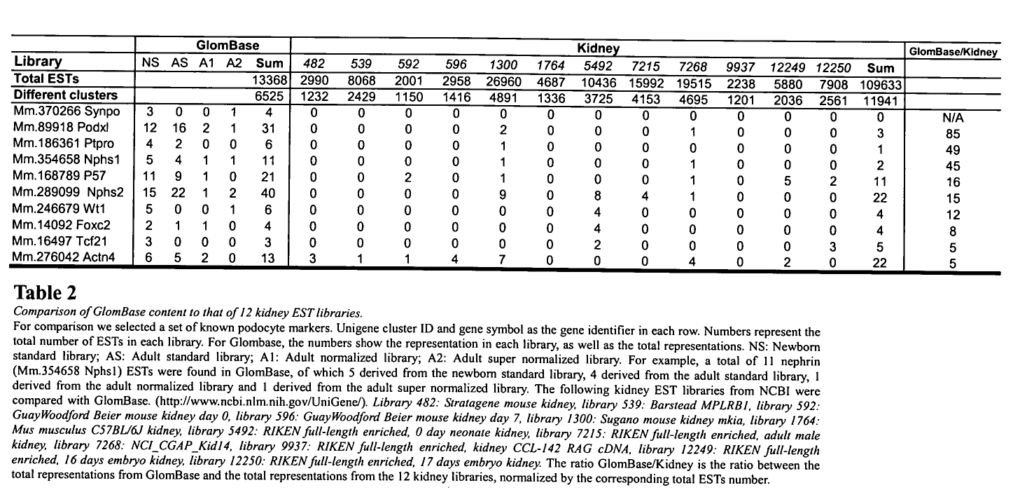

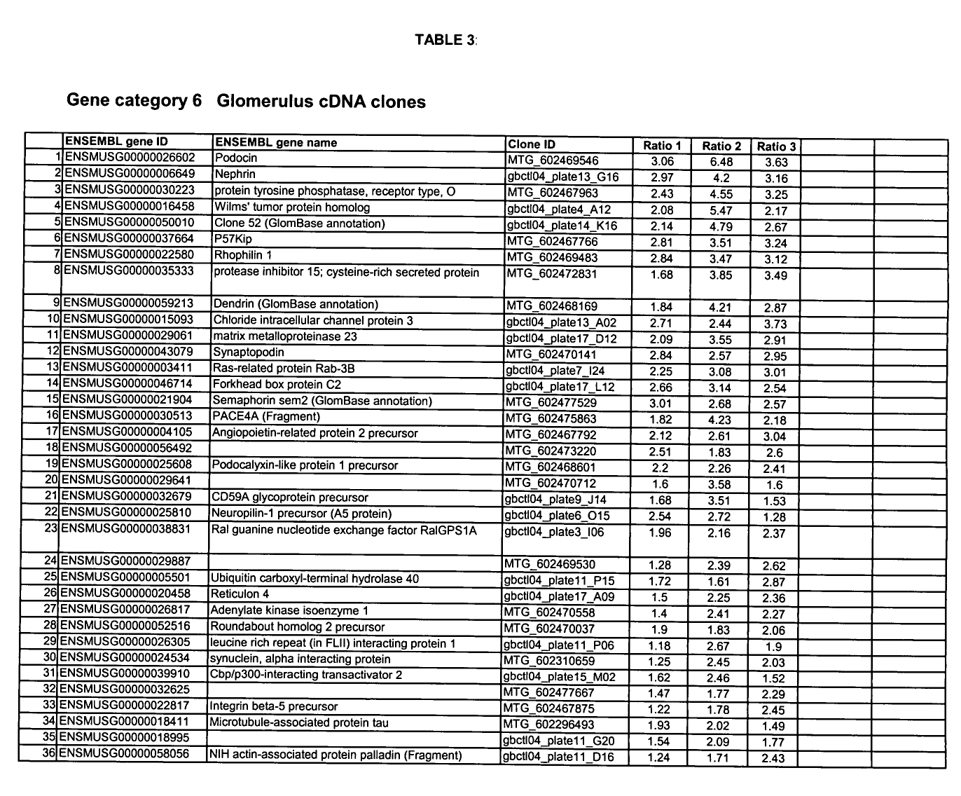

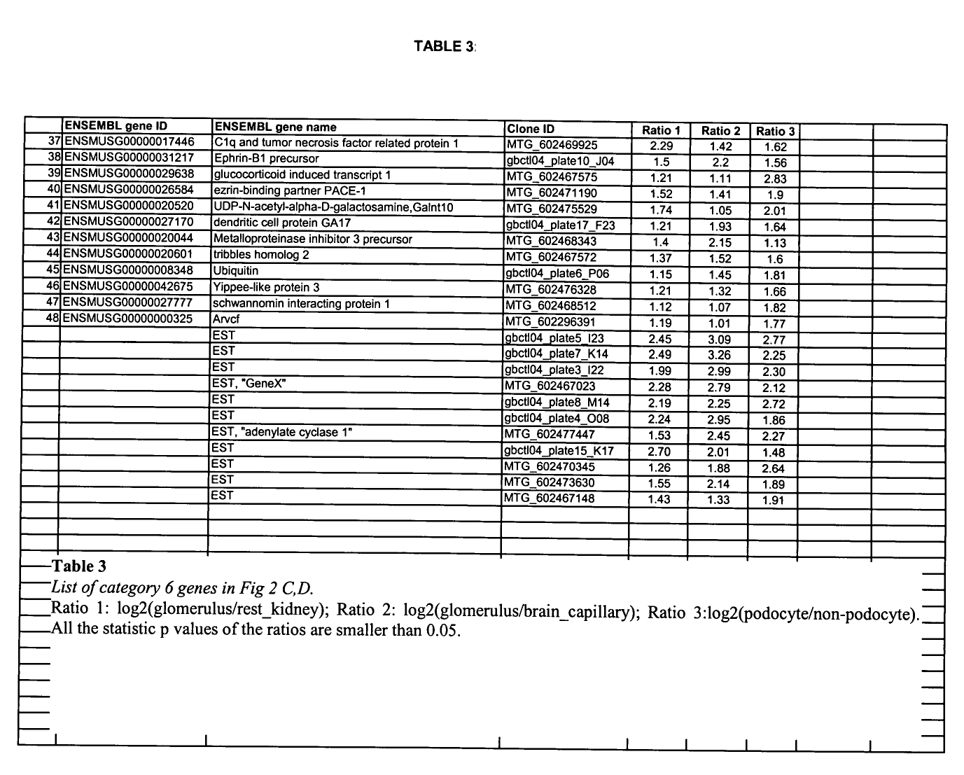

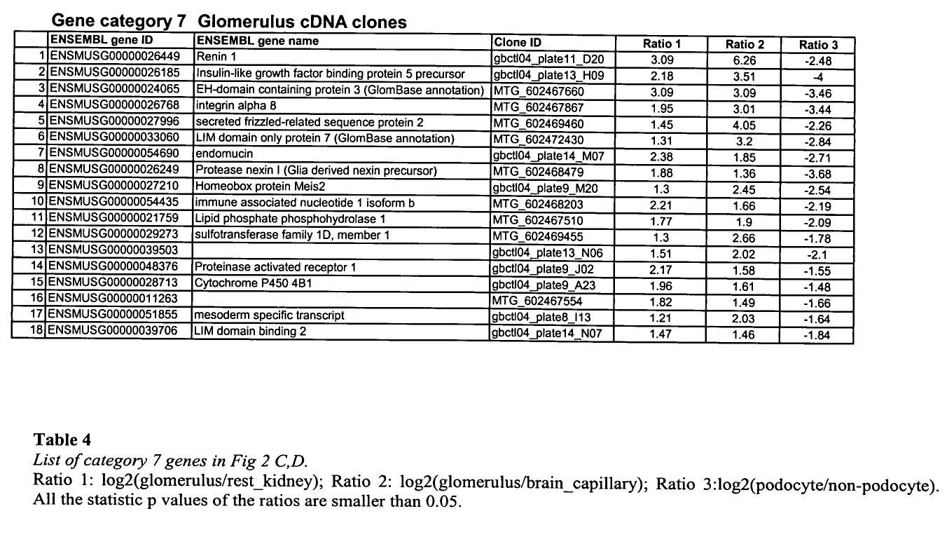

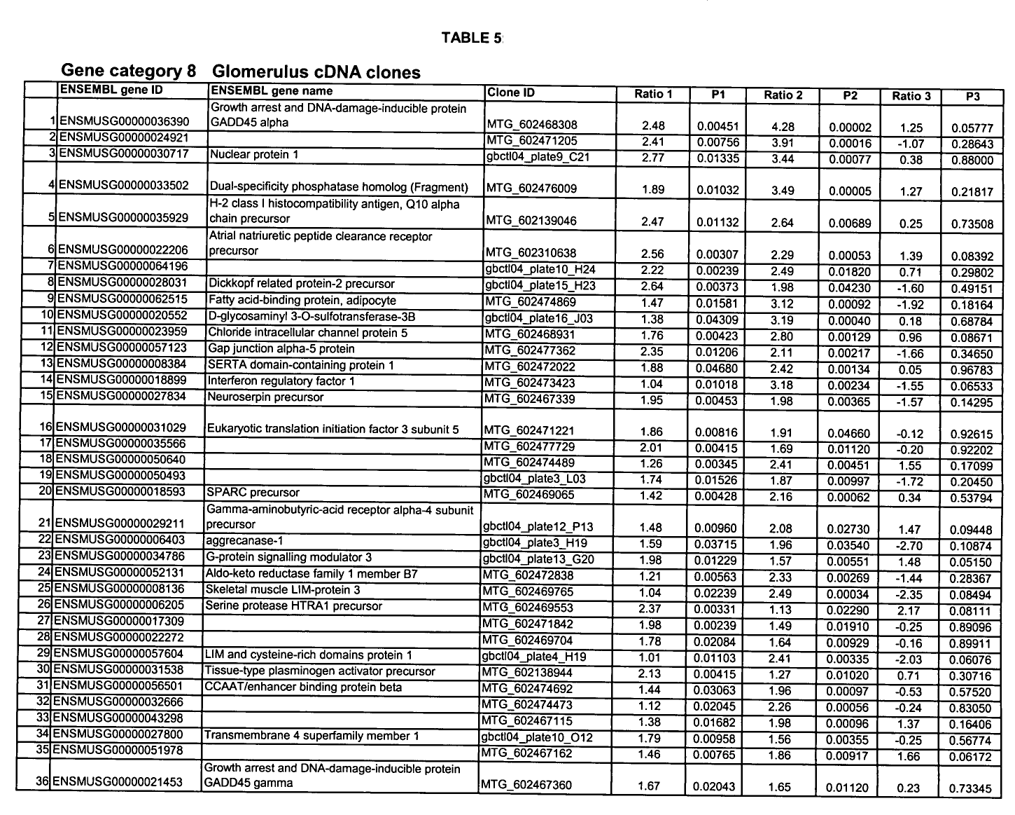

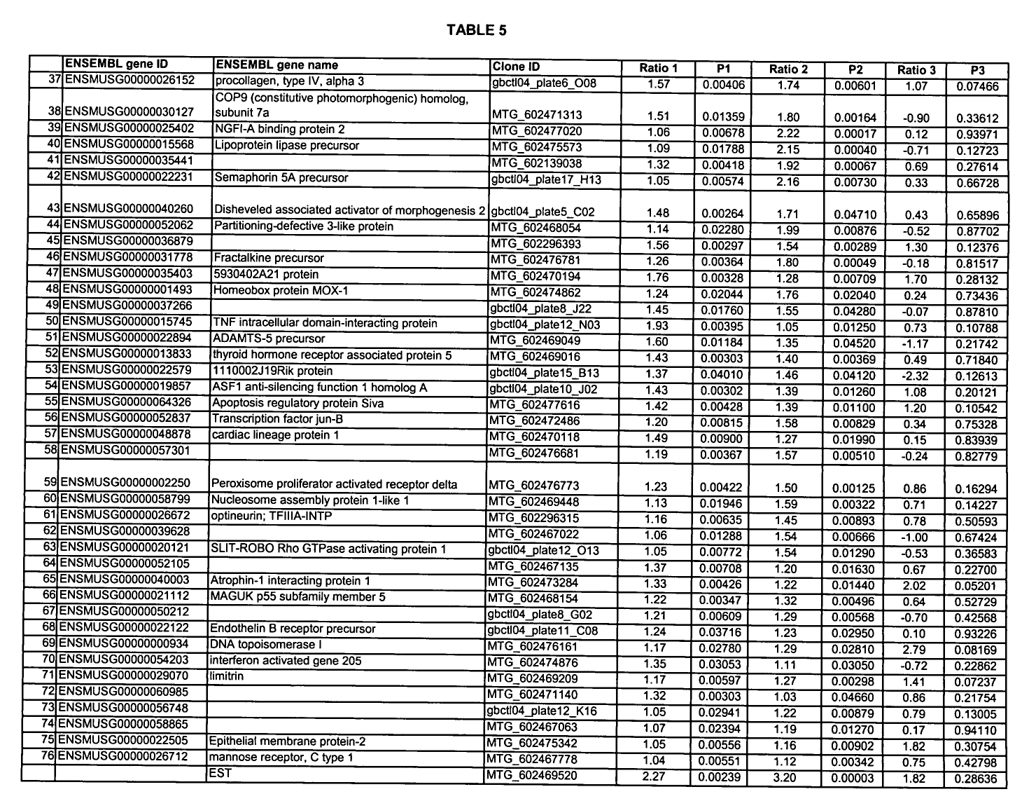

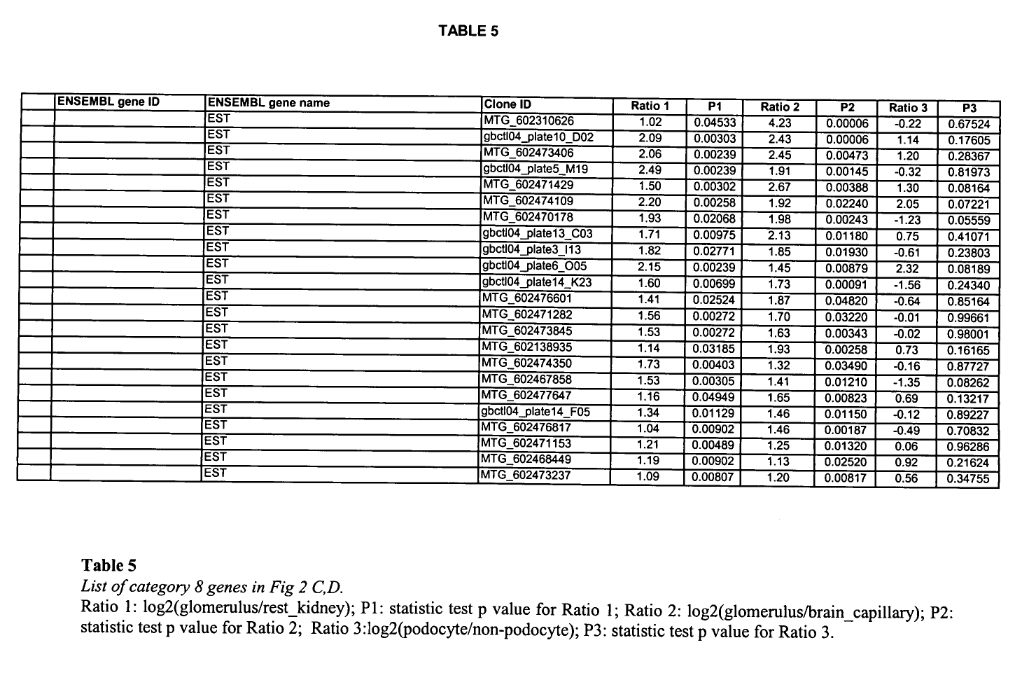

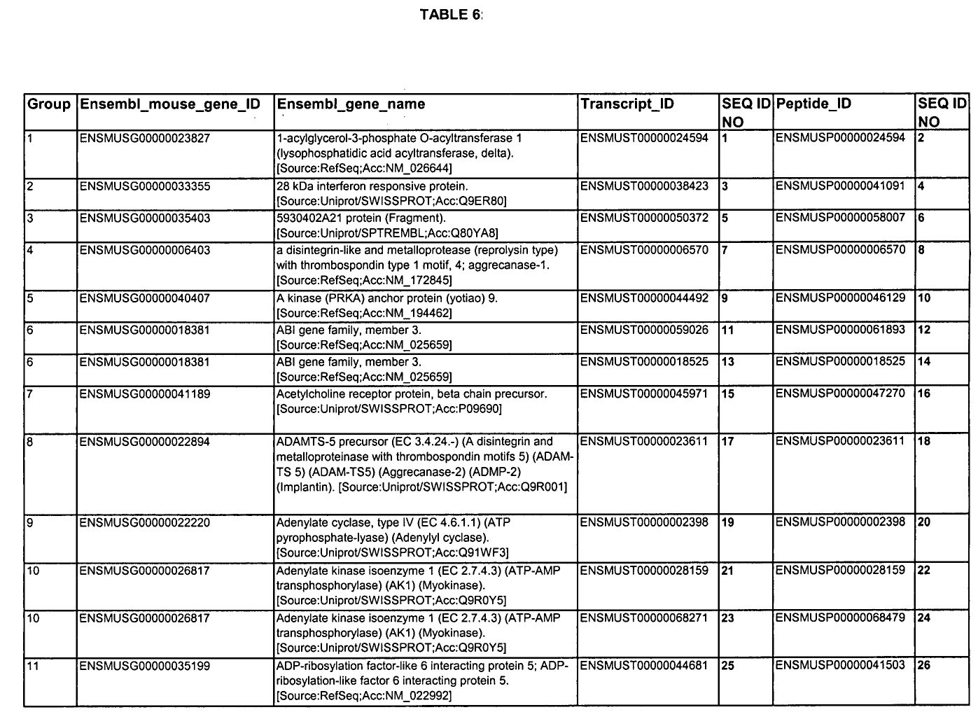

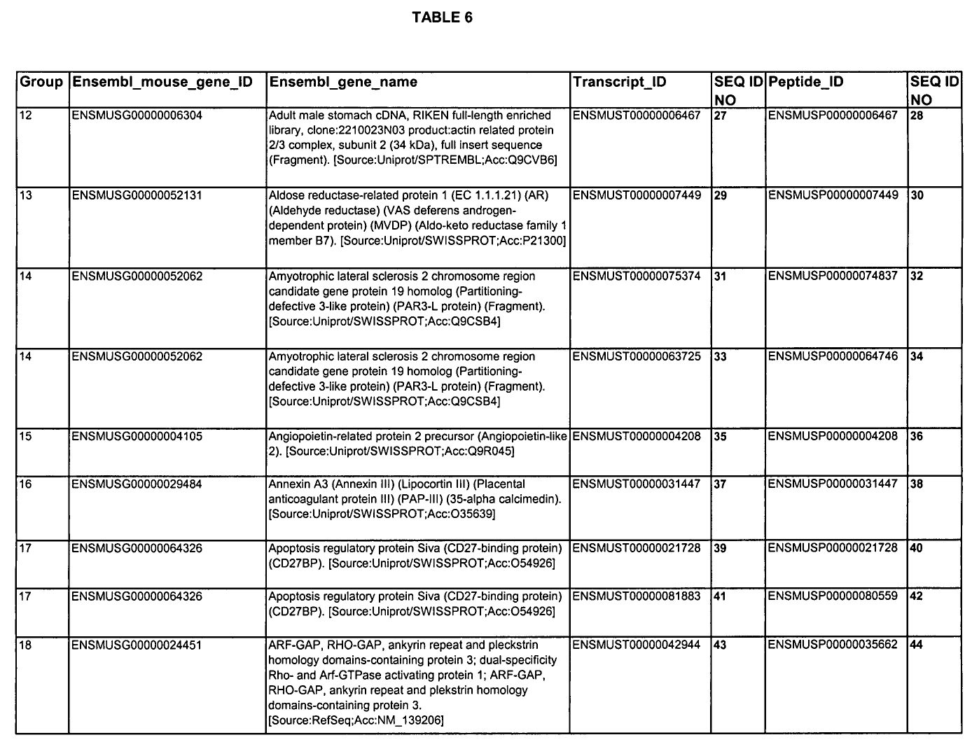

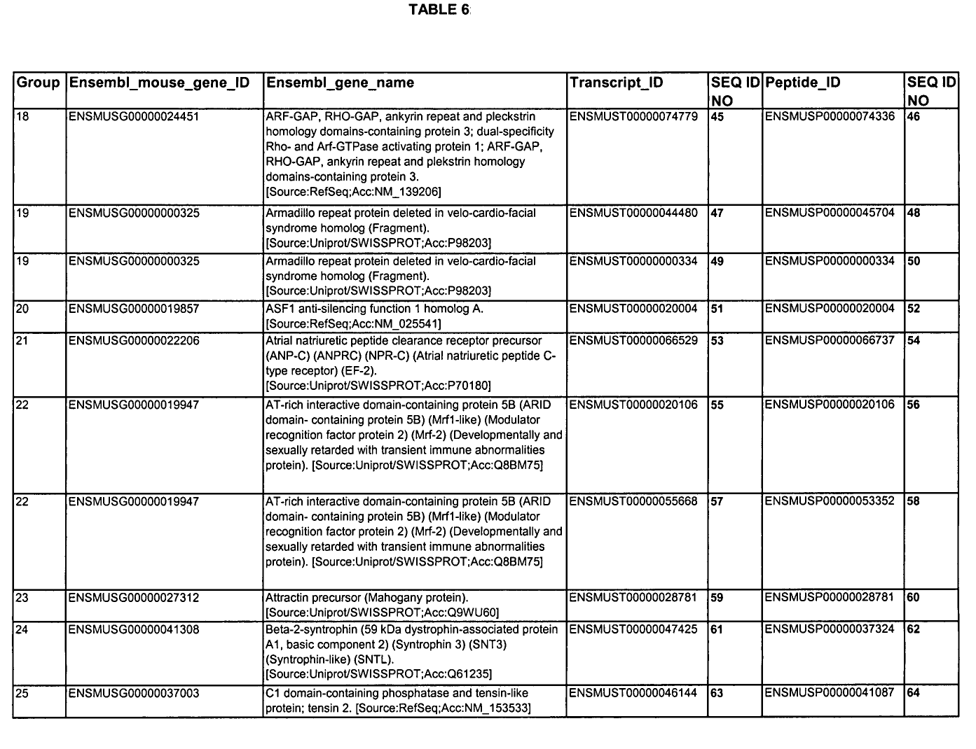

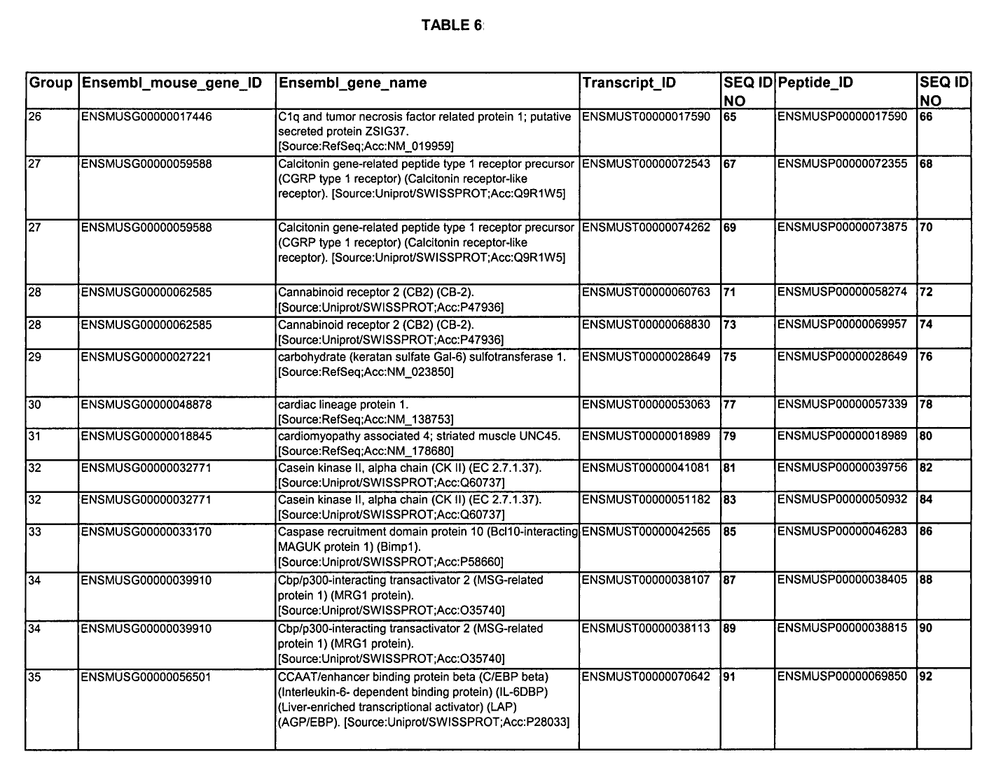

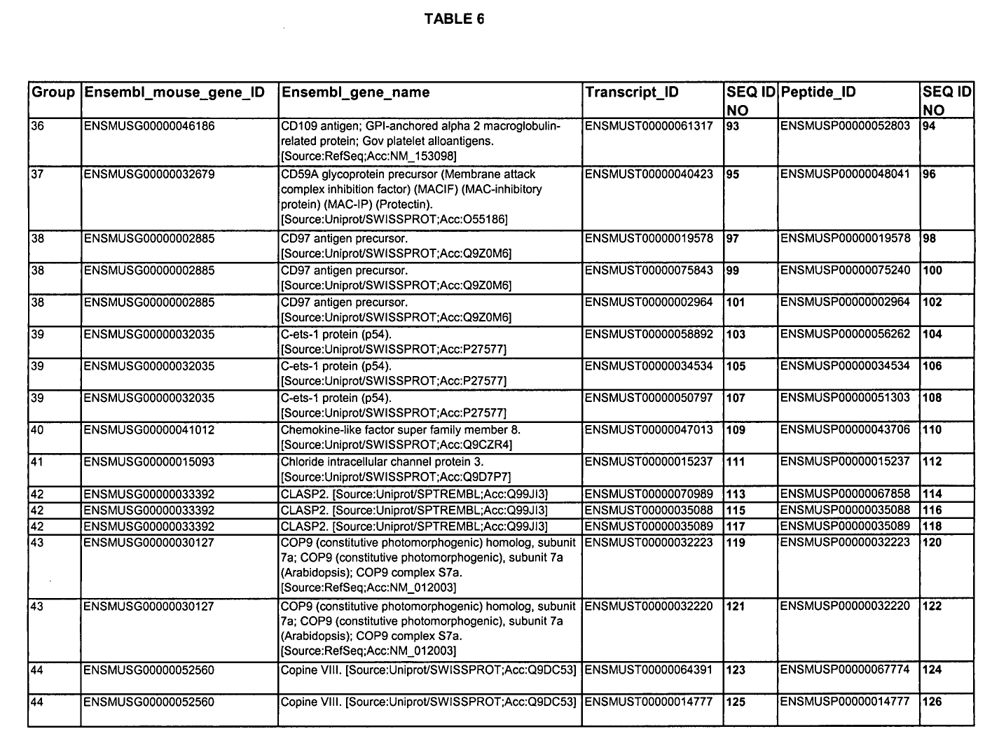

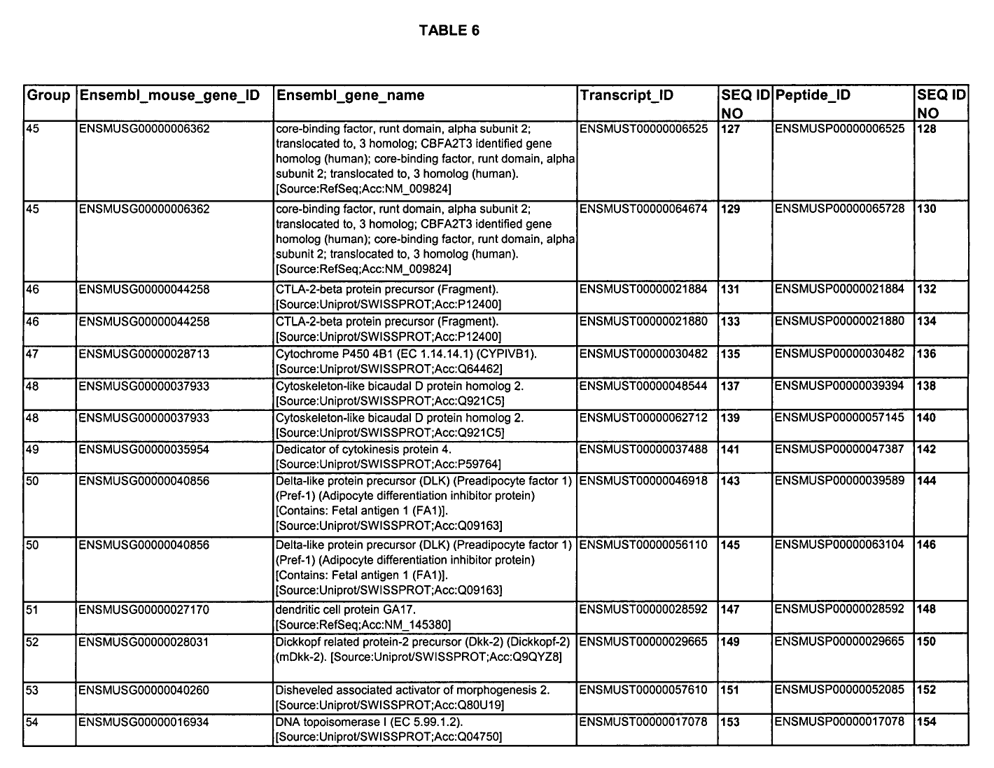

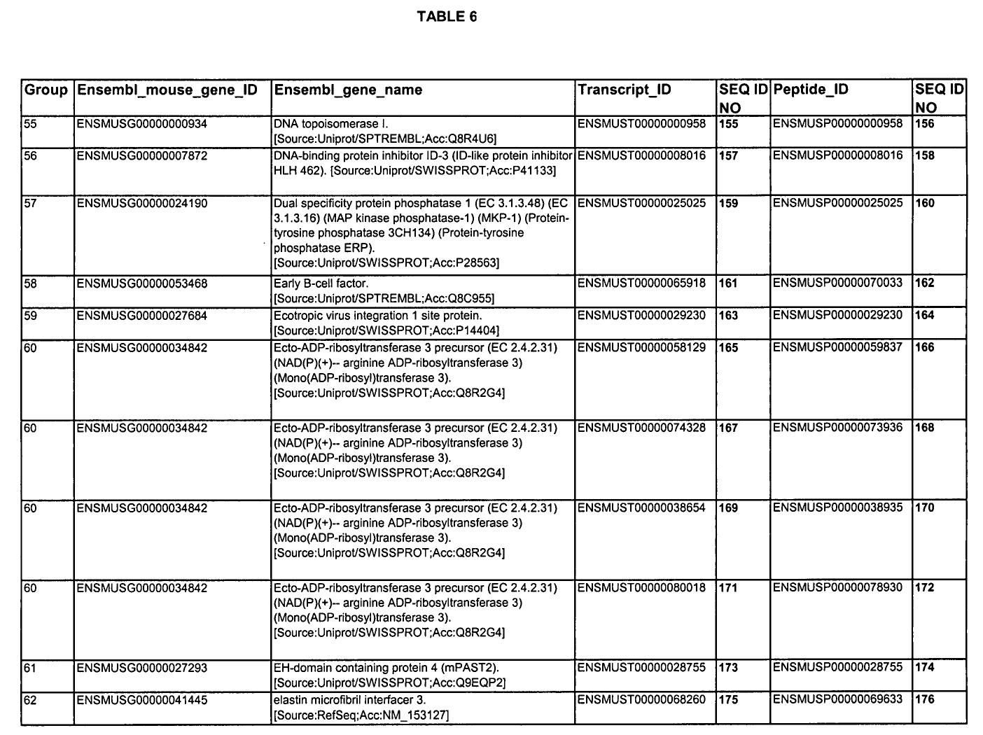

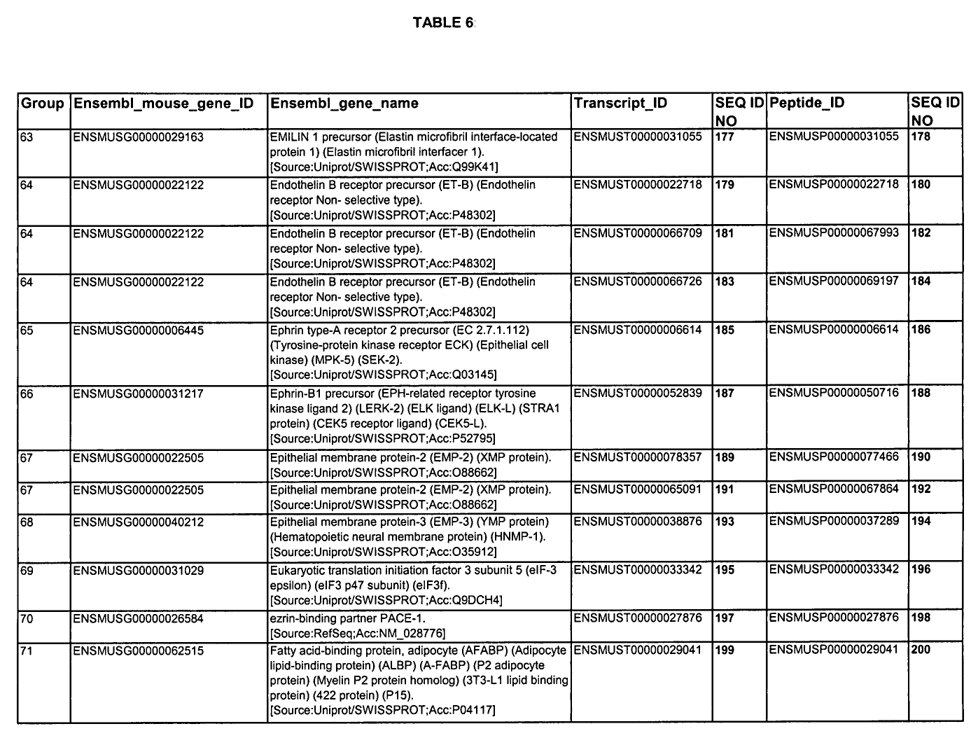

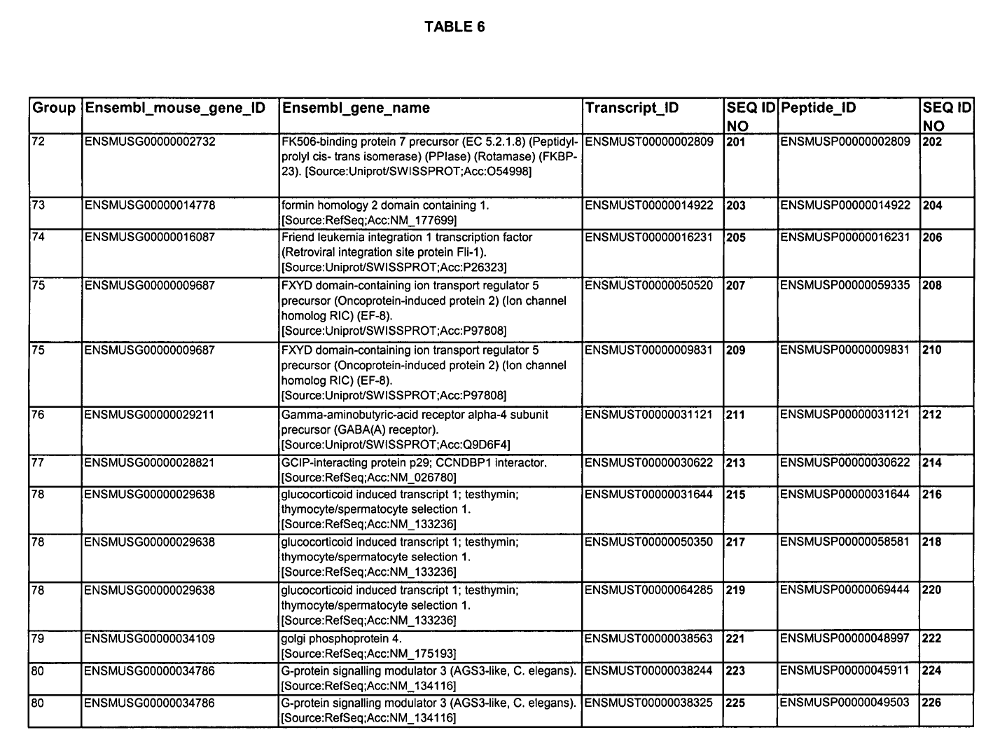

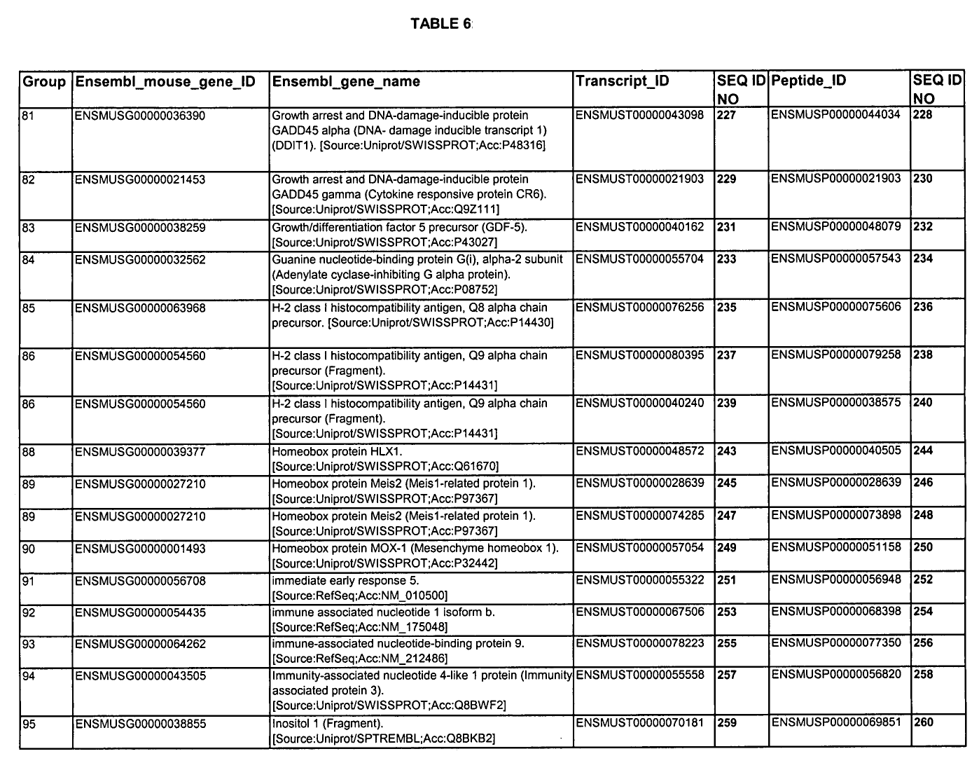

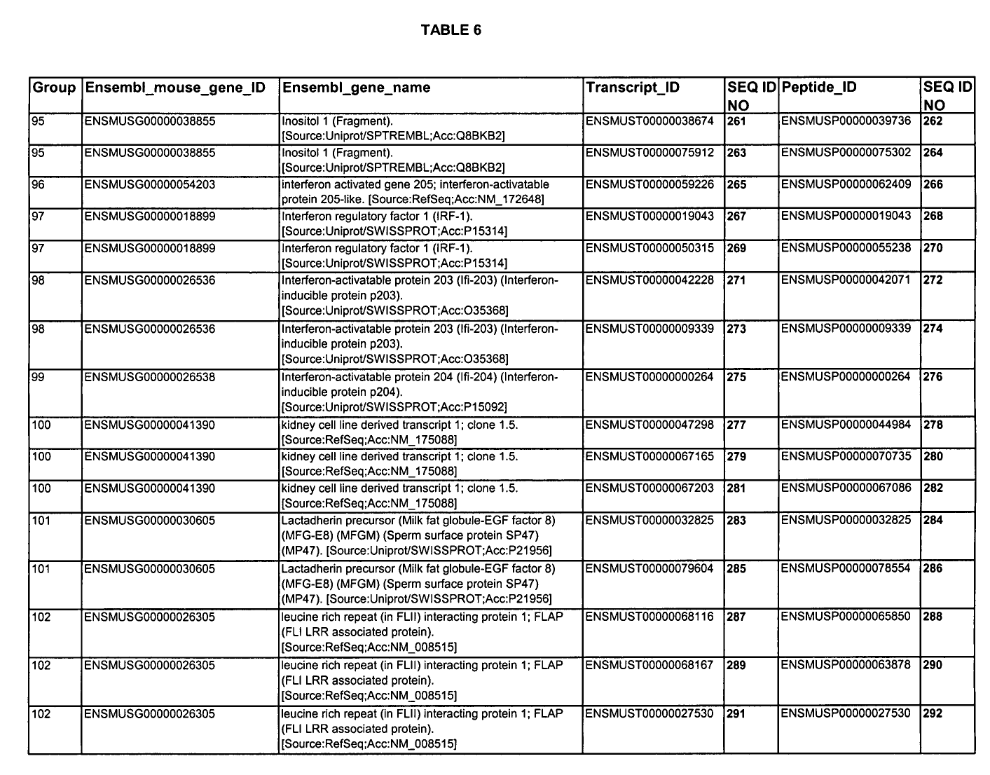

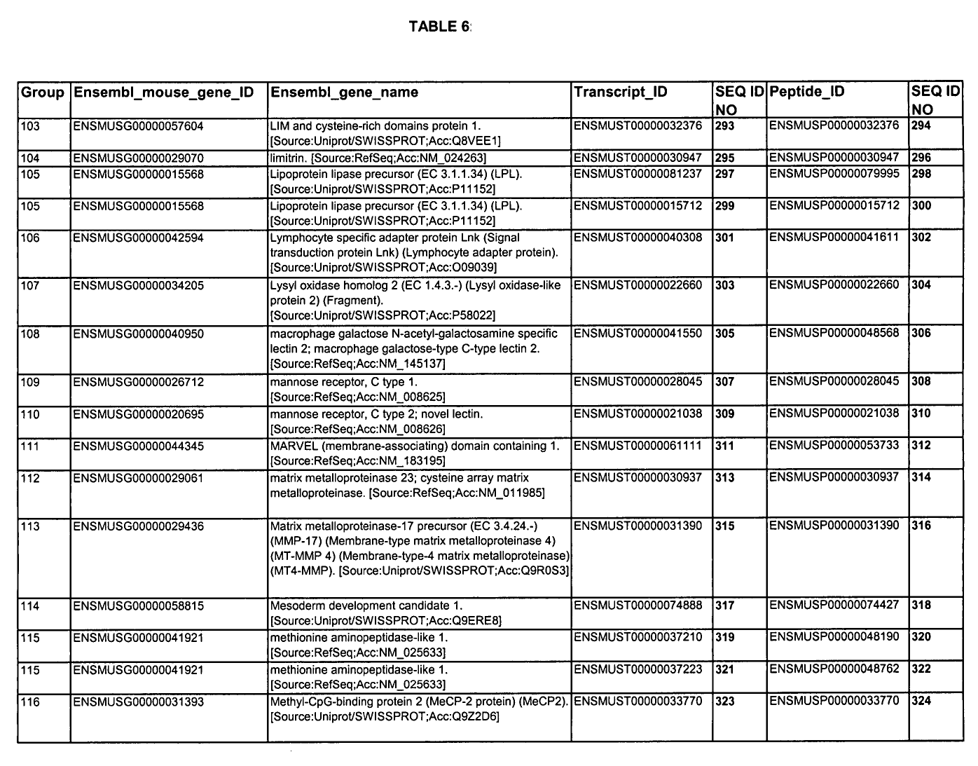

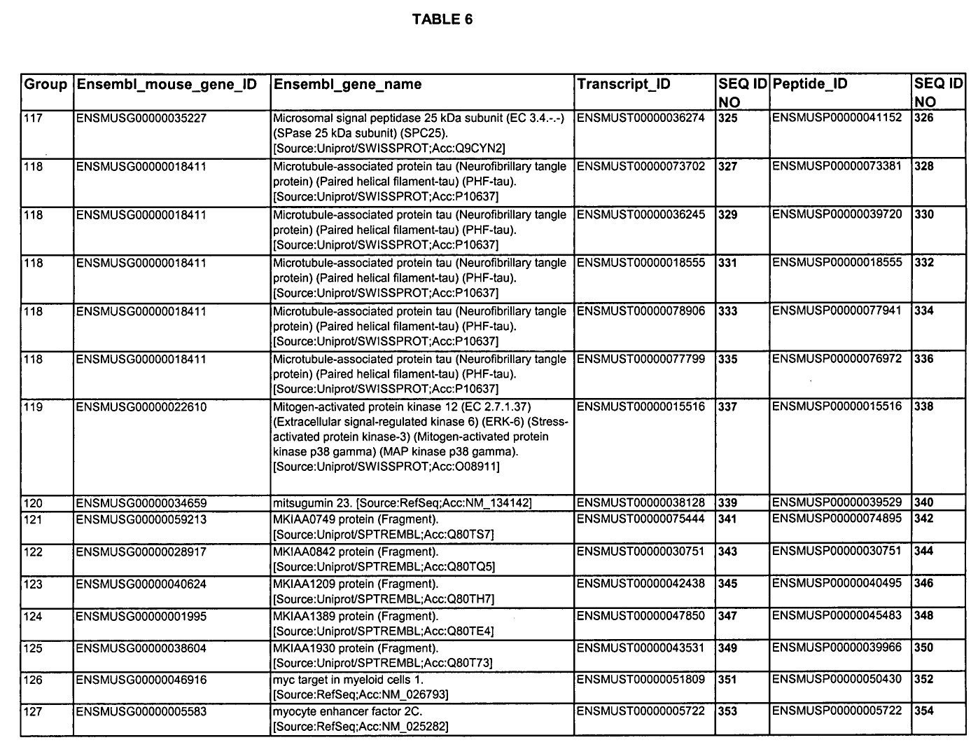

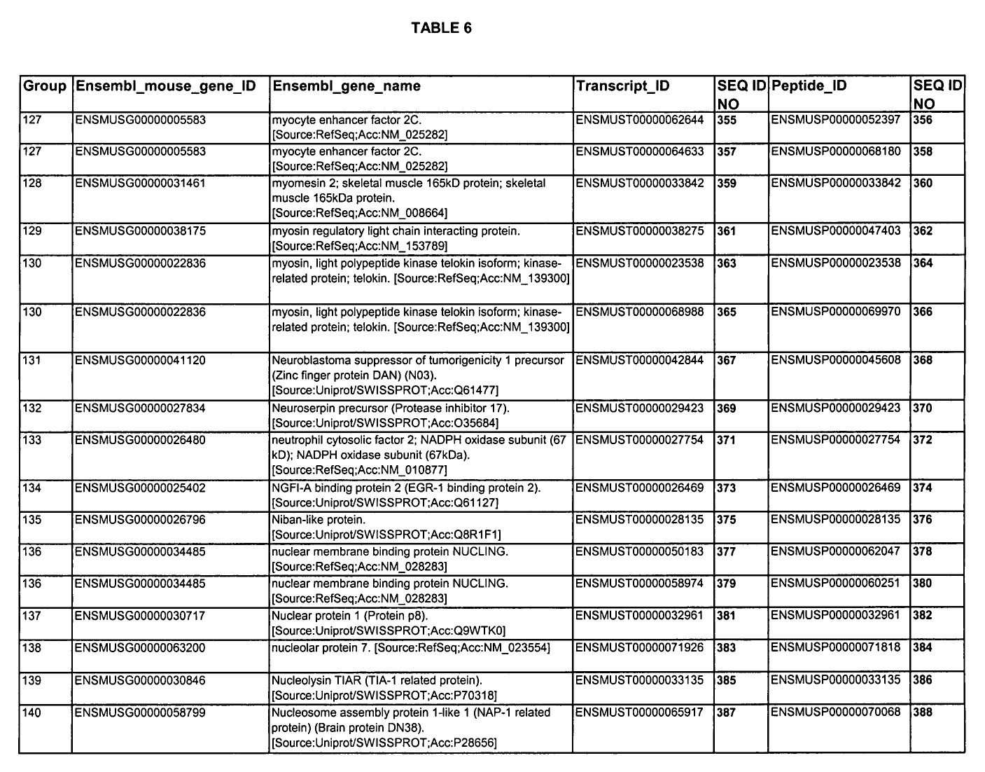

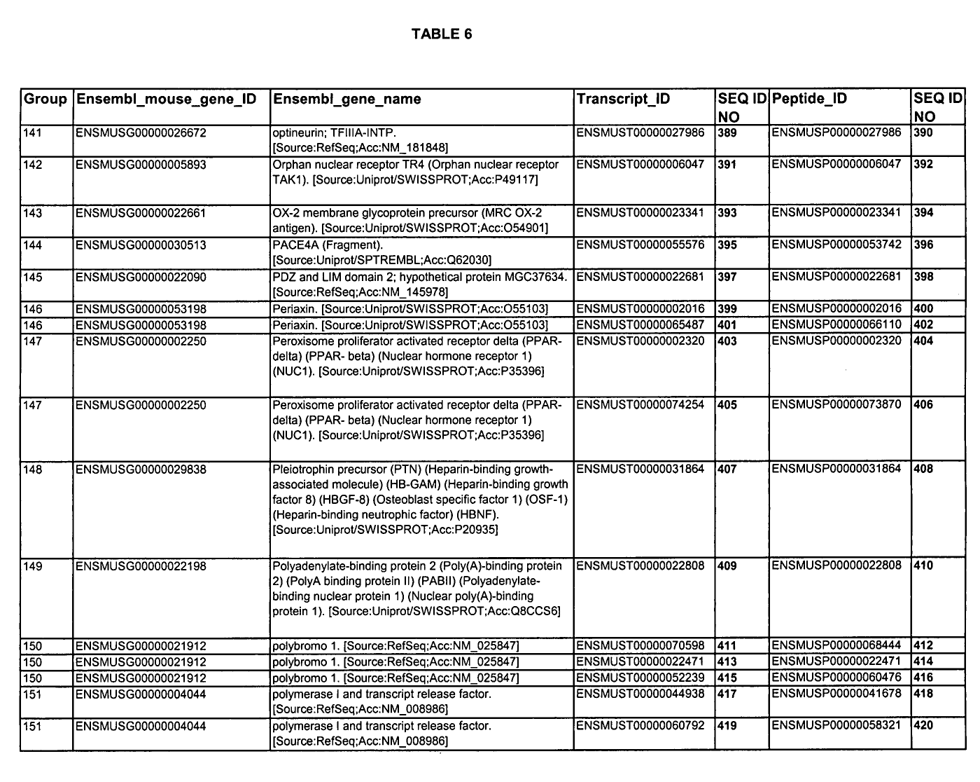

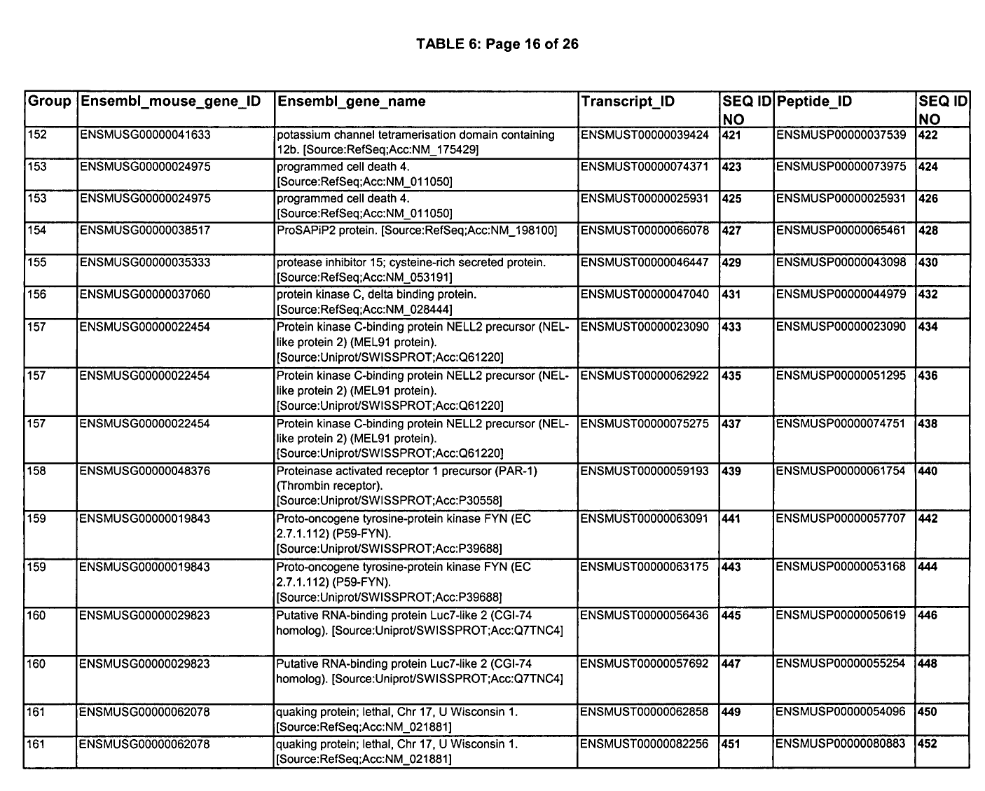

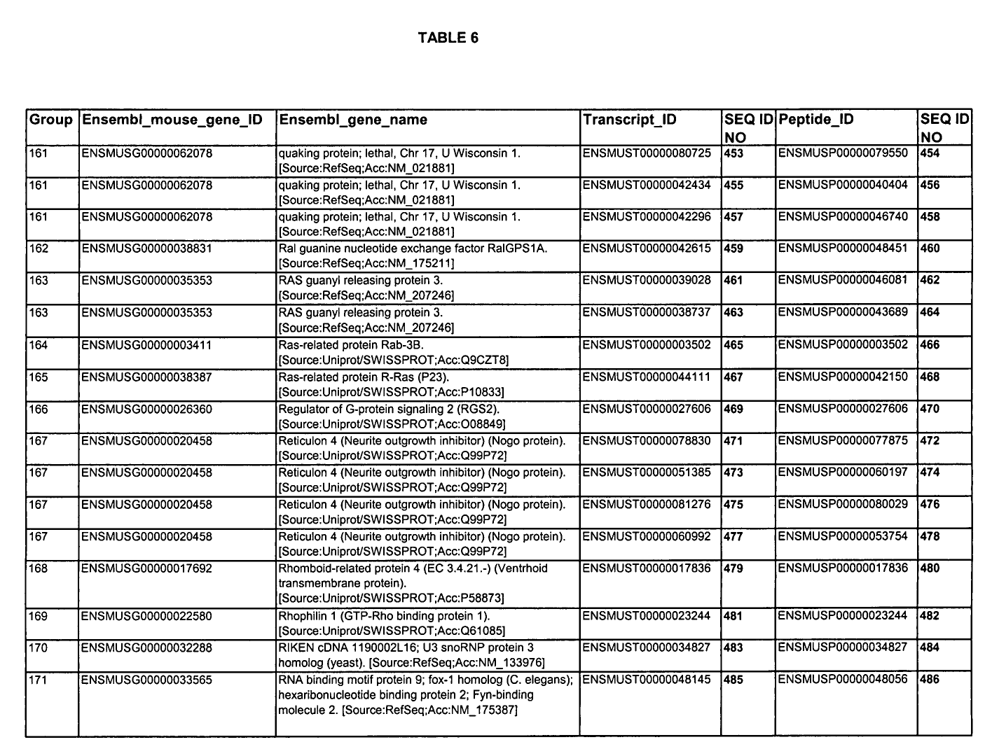

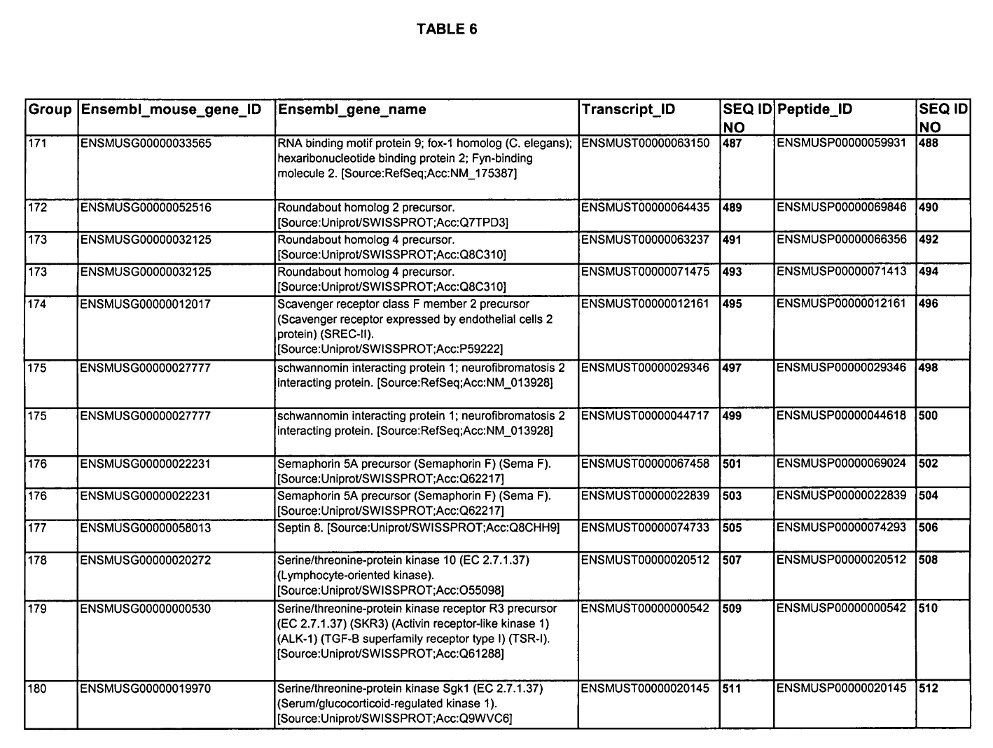

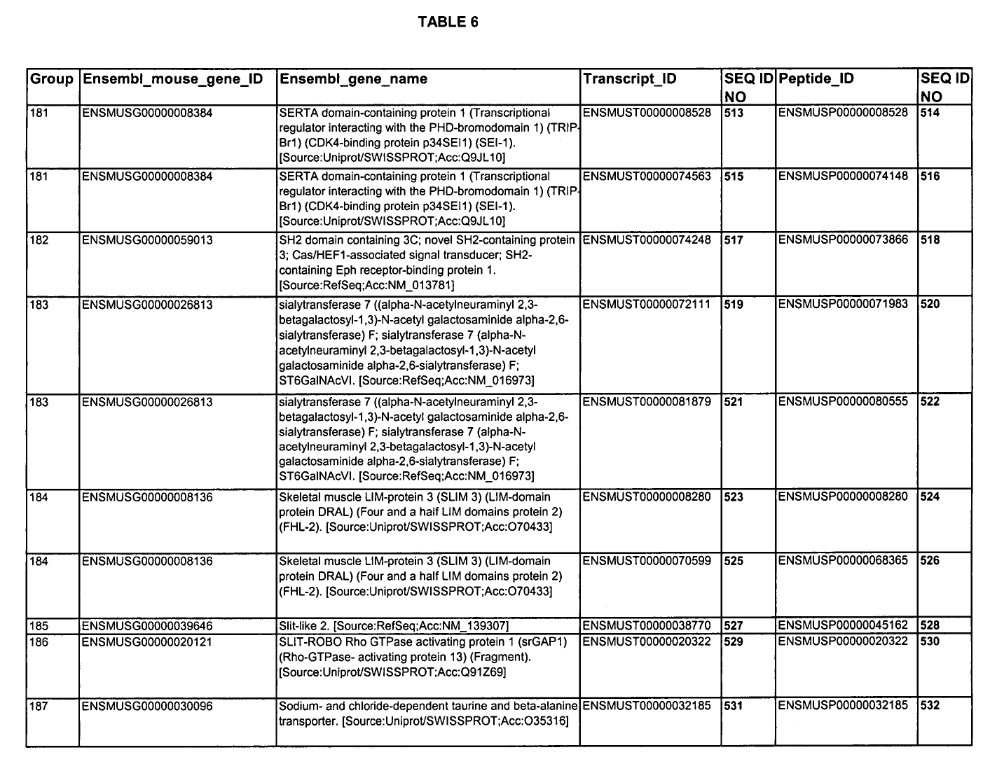

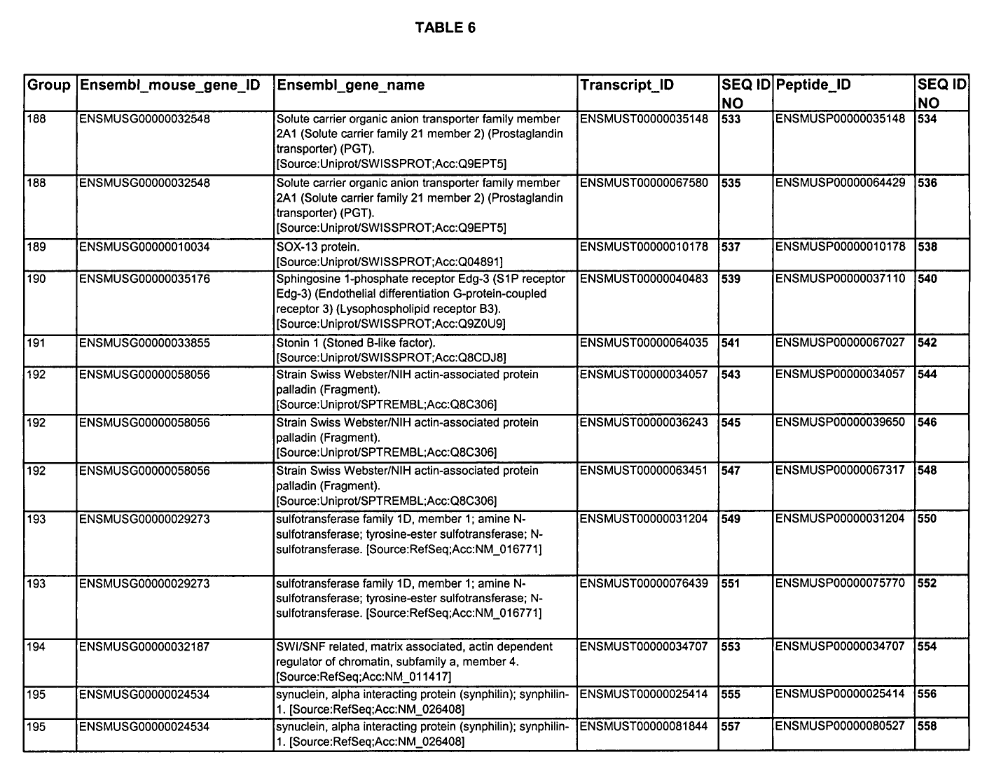

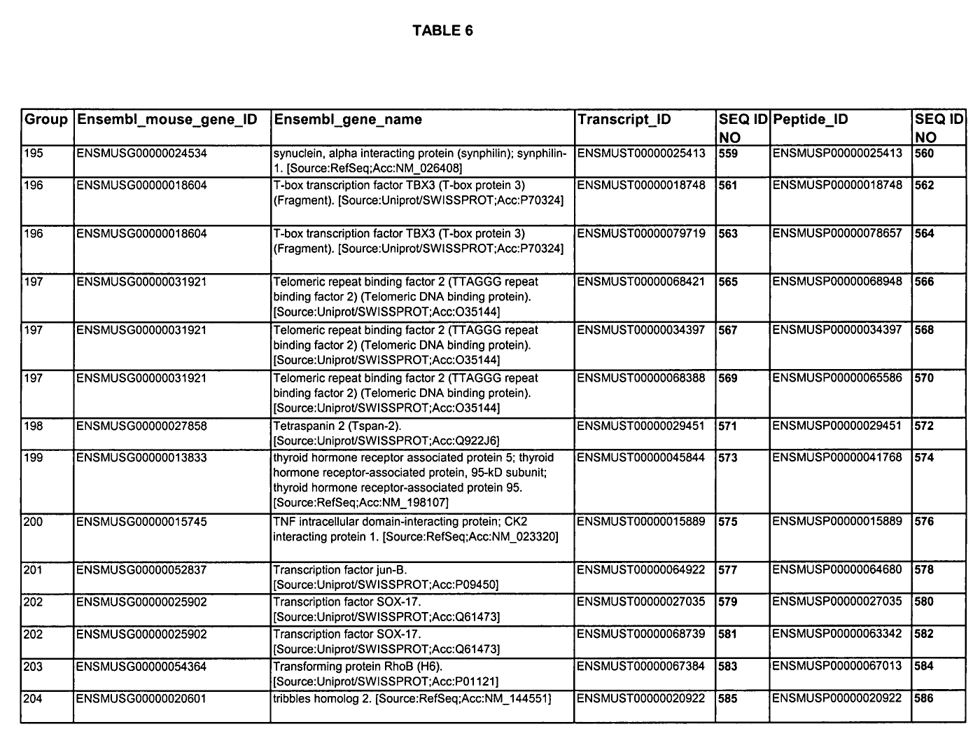

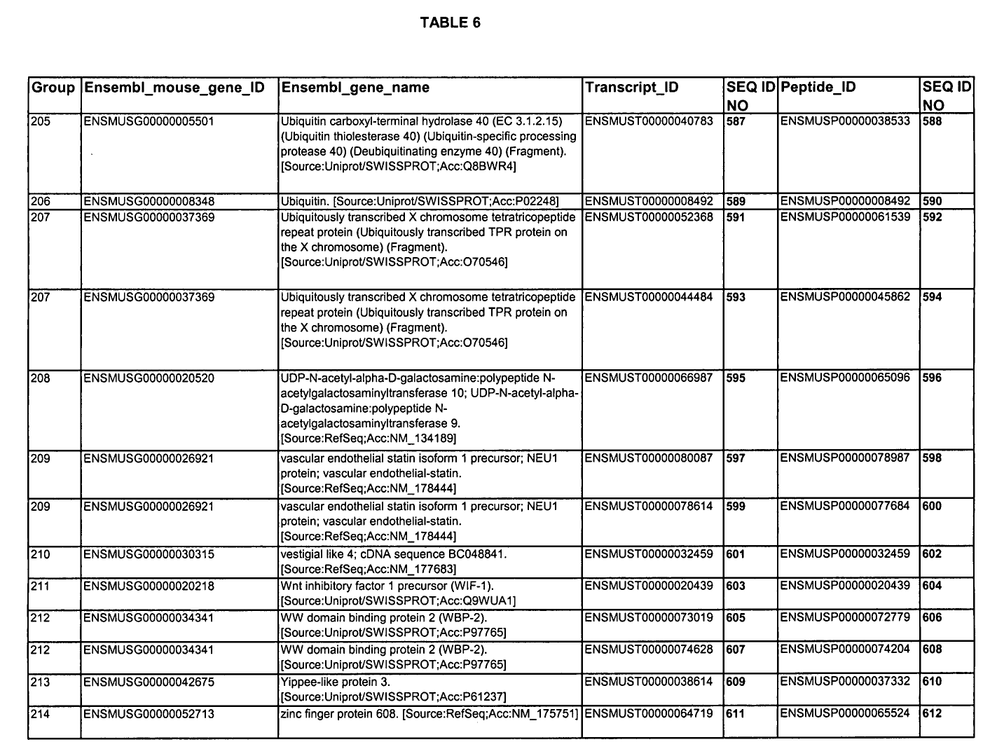

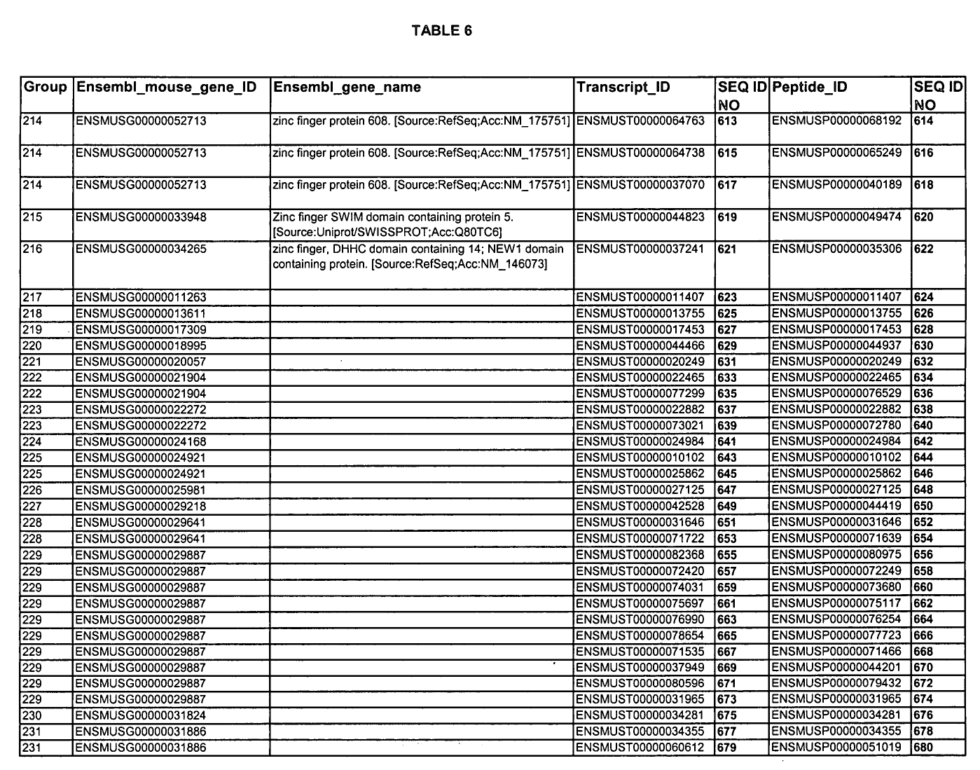

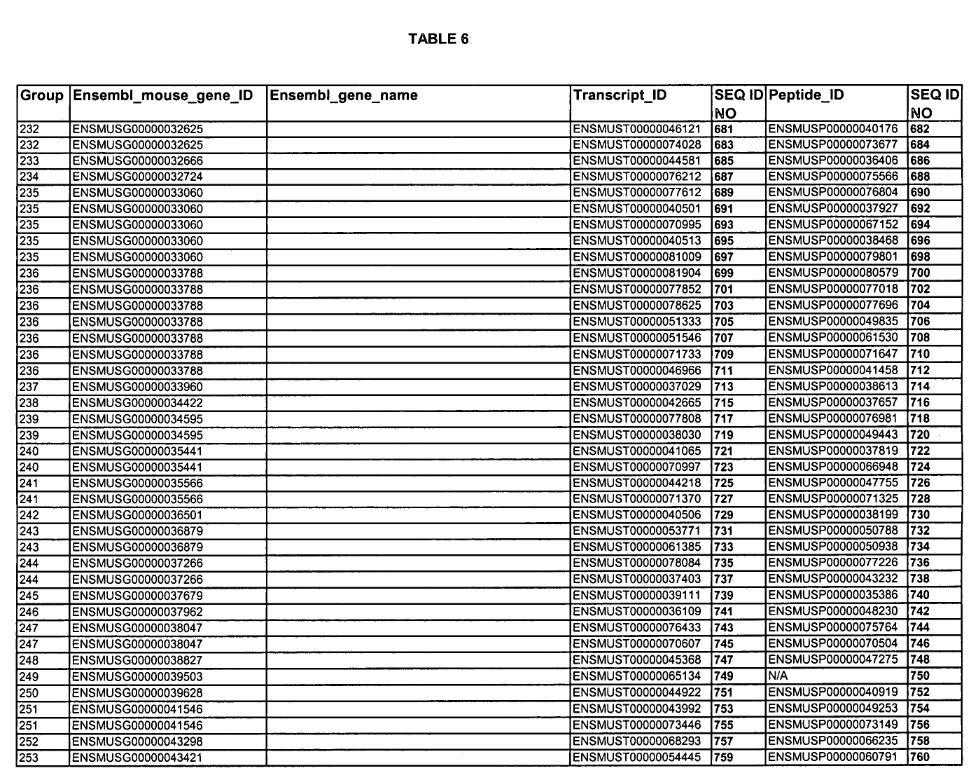

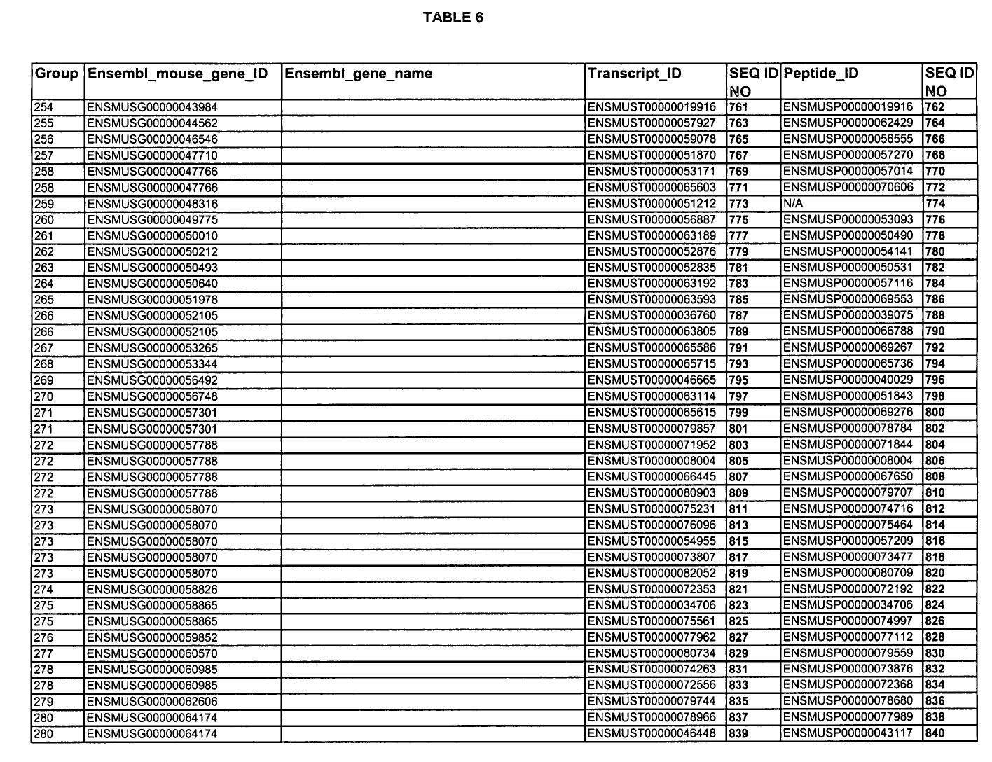

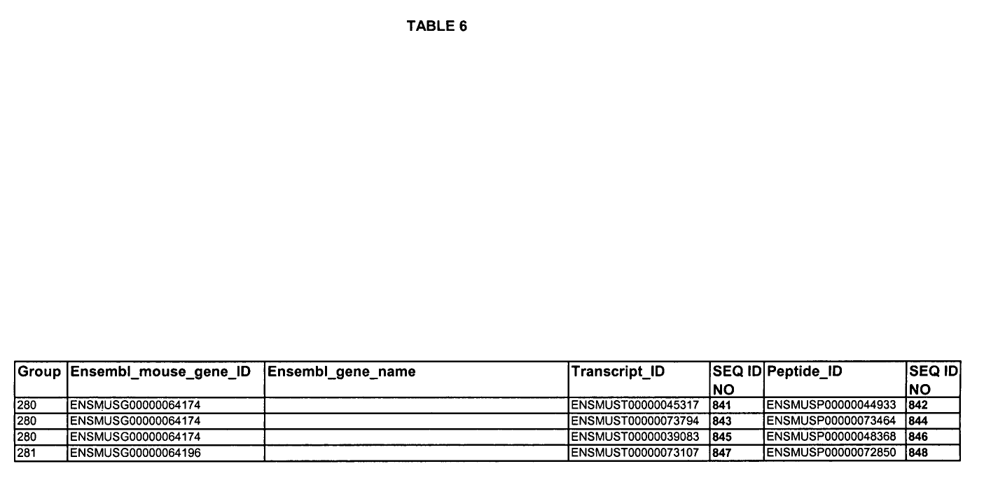

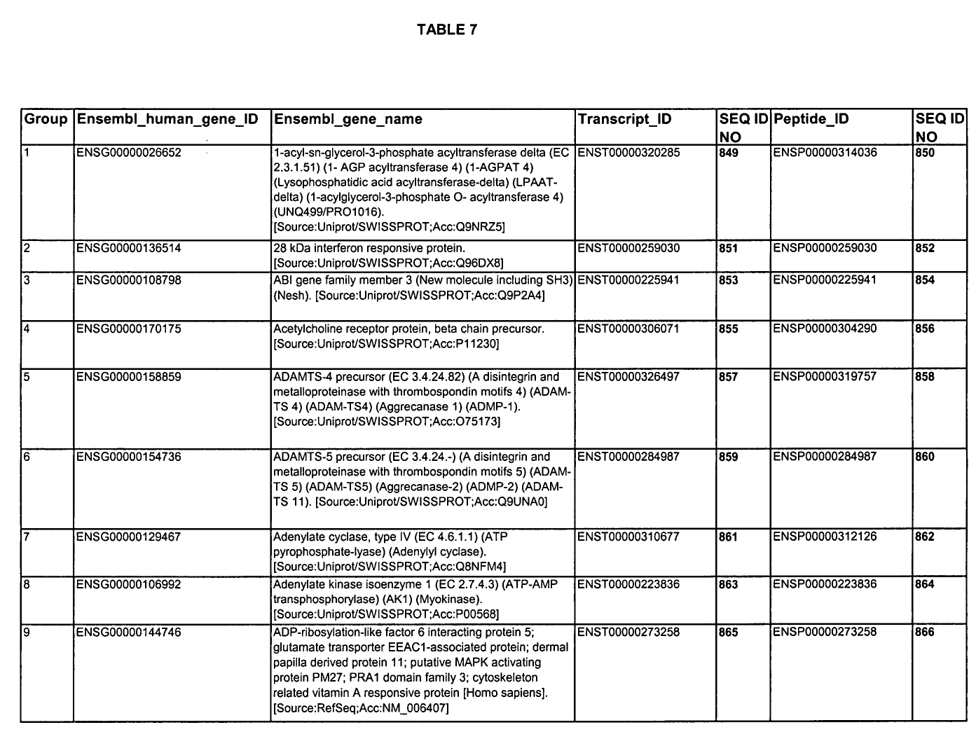

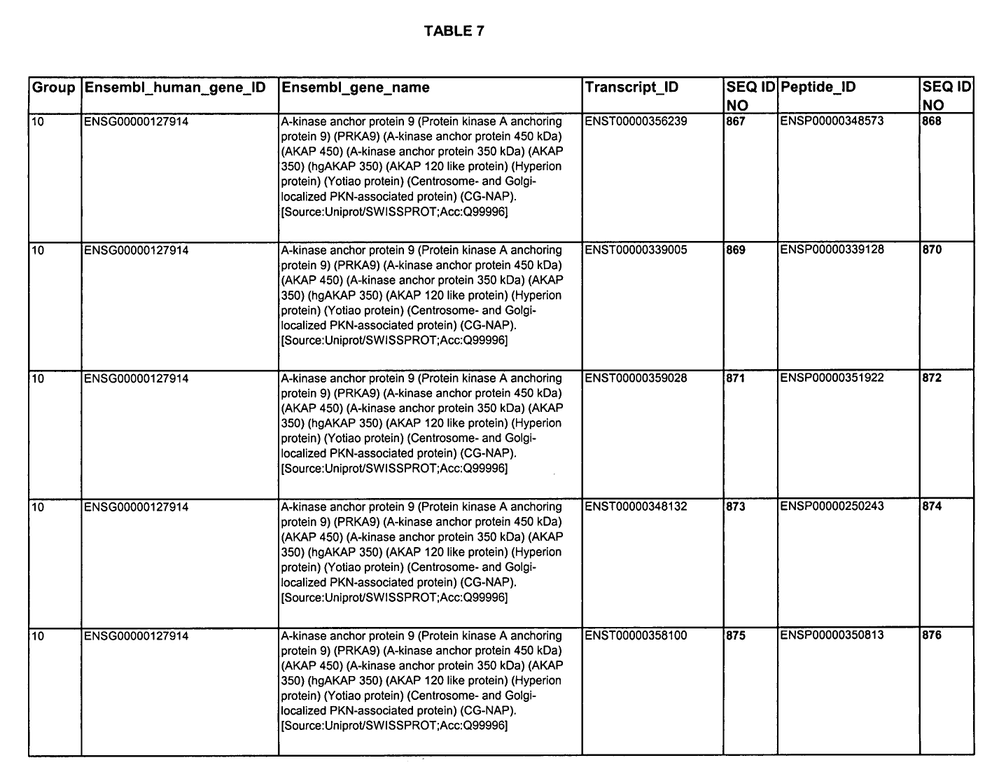

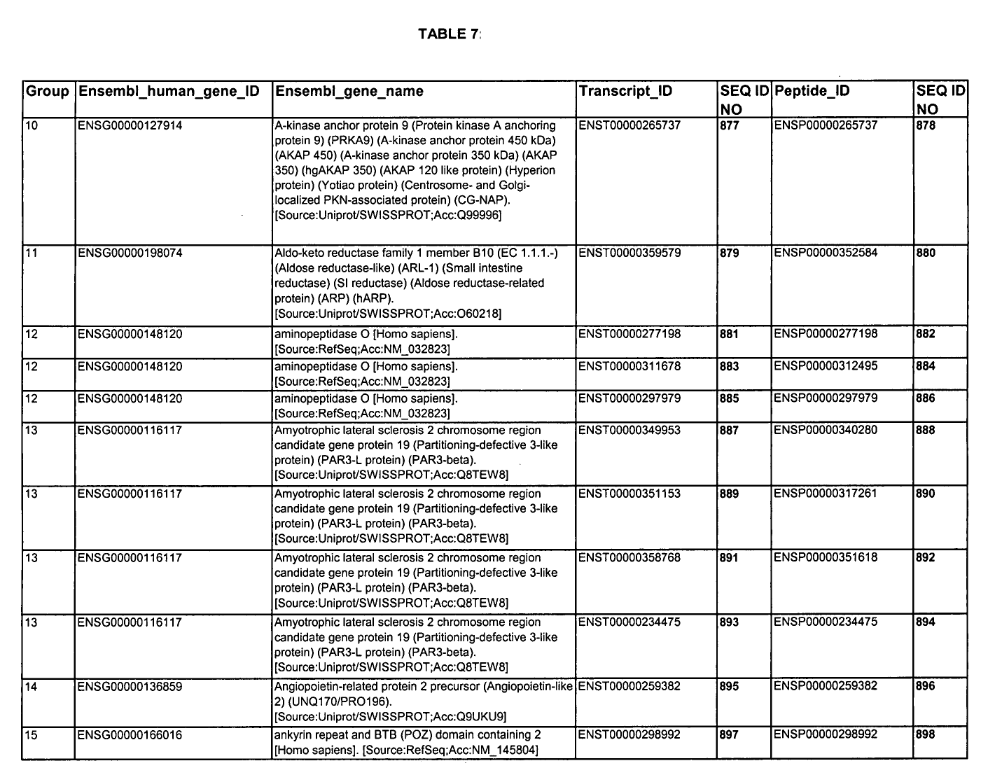

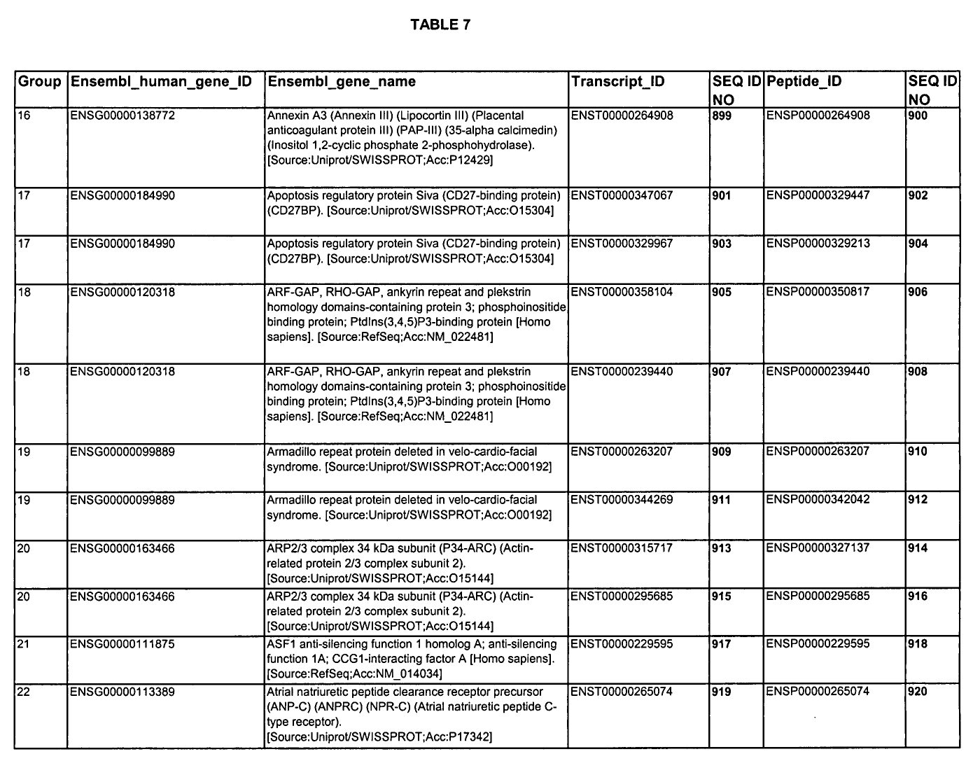

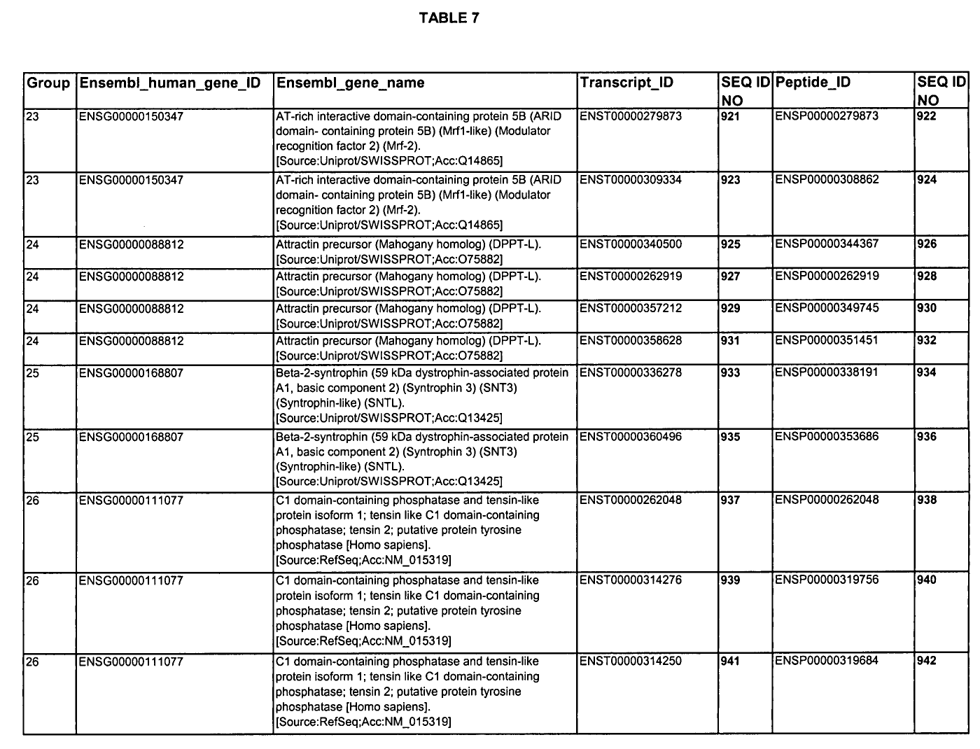

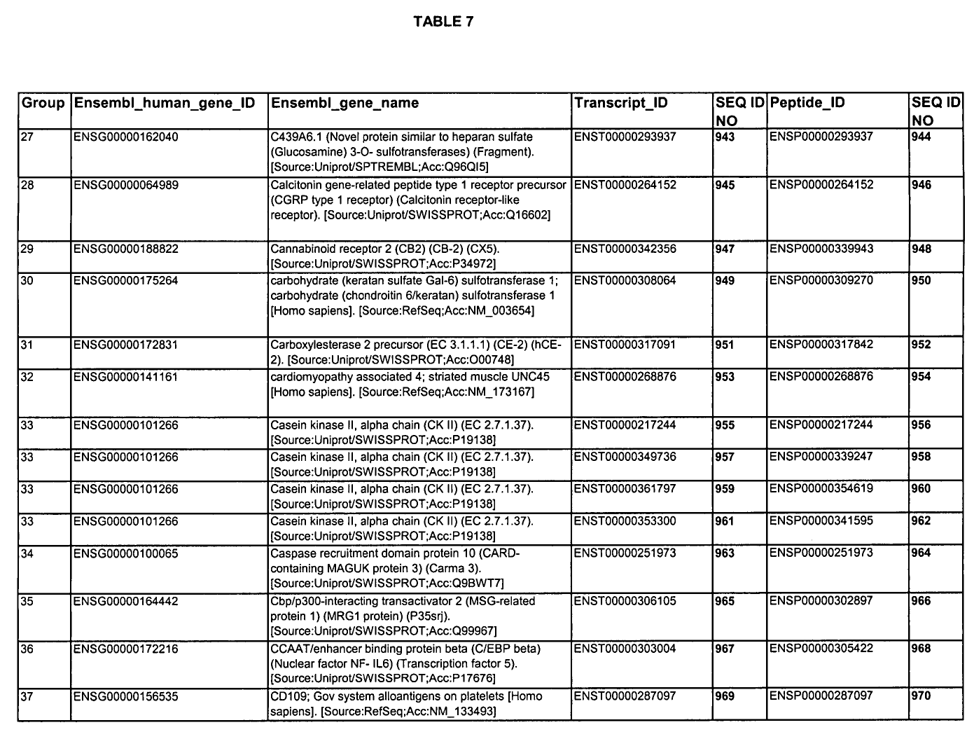

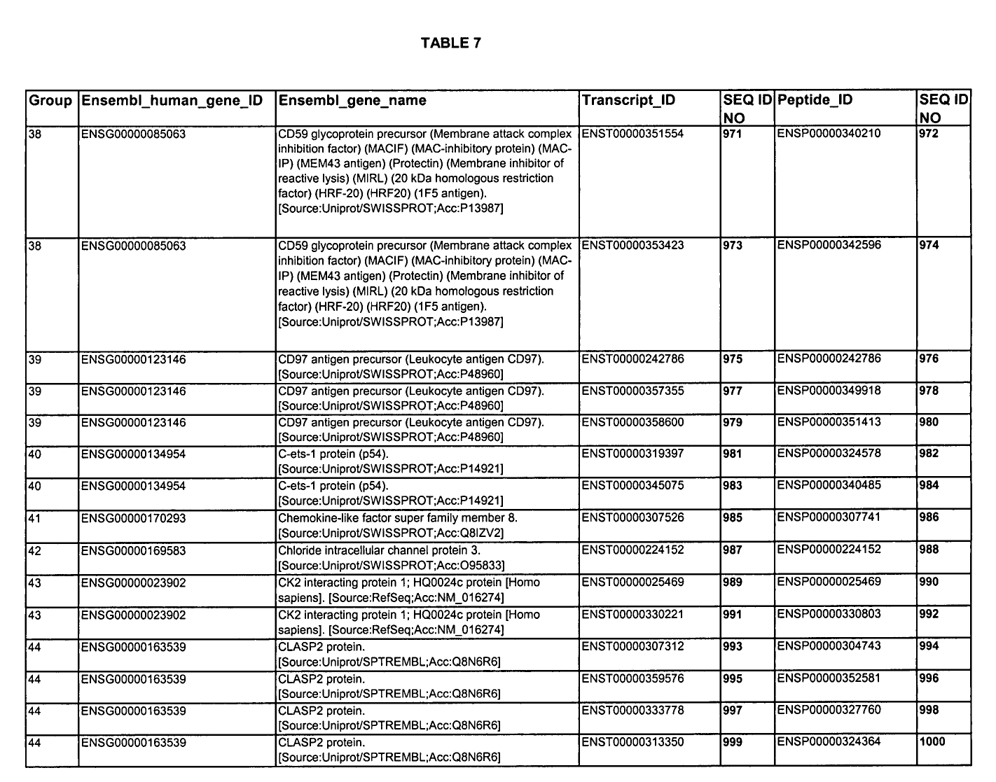

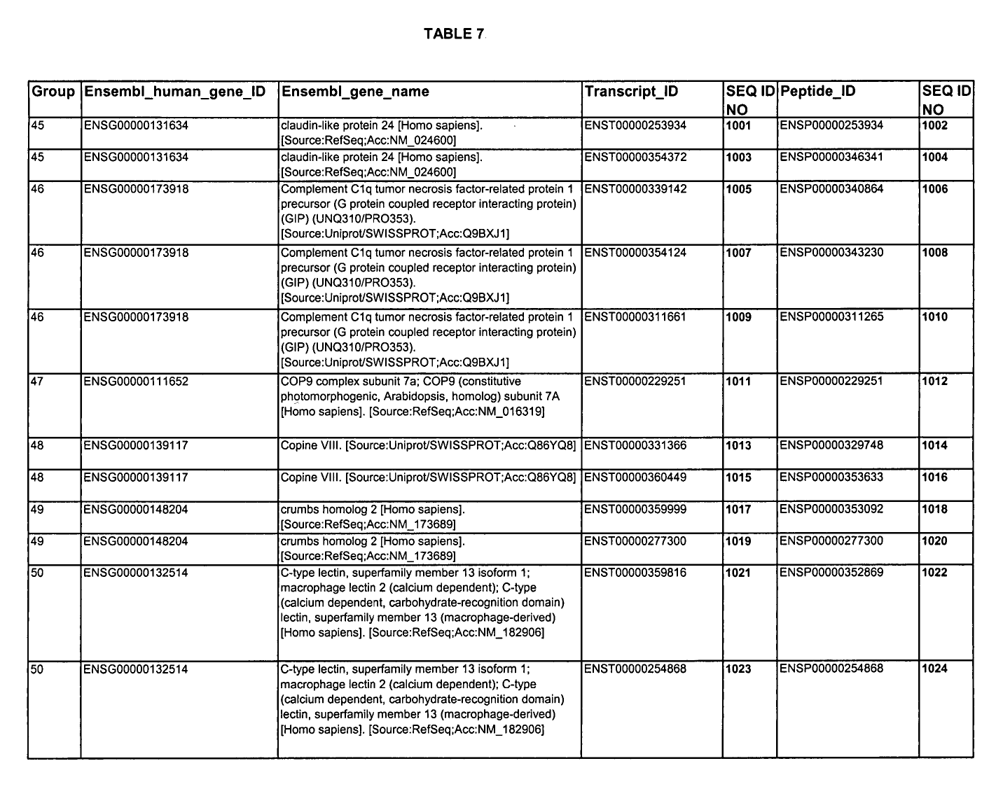

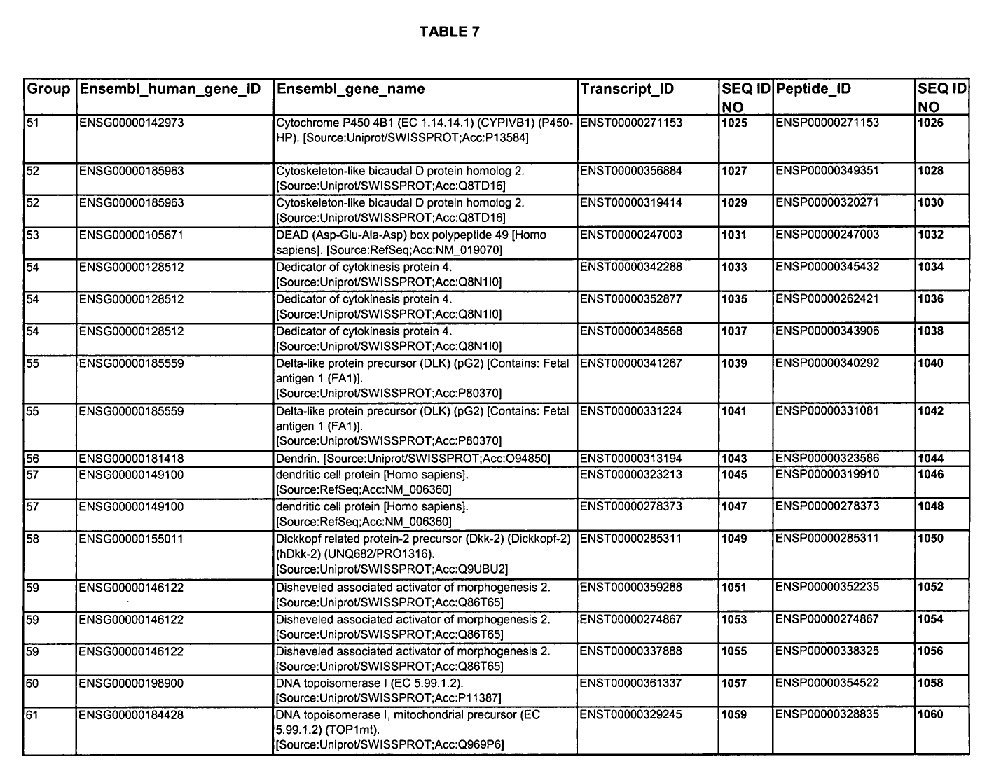

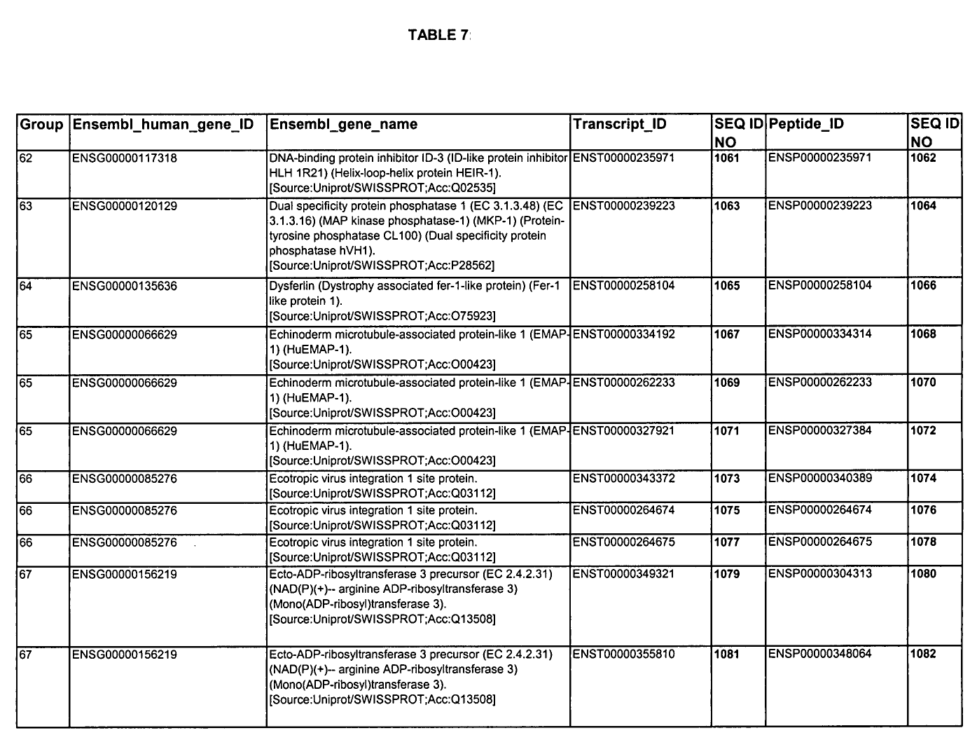

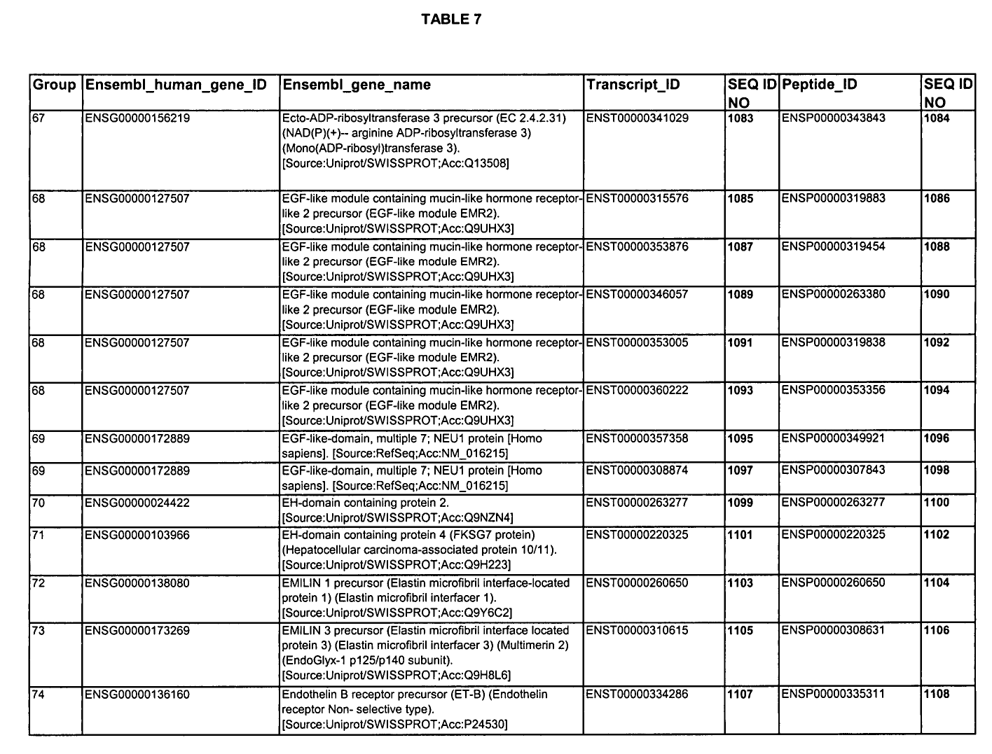

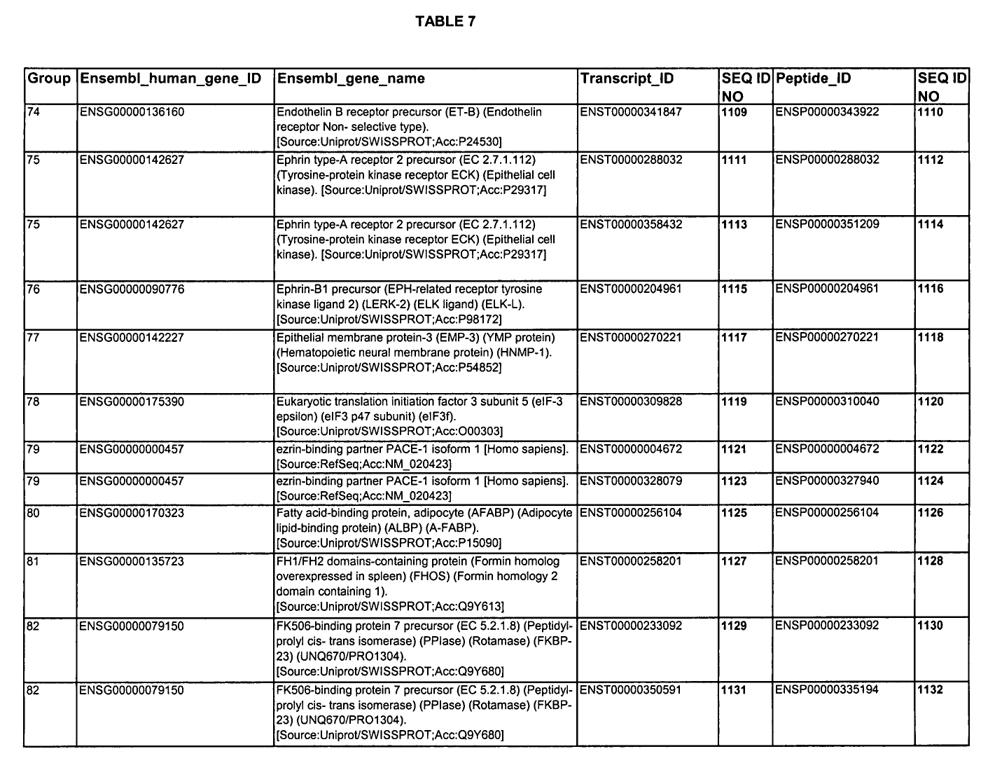

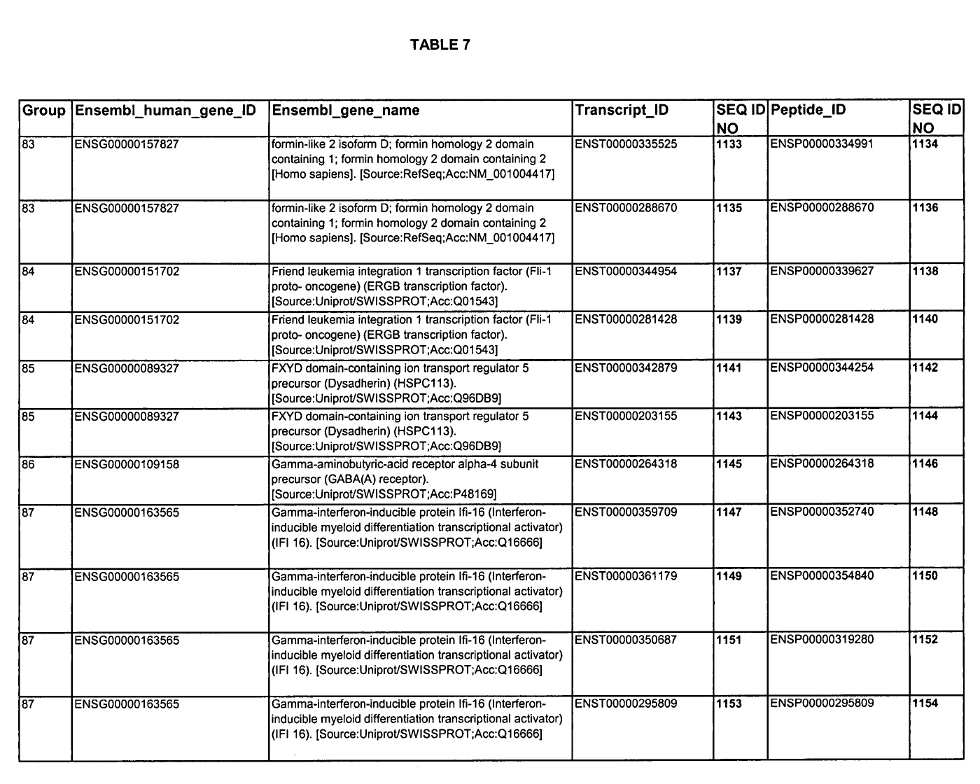

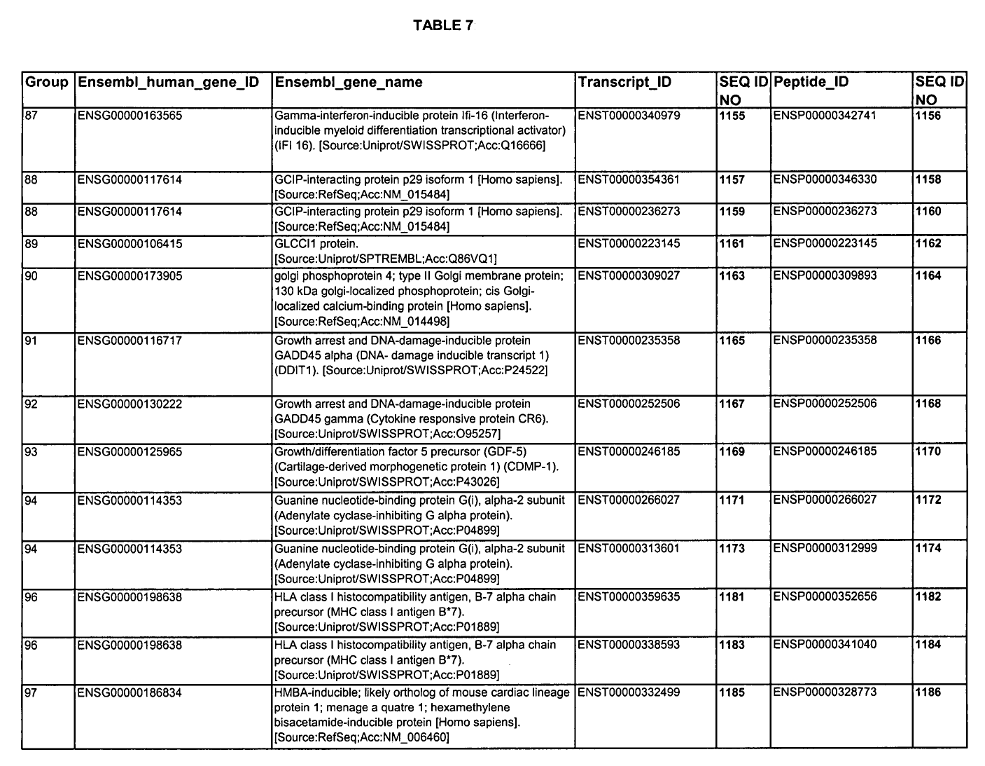

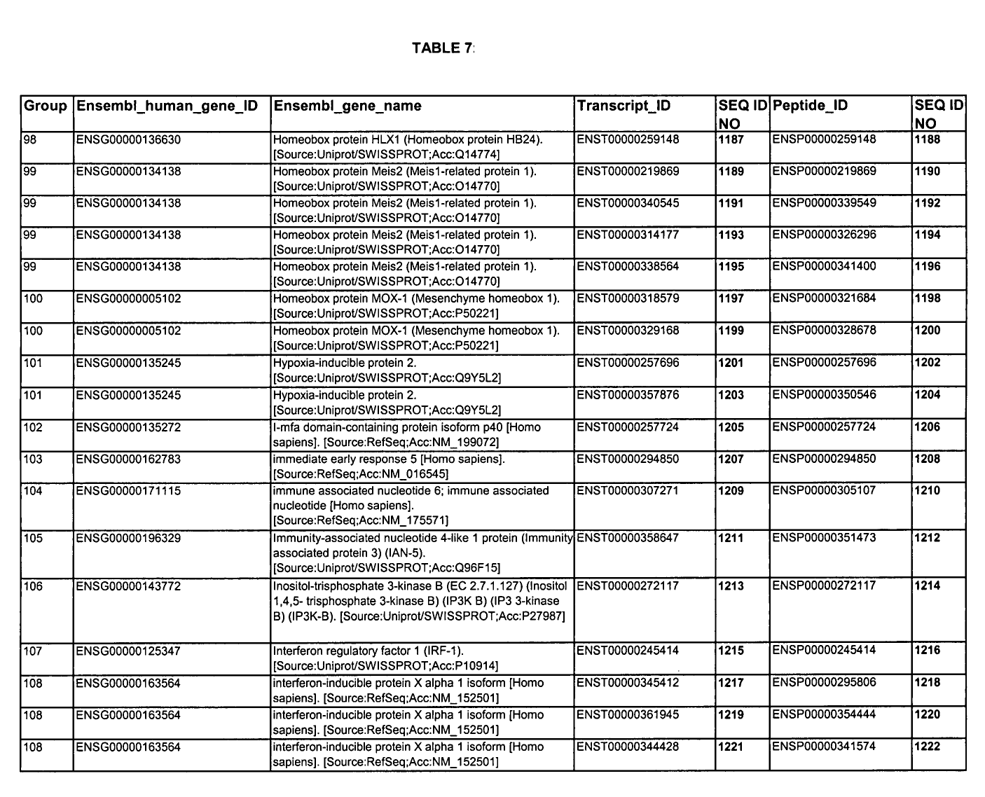

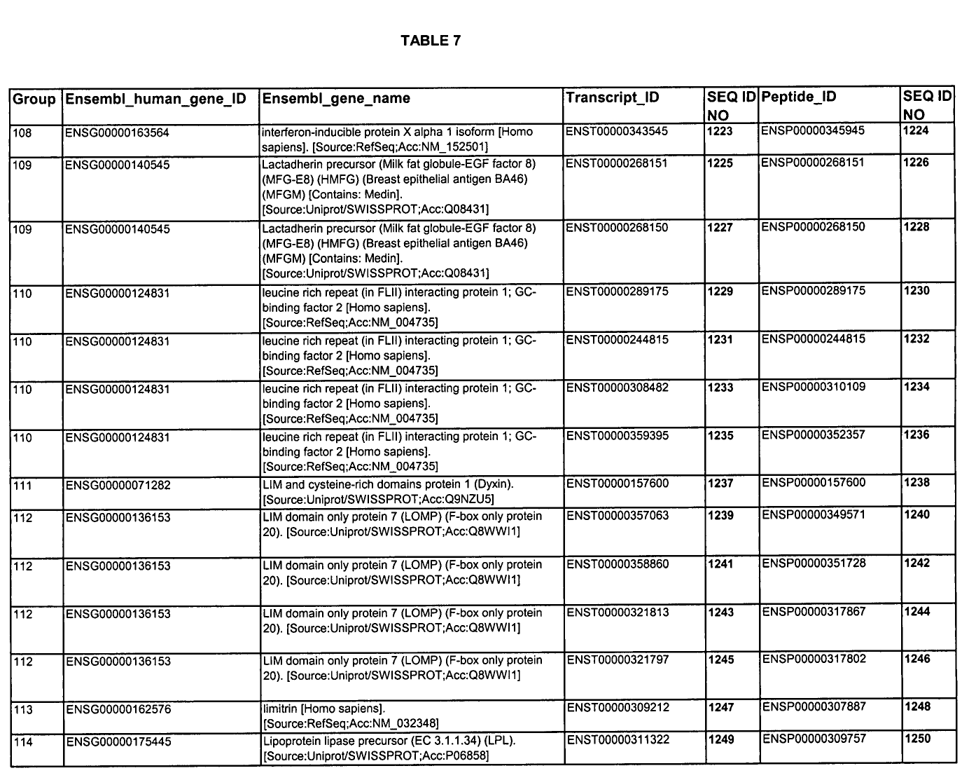

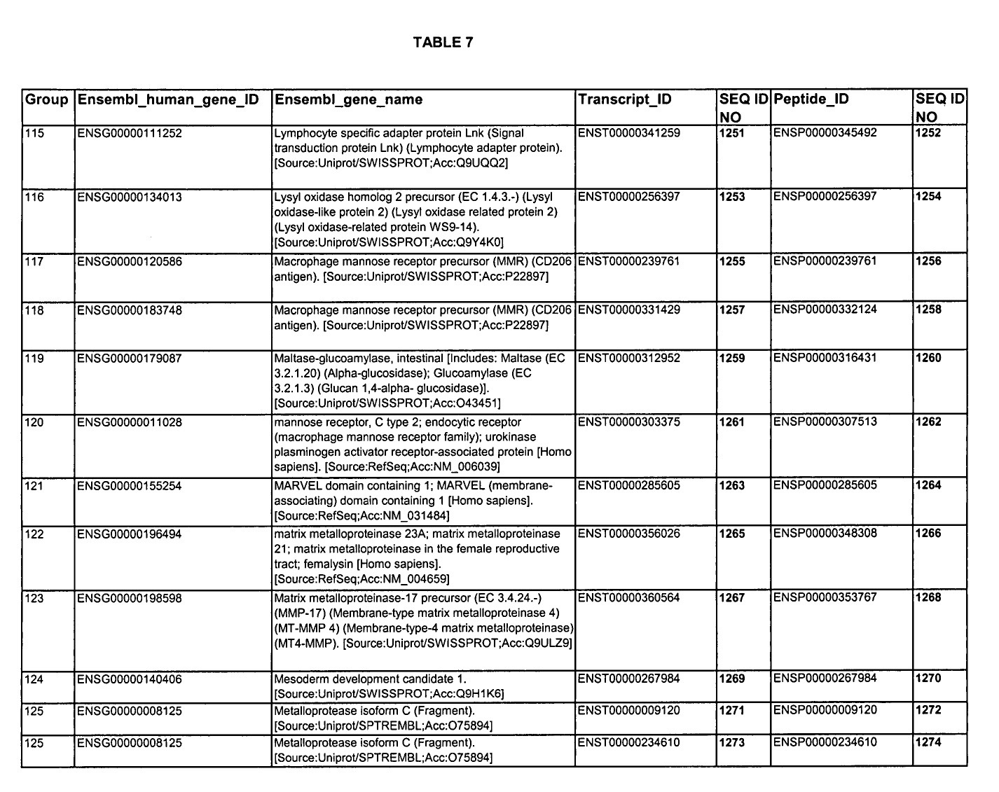

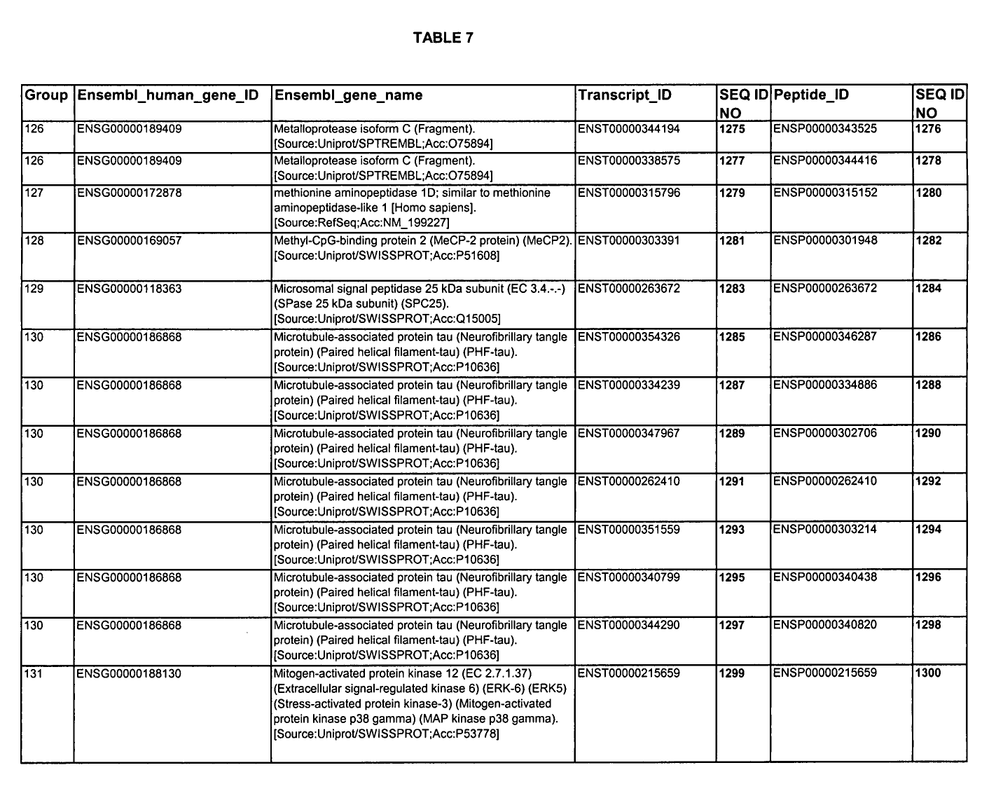

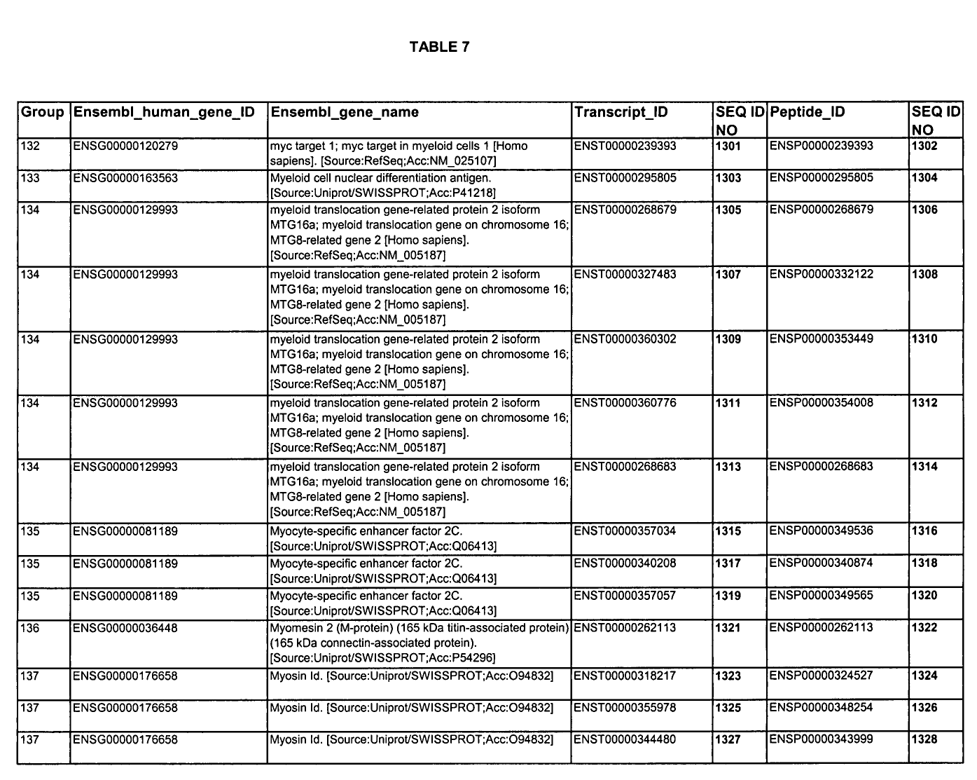

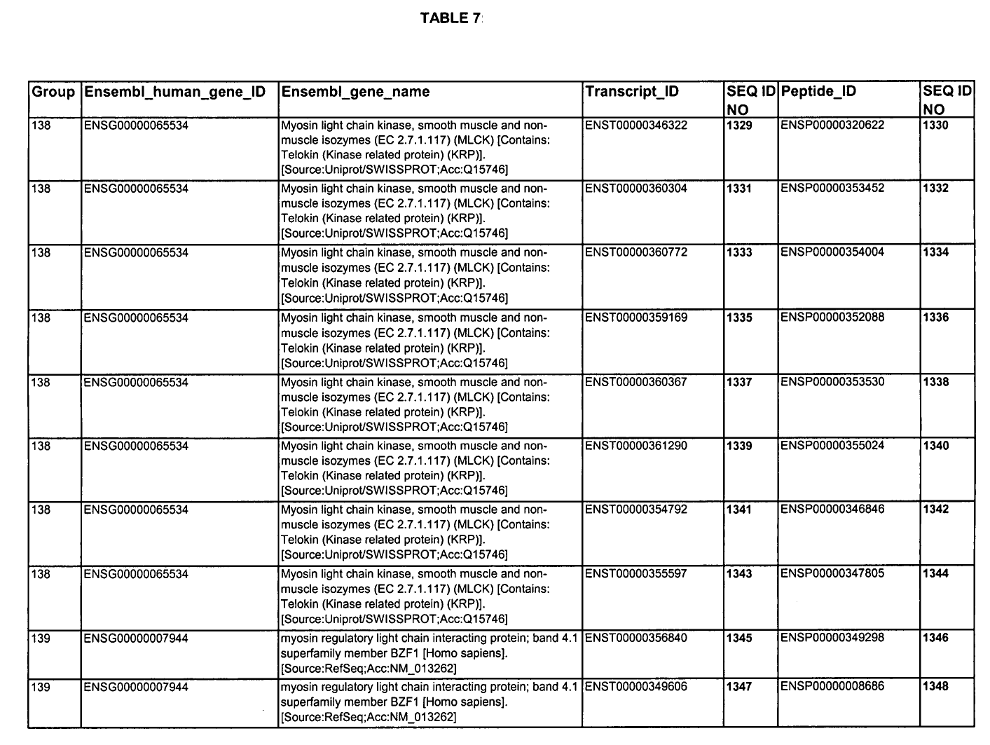

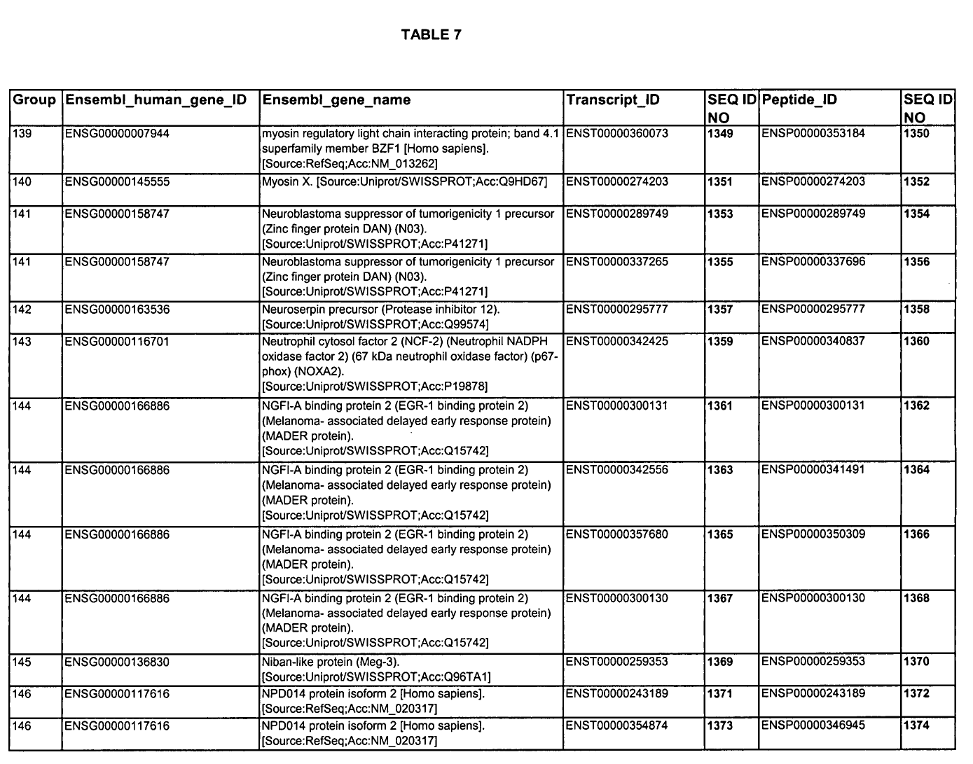

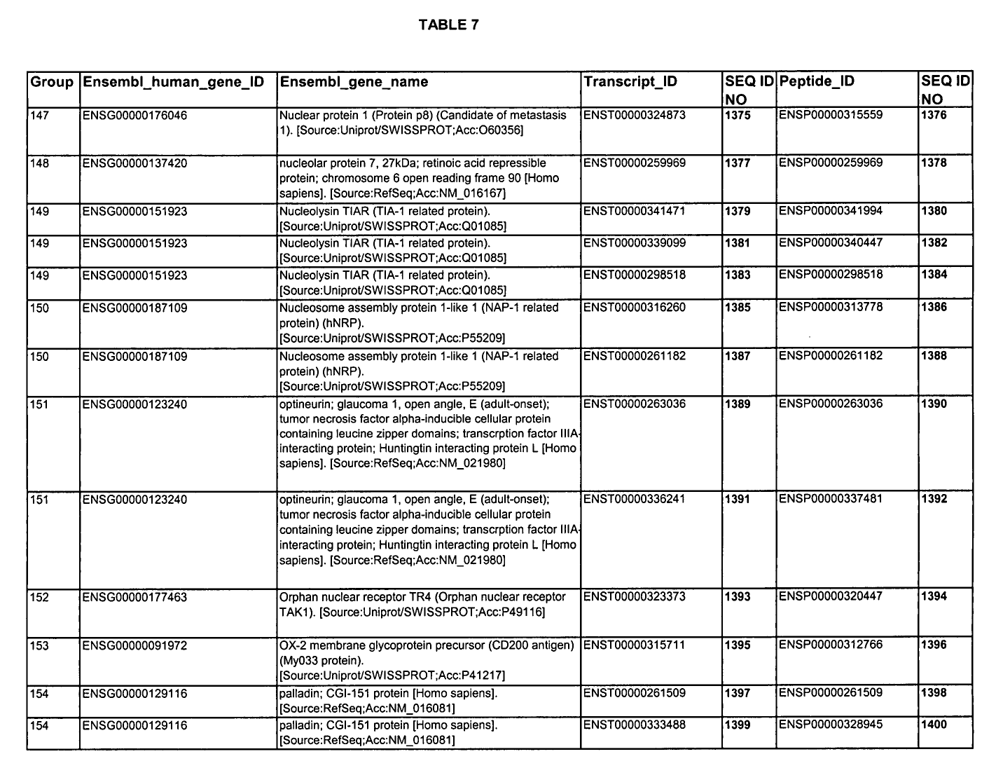

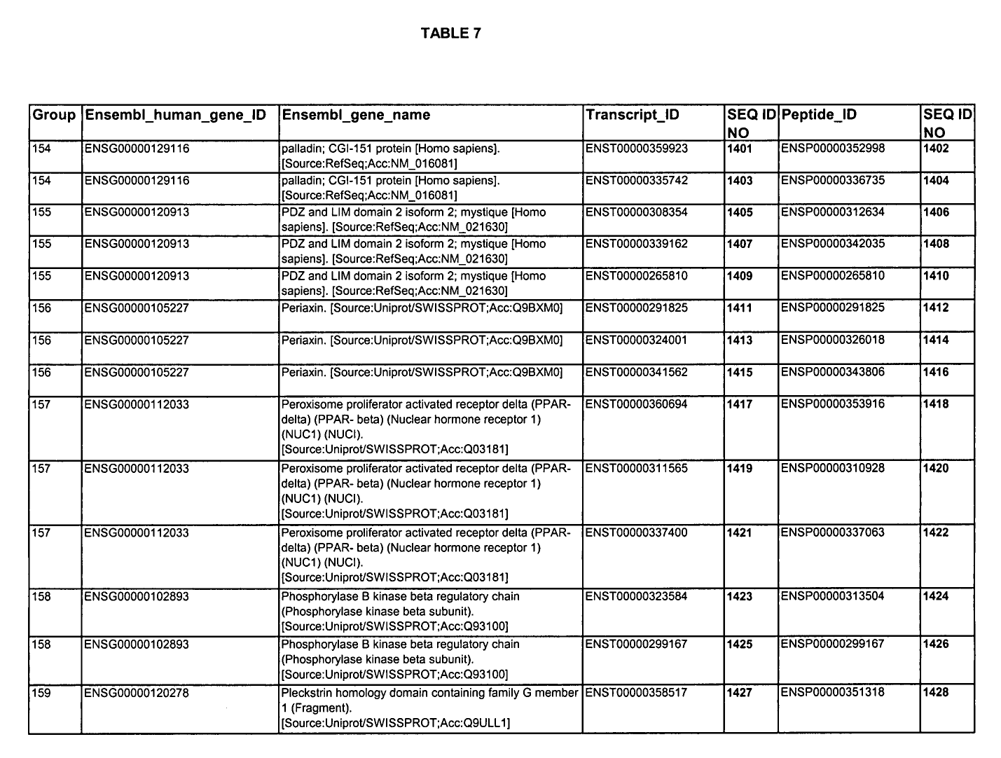

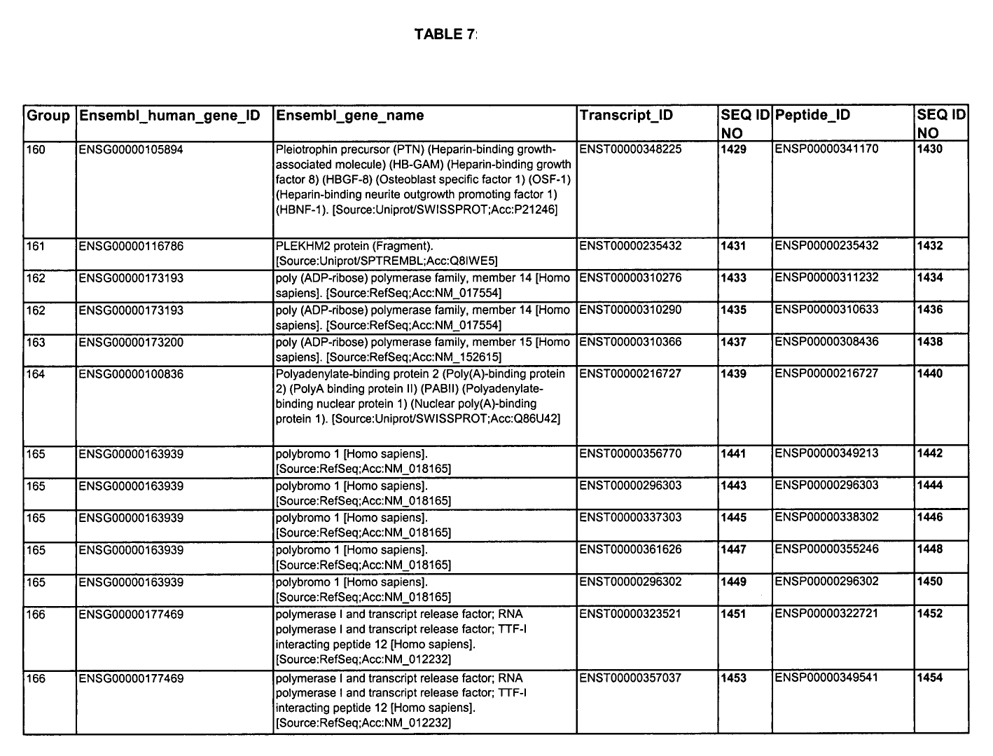

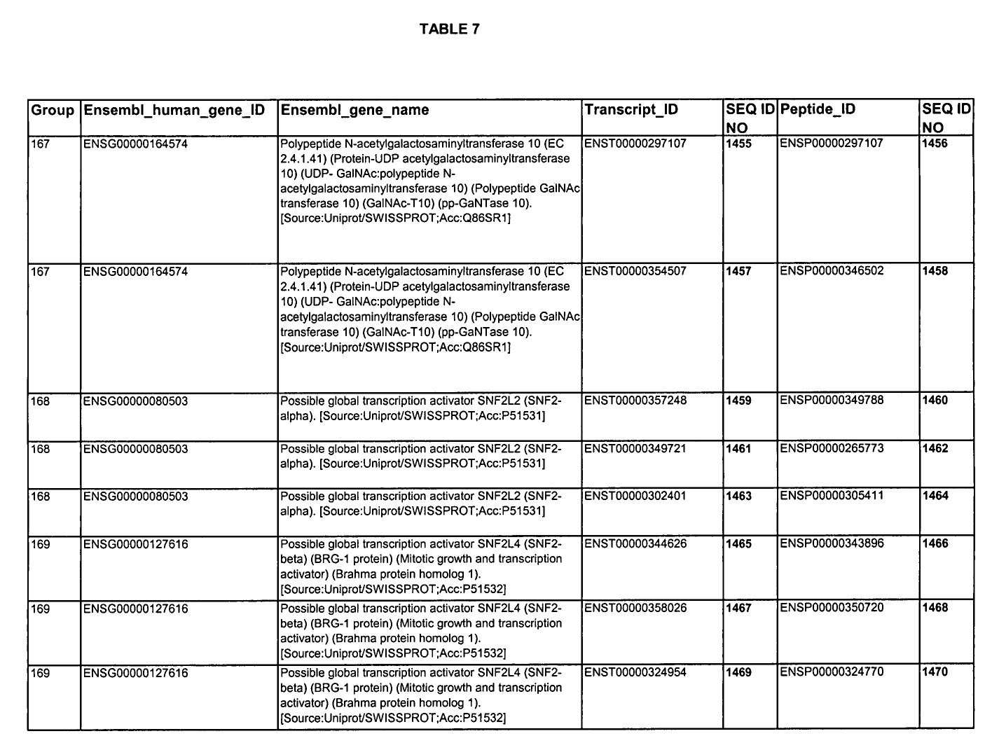

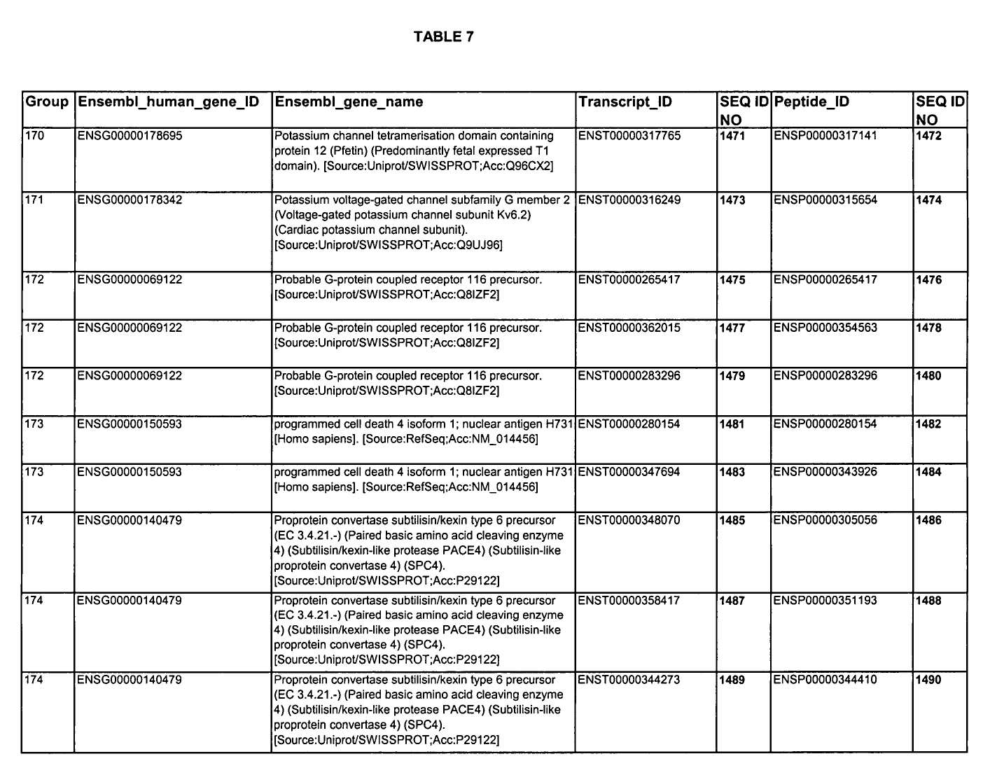

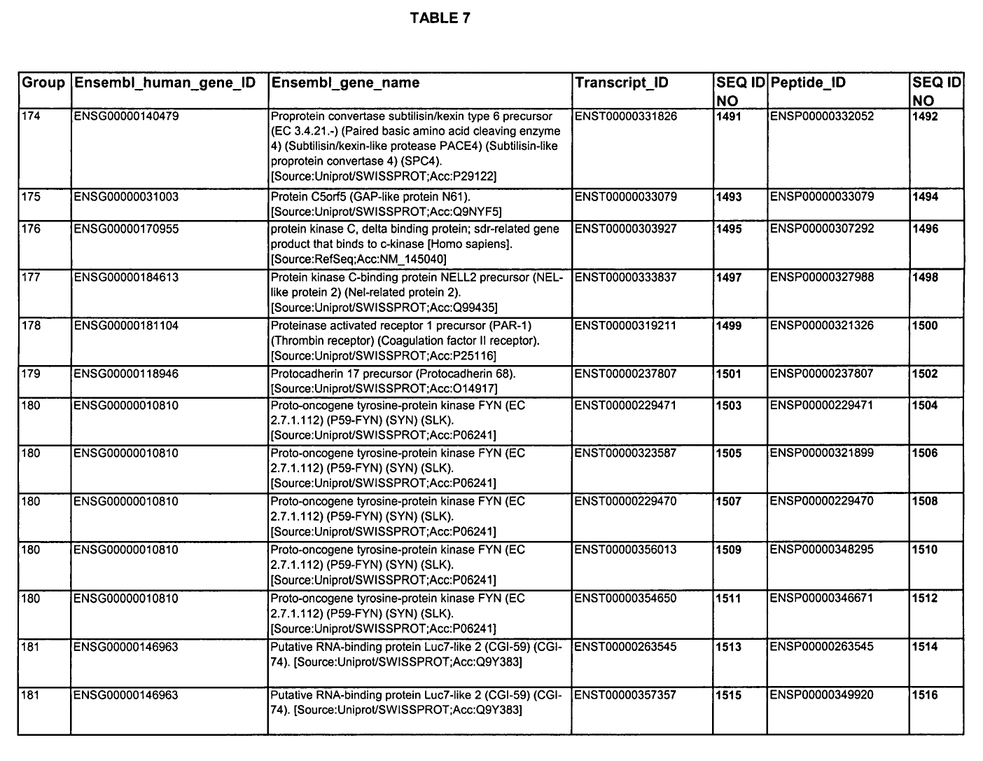

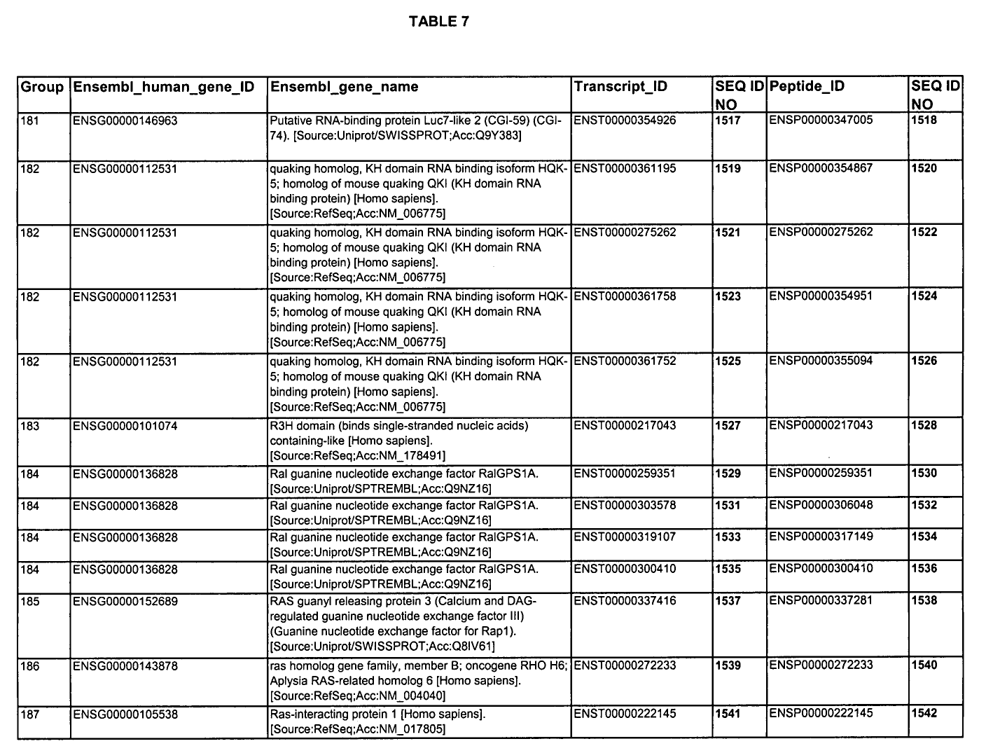

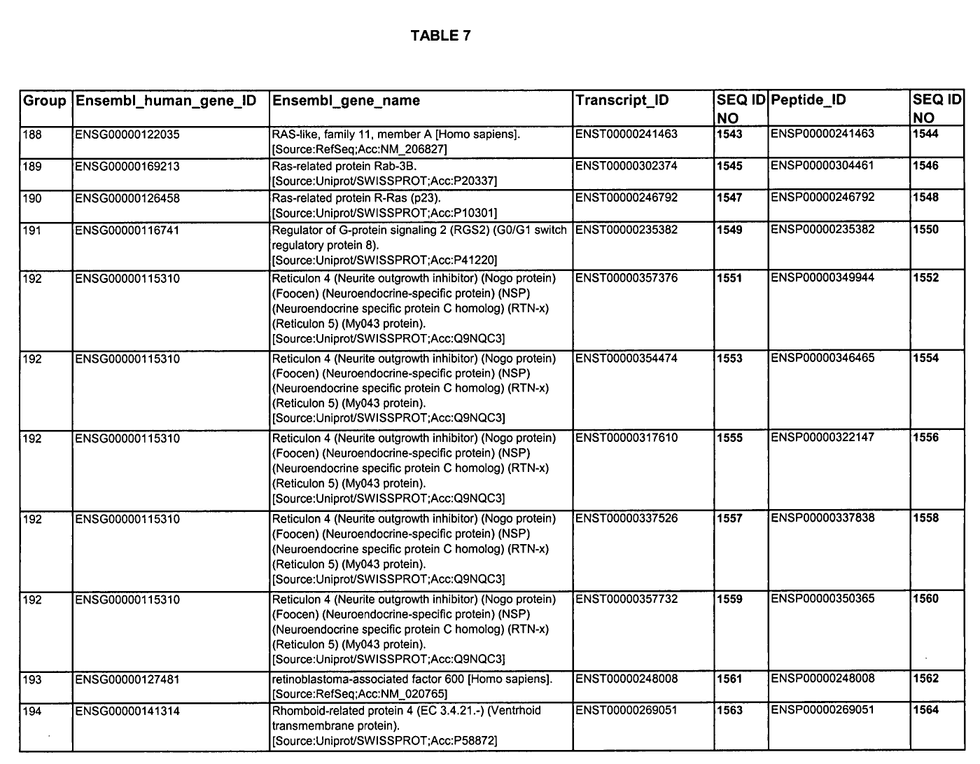

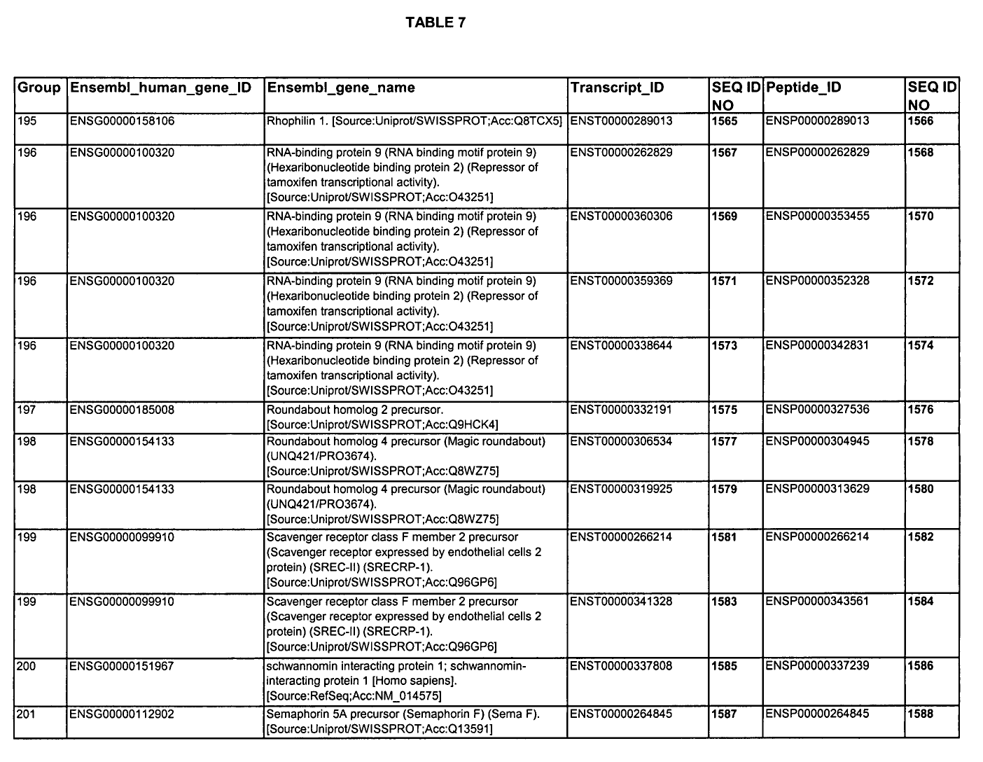

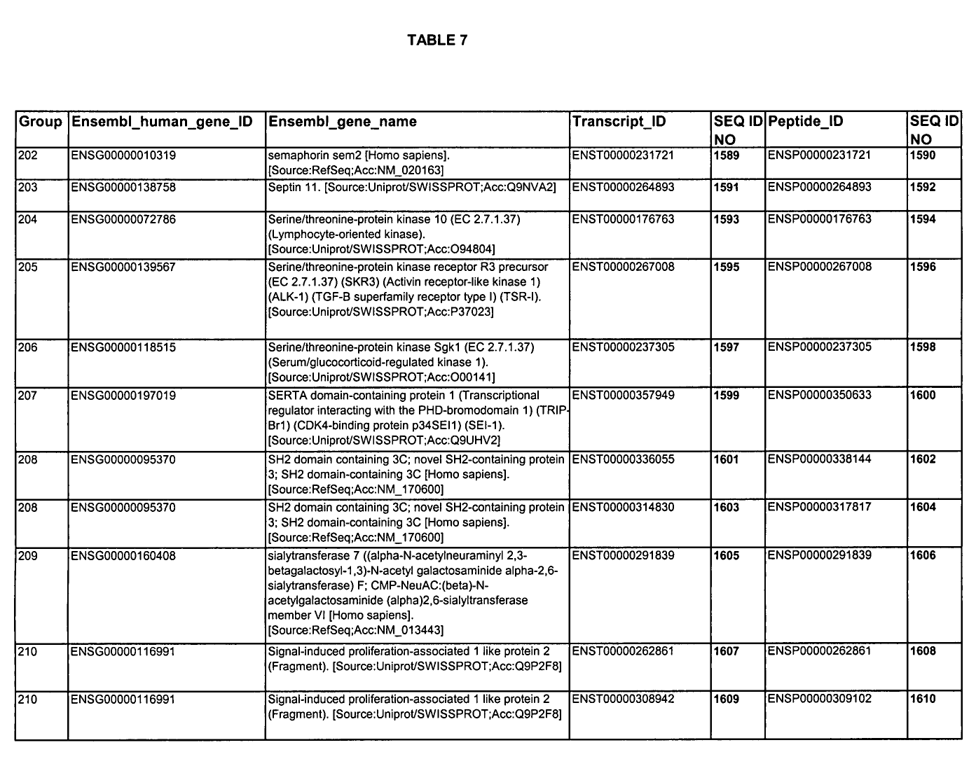

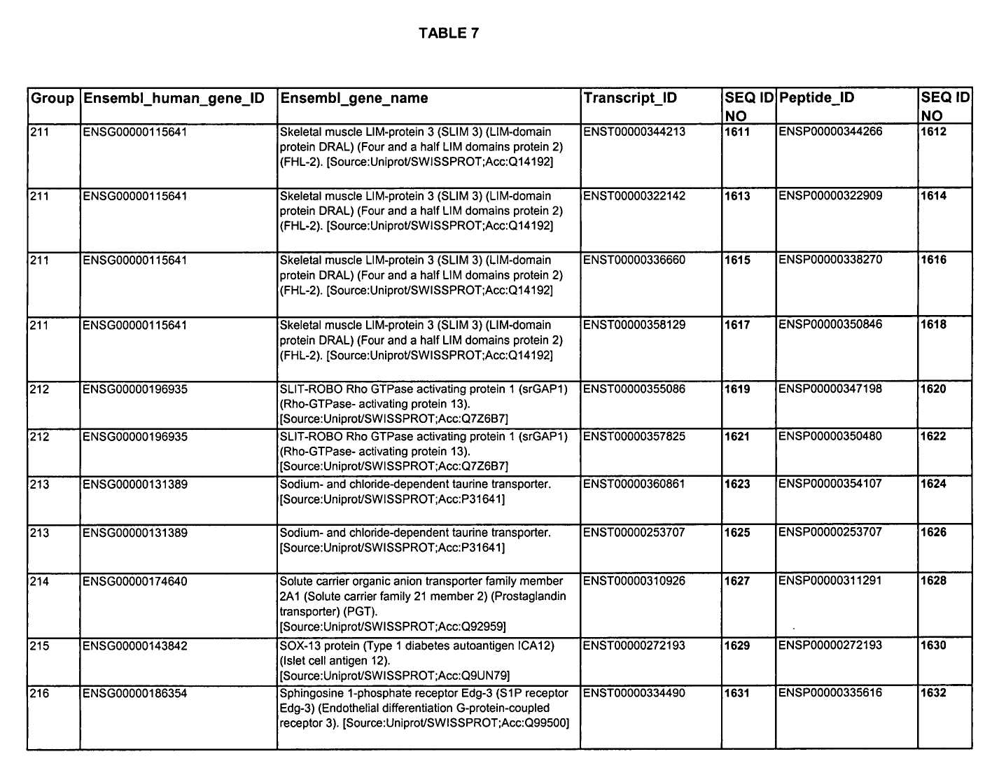

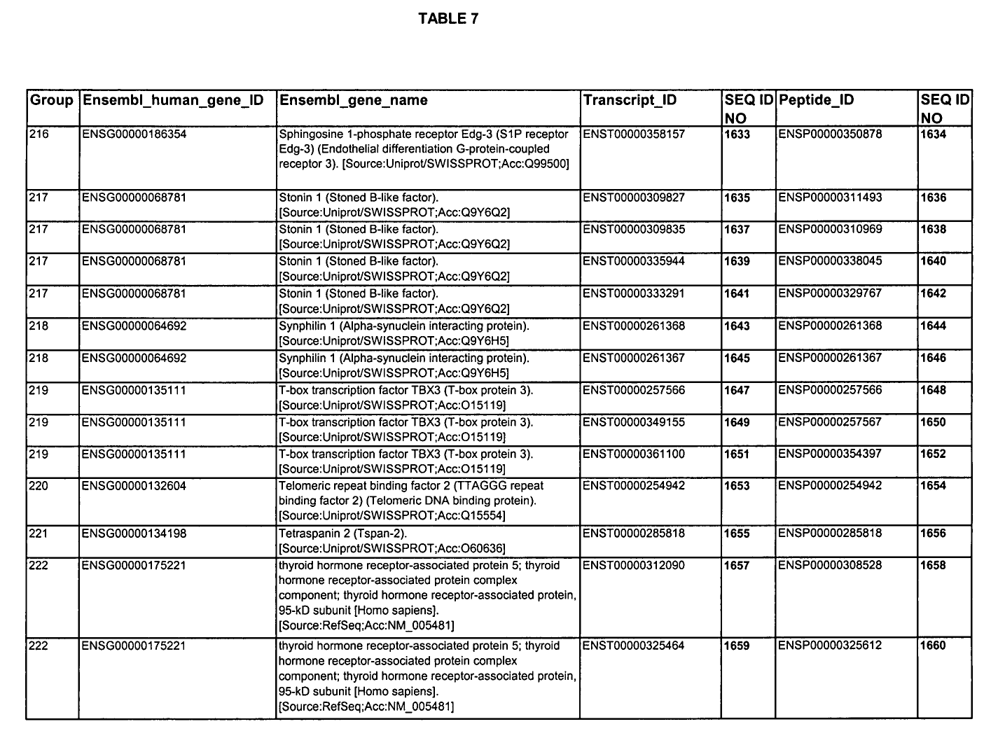

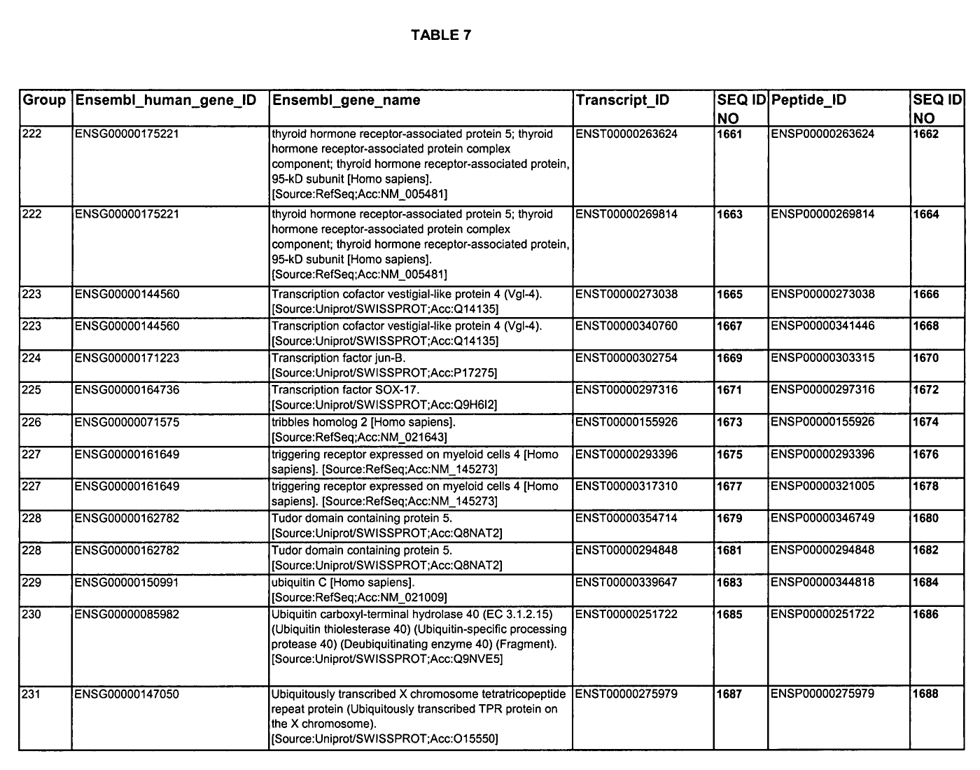

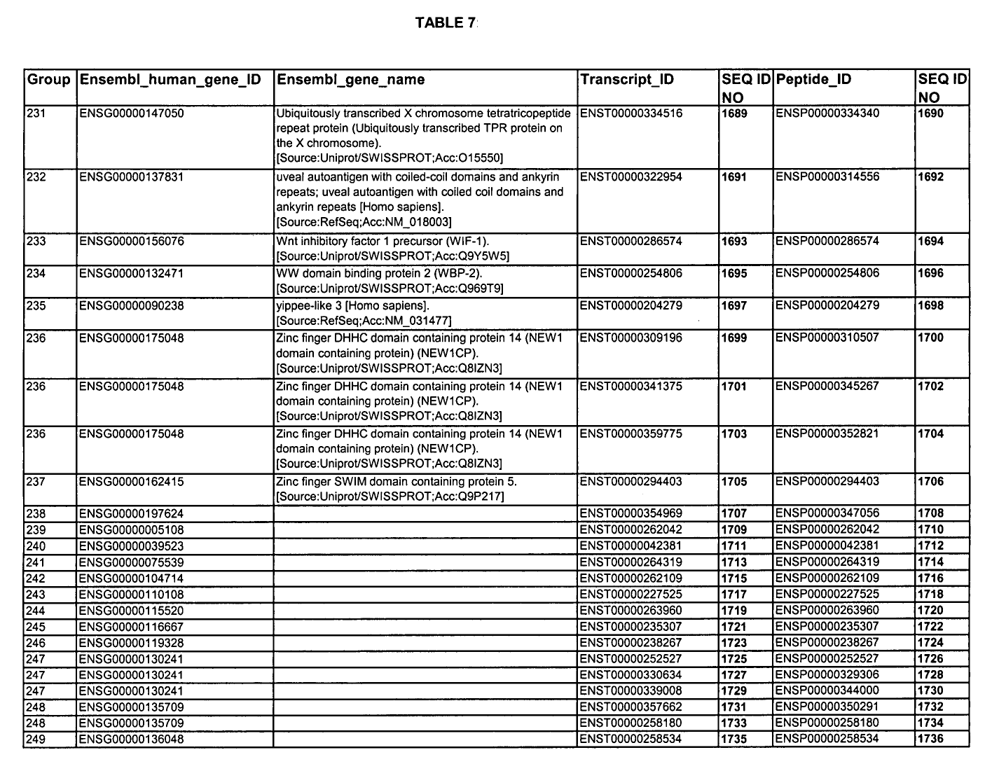

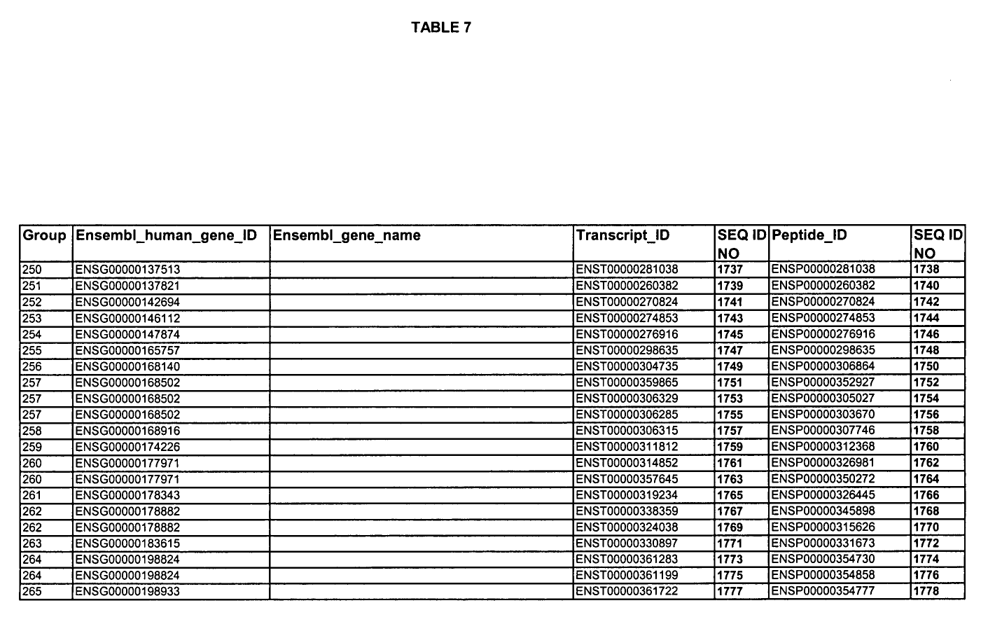

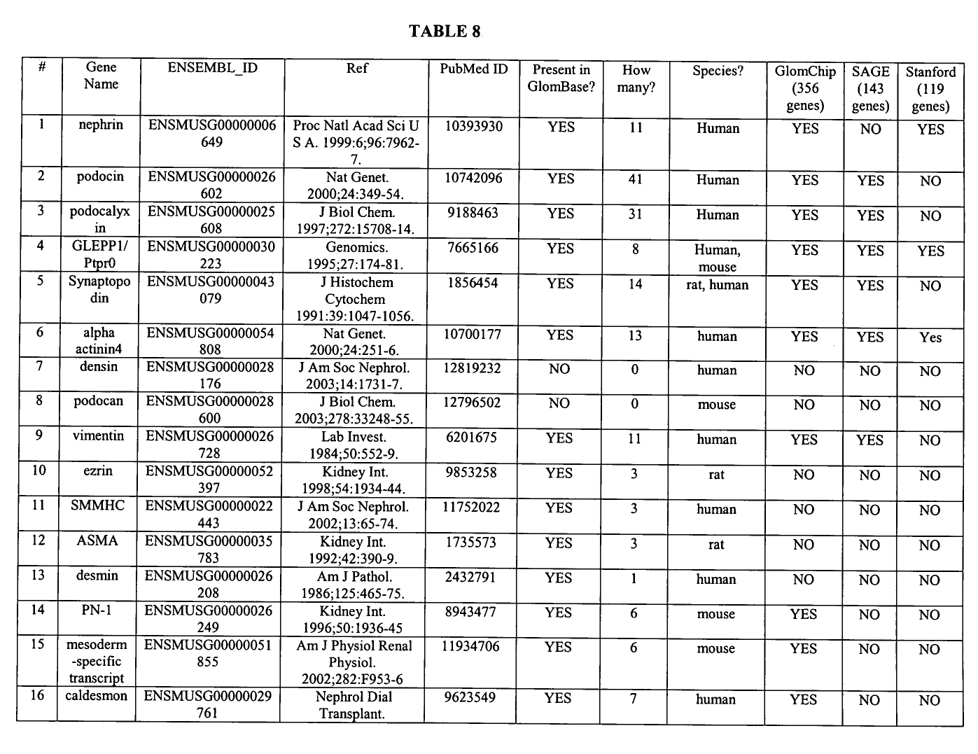

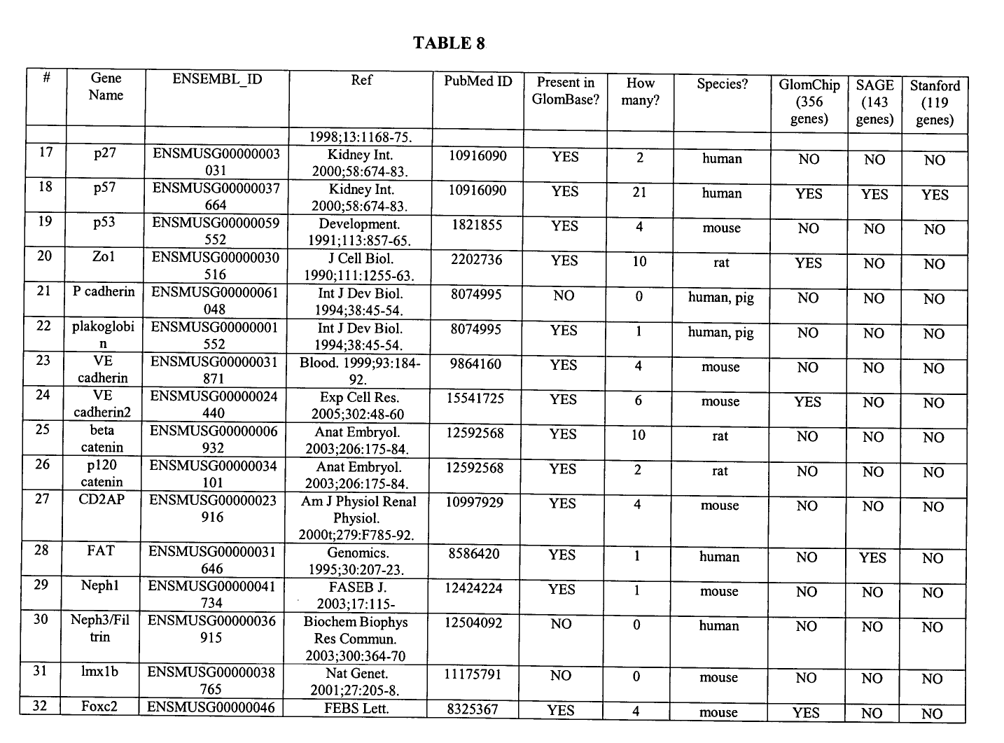

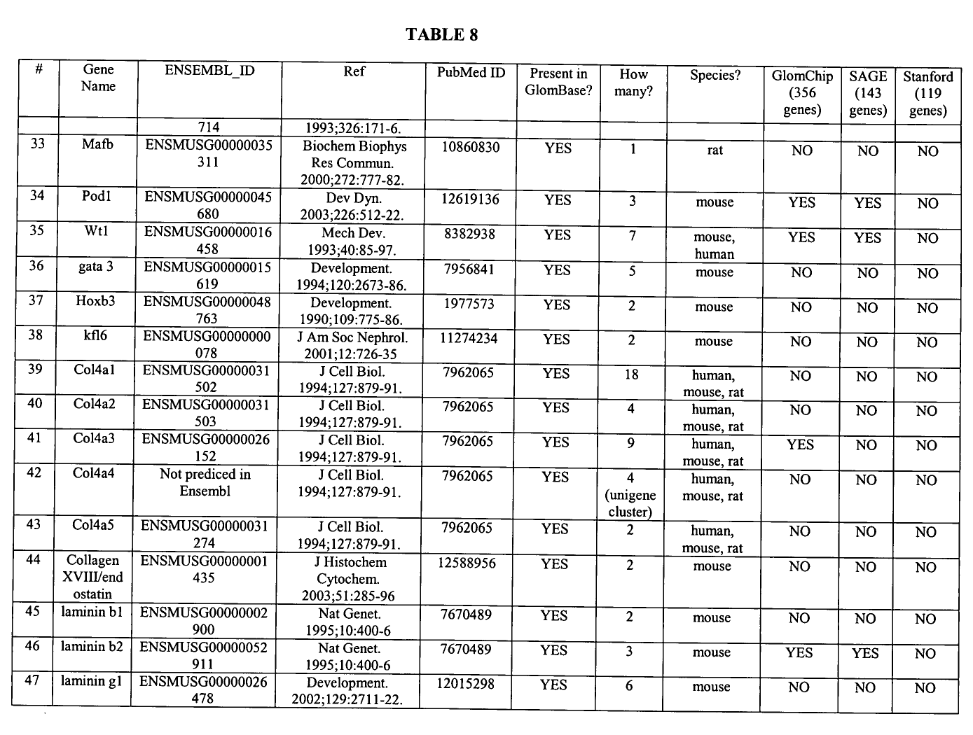

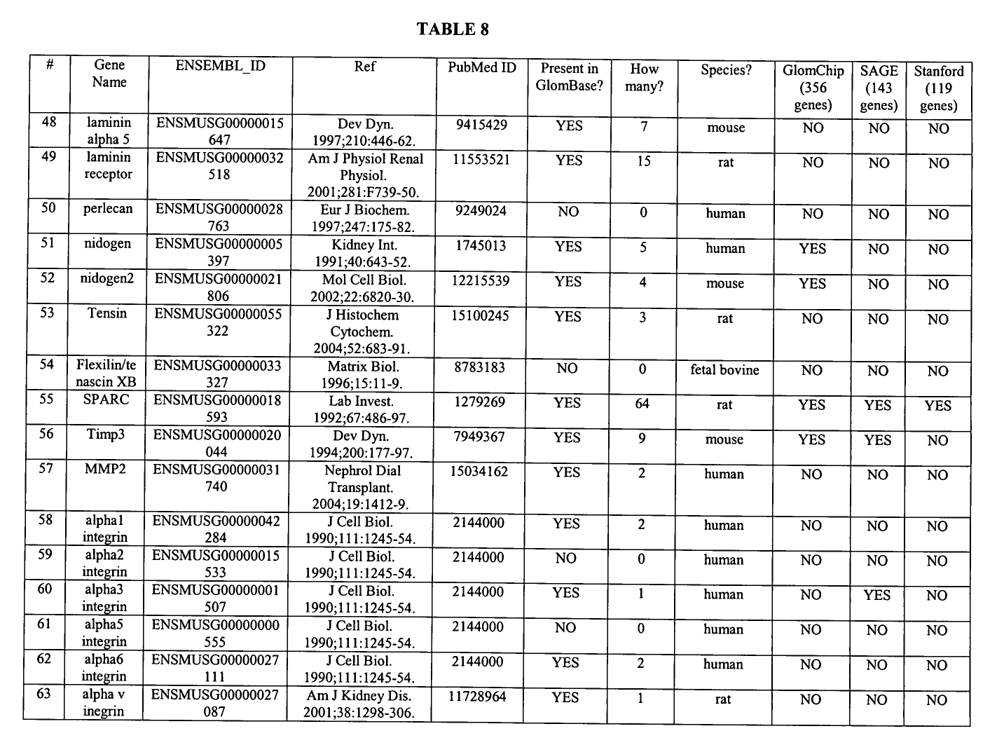

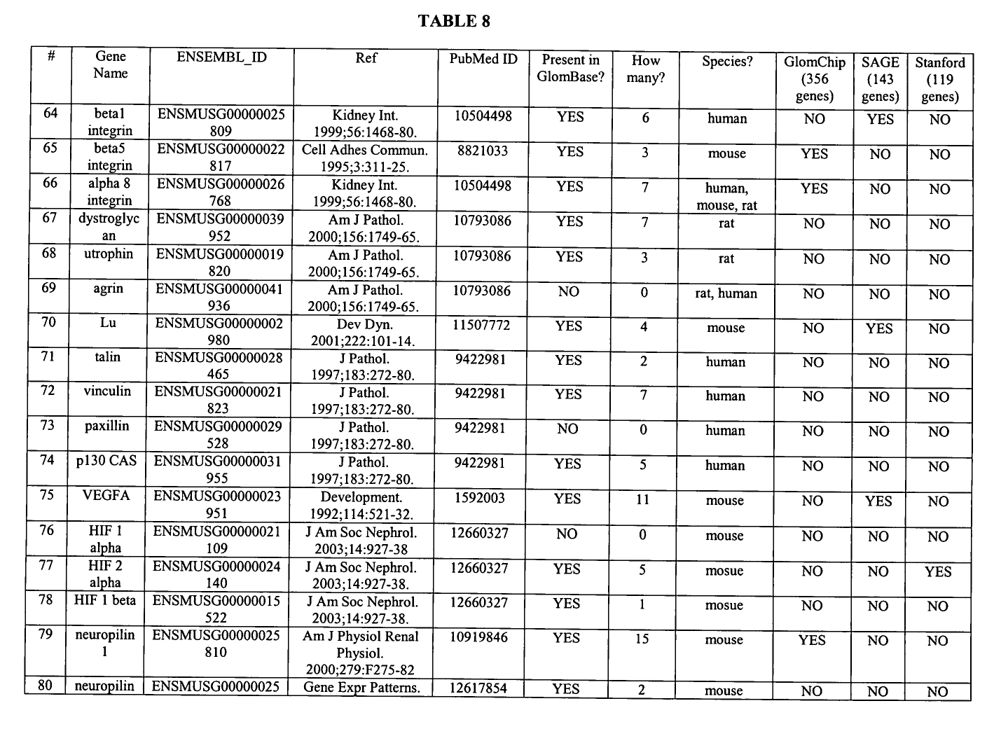

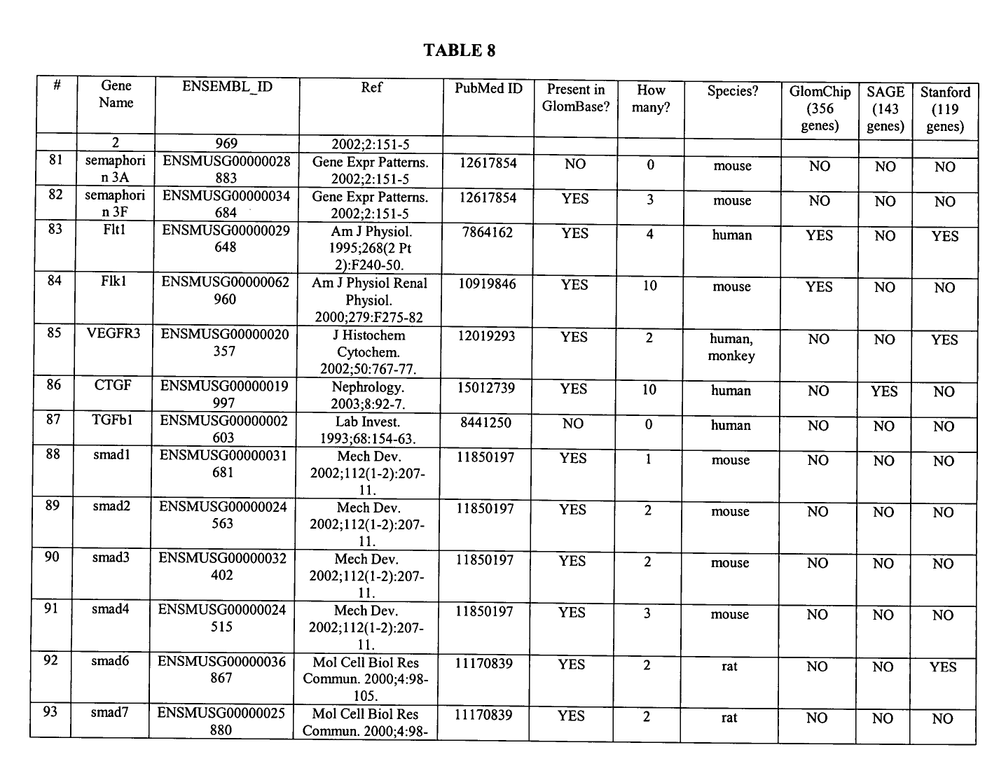

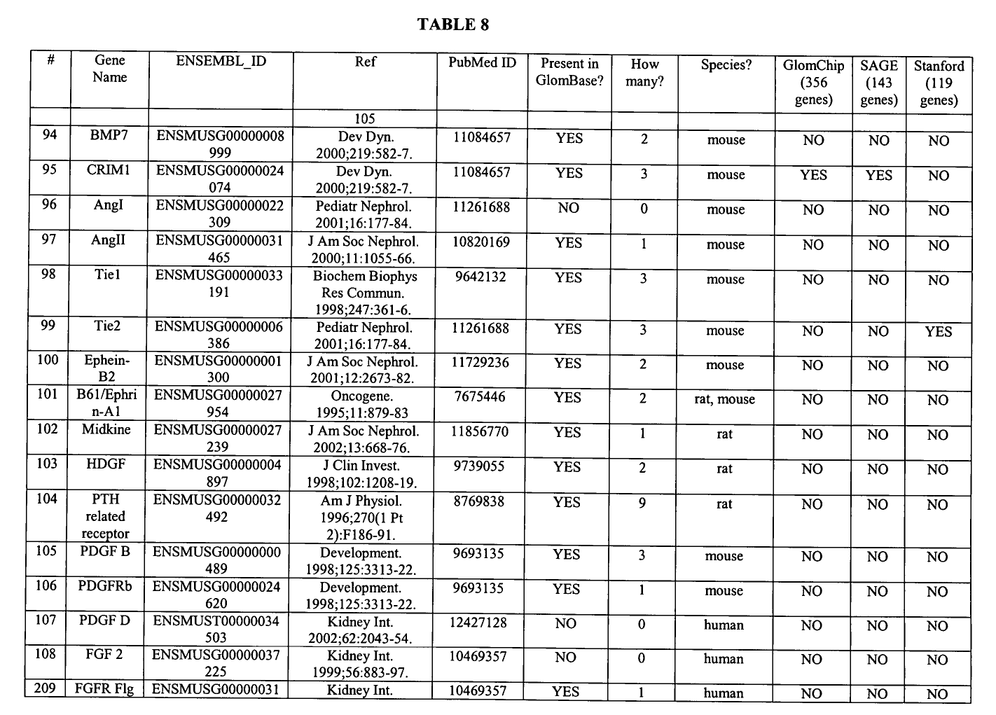

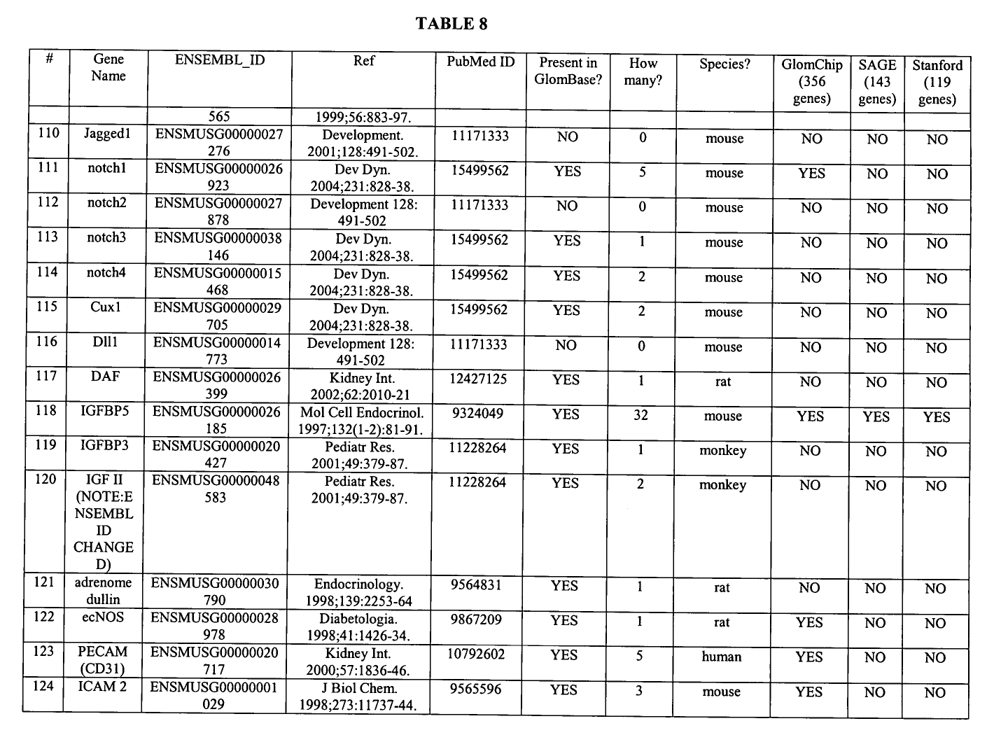

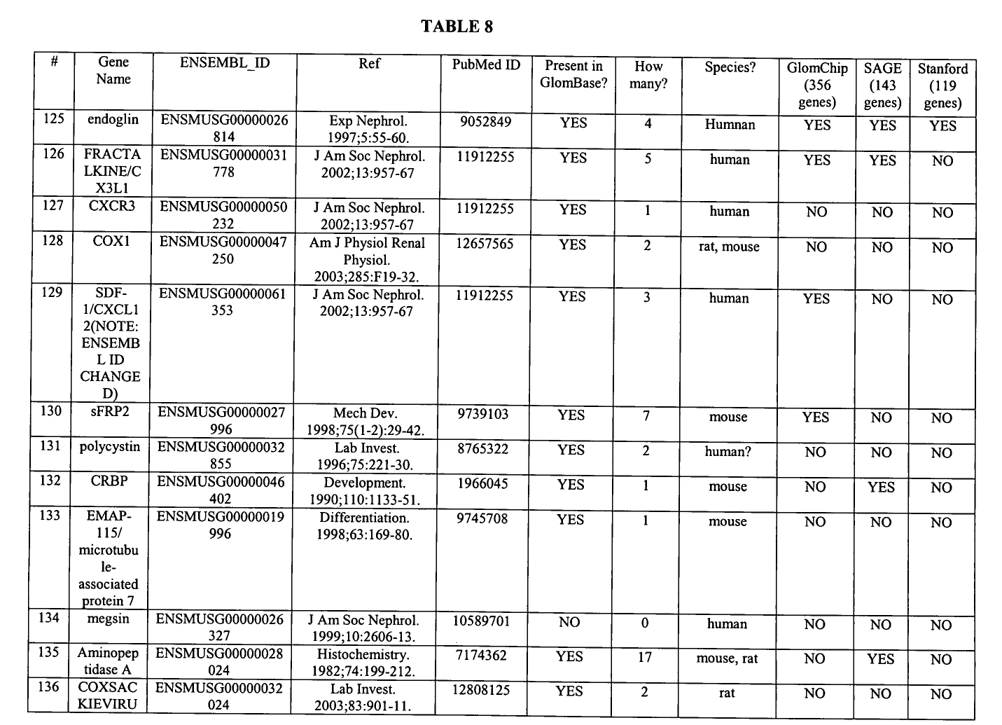

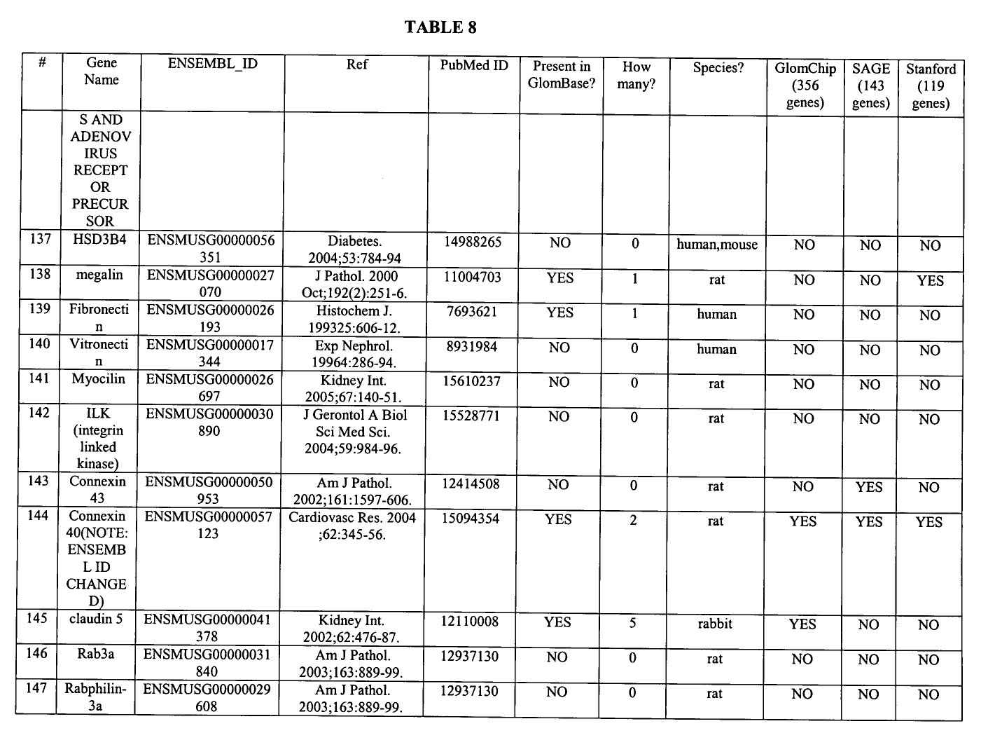

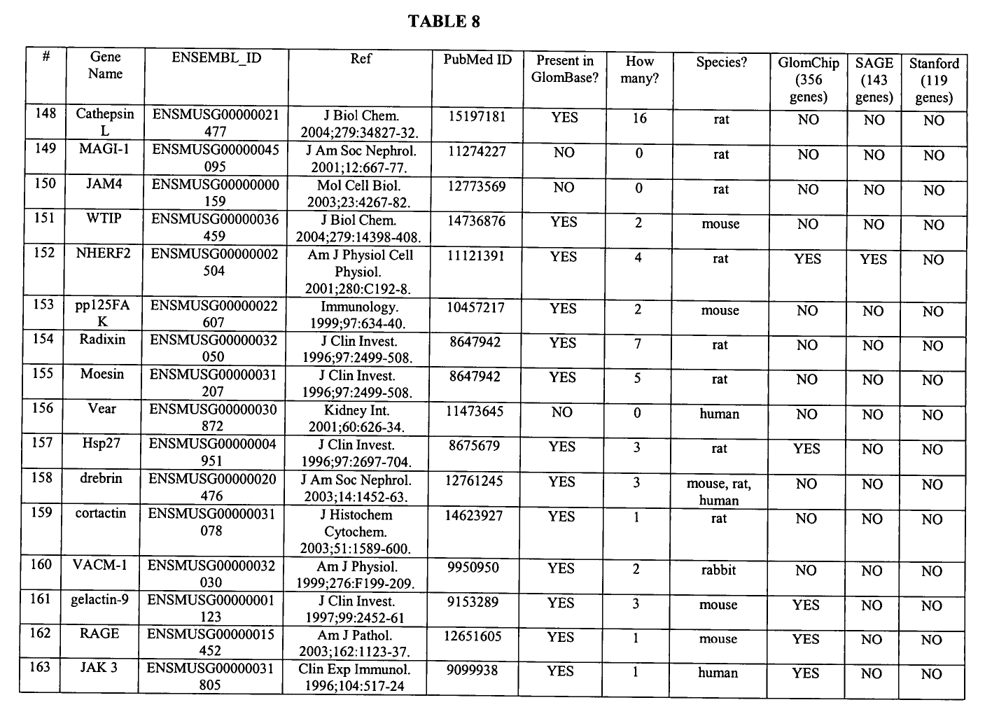

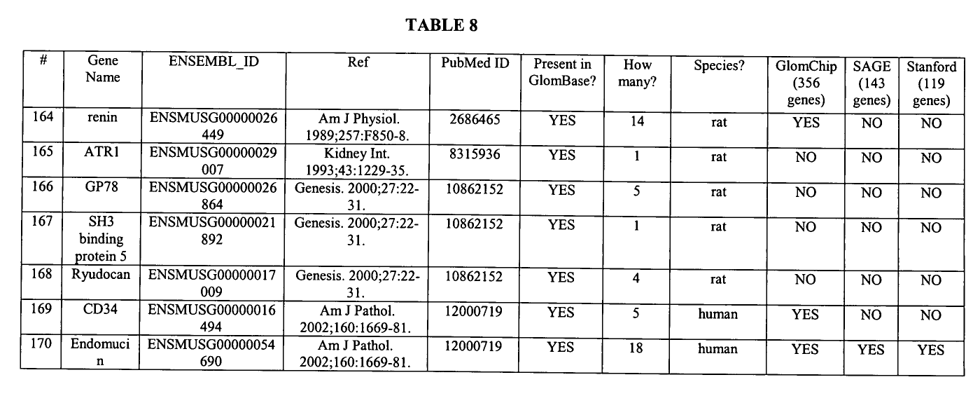

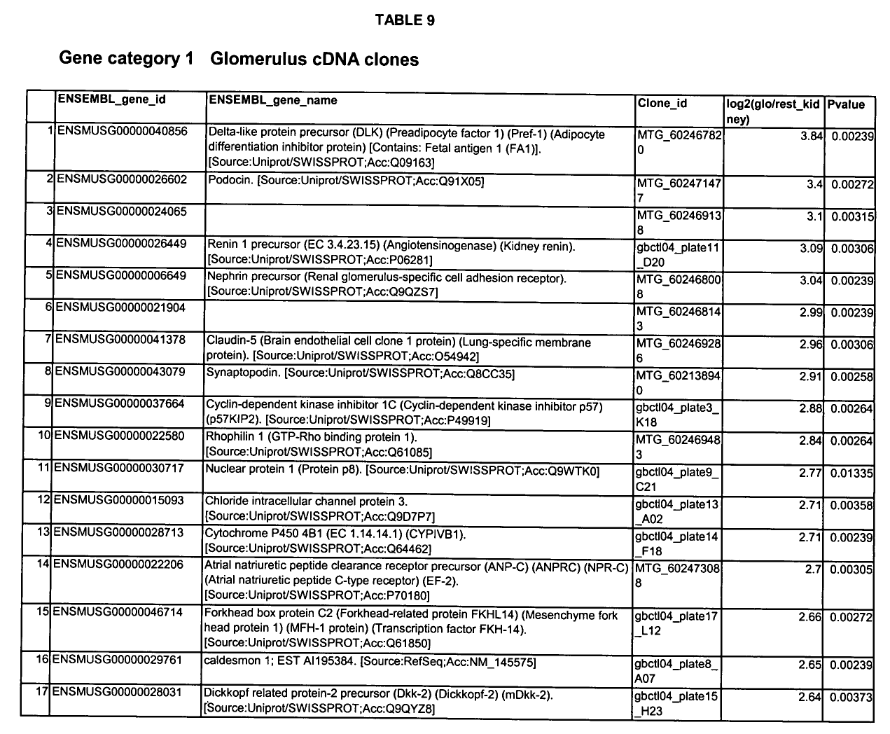

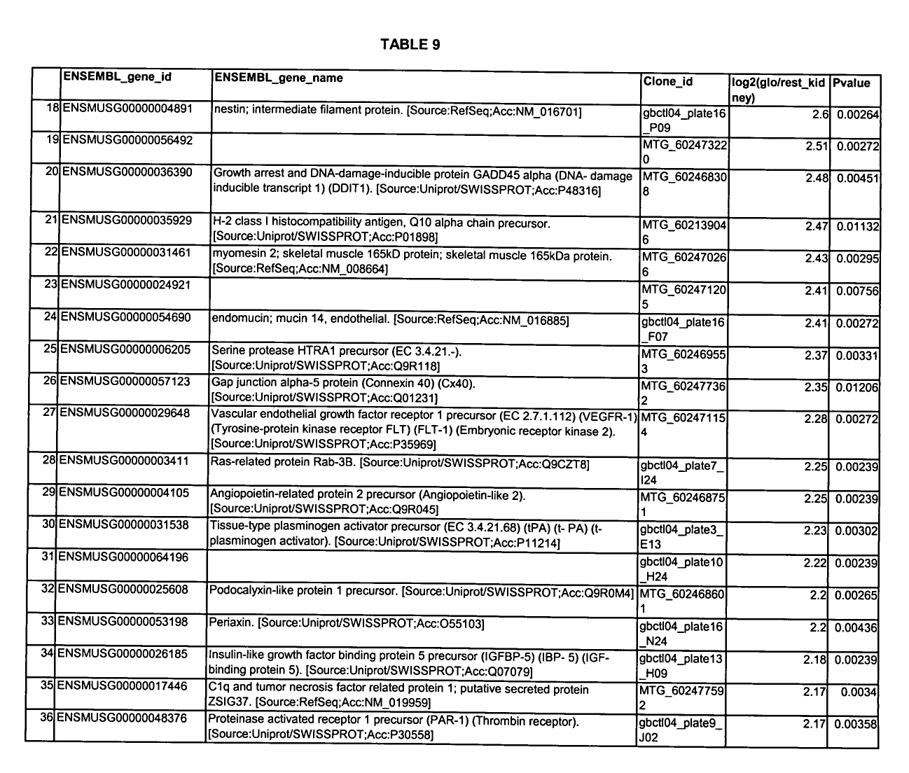

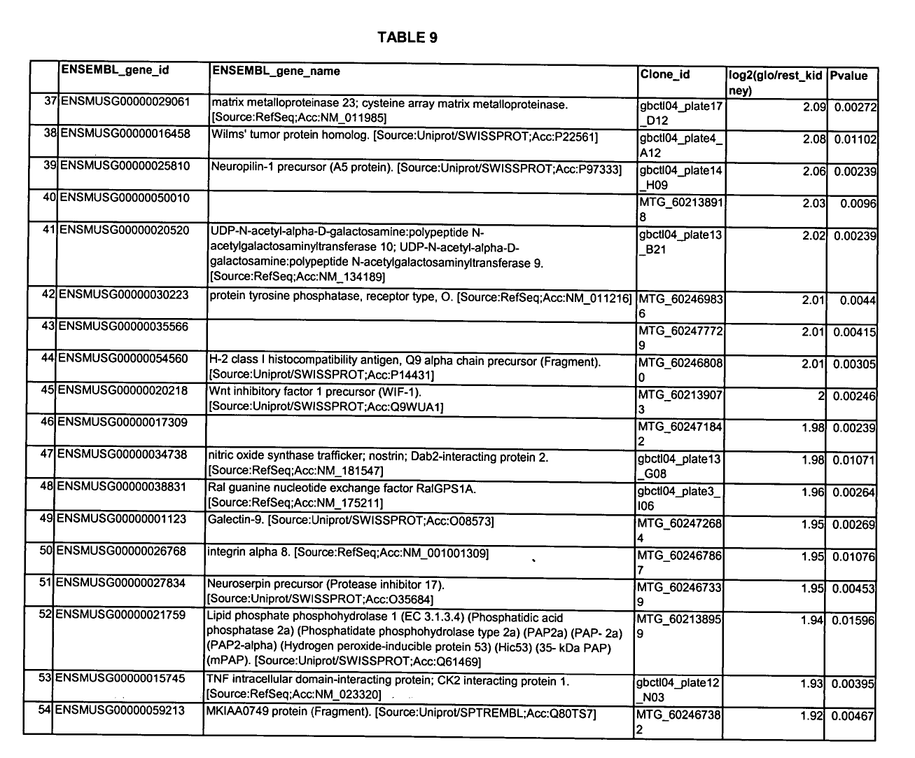

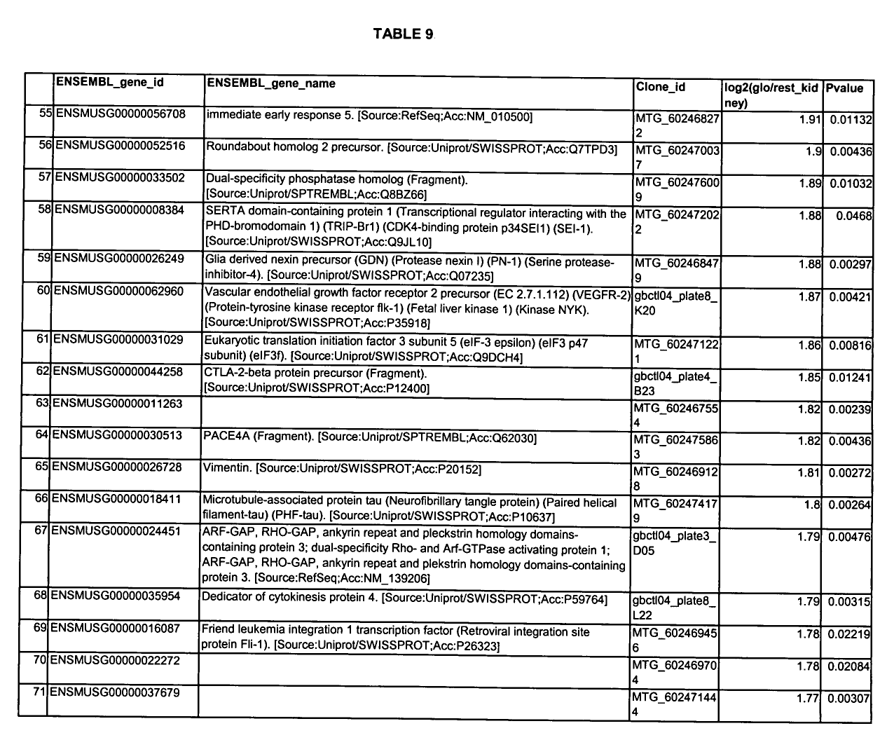

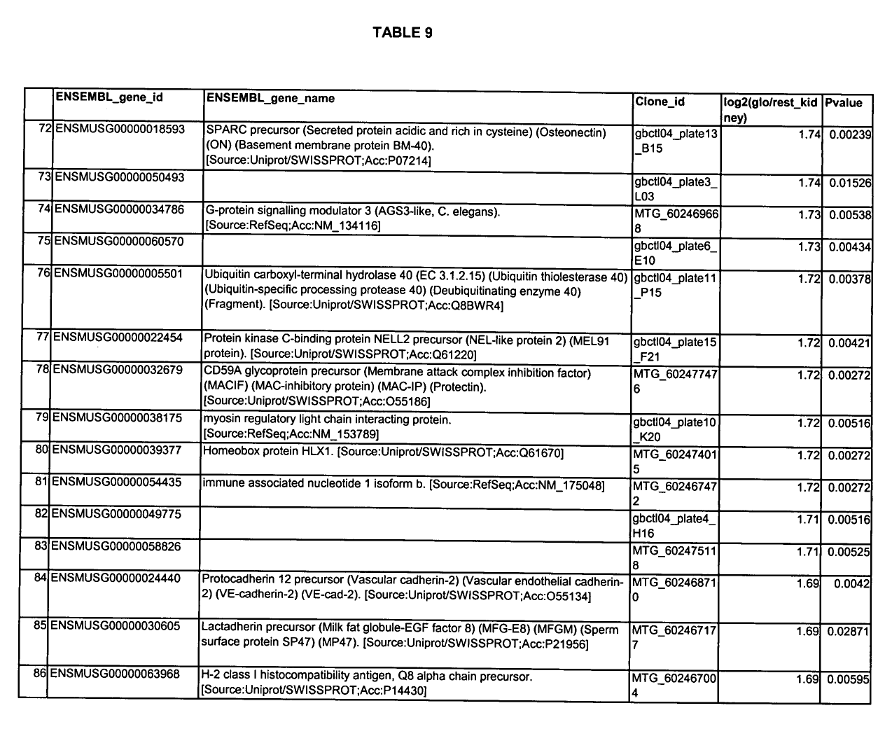

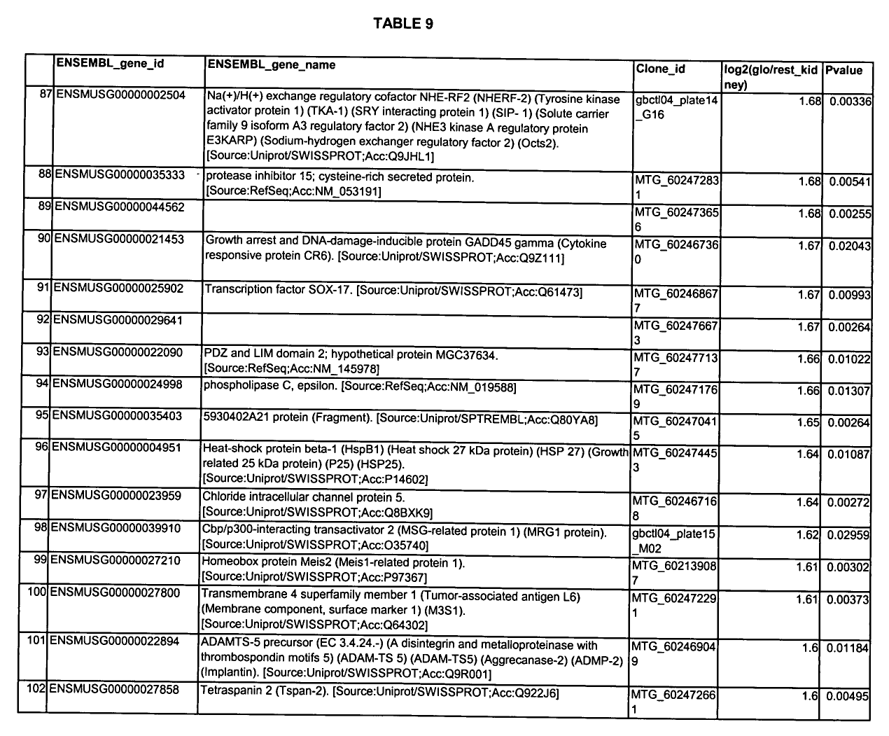

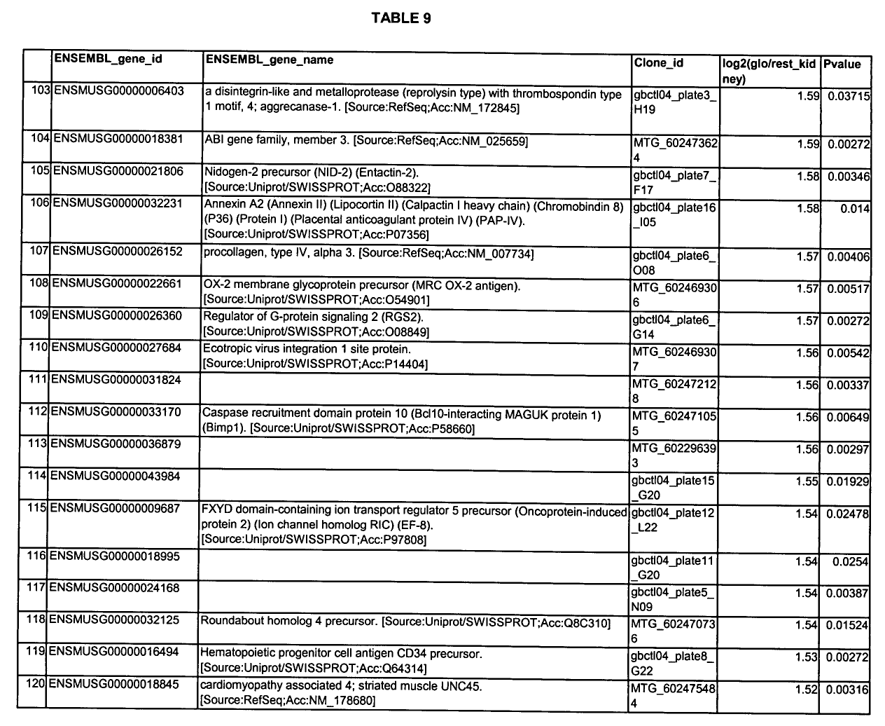

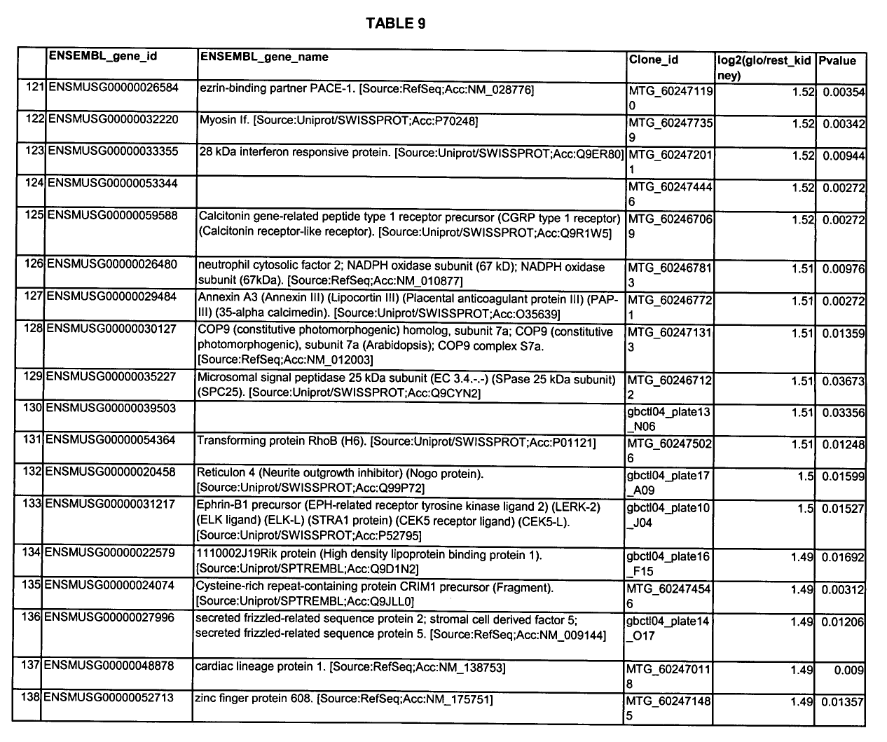

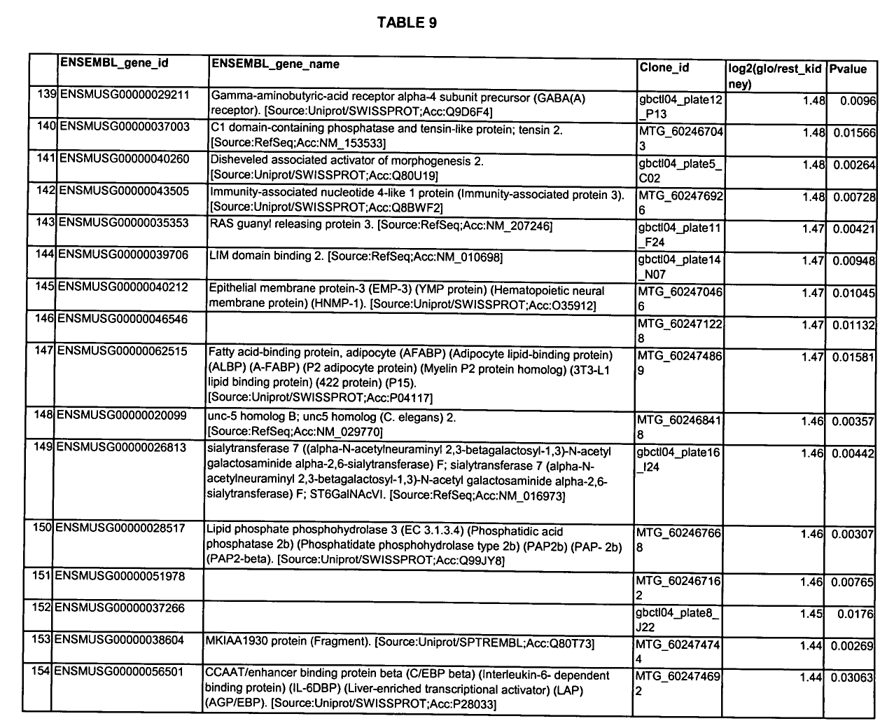

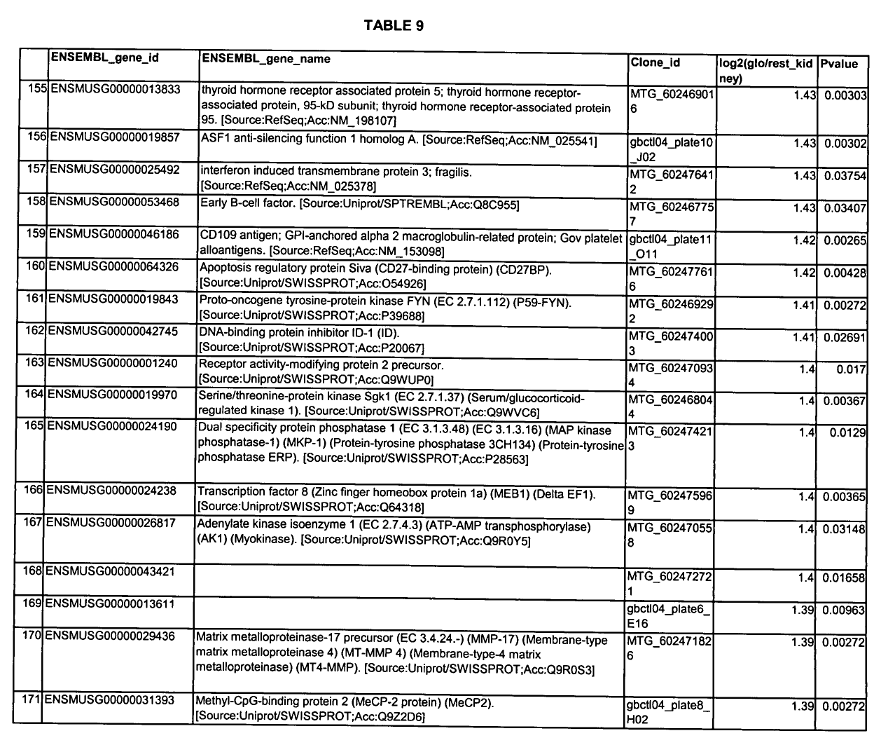

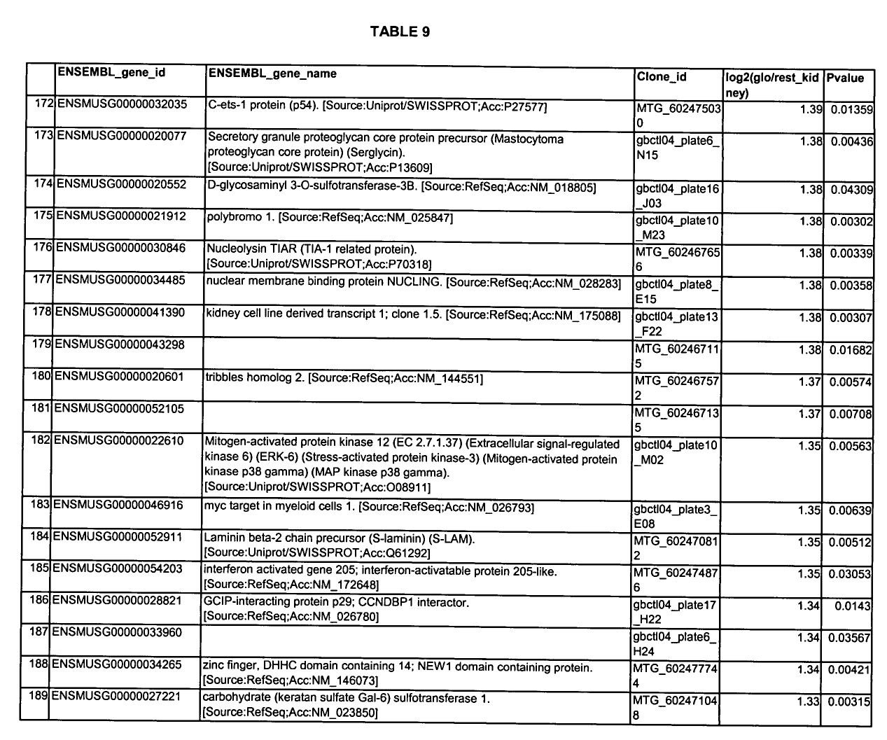

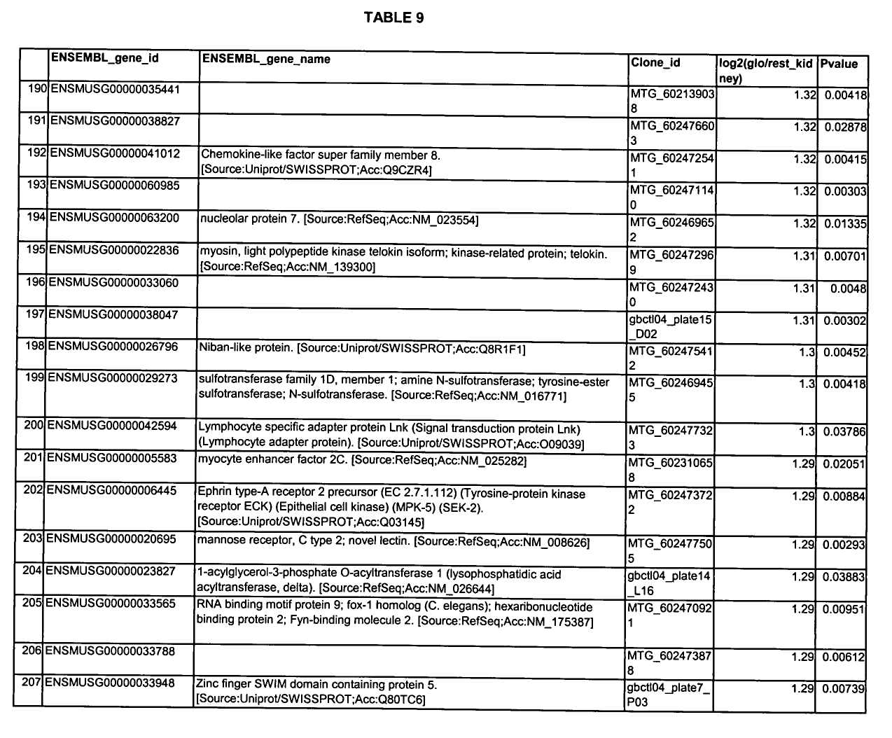

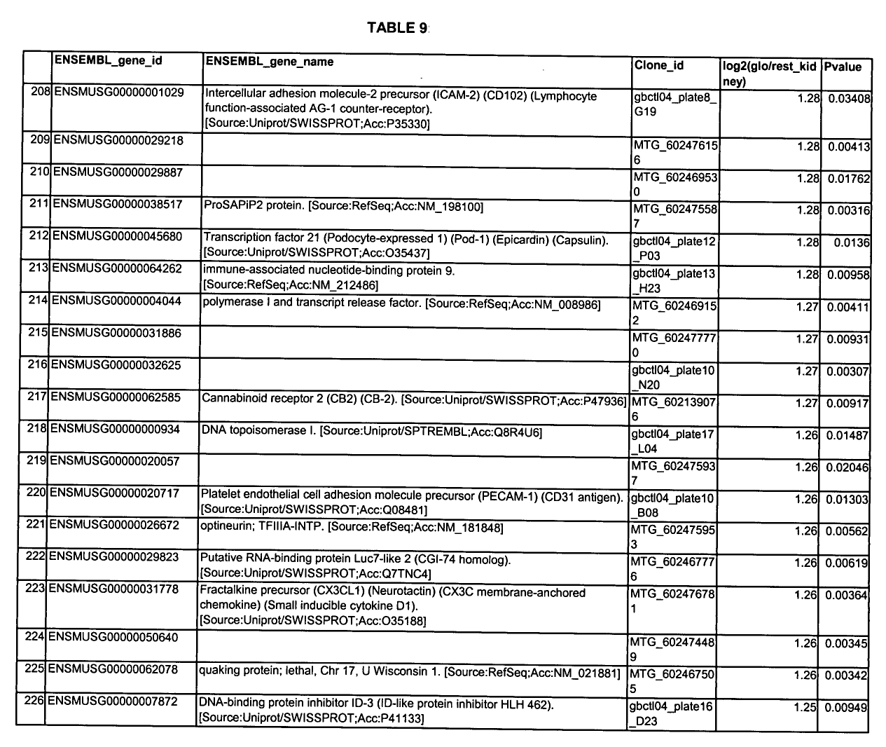

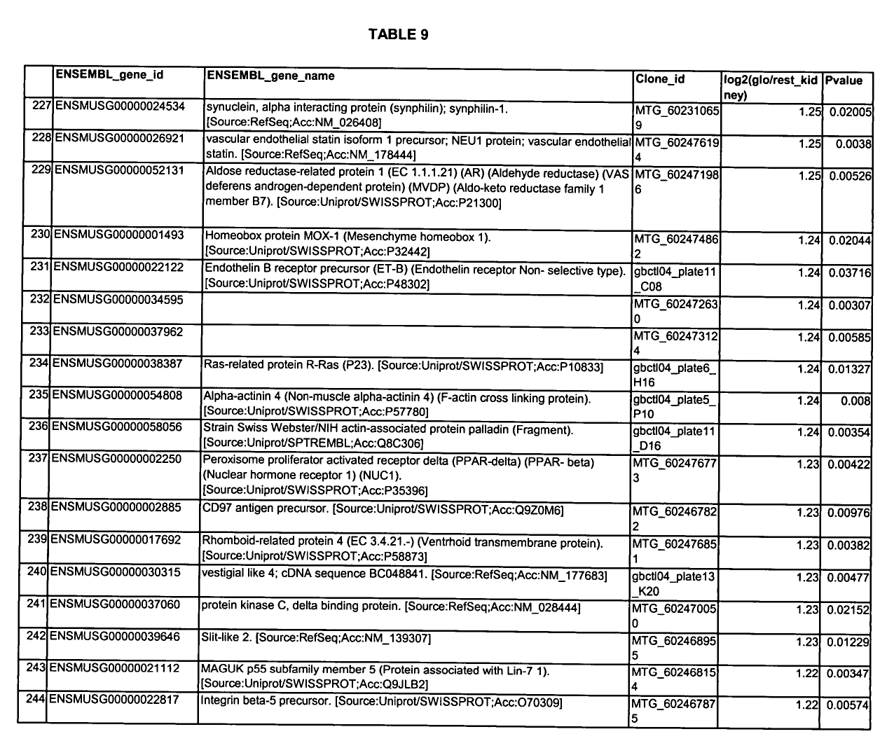

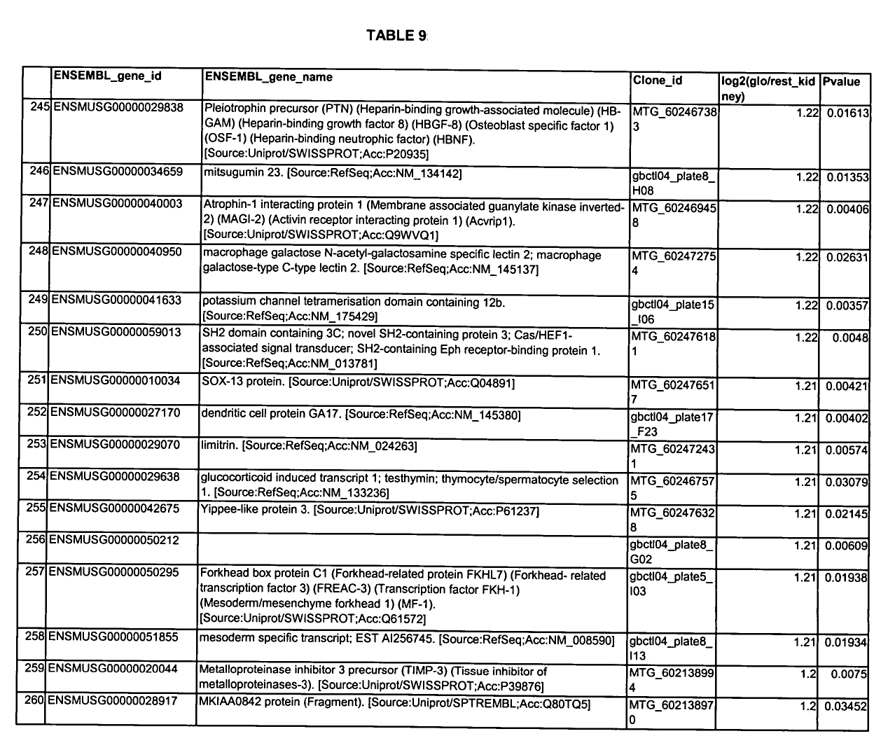

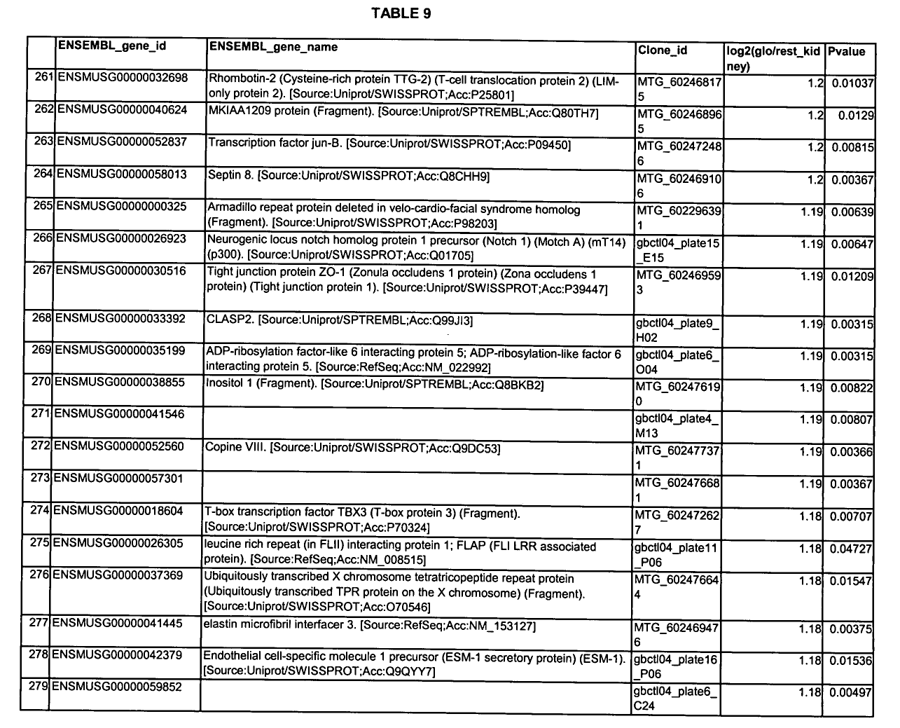

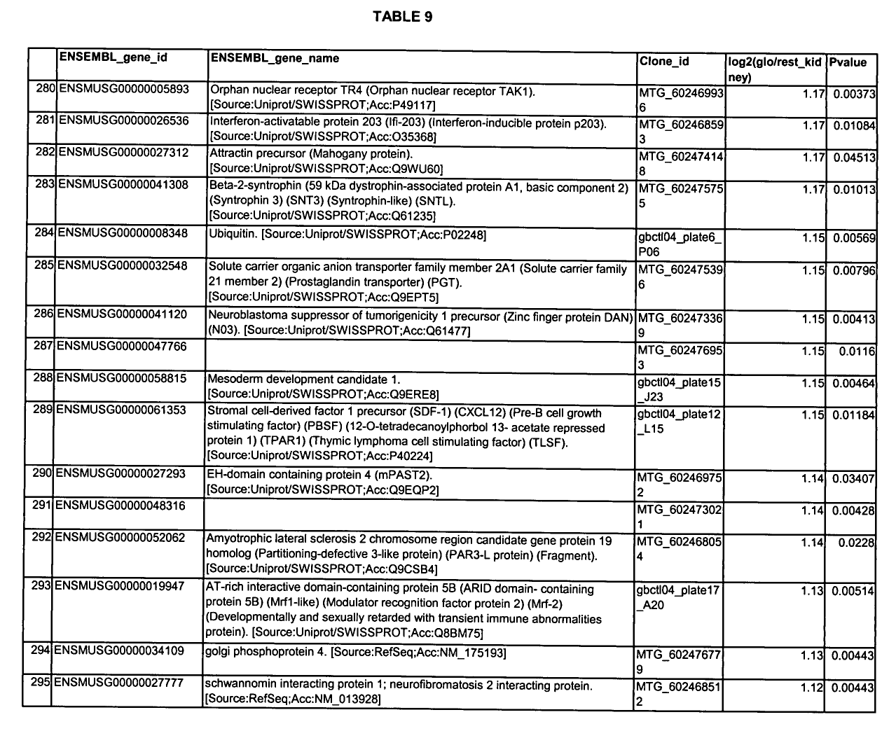

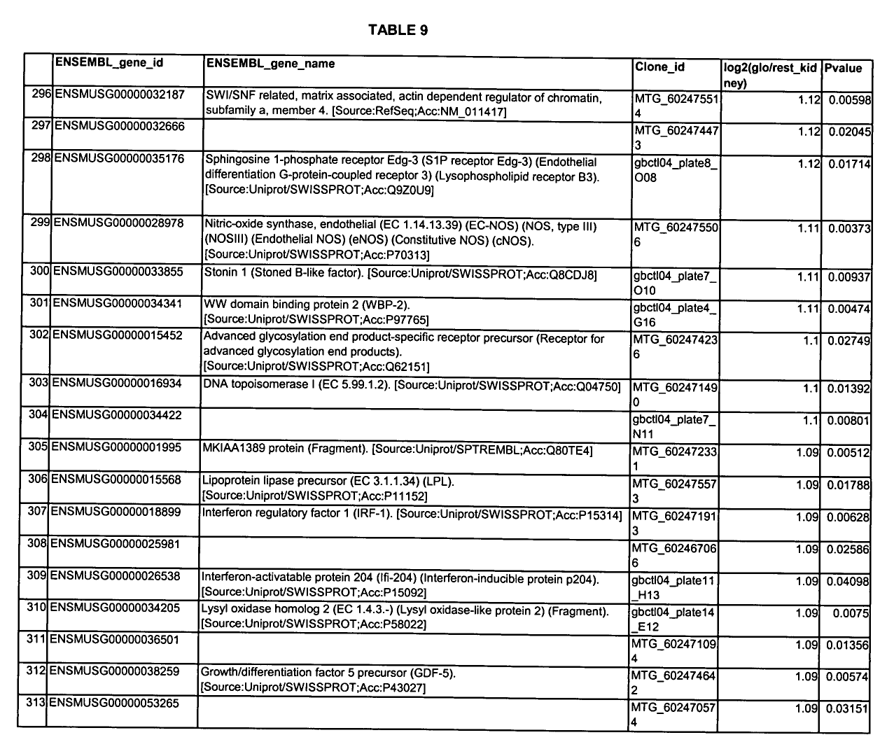

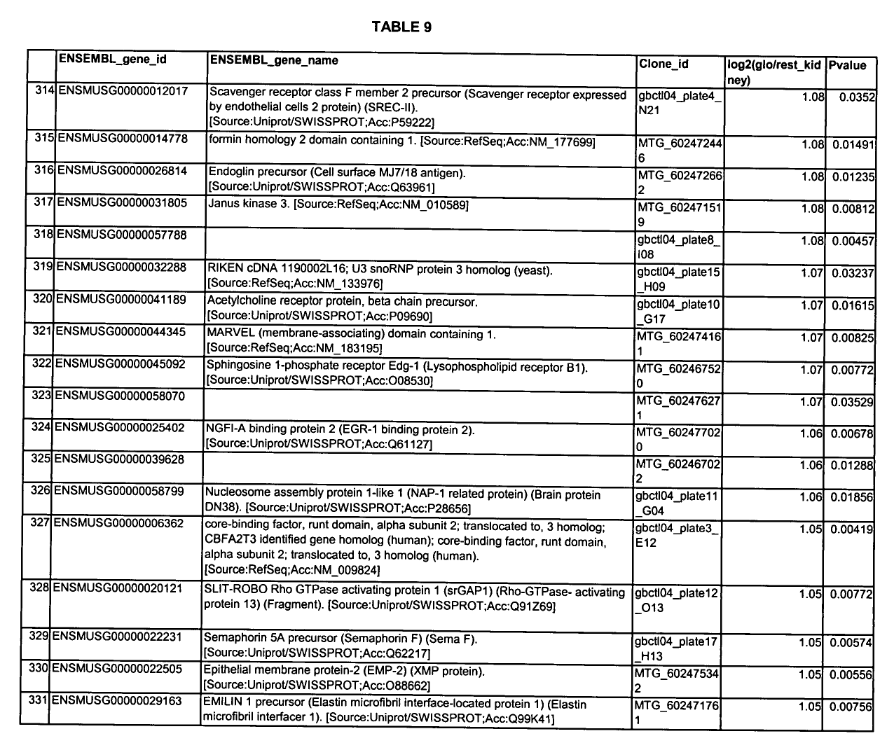

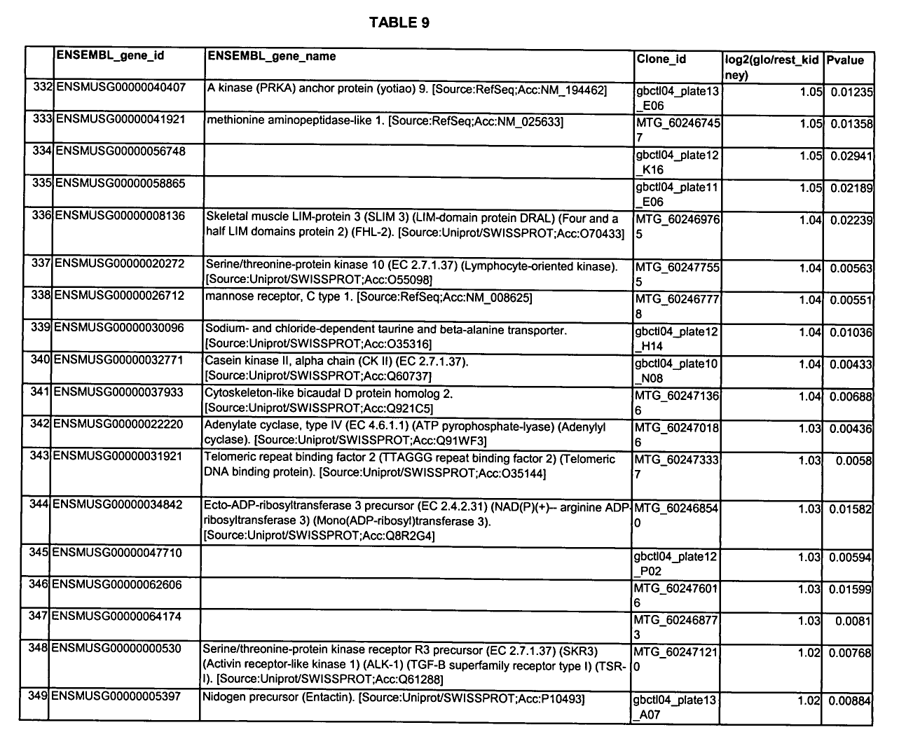

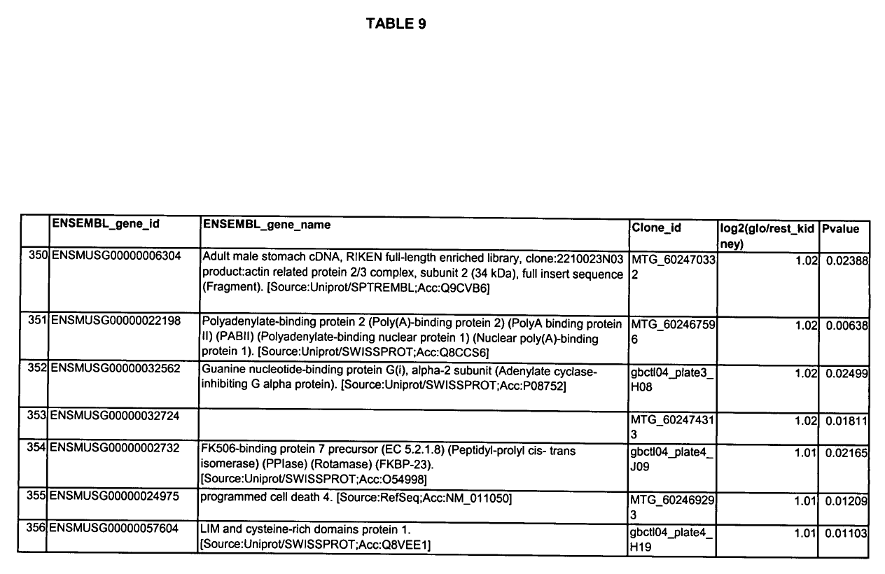

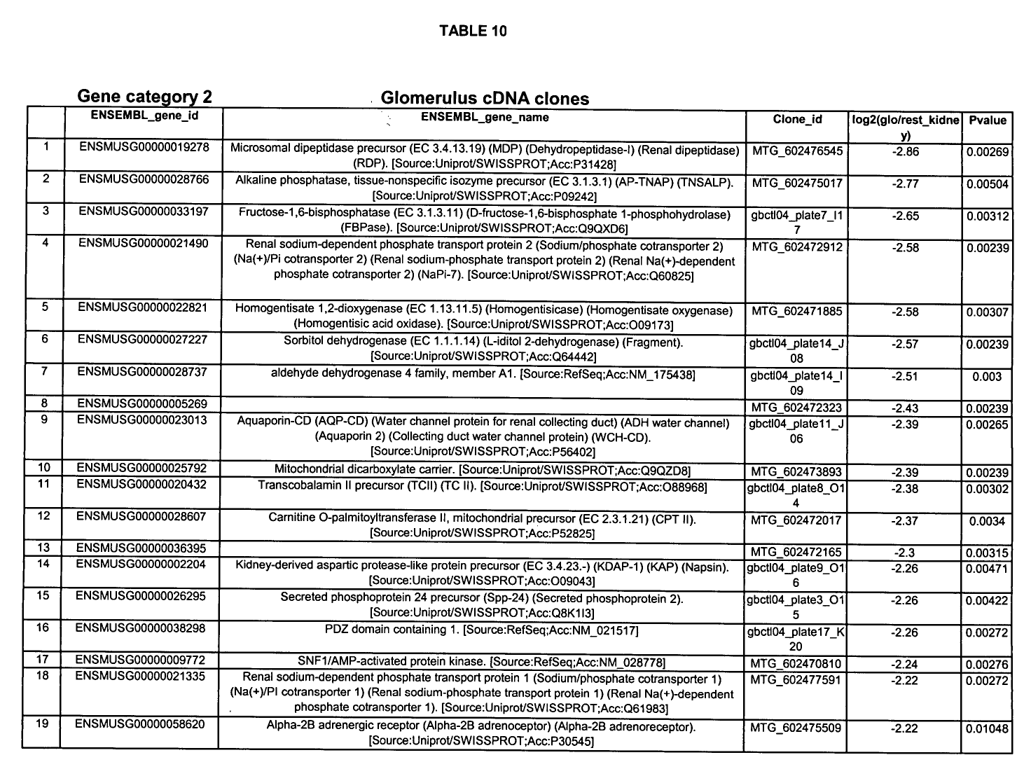

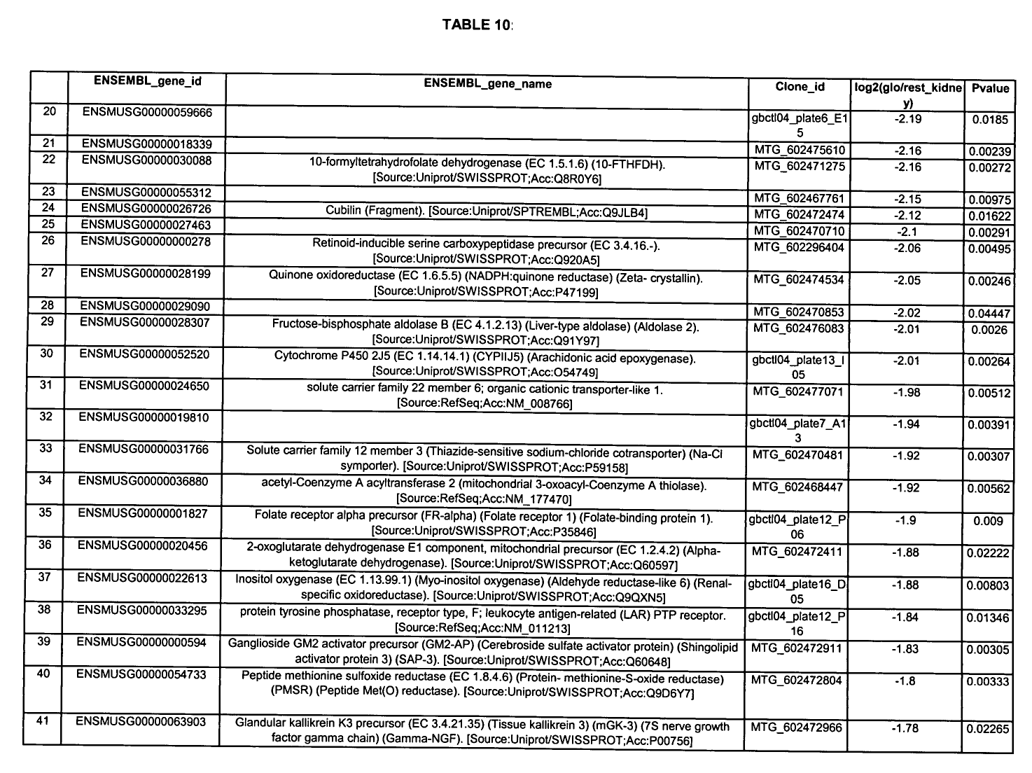

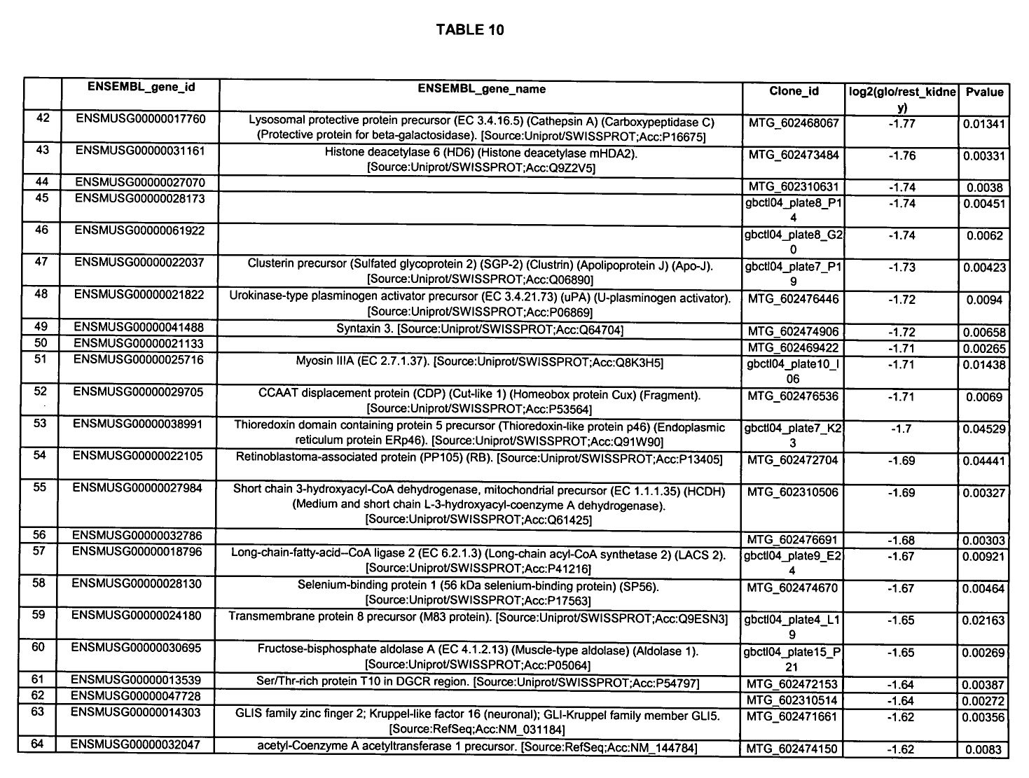

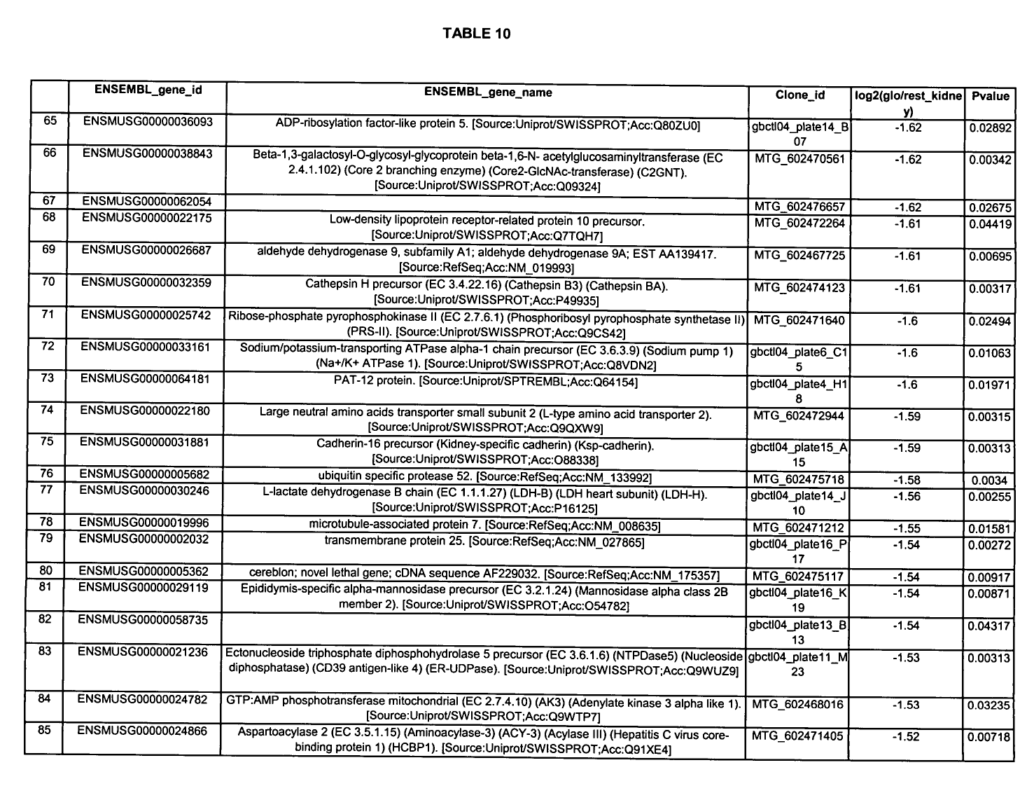

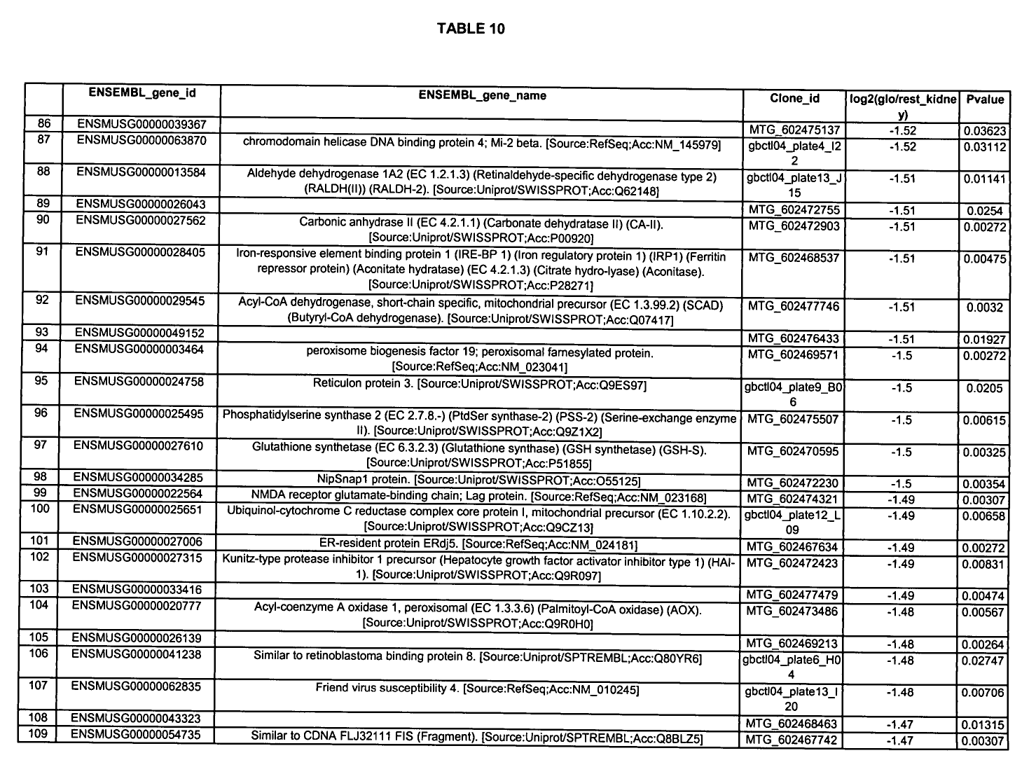

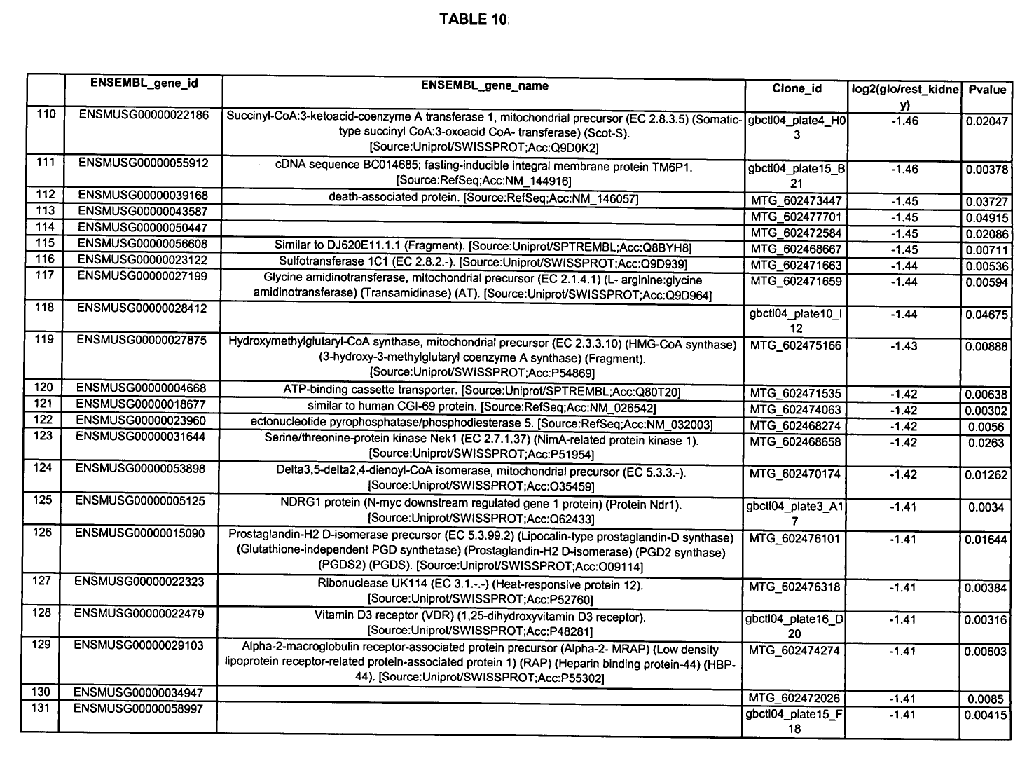

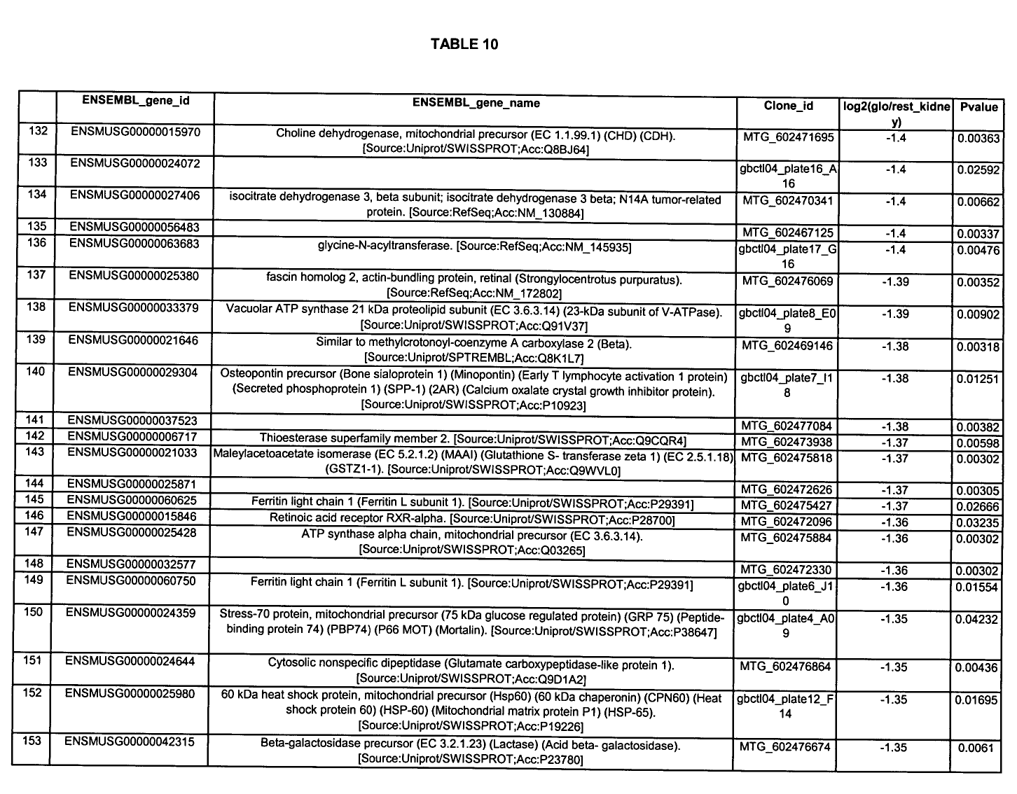

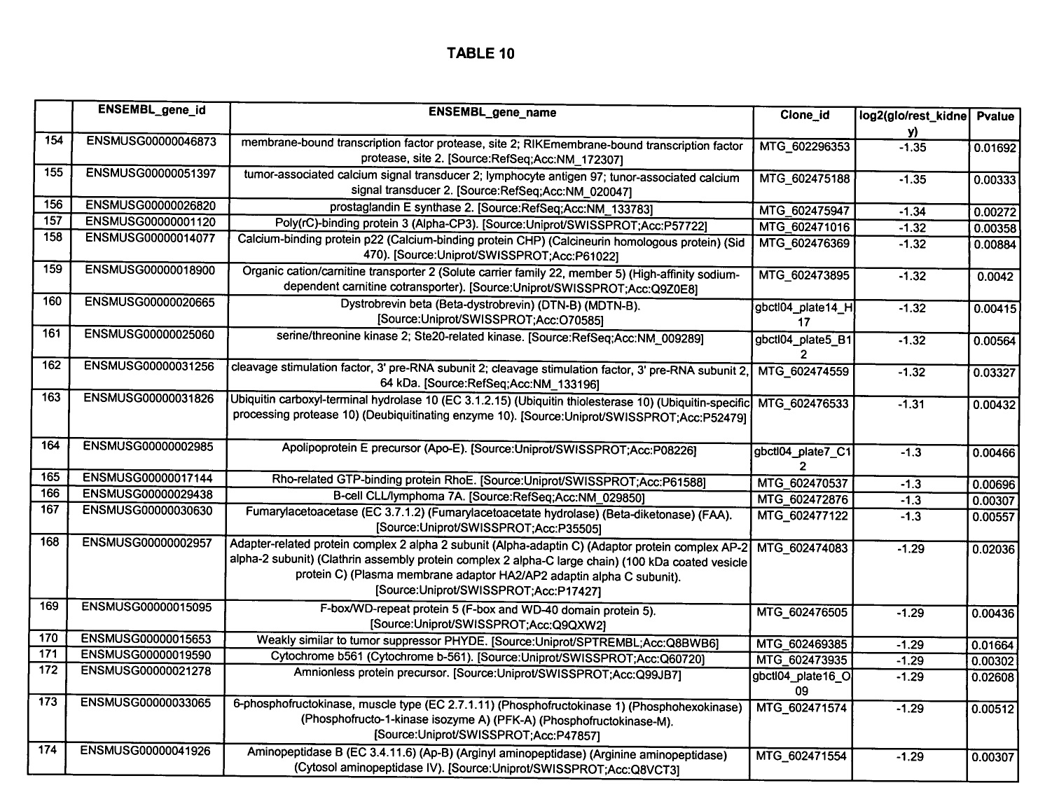

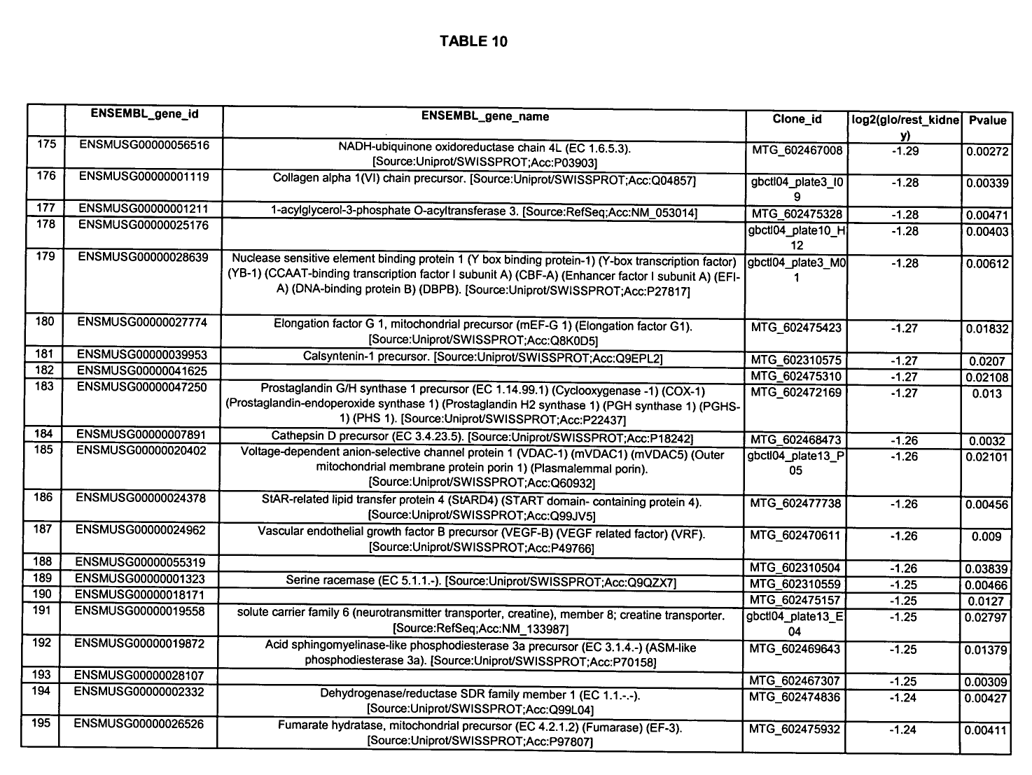

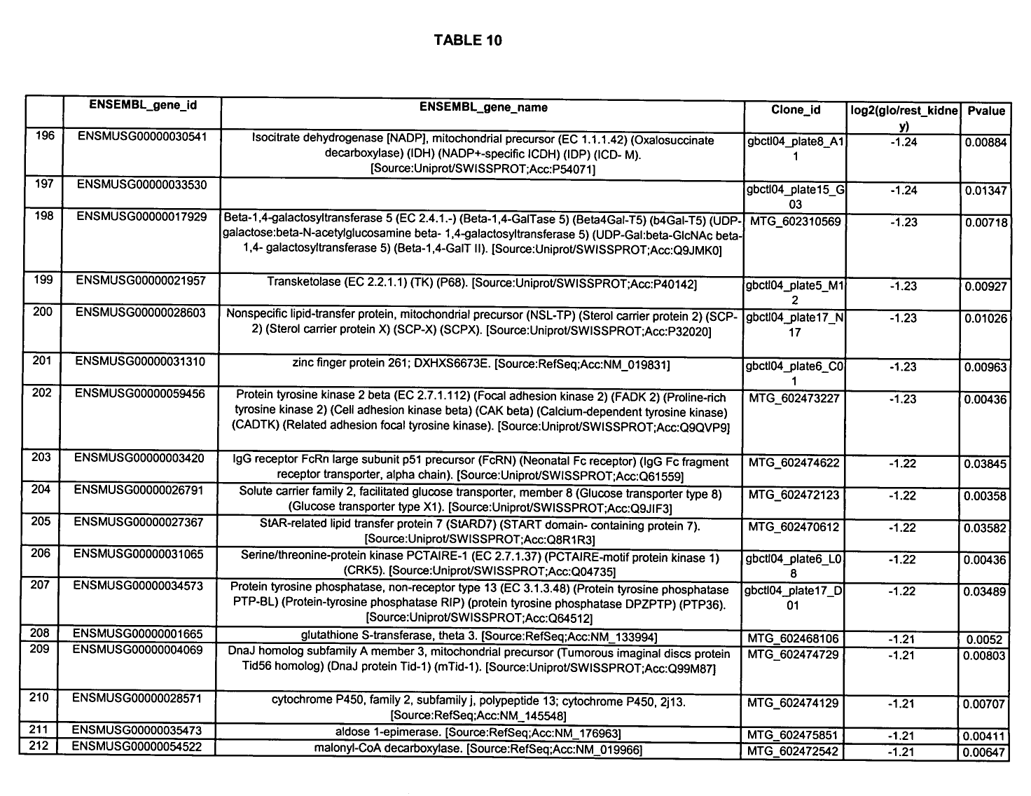

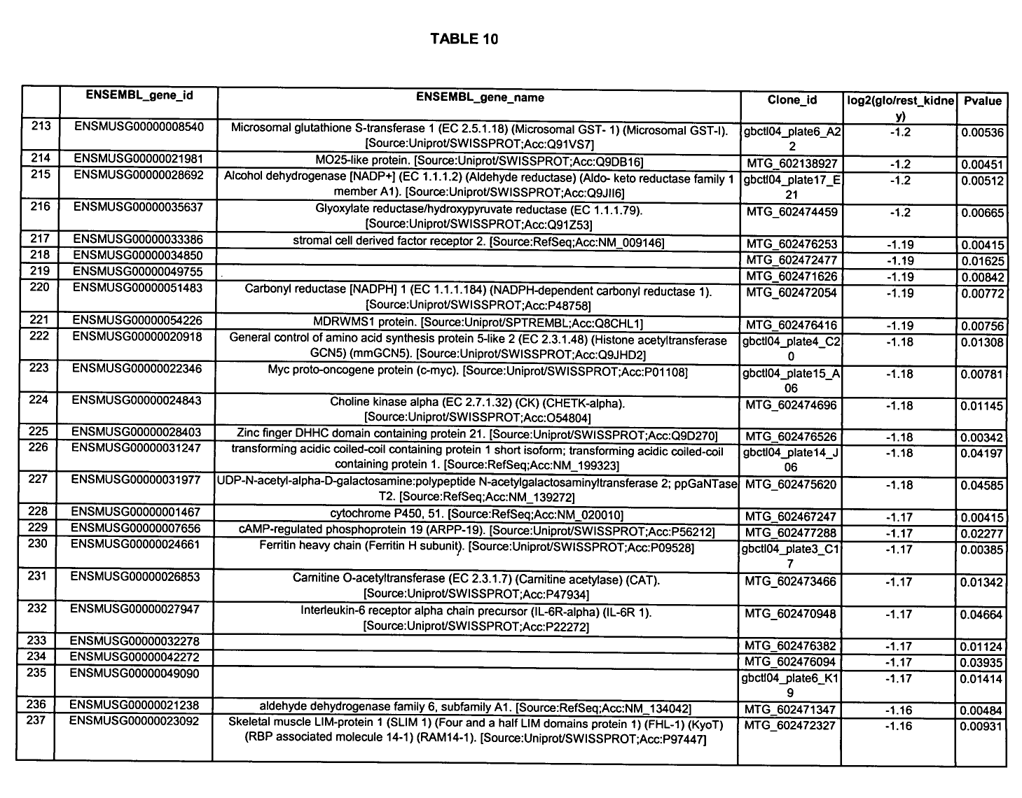

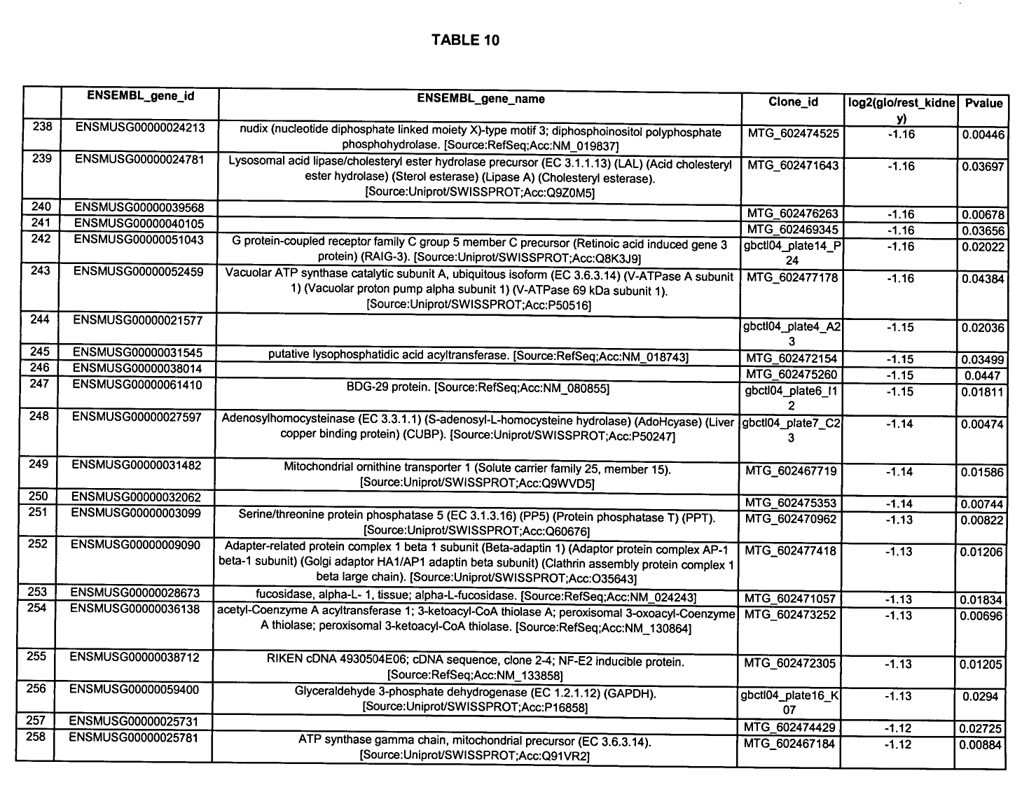

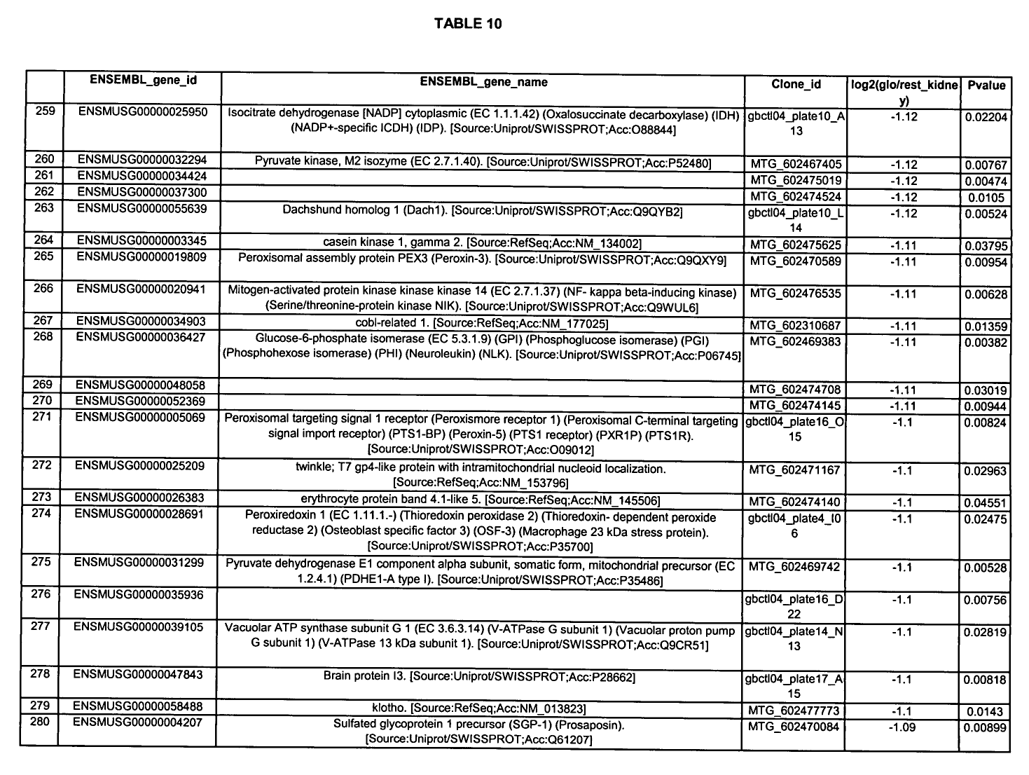

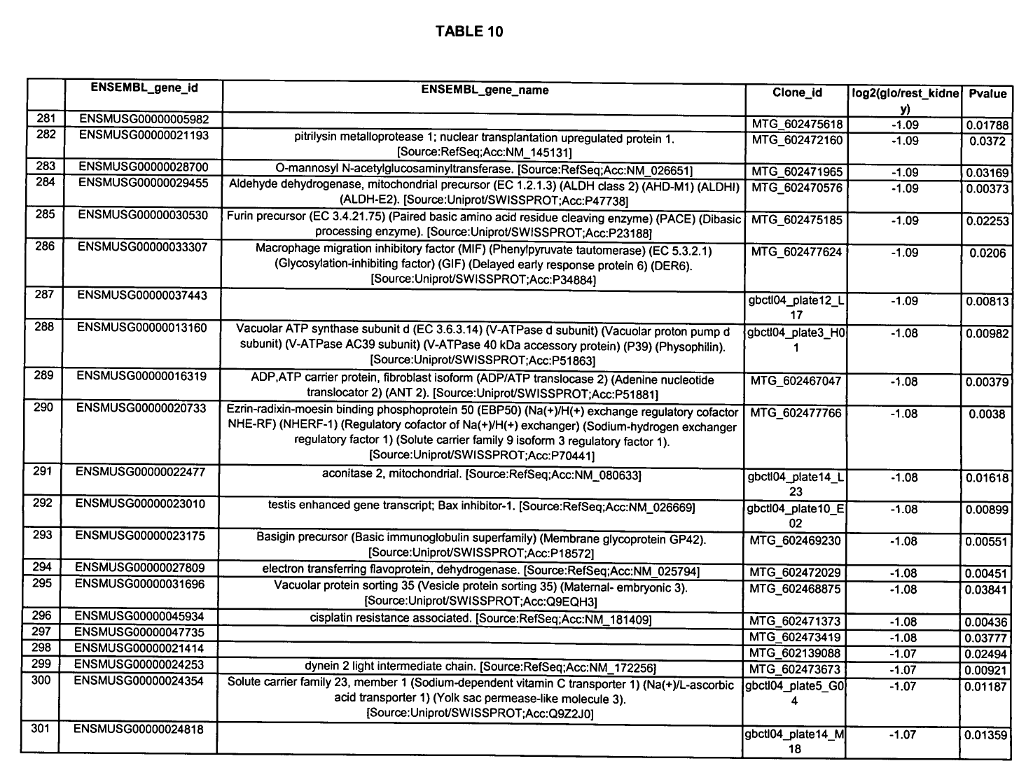

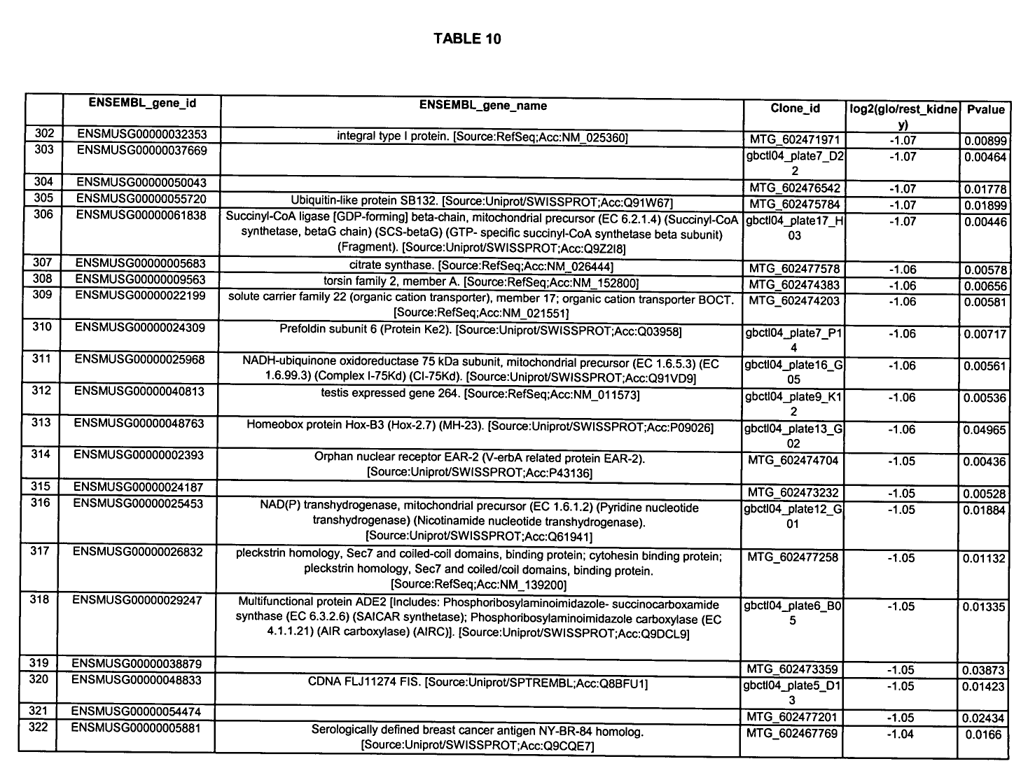

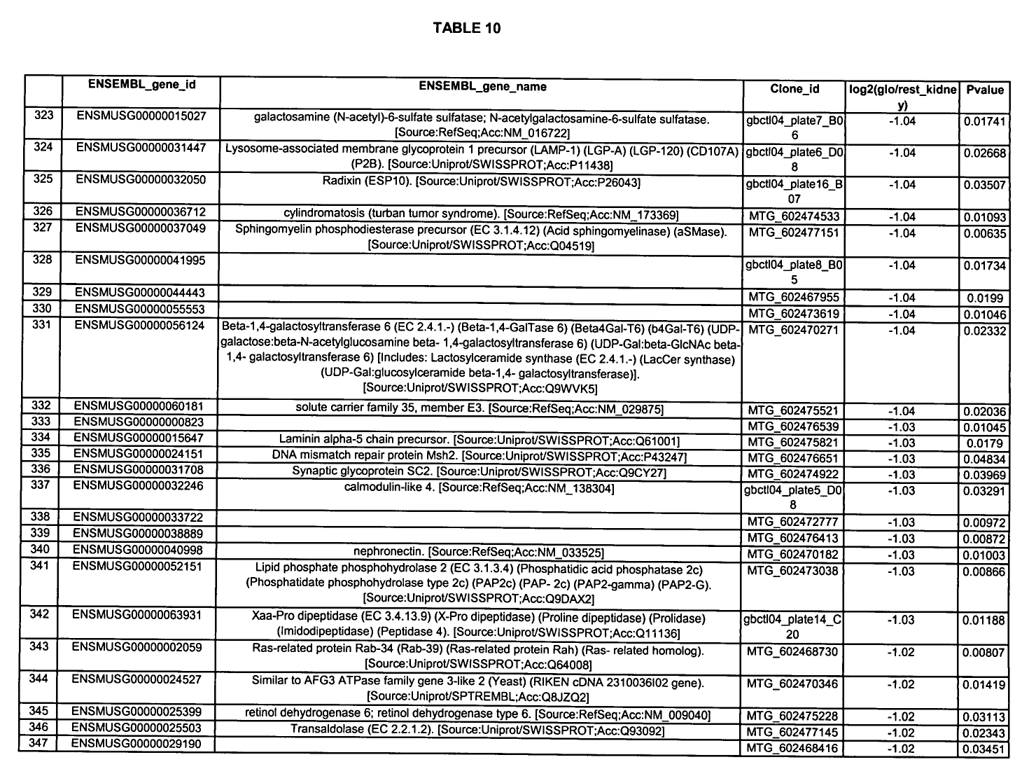

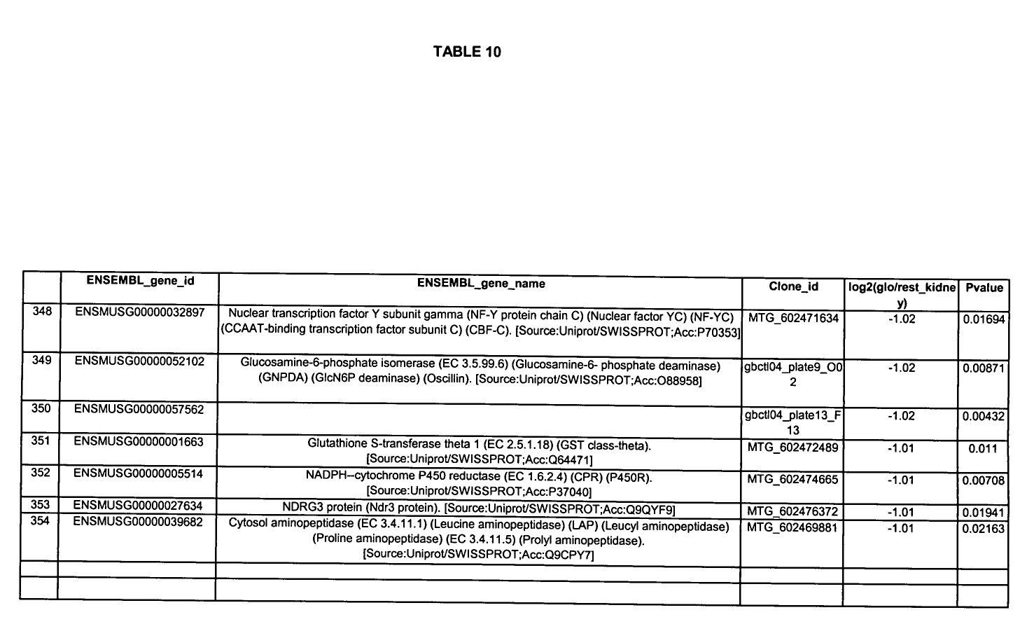

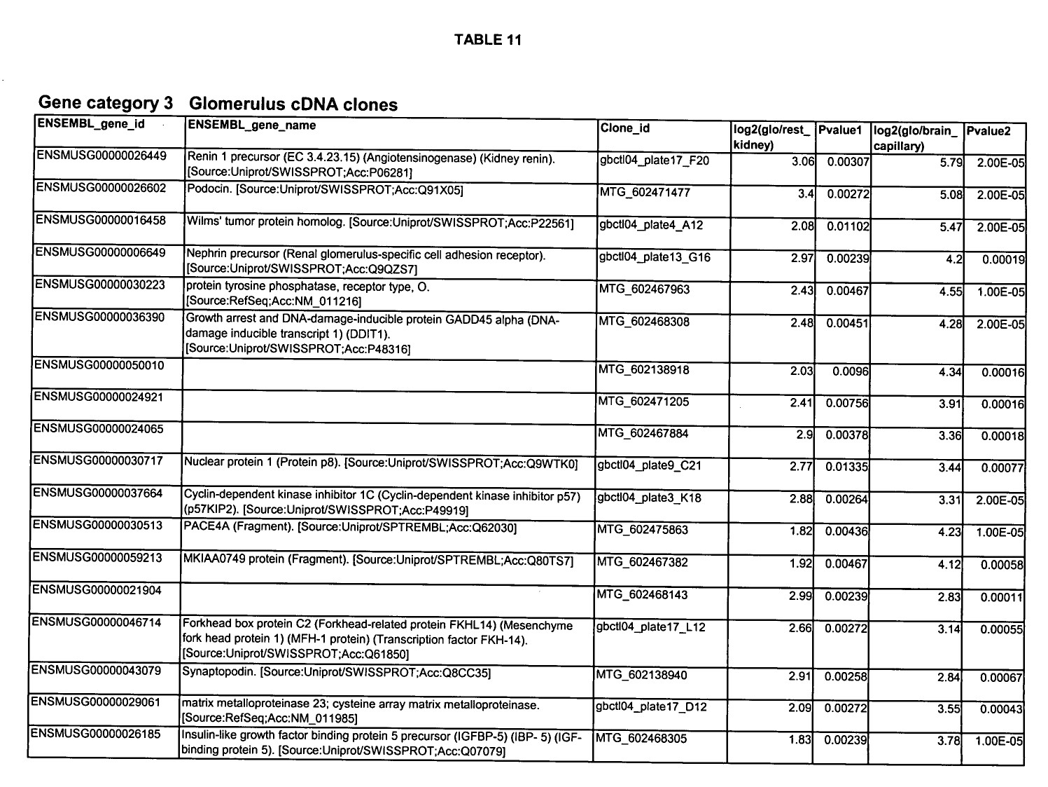

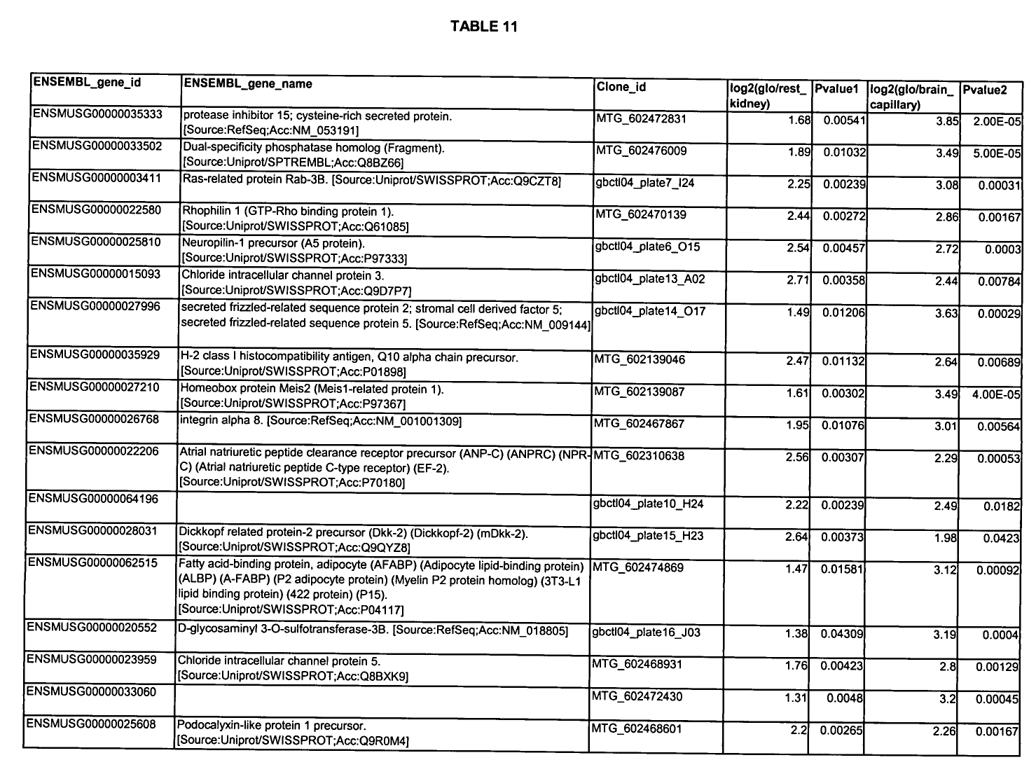

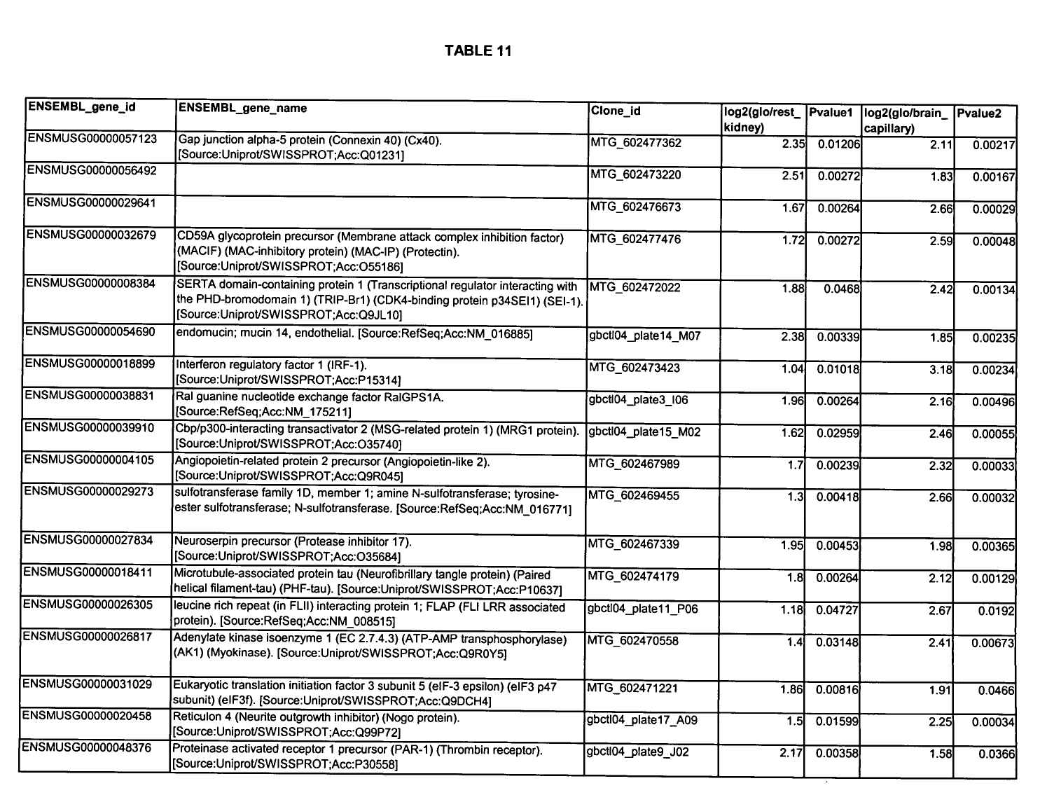

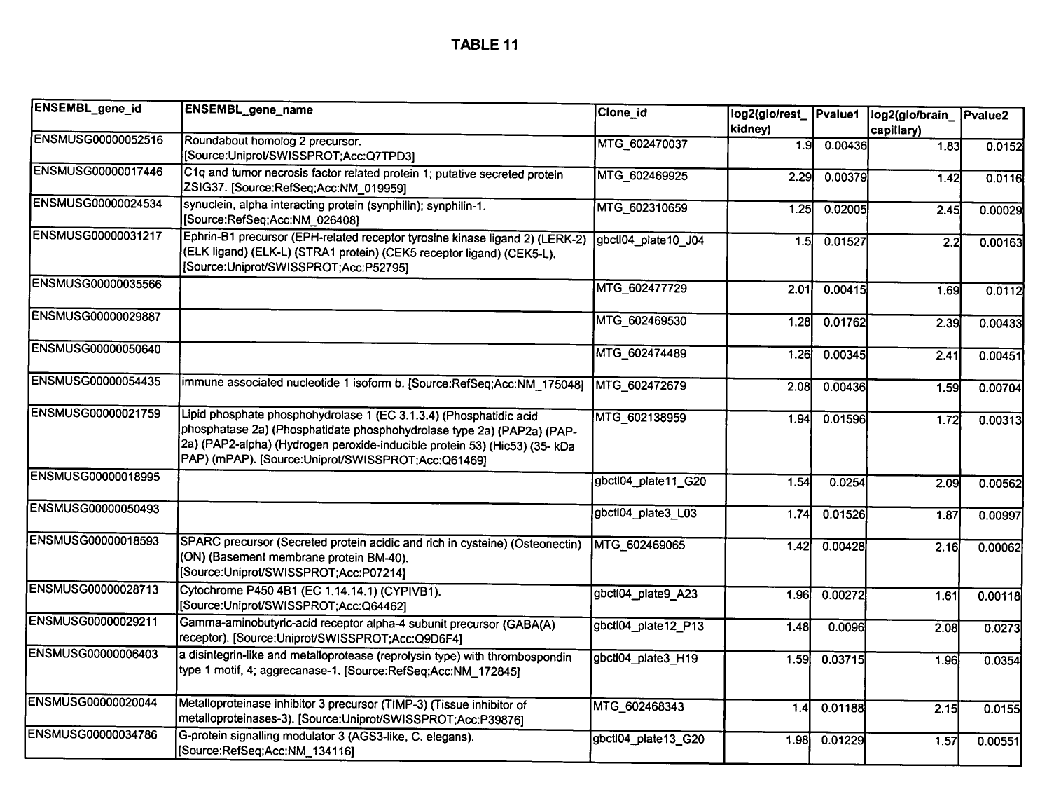

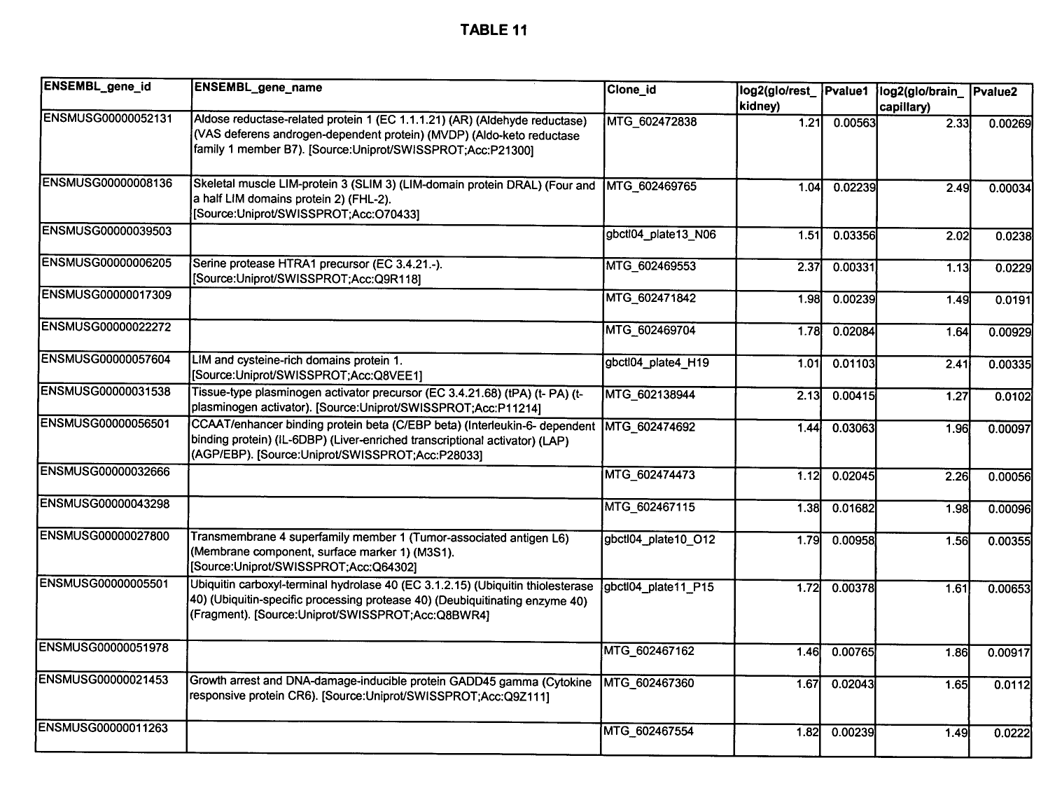

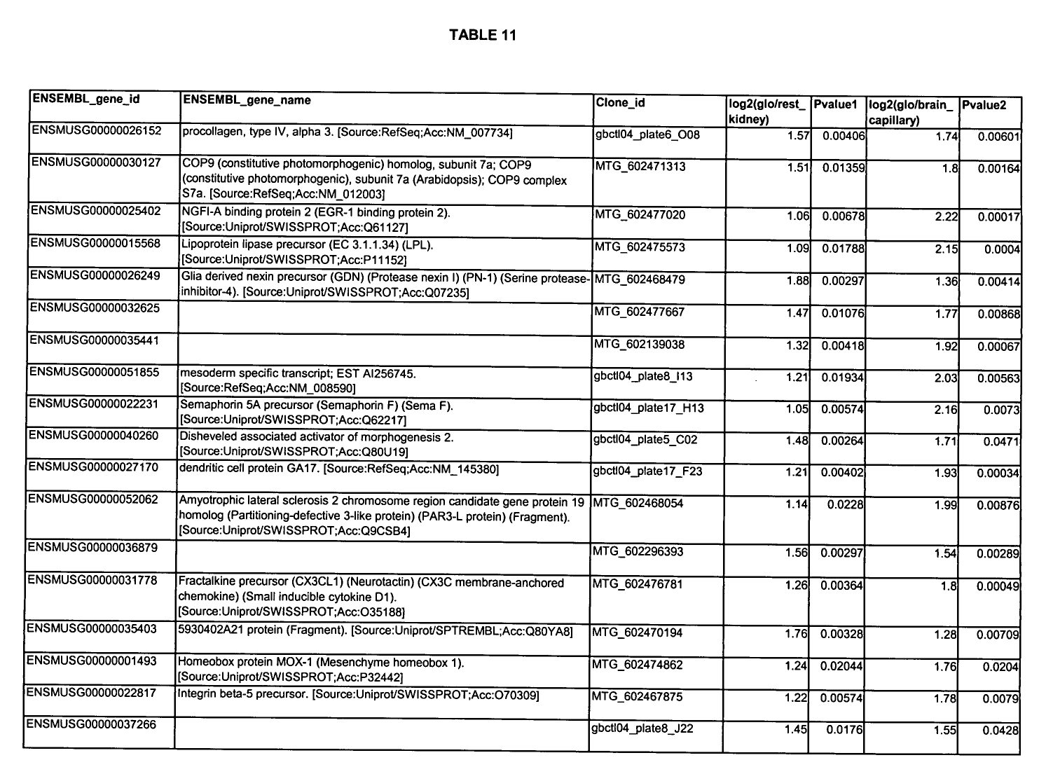

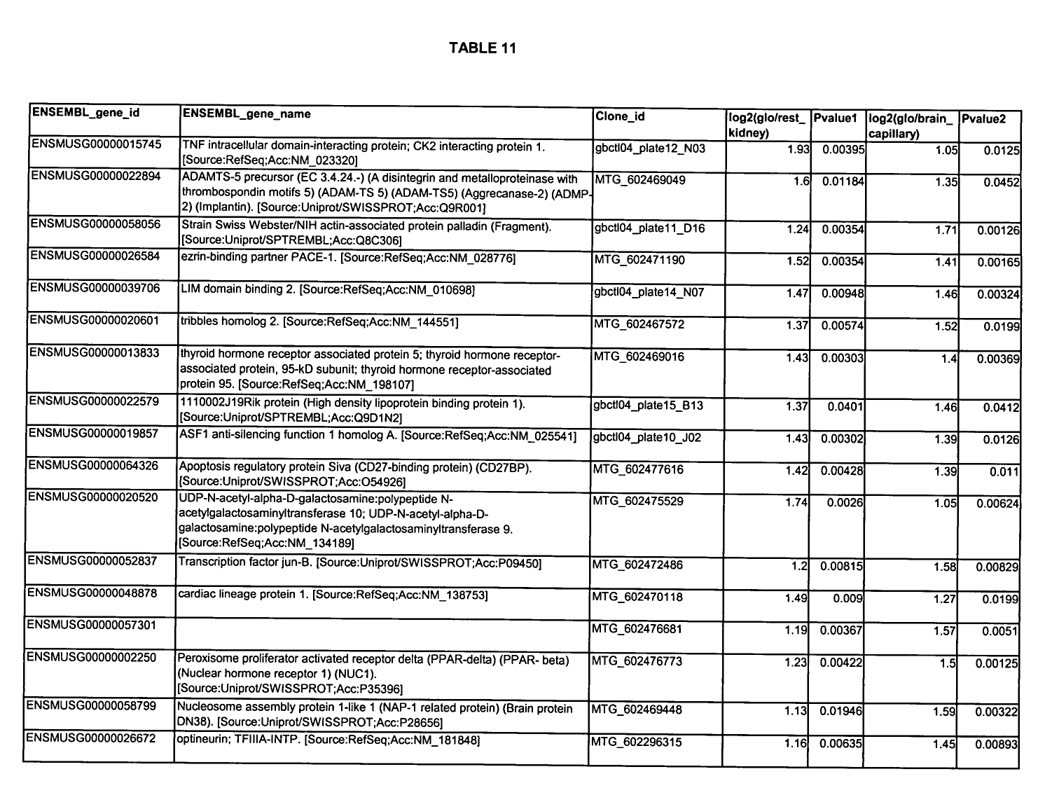

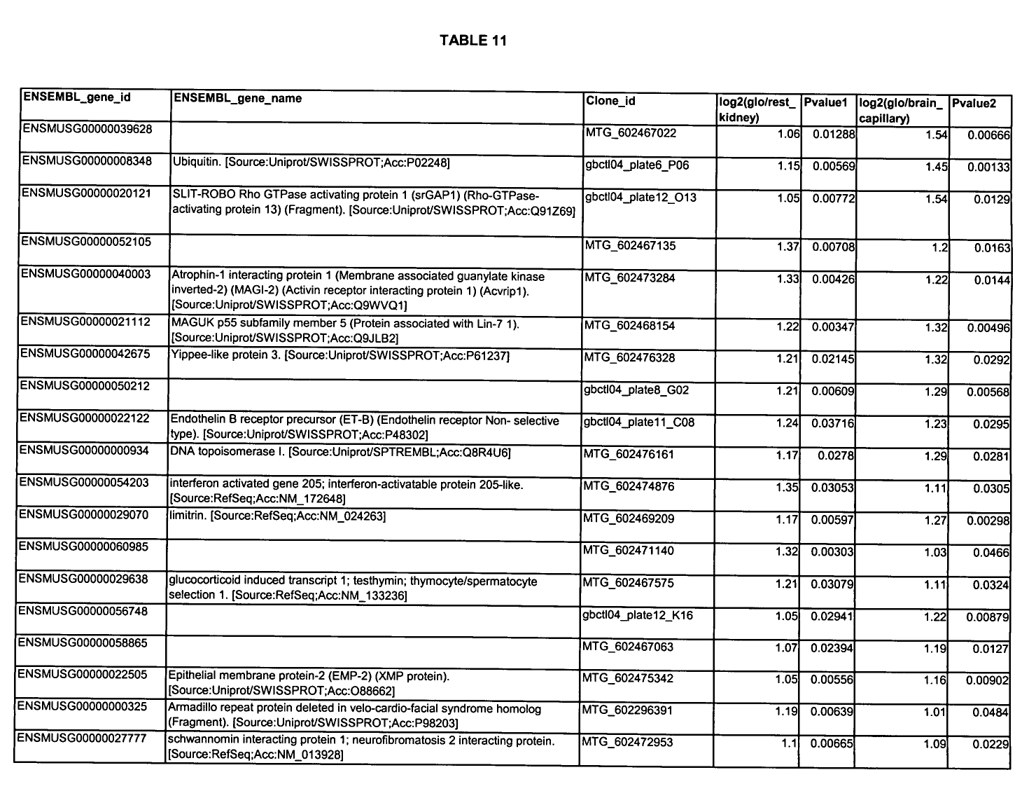

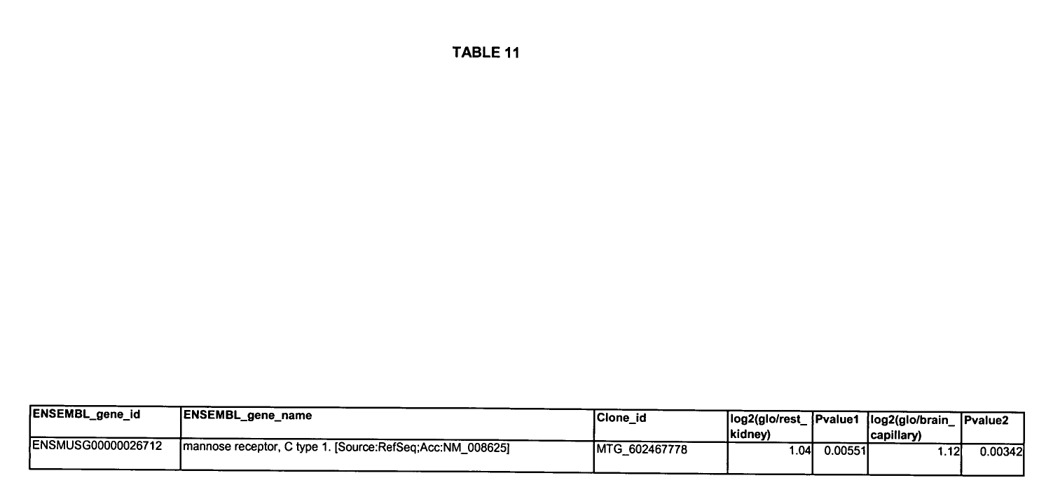

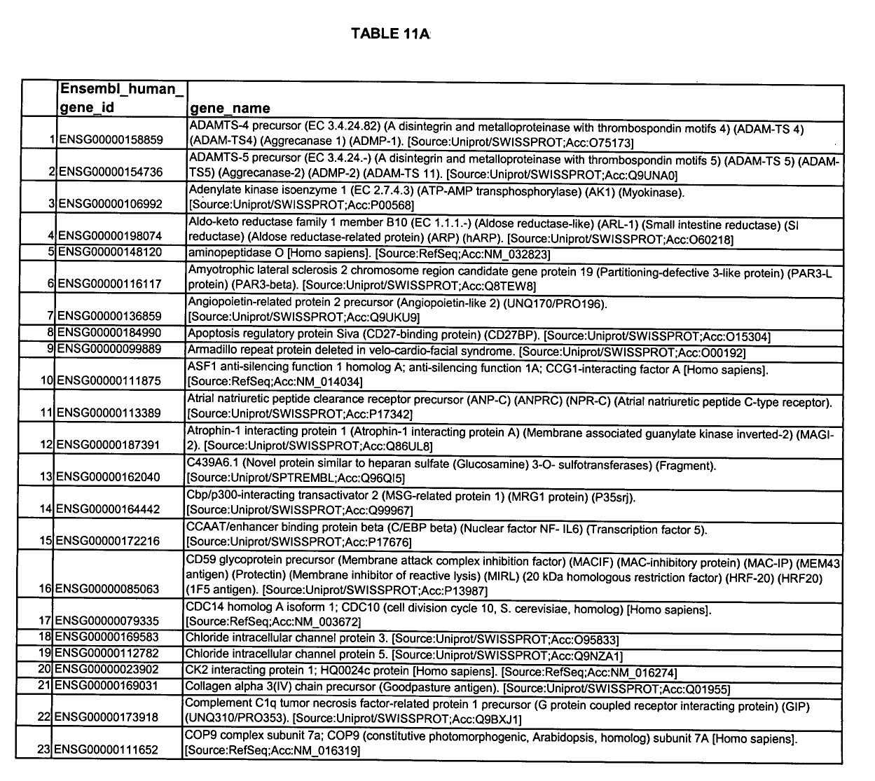

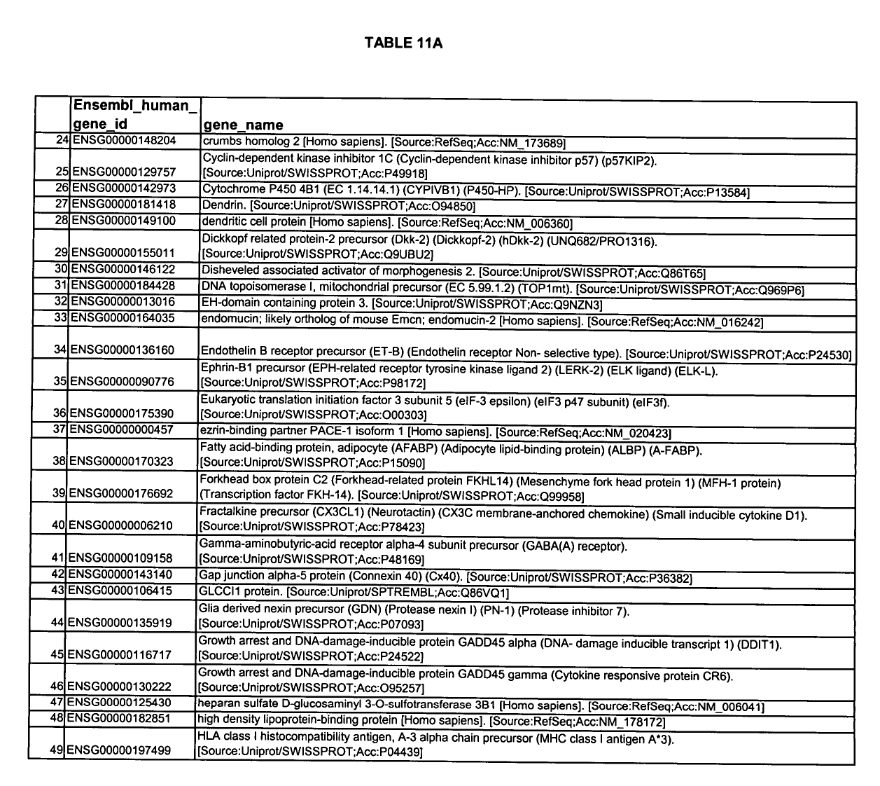

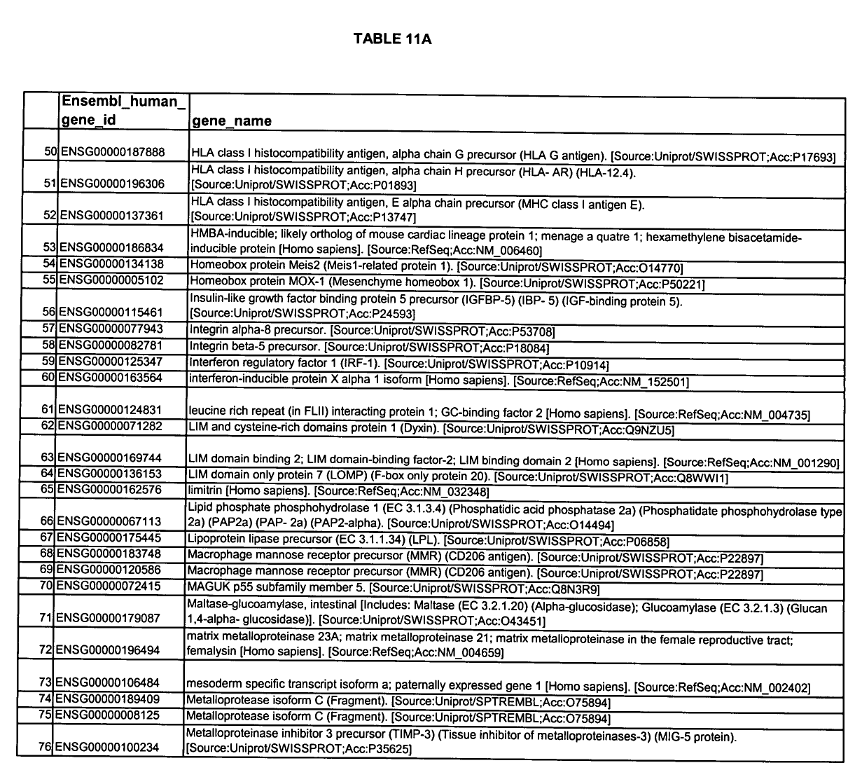

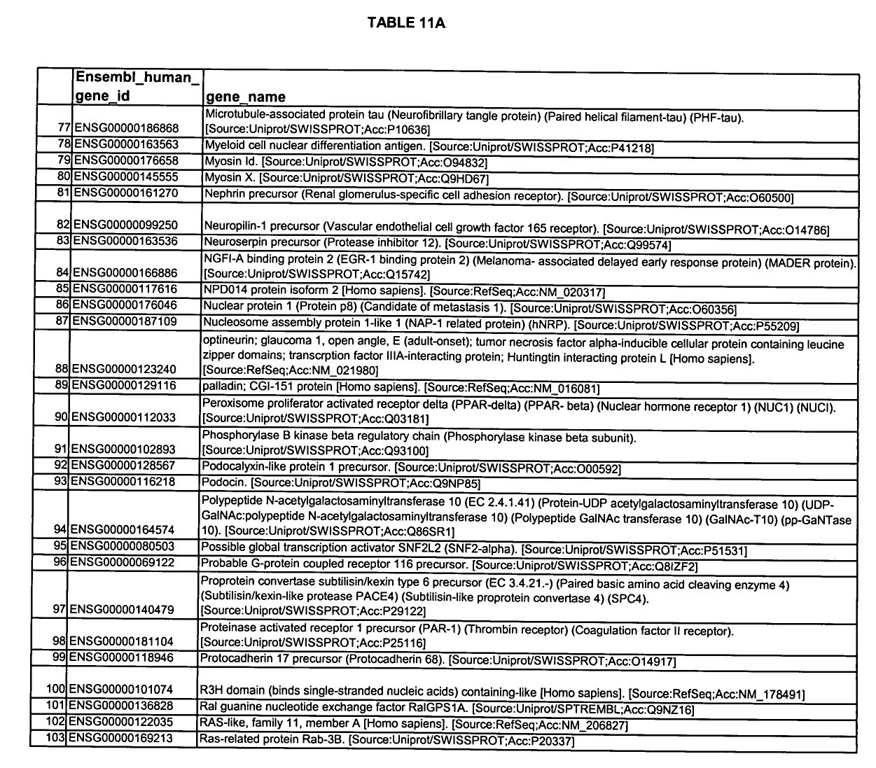

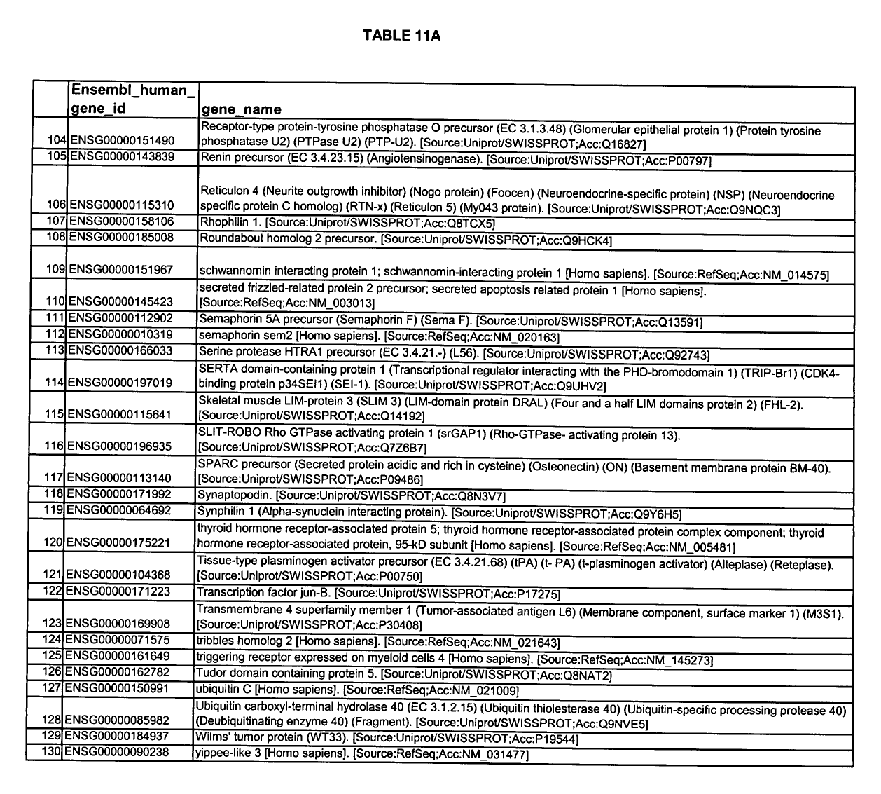

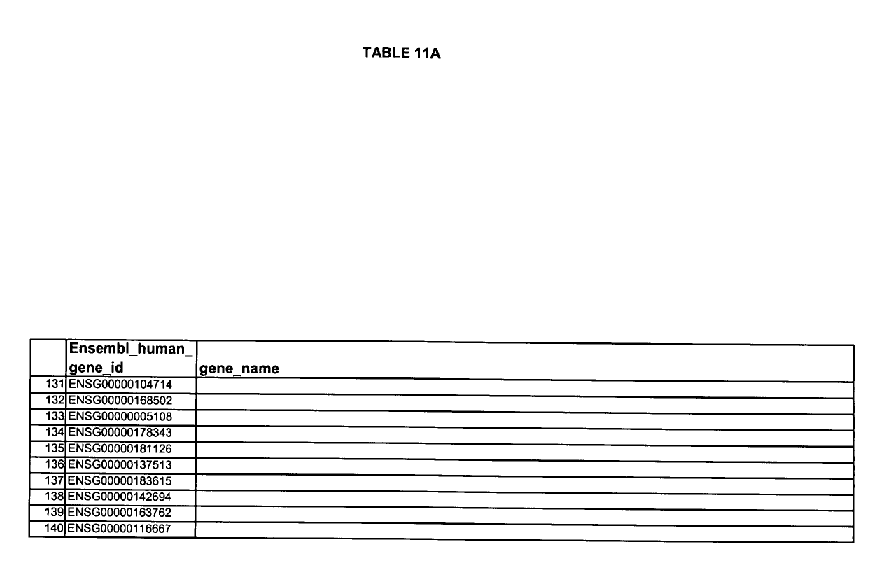

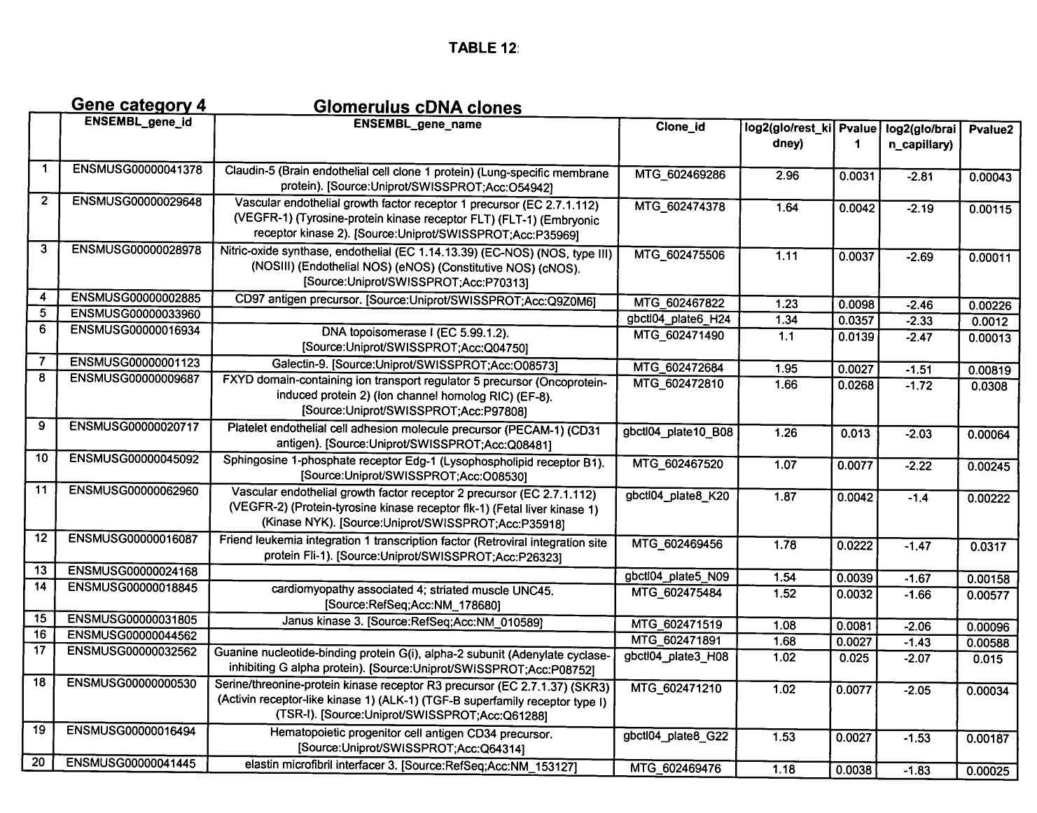

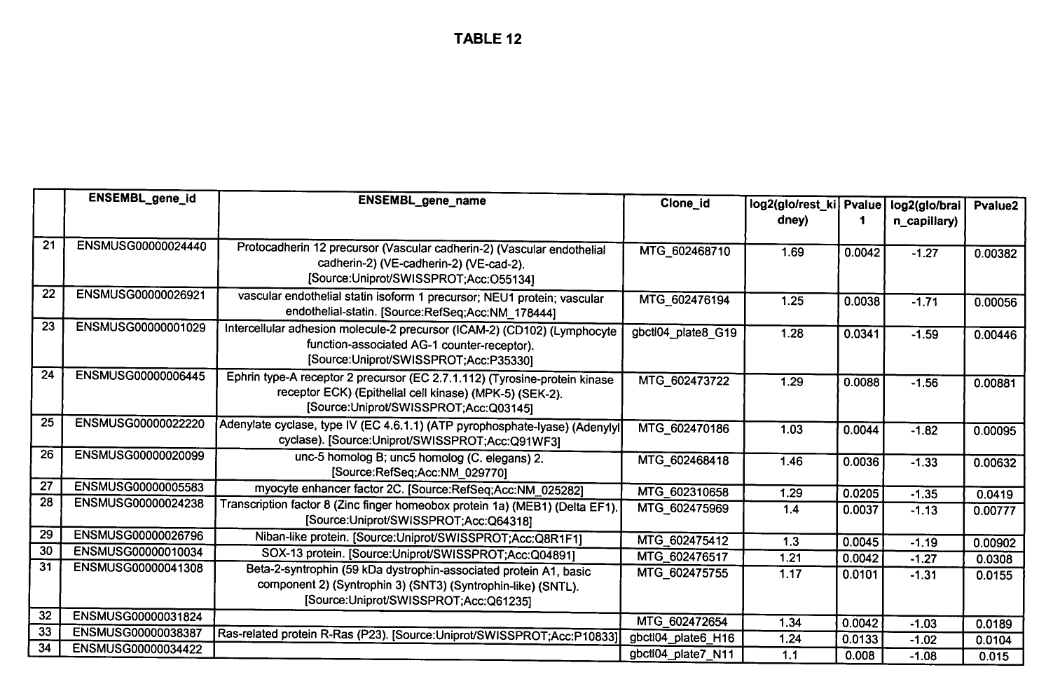

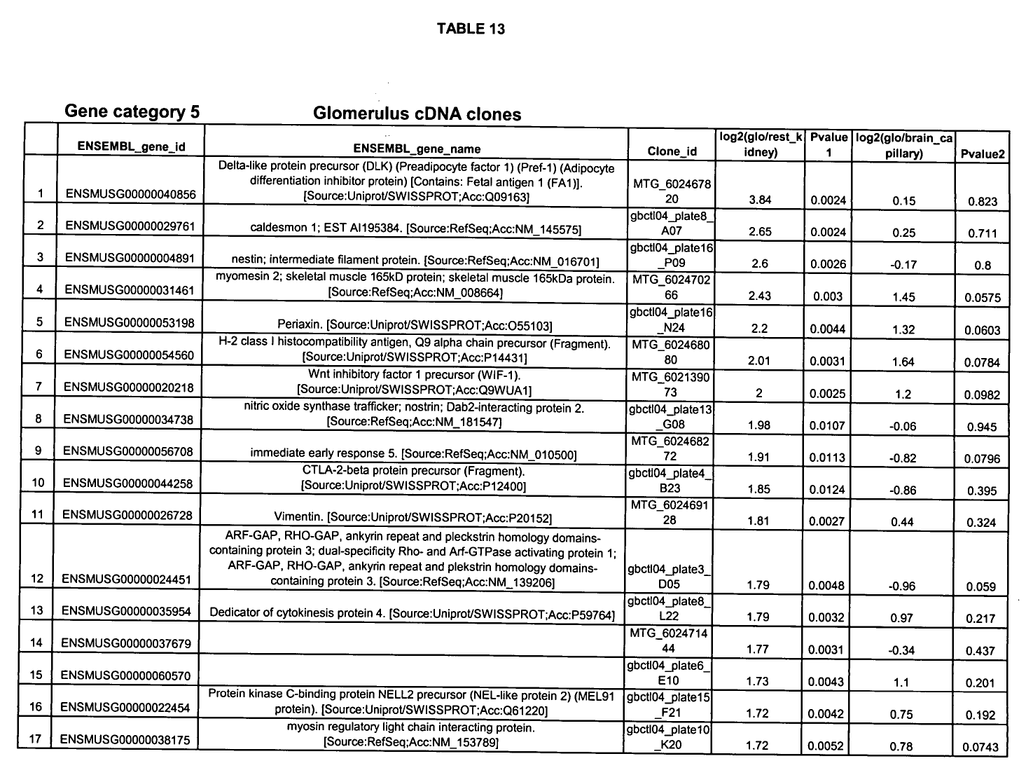

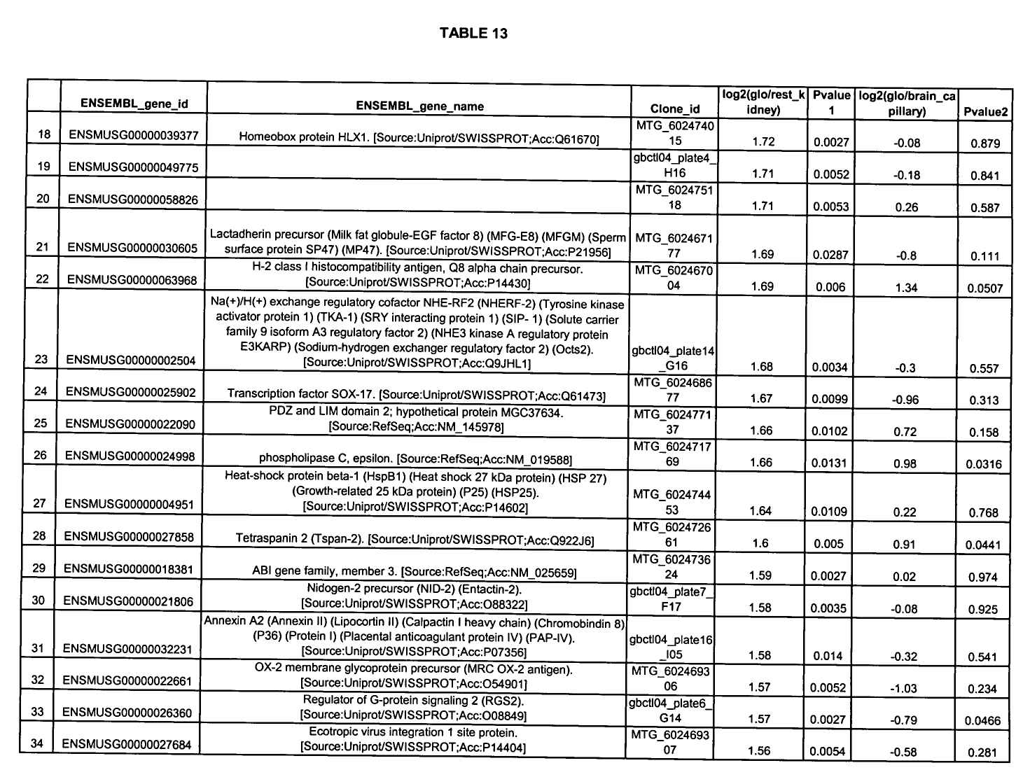

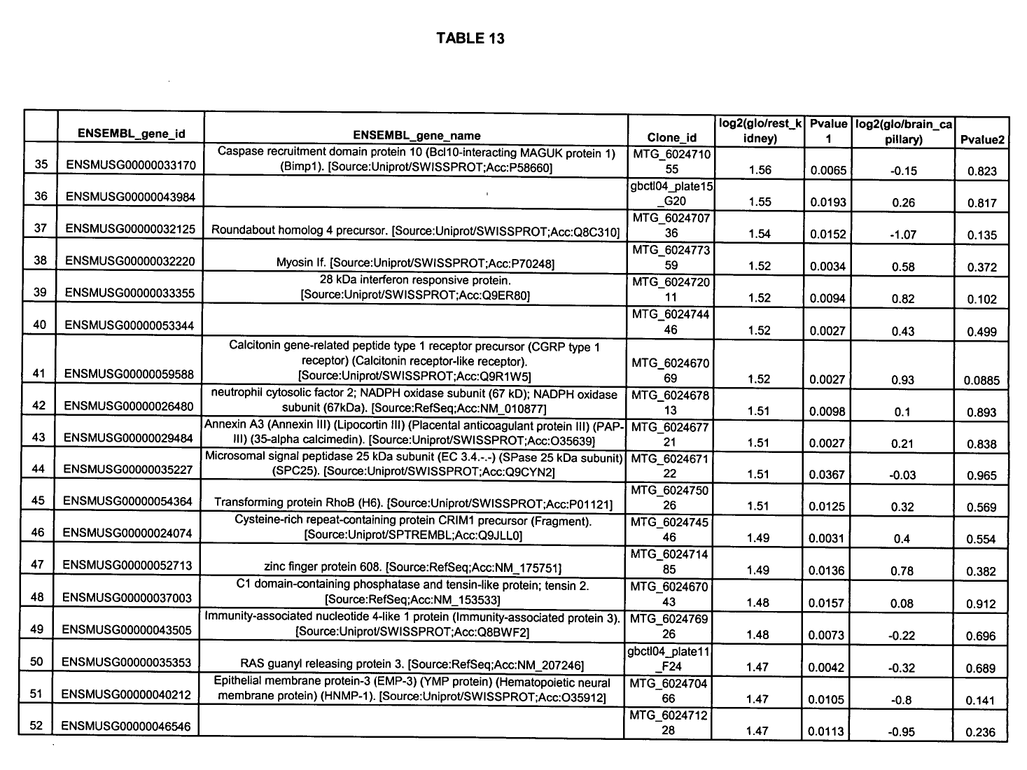

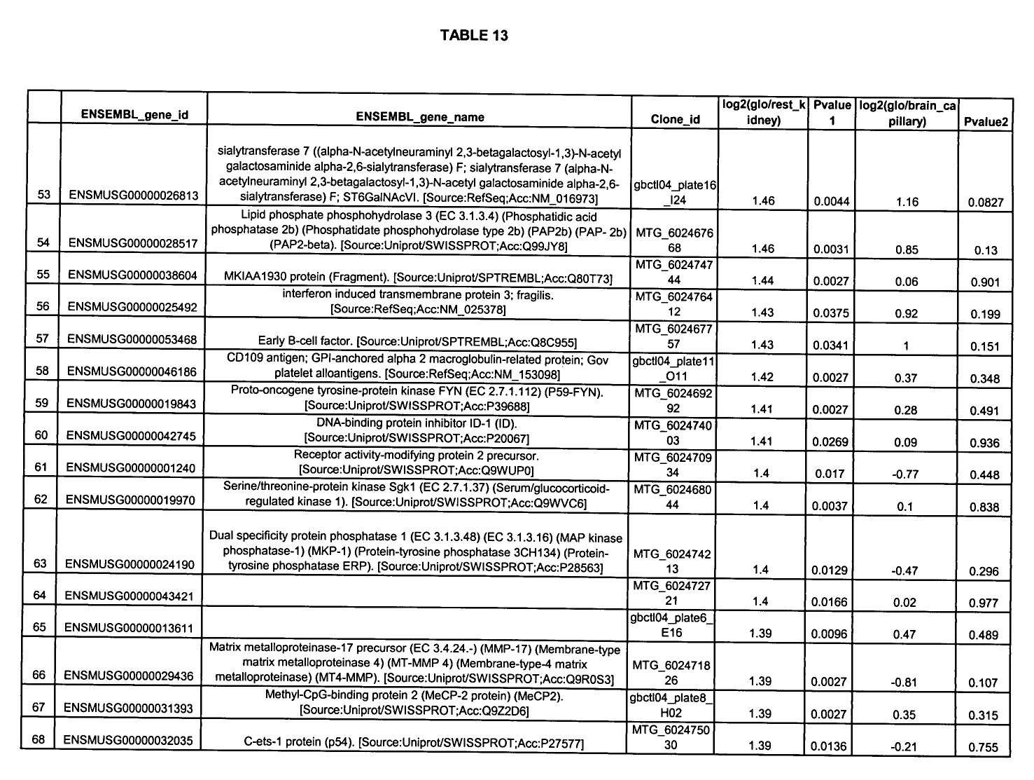

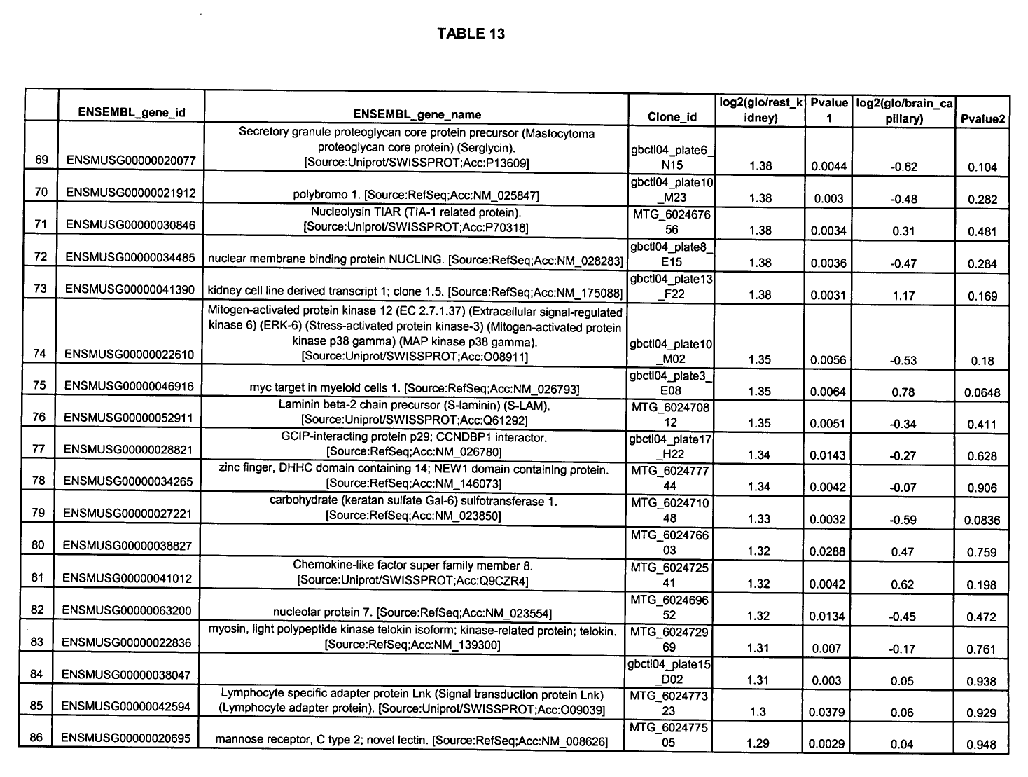

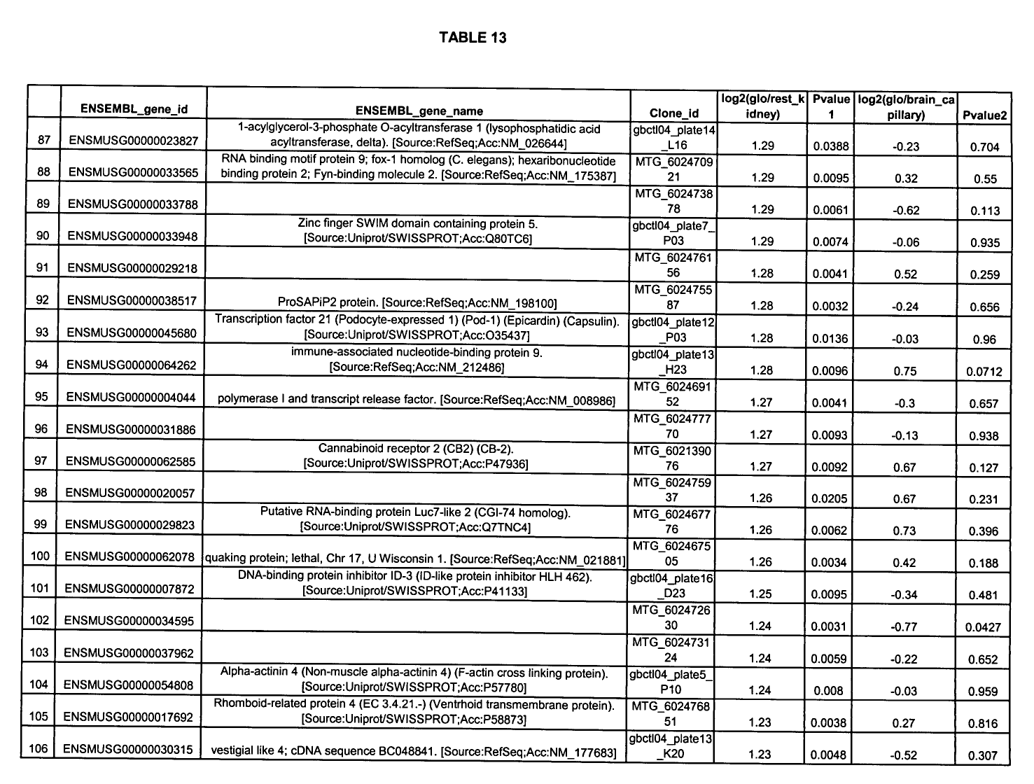

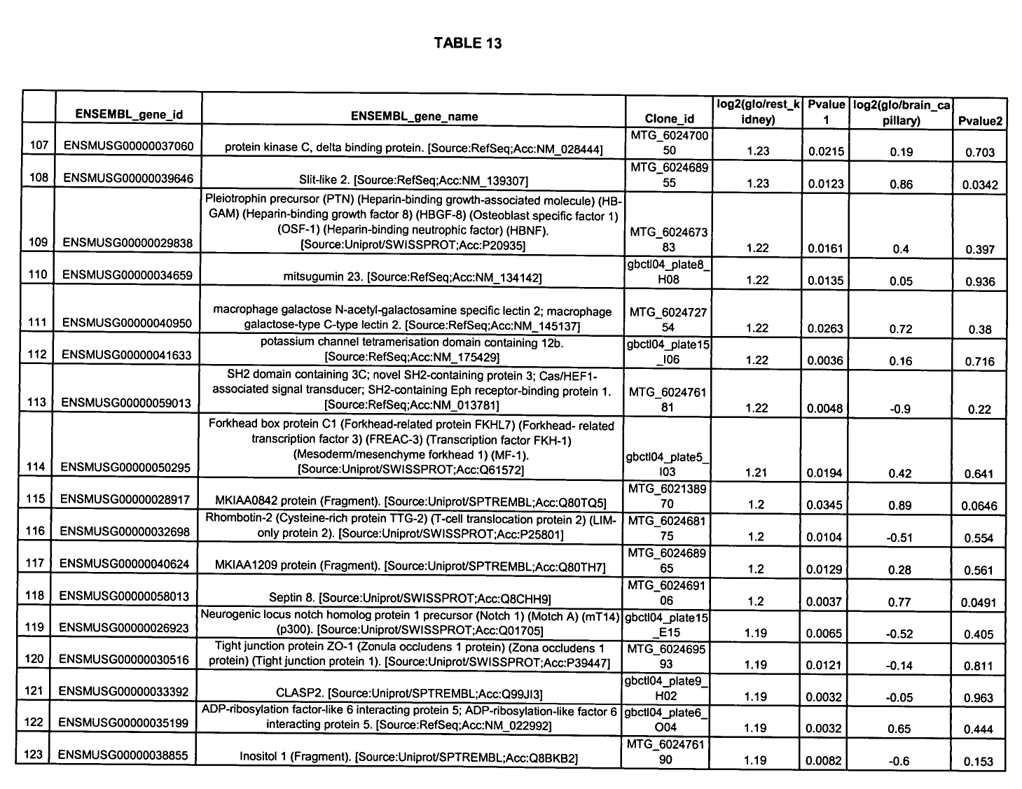

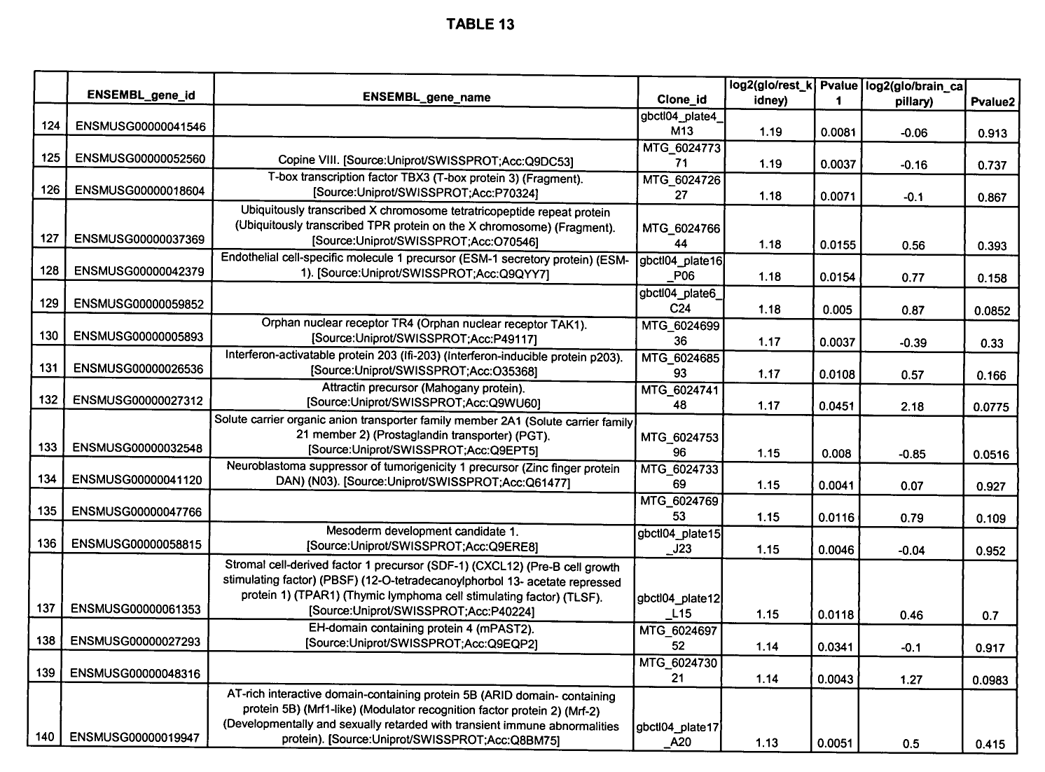

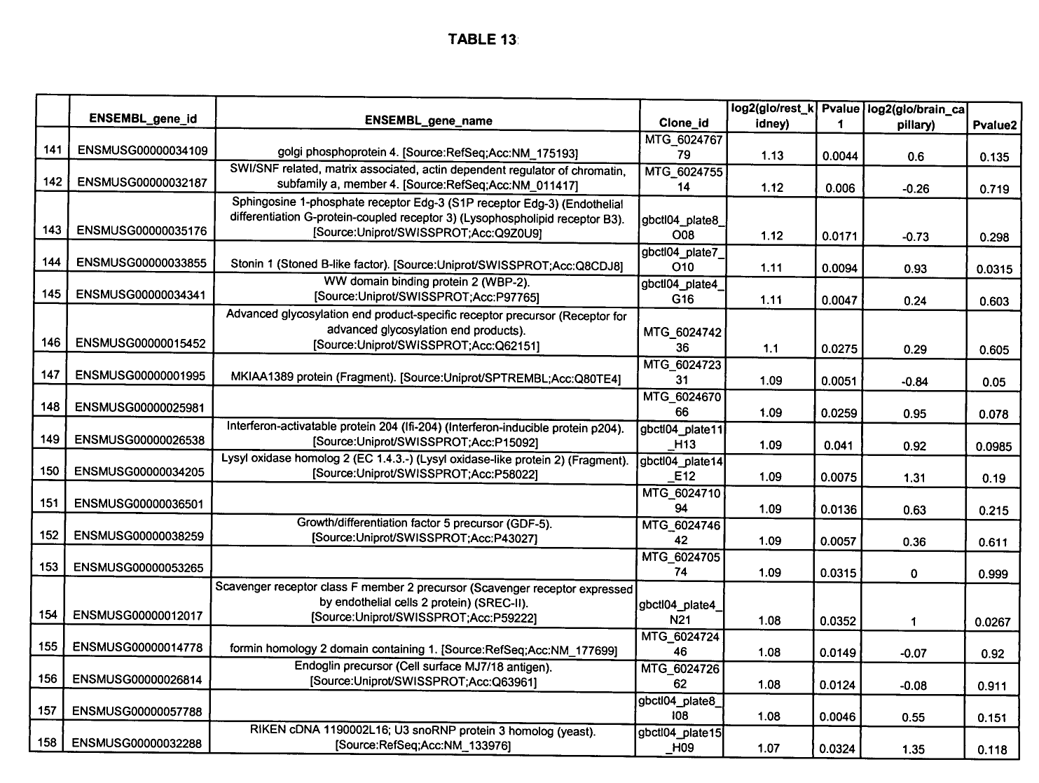

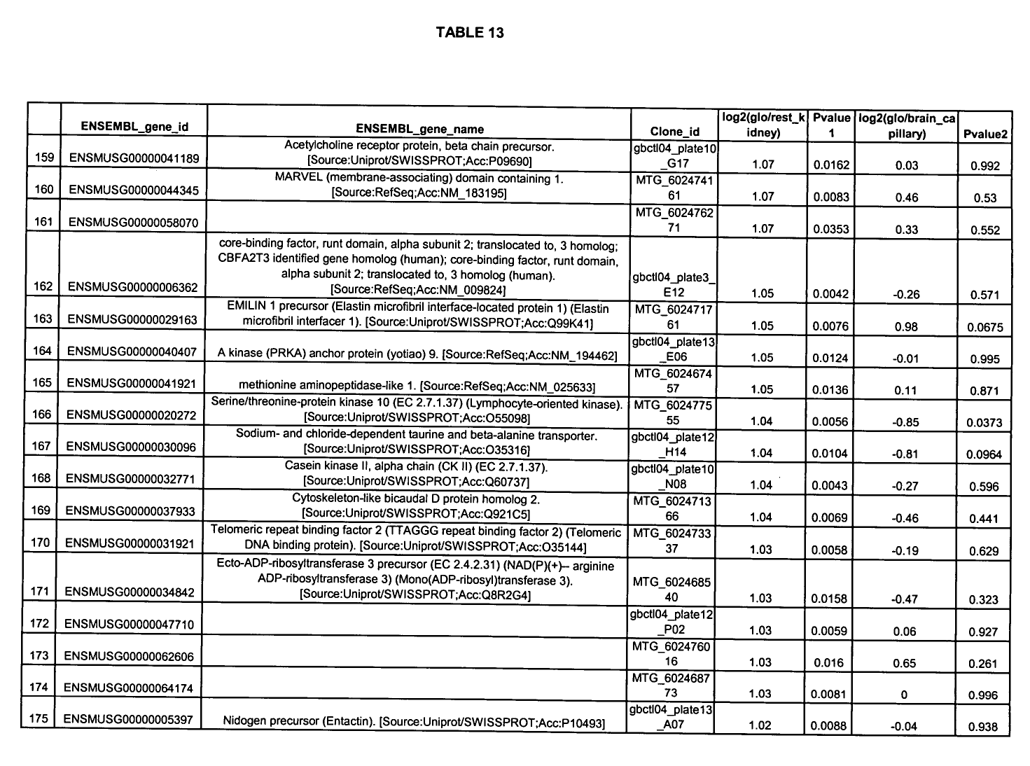

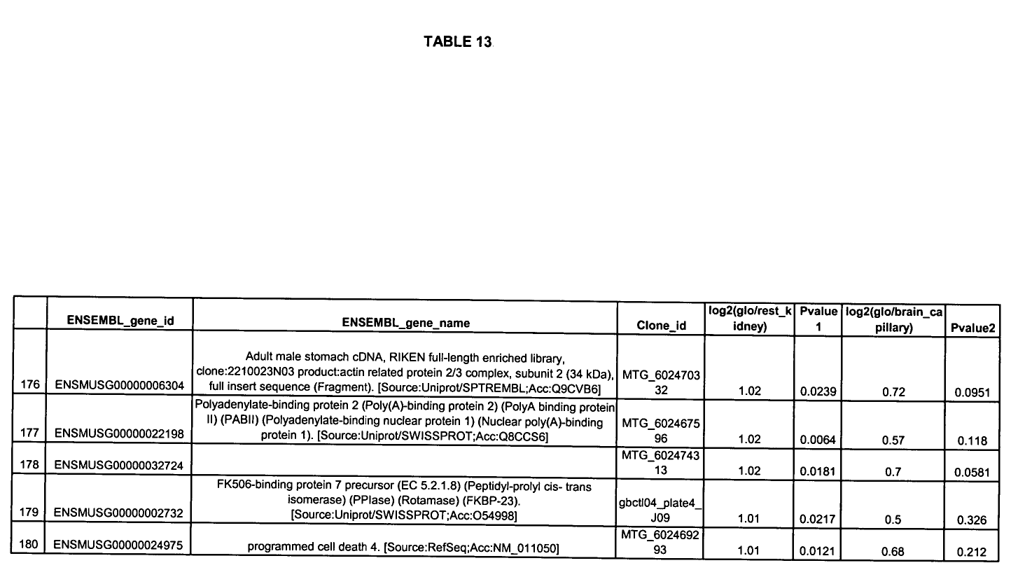

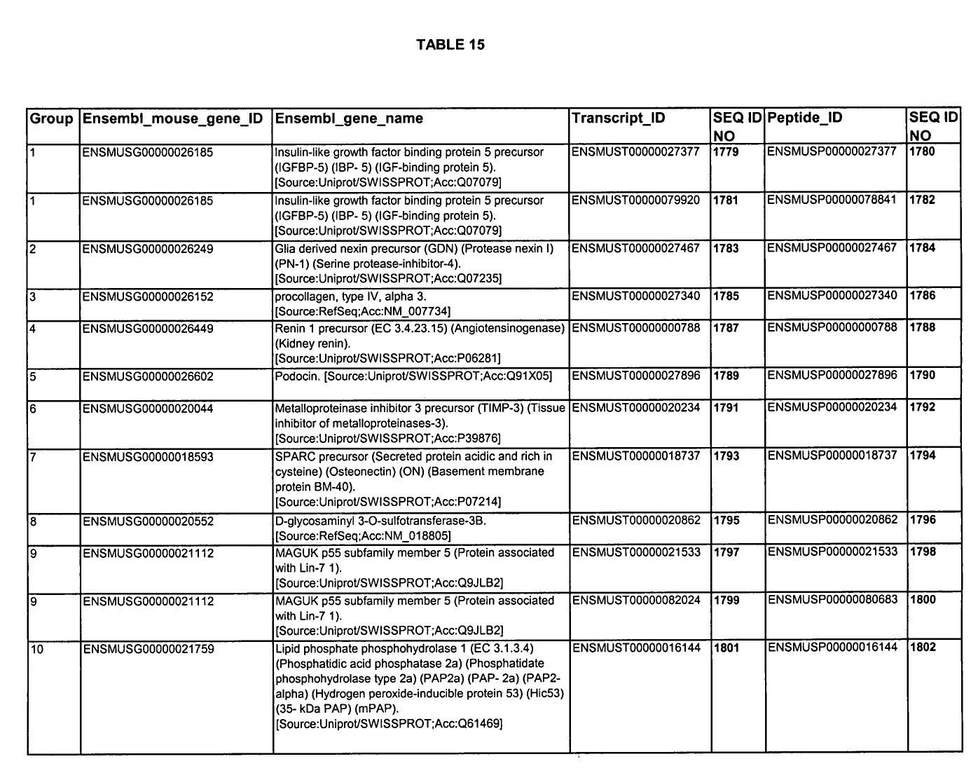

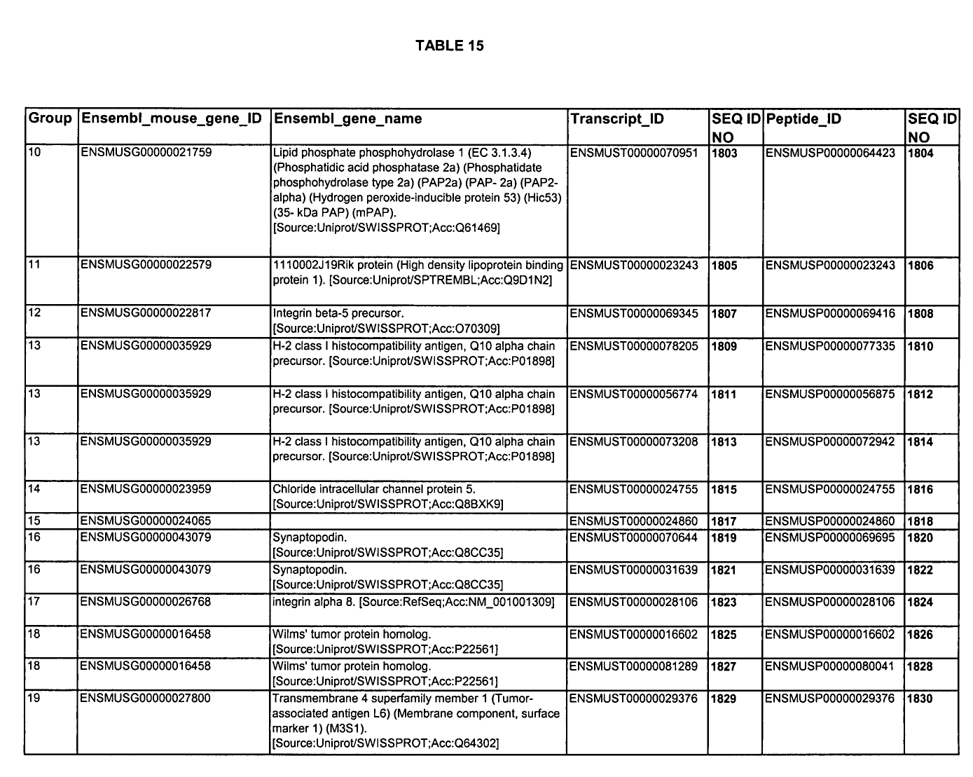

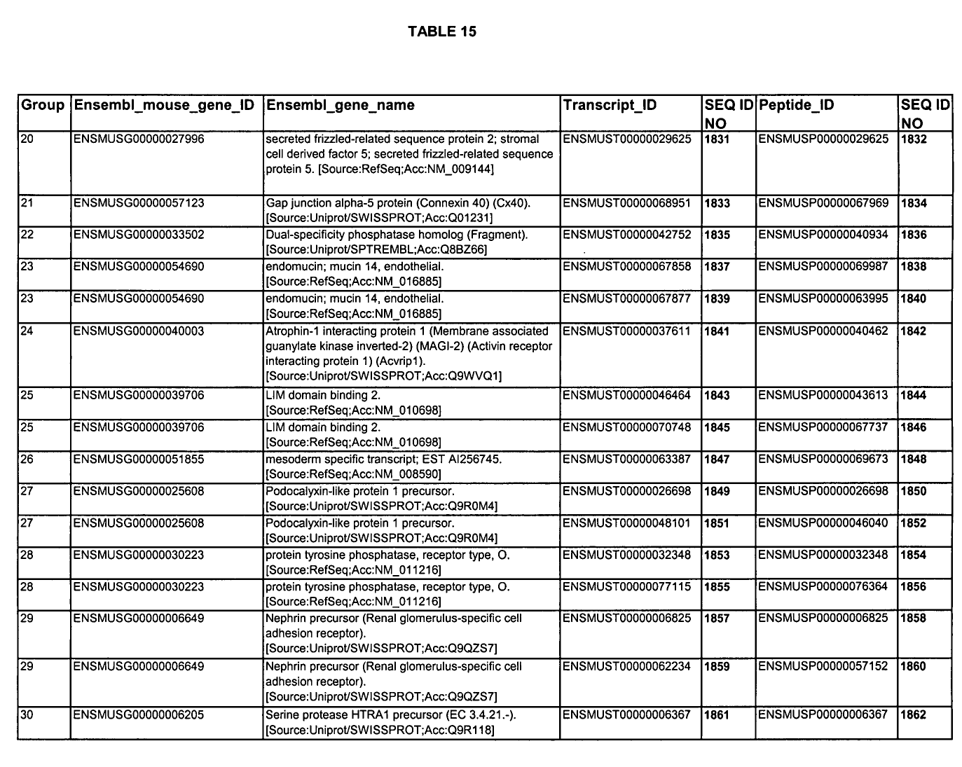

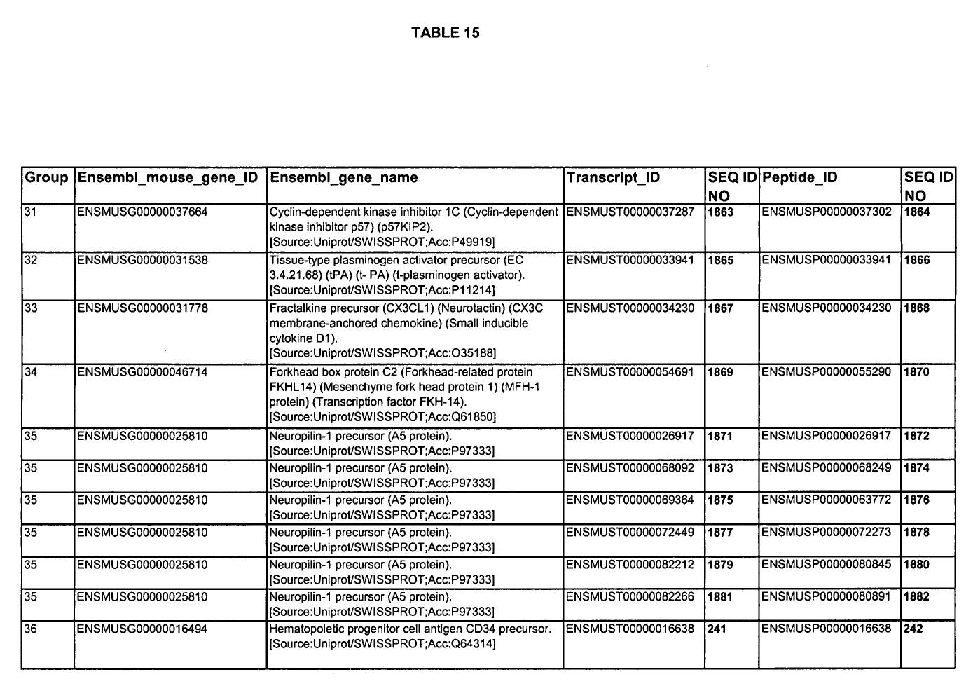

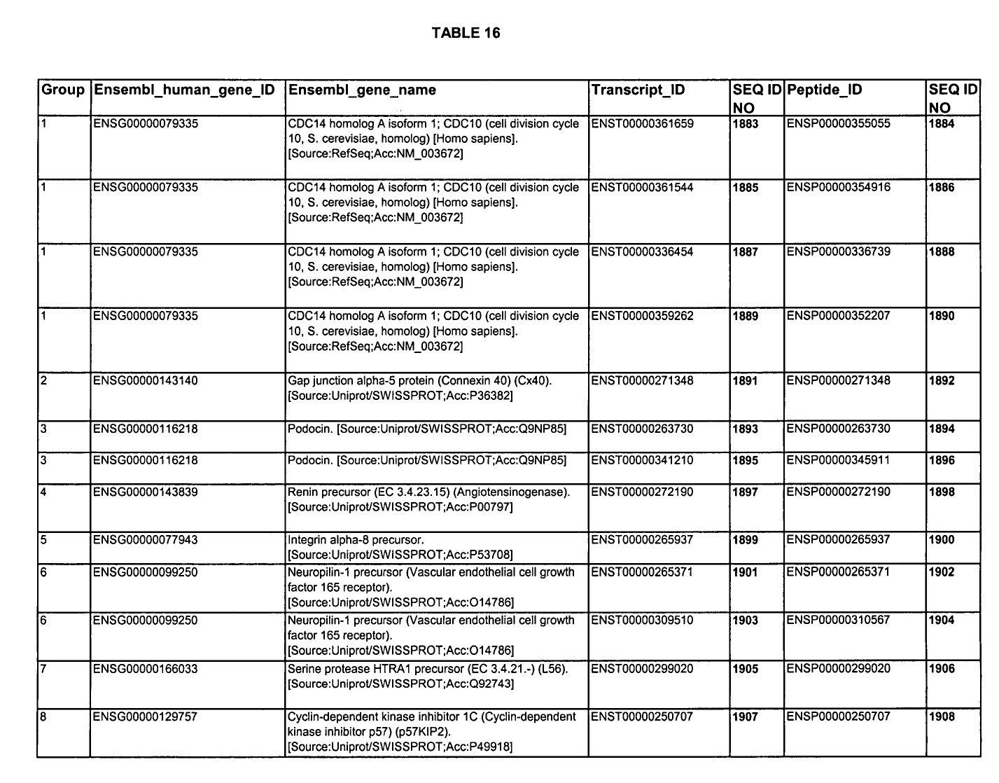

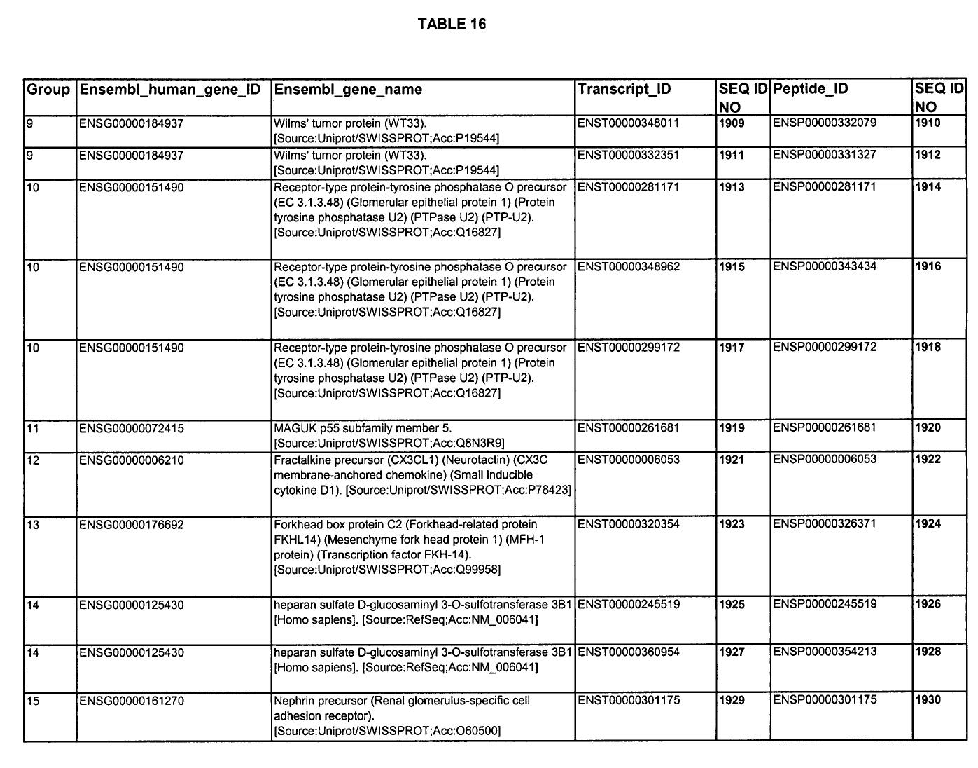

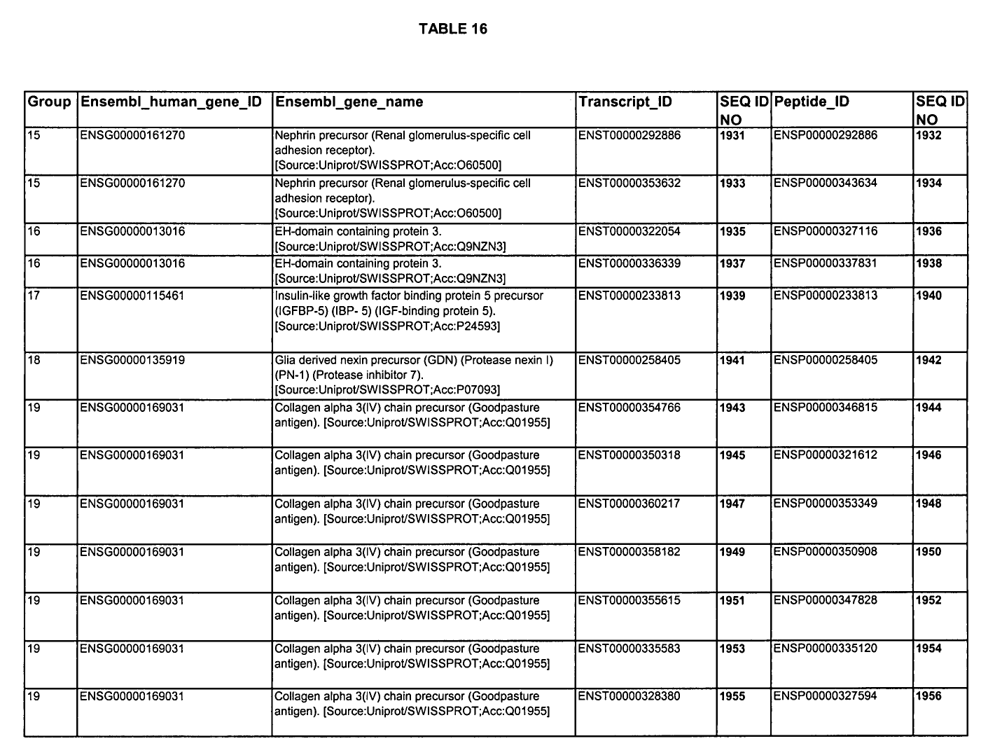

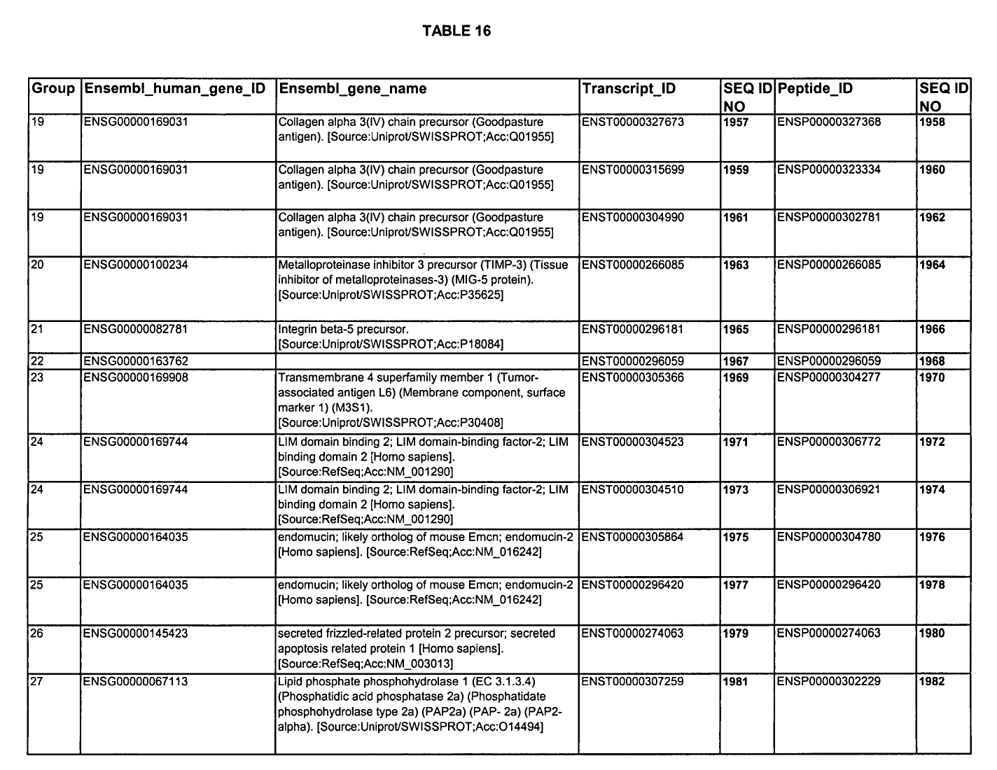

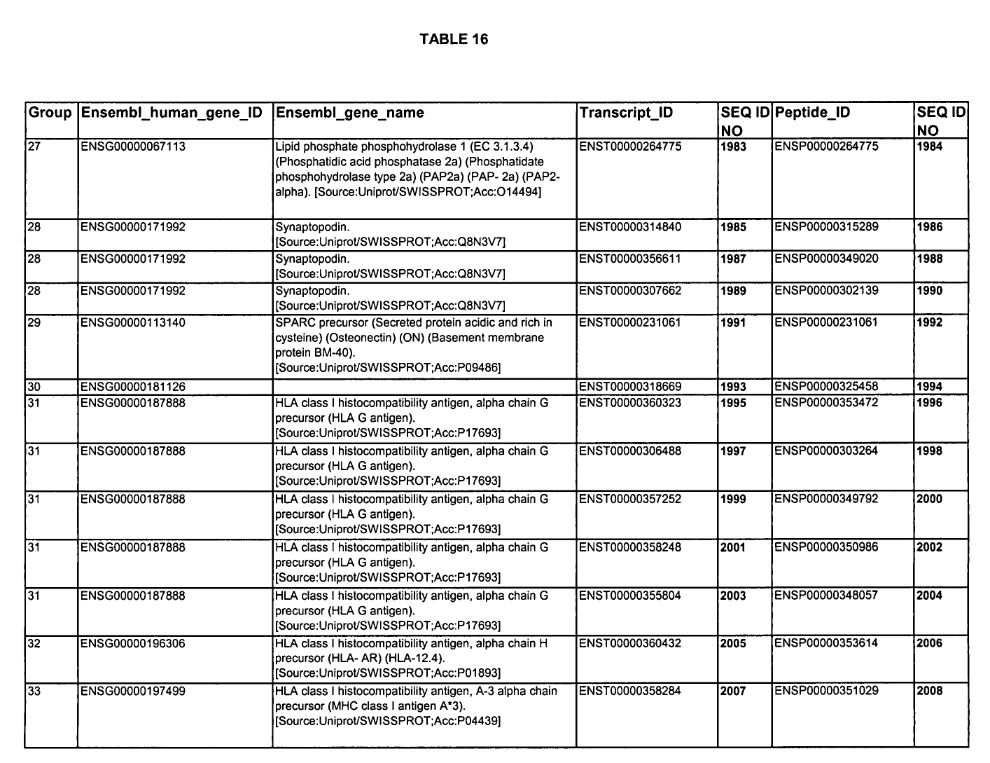

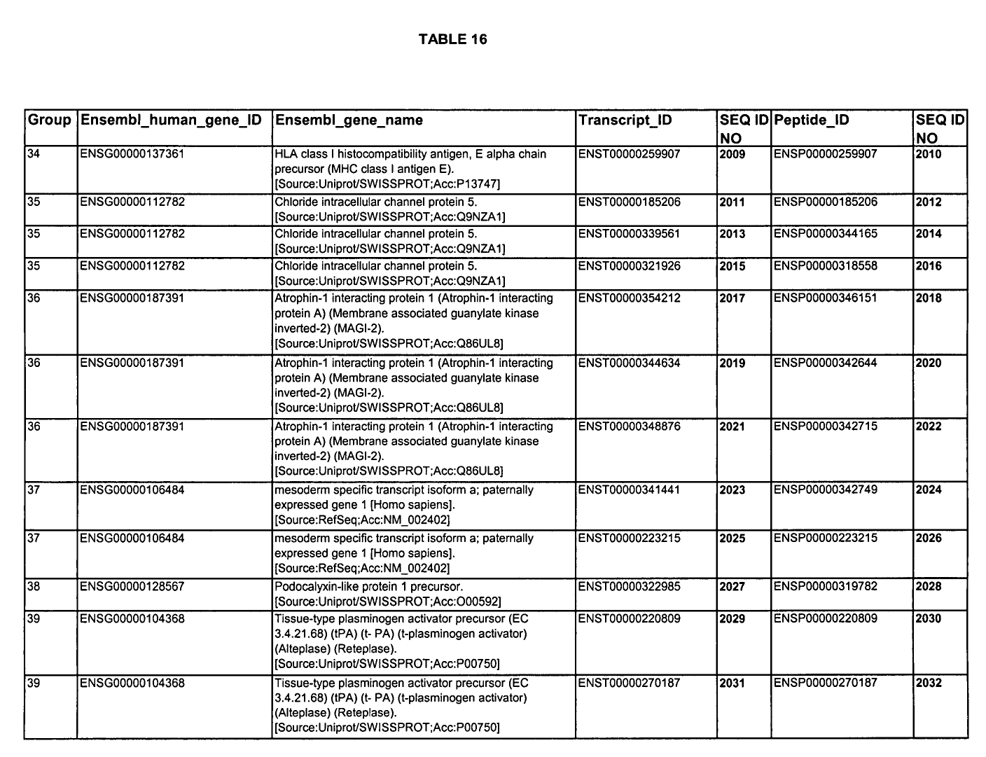

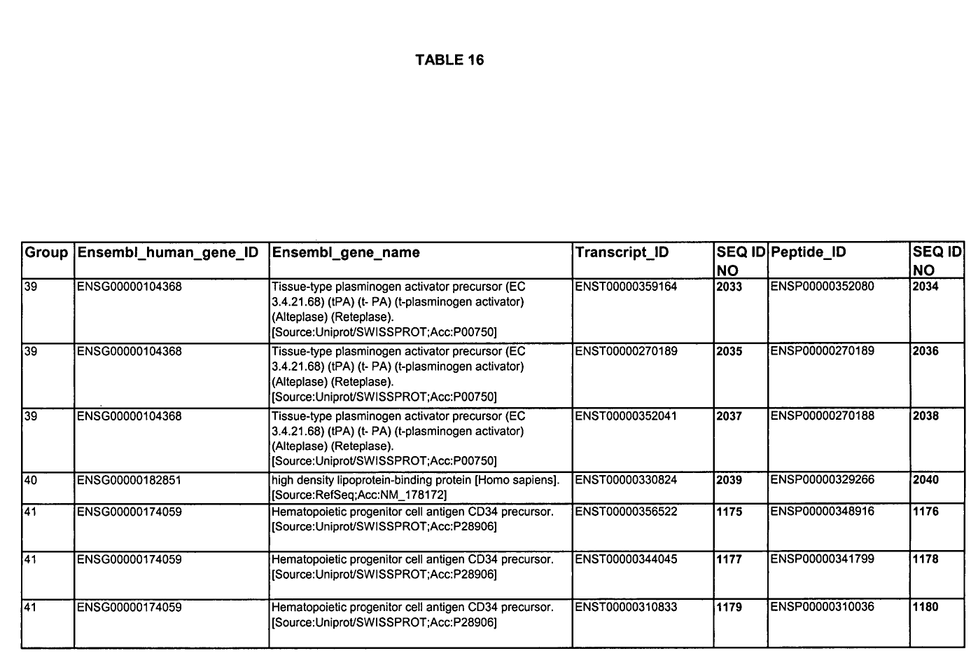

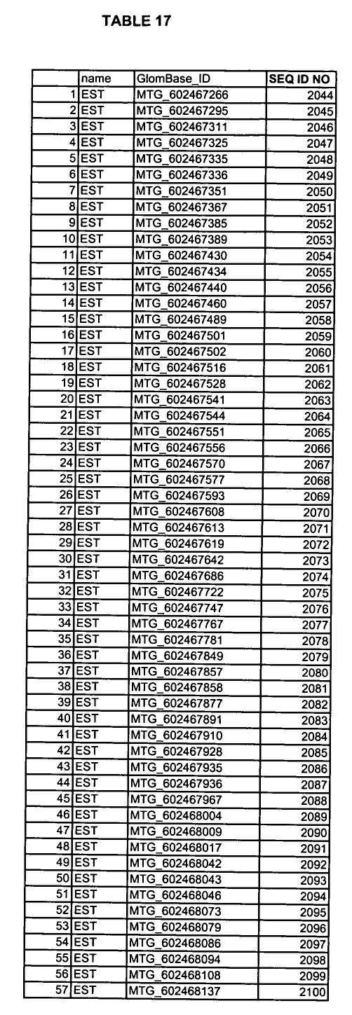

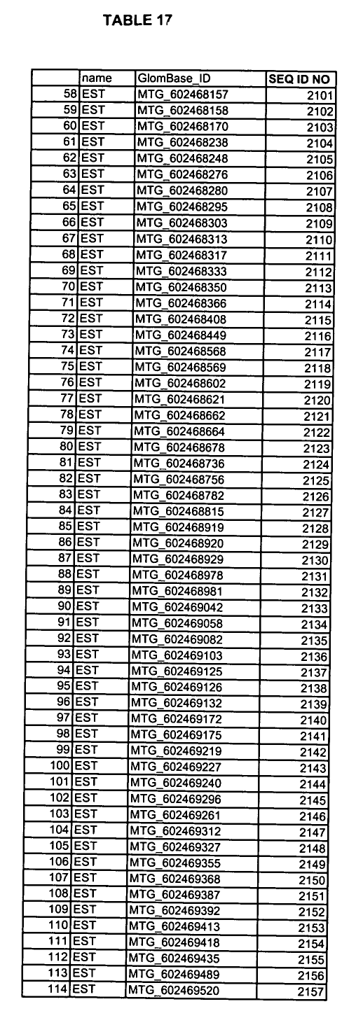

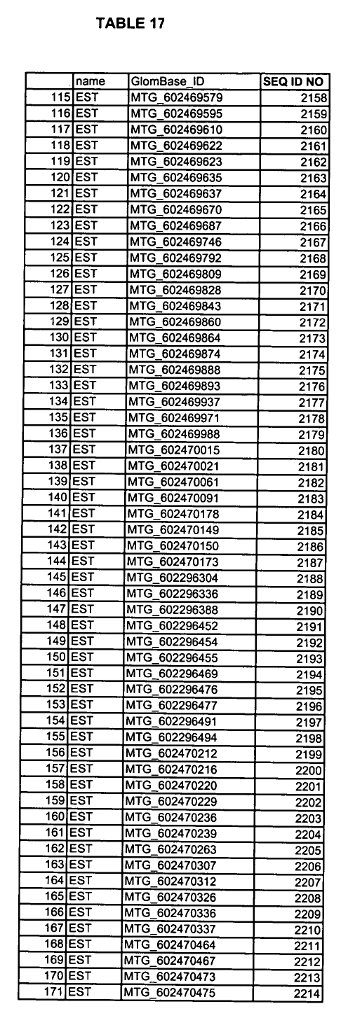

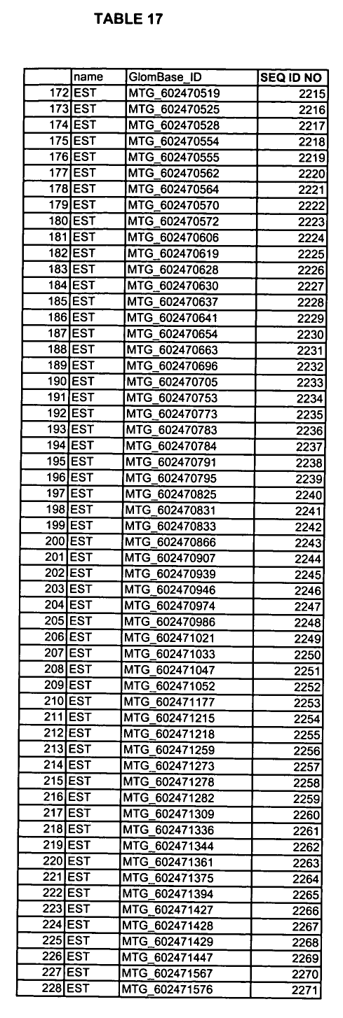

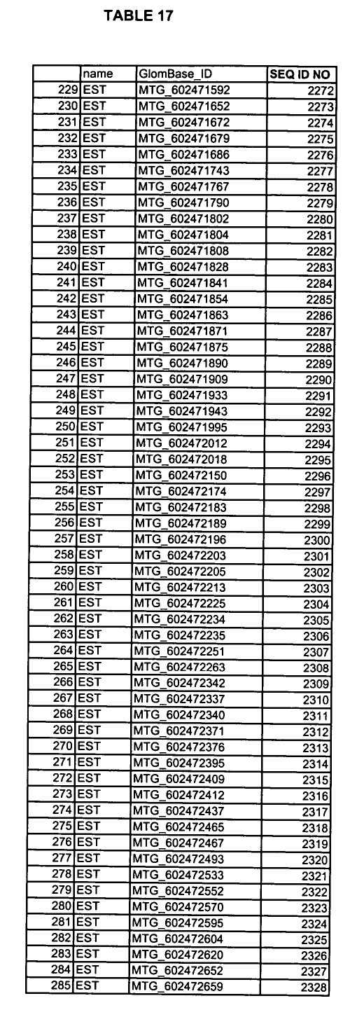

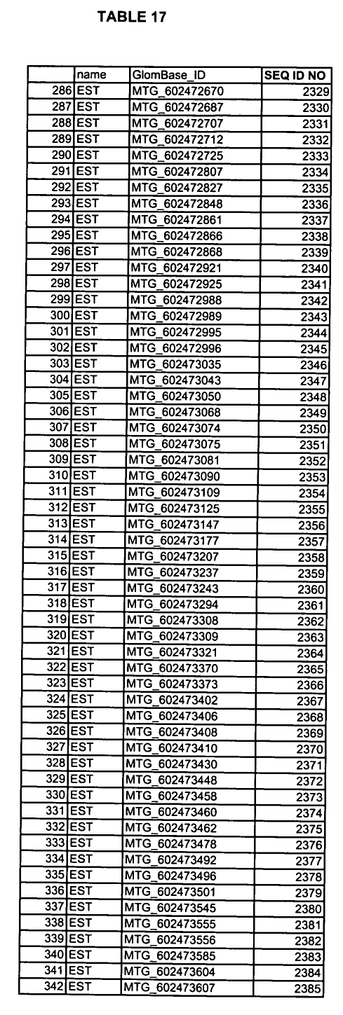

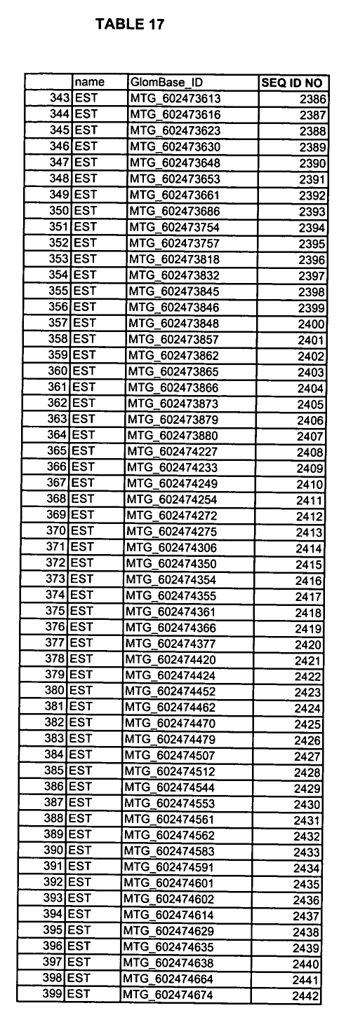

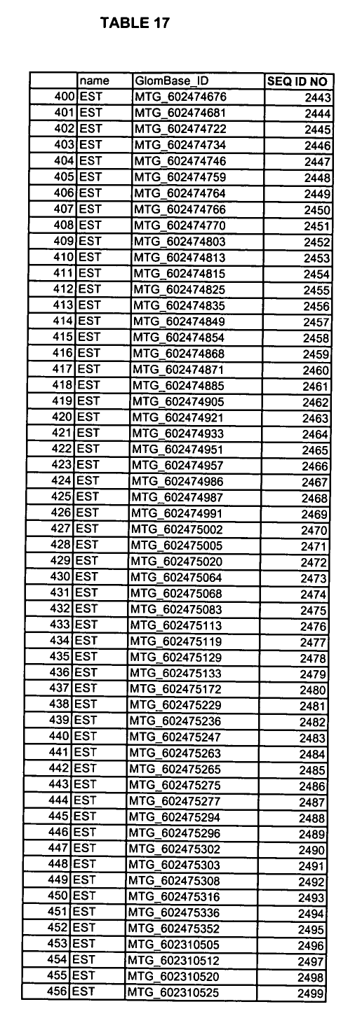

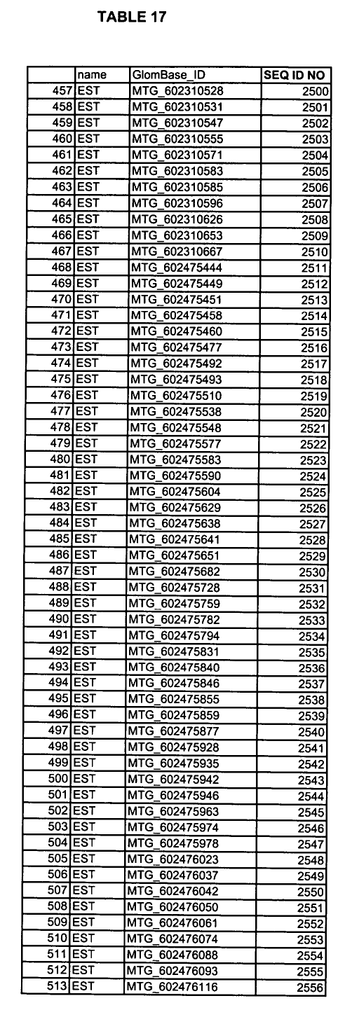

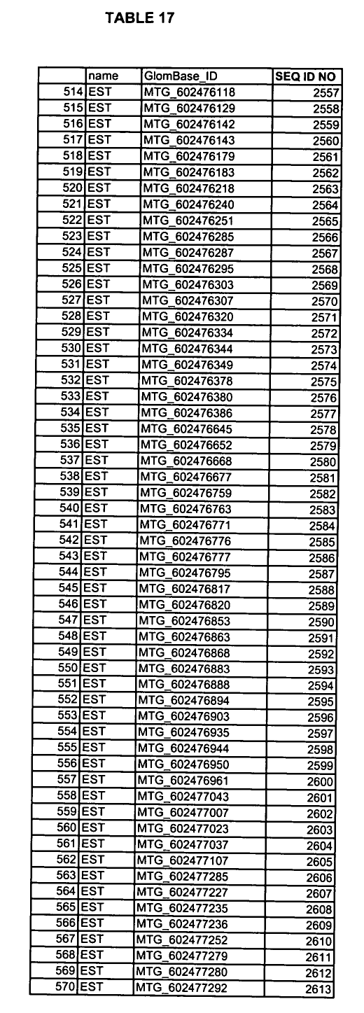

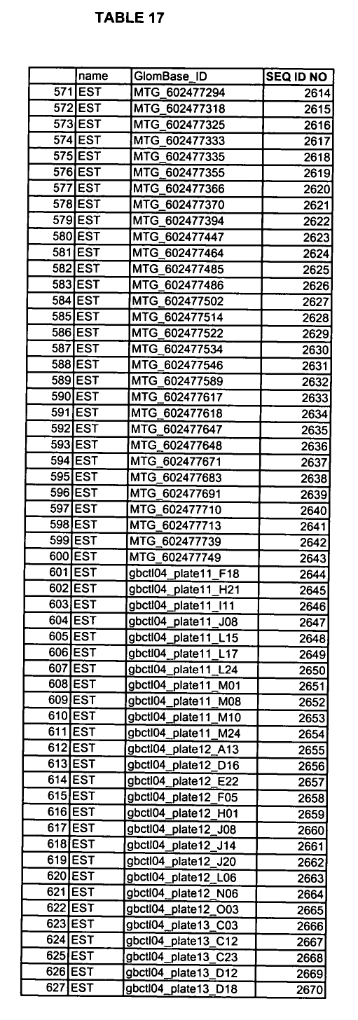

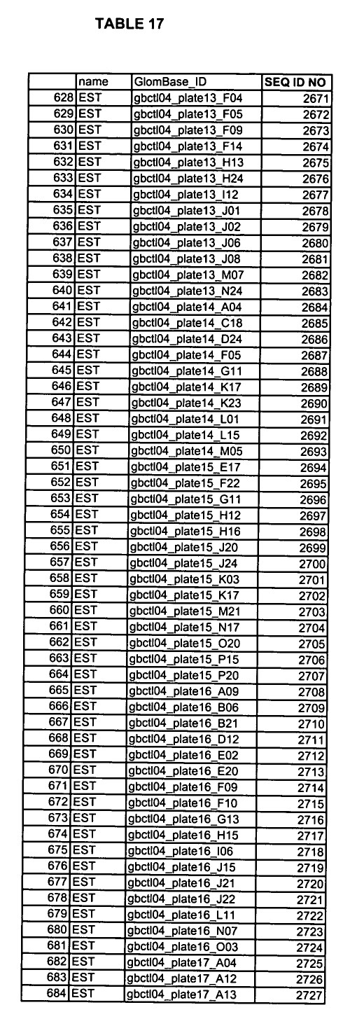

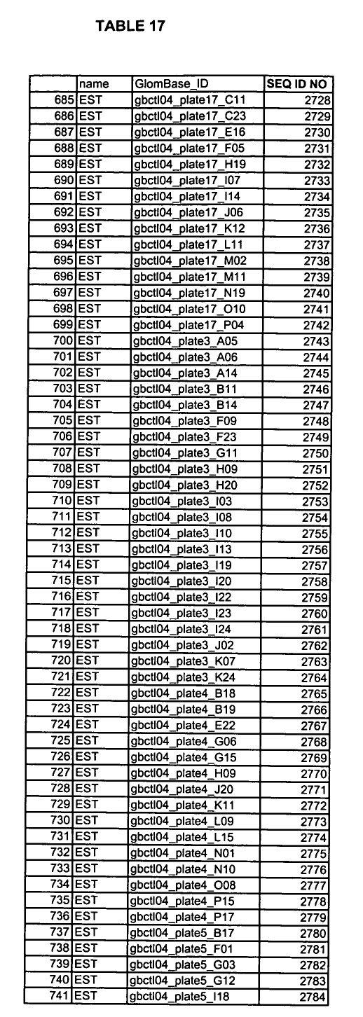

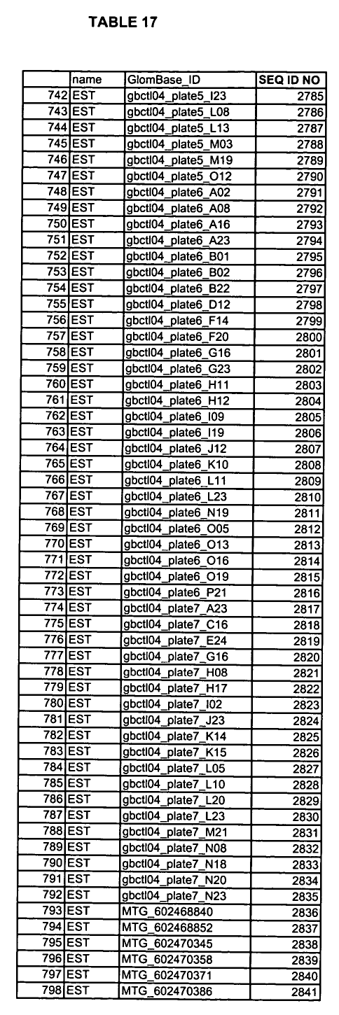

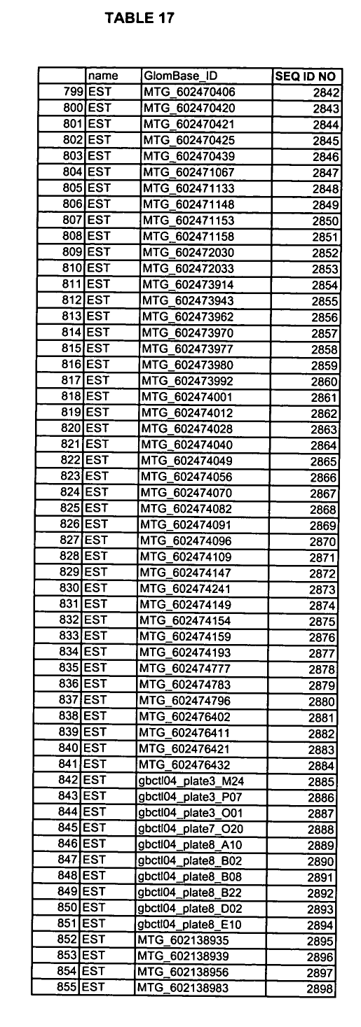

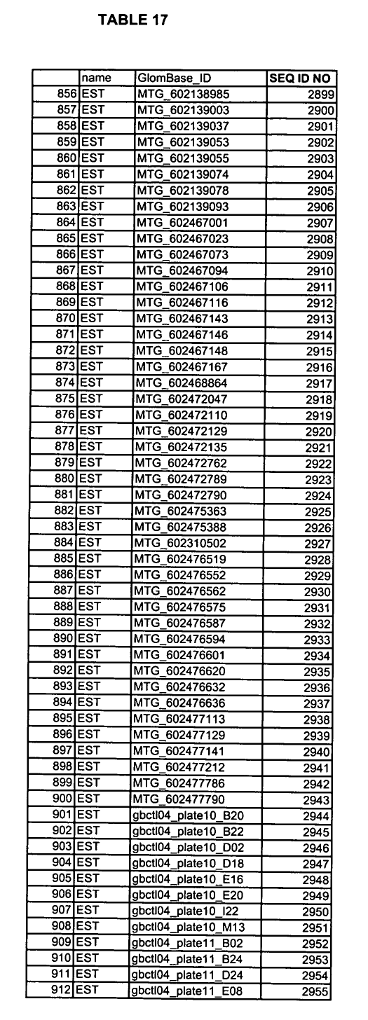

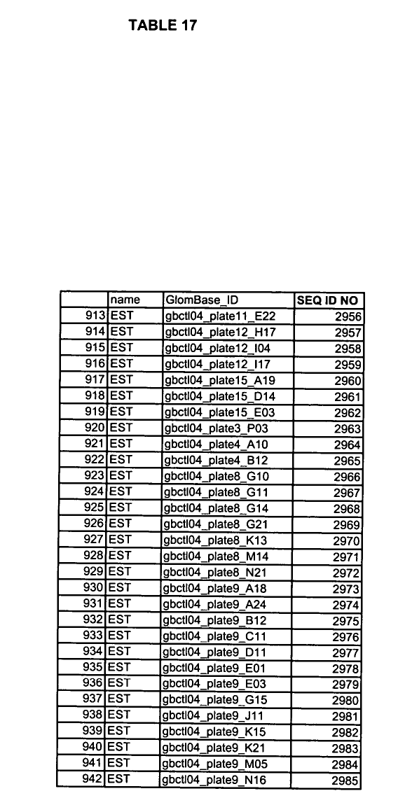

Using a magnetic bead perfusion method (45), highly purified kidney glomeruli were isolated from approximately 100 adult and 400 newborn mice in order to generate sufficient quantities of total RNA (approximately 400 μg from each group of glomeruli) for the synthesis of high-complexity cDNA libraries. Two standard oligo dT-primed cDNA libraries were generated from the newborn and adult glomerular RNA, respectively. In order to facilitate the identification of rare transcripts, two normalized libraries were also generated from the standard adult library. In the normalized libraries, high-abundance RNA transcripts were suppressed to different degrees (FIG. 1A). Test sequencing of 96 random clones from each library and analysis of the result in comparison with the Incyte Inc. mouse EST database indicated that the glomerular libraries were of high complexity. The analysis also provided an estimate of the number of sequences required to approach saturation in a large-scale sequencing effort. Based on these estimates, we attempted a total of 15,627 sequence reads from the four libraries, which provided 14,171 sequences of 500-800 bp length (91% readability). After vector trimming, a total of 13,368 cDNA sequences remained that were longer than 100 bp remained (data for the individual libraries shown in Table 1). Blast searches against the ENSEMBL mouse gene predictions (ENSEMBL mouse release 26.33b.1, 2004-09-03) resulted in 12,309 high quality hits (e-value <e⁻³⁰, alignment identity >85%) representing 6,053 different genes. The 13,368 cDNA sequences and their gene annotations were collected in a database, GlomBase (available as supplementary information online (http://www.mbb.ki.se/matrix/cbhome.htm) (See Table 14). 942 sequences did not match ENSEMBL annotated genes, but matched the mouse genome and may therefore represent putative novel gene transcripts, or contaminations by genomic DNA (See Table 17).

The gene and EST representation in the individual libraries is shown in FIG. 1A. To evaluate the quality of the cDNA library normalization procedure, we studied the distribution of a number of housekeeping genes among the different libraries. As shown in FIG. 1B, the relative abundances of housekeeping gene transcripts were decreased in the normalized libraries compared to the standard library. This shows that the normalization procedure suppressed the representation of high abundance and/or ubiquitously expressed transcripts.

The Glomerular Transcript Database (GlomBase) has a Unique Composition

Comparison with the ENSEMBL mouse gene predictions revealed that out of the total number of 28,055 hitherto annotated genes, 6,053 (21.6%) occur are present in GlomBase. Out of the 25,383 coding genes and 2,672 pseudogenes predicted in the mouse genome, 6,012 coding genes (23.7%) and 41 pseudogenes were found in GlomBase. Combined with the recent estimate of the coding capacity of the human genome, predicting only some 20,000-25,000 protein-coding genes (46), our data suggest that GlomBase, representing only three cell types, may contain cDNA sequences corresponding to 20-25% of the mammalian genome. In order to confirm that the content of GlomBase is enriched for glomerulus-expressed genes, and to estimate its coverage of the glomerular transcriptome, the 13,368 EST sequences of GlomBase were matched to mouse Unigene clusters (NCBI Mus musculus UniGene Build #144, 2005-01-20) and compared with the 109,633 EST sequences of 12 different cDNA libraries from mouse kidneys of different developmental stages and mouse strains (Table 2). We focused the comparison on transcripts known to be specific to, or enriched in podocytes, a unique epithelial cell type present only in the kidney glomerulus. Nephrin (Nphs1) (3), podocin (Nphs2) (4), podocalyxin (Podx1) (47), β-actinin-4 (Actn4) (30), synaptopodin (Synpo) (48), cyclin-dependent kinase inhibitor 1C (Cdkn1c) (49), protein-tyrosine phosphatase receptor o (Ptpn15, Ptpro) (50), Wilm's tumor protein Wt-1 (51), Transcription factor 21 (Tcf21, Pod1) (52) and forkhead box c2 (Foxc2) (53))transcripts encode structural proteins as well as cell-cycle regulators, receptors and transcription factors, and are hence expected to represent both high- and low-abundance podocyte mRNAs. The relative representation of all these genes was significantly higher (5 to 85-fold) in GlomBase compared to the kidney libraries (Table 2). Notably, the genes that are expressed exclusively in podocytes within the kidney showed an average higher relative representation in GlomBase (Nphs1, 45-fold; Nphs2, 15-fold; Podx1, 85-fold; Synpo, 4 hits in GlomBase, absent from kidney libraries; Ptpro, 49-fold; Wt-1, 12-fold) than the genes that are expressed also in other cell types of the kidney, albeit at lower levels than in the glomerulus (Actn4, 5-fold; Cdkn1c, 16-fold; Tcf21, 5-fold; Foxc2, 8-fold) (Table 2). Since glomeruli make up less than 10% of the kidney tissue, we expected to find more than 10-fold higher representation of the podocyte-specific cDNAs in GlomBase than in the whole kidney libraries. Indeed, the observed representation was higher than 10-fold, on average, ranging from 12 to 85 fold for the podocyte-specific transcripts.

The high representation of podocyte-specific transcripts confirms the high complexity and coverage of the glomerular transcriptome in GlomBase. The number of cDNA clones selected for sequencing was chosen to approach 90% saturation based on initial calculations. Extrapolation of the relationship between the number of EST sequences and the number of different ENSEMBL annotations in our standard cDNA libraries suggested a total complexity of about 7,100 genes, and hence, that approximately 85% (6,053/7,100) saturation was reached (data not shown). Based on the assumption that the glomerular cDNA libraries in total (>5 million clones) cover the glomerular transcriptome by 100%, we estimate that approximately 85% of the glomerular transcriptome is represented in GlomBase. In order to validate this estimate, we collected exhaustive information from the published literature on gene and protein expression patterns in the glomerulus, demonstrated with cellular resolution by either in situ hybridization or immunohistochemistry. Out of 170 such genes or proteins, we found 140 (82.4%) in GlomBase (Table 8). Based on these results, we conclude that GlomBase covers the glomerular transcriptome by more than 80%.

Construction of a Mouse Glomerulus cDNA Chip (GlomChip)

We amplified and printed the cDNA clones of GlomBase onto glass slides for transcriptional profiling experiments. We placed on the same chip a commercial unigene collection of 1,306 mouse cDNA clones from the IMAGE consortium (http://image.llnl.gov/), selected without tissue preference, as well as a small number of controls (see Methods). The overall design of GlomChip is illustrated in FIG. 2A. The printing of the entire clone set in triplicate on each slide, and the careful distribution of control spots, allowed for normalization and statistical evaluation of the result obtained from single chip hybridizations. A typical two-target hybridization is shown in FIG. 2B. The horizontal stripe of weak hybridization across each quadrant of 34×34 spots represents the position of the clones from normalized libraries, confirming that on average, these transcripts are of lower abundance than those represented by the standard libraries.

Transcriptional Profiling Using GlomChip Identifies Candidate Potential Novel Glomerulus- and Podocyte-Specific Genes

We used GlomChip for a series of experiments aiming at the identification of genes with glomerulus-enriched or glomerulus-specific expression (FIG. 2C,D). In a first experiment, we compared glomeruli isolated from 5-day-old mice kidney to the non-glomerulus fraction of kidney tissue that remained after magnetic separation of the glomeruli (FIG. 2C,D). A total of 937 GlomBase cDNA clones representing 356 different ENSEMBL genes and 64 ESTs were significantly upregulated more than 2-fold (category 1 genes in FIG. 2C; gene list available in Table 9) whereas 681 cones representing 354 different ENSEMBL genes and 34 ESTs were upregulated in non-glomerulus kidney tissue (category 2 genes in FIG. 2C; gene list available in Table 10). The list of category 1 genes contained most known podocyte markers, e.g. Nphs1, Nphs2, Ptpro, Wt-1, Cdkn1c, Podx1, Synpo, and many known markers for vascular endothelial cells, e.g. Pcam, Kdr, and Edg1. The concentration of vascular transcripts in the glomerulus was expected since vascular wall cells (endothelial cells and mesangial cells) together constitute about 85% of the glomerular cells, but only a small minority of the cells in the remaining kidney tissue. To further categorize the genes upregulated in the glomerulus, we compared the glomerulus transcript profile with that of capillary fragments isolated from mouse brain. These brain vessel fragments are composed to 90% of endothelial cells and pericytes (54). By this comparison, we subdivided the category 1 genes further into category 3 genes upregulated in glomeruli (430 cDNA clones representing 142 ENSEMBL genes and 35 ESTs; Table 11), category 4 genes upregulated in brain capillary fragments (67 cDNA clones, representing 34 genes and 1 EST; Table 12) and category 5 genes which were not significantly differentially expressed more than 2-fold between glomeruli and brain capillaries (440 cDNA clones, representing 180 ENSEMBLE genes and 28 ESTs; Table 13). As expected, most known podocyte markers collected into category 3, whereas known endothelial markers were found in category 4 and 5. For example, category 4 included many broad endothelial makers, such as Icam2, Cd34, Pecam, Flt1, Kdr and Edg1. While some of these are known to be expressed in glomerular endothelial cells, their expression is apparently higher in brain capillaries.

The category 3 genes represent candidate specific markers for any of the three cell types of the glomerulus. To assign these genes further to the individual glomerular cell types, we FACS-sorted GFP-positive podocytes from mice in which GFP expression was activated from the Z/EG transgene by Cre-recombinase expressed under the control of the podocin (Nphs2) promoter (55) (FIG. 3A). Glomeruli were isolated by Dynabead perfusion from 8-day-old podocin-Cre;Z/EG mice (FIG. 3B), and enzymatically digested into single cell suspensions (FIG. 3C). Before sorting, the frequency of GFP-positive cells was 2-5% (FIG. 2D, 3D). After sorting (FIG. 2E, 3E), the GFP-negative fraction contained <0.07% GFP-positive cells. Due to limited cell numbers in the sorted GFP-positive fraction these cells were all used for RNA preparation, and the percentage of GFP-positive cells was therefore not determined. RNA was extracted from 15,000 GFP-positive cells obtained from 3 mice, and from the same number of GFP-negative cells, and used for GlomChip analysis. This resulted in the further subdivision of the category 3 genes into those upregulated in GFP-positive cells (category 6 genes, podocyte-expressed, 138 cDNA clones, 48 different ENSEMBL genes and 11 ESTs; Table 3), and those upregulated in GFP-negative cells (category 7 genes, non-podocyte glomerular genes, 60 cDNA clones, 18 different ENSEMBL genes; Table 4), and those not significantly differentially expressed more than 2-fold between GFP+ and GFP− negative cells (category 8 genes, 233 cDNA clones, 76 different ENSEMBL genes and 24 ESTs) (Table 5).

Whereas the GlomChip IMAGE clones present on the GlomChip represent 1164 different genes (19.2% of the number of different GlomBase genes), only 33 IMAGE genes fell into category 1 (9.3% compared to GlomBase), whereas while 119 IMAGE genes fell into category 2 (33.6% compared to GlomBase) (FIG. 2D). This difference was expected since a “random” set of genes would be more likely to represent transcripts expressed in abundant tissue, such as whole kidney, than in scarce cell types, such as glomerular cells. Accordingly, the IMAGE set contributed only a single gene each to the most glomerulus-specific gene categories 6 and 7.

Category 6 genes represent candidate podocyte-specific transcripts. Indeed, most known podocyte-specific transcripts (e.g. Nphs1, Nphs2, Ptpro, Wt1, Synpo, Podx1) fell among the top 20 genes in category 6, and several other genes known to be highly expressed in podocytes (Cdnk1c, Foxc2, Microtubule-associated protein tau) were also present in category 6 (Table 3). Category 7 genes instead include several known mesangial cell and juxtaglomerular markers, such as renin1 (Ren1), insulin-like growth factor-binding protein 5 (Igfbp5), integrin alpha 8 (Itga8), Protease nexin I (Serpine2, PN-1), and mesoderm-specific transcript (Mest) (Table 4), and therefore represent a list of potential mesangial cell markers. Category 7 genes may also include markers that are specific to glomerular endothelial cells in comparison with other types of endothelium.

Identification of New Novel Glomerulus-Specific Genes

In order to establish the cellular expression of some of the novel candidates for podocyte- and non-podocyte-specific glomerular transcripts (selected from the category 6 and 7 lists) we employed in situ hybridization. FIG. 4A shows by non-radioactive in situ hybridization the expression of 5 novel podocyte markers, Semaphorin sem2 (Sem2), Rhophilin 1 (Rhpn1), Cbp/p300-interacting transactivator 2 (Cited 2), Protease inhibitor 15 (Pi15), and Gene X, in comparison with 3 known podocyte markers, Nphs2, Podx1 and Cdkn1c. FIG. 4B shows the expression of 3 novel mesangial markers, secreted frizzled-related protein 2 (Sfrp2 ), Aldo-keto reductase family 1 member B7 (Akr1b7), and Lim domain only protein 7 (Lmo7) in comparison with known mesangial and juxta-glomerular apparatus (JGA) transcripts Igfbp5 and Ren1. FIG. 4B also shows the expression of endomucin (Emcn), a vascular endothelial marker, in glomerular endothelial cells. In instances where non-radioactive in situ hybridization failed, we employed radioactive in situ hybridization. By this method, we localized 3 additional transcripts to podocytes; Schwannomin interacting protein 1 (Schip1), Clone52 and dendrin, and one additional transcript to mesangial/endothelial cells; EH-domain containing protein 3 (Ehd3) (FIG. 4C).

One should note that although the novel podocyte and mesangial/endothelial markers are restricted in their cellular expression in the kidney, extra-glomerular expression sites for some of these genes have been reported. In some cases, we confirmed the extra-renal expression sites by in situ hybridization, Northern blotting and EST database mining. However, by their extra-renal expression, the novel glomerular cell markers do not distinguish from known ones (e.g. Nphs1, Nphs2 and Podx1) all of which show restricted sites of extra-renal expression. Below follow brief commentaries on some of the available information regarding the above-mentioned novel glomerular cell markers:

Rhophilin 1 was originally identified as a small GTPase Rho binding protein using a yeast two-hybrid system (56). Expression in germ cells in the mouse testis and localization in the principal piece of the spermatozoa has been documented (57), but its function is unclear.

Semaphorin sem 2 cDNA sequences have previously been identified only in an human adult spleen library, but nothing has been reported further on its expression pattern or function. Semaphorins are members of a large, highly conserved, family of molecular signals that were identified initially through their role in axon guidance (58), and later, in angiogenesis (59, 60).

Protease inhibitor 15 has previously been identified as a trypsin inhibitor secreted by human glioblastoma cells (61).

Cbp/p300-interacting transactivator 2 (Cited2; or Melanocyte-specific gene 1-related gene1) transcripts have previously been identified in human endothelial cell and neonatal brain (62). It has been proposed that Cited2 acts as a negative regulator of hypoxia-inducible factor (HIF)-1-alpha through competitive binding to CBP/p300. Cited2 knockout mice die at late gestation (63).

Dendrin has previously been identified as a brain-specific gene (64) of unknown function.

“Clone 52” is newly annotated gene (ENSMUSG00000050010) predicted to encode a transmembrane protein. Its expression pattern has not previously been described.

Schwannomin interacting protein I was originally identified as a partner of schwannomin, a candidate gene for type II neurofibromatosis, using yeast two-hybrid methodology (65). Schip 1 may regulate the activity of schwannomin, however, its exact physiological function is unclear.

“Gene X” is a GlomBase EST (SEQ ID NO: 2043; MTG_{—}602467023) without current annotation or prior information about its protein coding capacity or expression.

Eh domain-containing protein 3 was originally identified as a homologue of human EHD1 (testilin/HPAST) in a human fetal brain cDNA library (66). It has been proposed that EHD3 together with EHD1 may be involved in regulating the movement of recycling endocytotic vesicles along with microtubule-dependent tubular tracks (67).

Secreted frizzled-related protein 2 (Sfrp2) or secreted apoptosis related protein 1 (SARP1) was identified by differential display as a gene that is expressed in quiescent but not in exponentially growing 10T1/2 cells (68) and has been reported that acts as soluble modulators of Wnt signaling (69). The expression of sFRP2 in aggregating mesenchyme and glomerulus has been reported (70).

Aldo-keto reductase family 1 member B7 or mouse vas deferens protein was initially described as a major secretary protein of the vas deferens (71). A role in steroidogenic activity has been proposed.

LIM domain only protein 7 (LMO7) was identified in a human pancreatic cDNA library and encodes a single LIM domain (72). A possible role in assembling adhesion junction in epithelial cells has been reported (73), however functional roles in vivo remain unclear.

Temporal Expression Patterns of Glomerular Cell Markers During Nephron Development

We next compared the expression of the novel glomerular markers at different stages of glomerular development. None of the podocyte markers was expressed during the comma-shaped stage of nephron development, i.e. before morphological distinctions can be made between prospective podocytes and tubular epithelium (FIG. 5). Morphologically distinguishable podocytes appear during the S-shaped stage of nephron development. At this stage, the known podocyte markers Nphs2, Podx1 and Cdkn1c began to appear in developing podocytes, together with the novel markers Sem2, P15 and gene X. During the capillary loop and mature stages, all podocyte markers were expressed (FIG. 4C and 5). PI15 was the only marker showing a peak of expression during S-shaped and capillary loop stages followed by downregulated expression (FIG. 5), suggesting that this protease inhibitor might have a particularly important role during development of the glomerulus.

The previously known as well as the novel mesangial markers were expressed first during the capillary loop and mature stages, with the exception of Sfrp2, which was first expressed in the epithelium of the developing nephron during comma and S-shaped stages, and then switched to the mesangium during the capillary loop and mature stages (FIG. 5). In addition to the mesangial cells, expression of all these markers were also noticed in smooth muscle cells of the feeding and draining arterioles in the juxtaglomerular region.

Dendrin Localizes to the Podocyte Foot Process Region

The podocytes are atypical epithelial cells in the sense that they form foot processes linked by slit diaphragms rather than typical epithelial junctions. The critical role of the foot process and the slit diaphragms for filtration has been well established, and hence it is important to establish if the novel podocyte marker genes encode proteins that play a role in the establishment, function and maintenance of these structures. As a first step towards this goal, we have started to systematically generate antibodies to these proteins and map the protein expression sites and subcellular distriubution. Here, we report the example of dendrin, predicted as a cytoplasmic protein without apparent homology to other proteins or protein domains. A mouse dendrin cDNA clone was derived by PCR and expressed in order to generate recombinant his-tagged dendrin protein. This protein was used to generate polyclonal rabbit antisera. The specificity of the antiserum was confirmed by transfecting COS-7 cells with full length dendrin cDNA and control cDNA (data not shown). Western blotting (FIG. 6D) and immunohistochemistry (FIG. 6B) on E18.5 mouse kidneys localized the dendrin protein exclusively to glomeruli within the kidney, and high power views revealed a staining pattern consistent with the distribution of podocytes (FIG. 6B inset). The overall distribution of the dendrin protein was in accordance with the distribution of its mRNA (FIG. 6A and inset). By immuno-electron microscopy, the dendrin protein was further sub-localized to the inner leaflet of the foot process membrane (FIG. 6C) and was concentrated to regions where the foot processes appose and are bridged by slit diaphragms (FIG. 6C, arrows).

Discussion

In spite of a diversity of etiologies of kidney diseases, the glomerulus is often the primary target of the pathological process. Proteinuria, uniform or focal expansion of the mesangial matrix, thickening of the GBM and effacement of podocyte foot processes are frequently observed pathologic hallmarks of glomerular disease. The inability of the terminally differentiated podocytes to proliferate and repopulate a damaged glomerulus is believed to contribute to glomerular scarring (74), possibly by triggering changes in the proliferation and/or matrix deposition by endothelial and mesangial cells. The intimate interplay and interdependence between the three glomerular cell types during glomerular development is, therefore, also reflected in pathogenic processes, causing difficulties in defining primary molecular and cellular pathogenic events and cause-effect relationships. Since the renal diseases of glomerular origin constitute a huge burden to society and because the prevalence of glomerular disease is increasing, there is an urgent need for a deeper understanding of glomerular development and biology, and insights into the different mechanisms of glomerular disease. We need to identify new therapeutic drug targets, as well as markers that improve disease classification and help in monitoring disease progression and response to therapy. Understanding the molecular basis of glomerular function and injury is a prerequisite for such advances. Molecular profiling of the glomerulus is likely to contribute to both identification of novel diagnostic markers and candidate drug targets.

The present glomerular profiling study has revealed extensive new information about genes and proteins that, in the kidney, are preferentially or specifically expressed in cells of the glomerular filtration apparatus. First, a unique glomerulus isolation technique was used to collect high quality RNA from mouse glomeruli to allow construction of specific glomerular cDNA libraries. Importantly, these libraries were shown to represent both high and low abundancy transcripts from all developmental stages of the glomerulus. Second, a large-scale cDNA sequencing effort generated GlomBase, a database of about 6,053 glomerulus-expressed genes with over 80% coverage of the glomerular transcriptome. GlomBase is accessible online (http://www.mbb.ki.se/matrix/cbhome.htm). This database will be continuously updated as additional glomerular transcripts are identified, such as with global microarrays or in other studies. This database should be useful to investigators interested in the renal filtration system.

Third, spotted microarrays (GlomChip) containing the GlomBase cDNA collection were generated and used to perform a series of hybridizations leading to the identification of over 300 novel glomerular transcripts, most of the corresponding protein products, as yet, having an unknown function. Fourth, in situ hybridization and immunostaining procedures localized many of the novel transcripts to one of the three glomerular cell types, i.e. podocytes, mesangial and endothelial cells. Fifth, detailed analysis of one novel podocyte marker protein, dendrin, was shown by immunelectron microscopy to be associated with the podocyte foot processes. We are convinced that the results of this study will provide new tools and opportunities for kidney research, such as for addressing various questions of glomerular development and biology, and providing new unique means for studying the development and pathomechanisms of glomerular disease.

While transcription profiling studies have previously been performed on healthy and diseased kidneys, (39-41, 43, 75-79), including isolated glomeruli (80, 81), only two previous studies have, to our knowledge, reported efforts to map the glomerular transcriptome in comparison to other parts of the nephron (42, 44). When comparing the present results with those of the two earlier reports, it is apparent that the sets of glomerulus-enriched genes predicted independently by the three studies that show a relatively limited overlap (FIG. 7). This cannot be explained only by different gene representation on the arrays, or bias in the SAGE method, as approximately 60% of the gene transcripts predicted to be upregulated in the glomerulus by both of the other studies (65/119 and 88/143 respectively) are present in GlomBase, however, many of these genes were not found by us to be statistically significantly overexpressed >2-fold in the glomerulus (we even found about 20% of them to be downregulated in glomeruli relative to the rest of kidney). Some of the discrepancy therefore likely reflects differences in cut-off thresholds between the studies. For example, many of the genes listed by the other studies fall just below our cut-off for fold difference or statistical significance (for example Vegfa, which is known to be expressed by podocytes, see FIG. 7). It is also possible that some of the discrepancy relates to species differences in the studies (human vs mouse). Other possible confounding variables include differences in tissue handling, which may induce selective degradation of RNA species, and in target preparation. Notably, the SAGE study (42) failed to detect the podocyte transcript nephrin, whereas the microarray study by Higgins et al. (44) failed to identify several known podocyte markers, including Nphs2, which is abundantly expressed in podocytes. Positioning 170 literature-validated markers for glomerular cells (full list and references available as Supplementary Information, Table S1) in the diagram of FIG. 7 shows that our study accurately predicts more of the glomerular markers than the two other studies combined. Importantly, none of the new podocyte markers validated in this study by in situ hybridization were found in the previous reports.

Undoubtedly, the identification of numerous novel highly glomerulus-associated or -specific genes will eventually increase our understanding of glomerulus biology and mechanisms of glomerular disease. The mutations identified thus far that cause hereditary monogenic glomerular diseases encoded glomerulus-specific or -associated proteins with critical roles in glomerulus development and function. Thus, the pathological findings in Alport and congential nephrotic syndromes have provided unique insight into the molecular nature and properties of the GBM and the podocyte slit diaphragm. The present in situ hybridization analyses revealed the existence of several novel podocyte protein-coding mRNAs, as well as additional mRNAs in mesangial cells. The more detailed expression analysis and immuno-electron microscopic localization of the novel podocyte protein dendrin to the slit diaphragm region, imply that this intracellular protein, that was previously considered to be brain specific, also has a role in renal filtration. To understand the physiologic roles of the novel glomerular proteins, they also need to be explored individually, e.g. by mutagenesis in animal models. Interestingly, some of the glomerular genes identified in this study, such as adenylate cyclase 1 and Foxc2, that also have restricted extrarenal expression, have already been inactivated in mice, and we have recently shown that these mutants develop both mild and severe renal phenotypes (Patrakka et al. and Takemoto et al., manuscripts in preparation). Accordingly, it is very likely that many of the other novel glomerular proteins identified in this study are involved in glomerular disease, both as primary targets and bystanders.

It is now a major task to examine the physiological role and disease involvement of the many novel glomerulus proteins using animal and cellular model systems, as well as pathologicalal specimens. To study the function of such a large number of genes in mice using gene targeting in embryonic stem cells is a huge task, but an attractive alternative is to apply the approach of gene knock-down using morpholino oligonucleotides in zebrafish embryos. Zebrafish embryos develop a fully functioning mesonephros containing a single glomerulus already 48-72 hours post fertilization.

Global transcript profiling of kidney diseases has already facilitated advancement of the categorization of renal dysfunction (76), and it is likely to help in predicting individual responsiveness to therapy, delineation of molecular pathways controlling renal physiological and pathological processes, including identification of putative future molecular targets of pharmacological therapy. From the point of view of glomerular disorders, renal tissue heterogeneity and scarcity of the relevant cell types will inevitably confound data interpretation. It is therefore important to make sure that the profiling platform meets the demands required for transcriptome analysis in the relevant cell types. The present study provides an important step towards such goals with regard to mouse models of glomerular diseases.

### REFERENCES

1. Deen, W. M. 2004. What determines glomerular capillary permeability? J Clin Invest 114:1412-1414.
2. Barker, D., Hostikka, S. L., Zhou, J., Chow, L. T., Oliphant, A. R., Gerken, S. C., Gregory, M. C., Skolnick, M. H., Atkin, C. L., and Tryggvason, K. 1990. Identification of mutations in the COL4A5 collagen gene in Alport syndrome. Science 248:1224-1227.
3. Kestilä, M., Lenkkeri, U., Männikkö, M., Lamerdin, J., McCready, P., Putaala, H., Ruotsalainen, V., Morita, T., Nissinen, R., Herva, R., et al. 1998. Positionally cloned gene for a novel glomerular protein—nephrin—is mutated in congenital nephrotic syndrome. Mol. Cell 1:575-582.
4. Boute, N., Gribouval, O., Roselli, S., Bennessy, F., Lee, H., Fushshuber, A., Dahan, K., Gubler, M. C., Niaudet, P., and Antignac, C. 2000. NPHS2, encoding the glomerular protein podocin, is mutated in autosomal recessive steroid-resistant nephrotic syndrome. Nat Genet. 24:349-354.
5. Somlo, S., and Mundel, P. 2000. Getting a foothold in nephrotic syndrome. Nat Genet 24:333-335.
6. Haber, D. A., Buckler, A. J., Glaser, T., Call, K. M., Pelletier, J., Sohn, R. L., Douglass, E. C., and Housman, D. E. 1990. An internal deletion within a 11p13 zinc finger gene contributes to the development of Wilm's tumor. Cell 61:1257-1269.
7. Mochizuki, T., Lemmink, H. H., Mariyama, M., Antignac, C., Gubler, M. C., Pirson, Y., Verellen-Dumoulin, C., Chan, B., Schroder, C. H., Smeets, H. J., et al. 1994. Identification of mutations in the alpha3(IV)and alpha4(IV) collagen genes in autosomal recessive Alport syndrome. Nat Genet 8:77-82.
8. Zenker, M., Aigner, T., Wendler, O., Tralau, T., Muntefering, H., Fenski, R., Pitz, S., Schumacher, V., Royer-Pokora, B., Wuhl, E., et al. 2004. Human laminin beta2 deficiency causes congenital nephrosis with mesangial sclerosis and distinct eye abnormalities. Hum Mol Genet 13:2625-2632.
9. Dreyer, S. D., Zhou, G. Baldini, A., Winterpacht, A., Zabel, B., Cole, W., Johnson, R. L., and Lee, B. 1998. Mutations in LMX1B causes abnormal skeletal patterning and reanal dysplasia in nail patella syndrome. Nat Genet 19:47-50.
10. Klamt, B., Koziell, A., Poulat, F., Wieacker, P., Scambler, P., Berta, P., and Gessler, M. 1998. Frasier syndrome is caused by defective alternative splicing of WT1 leading to an altered ratio of WT1±KTS splice isoform. Hum Mol Genet 7:709-714.
11. Tryggvason, K., and Wartiovaara, J. 2005. How does the kidney filter plasma? Physiology 20:96-1001.
12. Hudson, B. G, Tryggvason, K., Sundaramoorthy, M., and Neilson, E. G 2003. Alport's syndrome, Goodpasteur's syndrome, and type IV collagen. New Engl. J Med. 348:2543-2556.
13. Miner, J. H., Patton, B. L., Lentz, S. I., Gilbert, D. J., Snider, W. D., Jenkins, N. A., Copeland, N. G., and Sanes, J. R. 1997. The laminin alpha chains: expression, developmental transitions, and chromosomal locations of alpha 1-5, identification of heterotrimeric laminins 8-11, and cloning of a novel alpha3 isoform. J Cell Biol 137:685-701.
14. Pavenstadt, H., Kriz, W., and Kretzler, M. 2003. Cell biology of the glomerular podocyte. Physiol Rev 83:253-307.
15. Rodewald, R., and Karnowsky, M. J. 1974. Porous substructure of the glomerular slit diaphragm in the rat and mouse. J Cell Biol 60:423433.
16. Wartiovaara, J., Öfverstedt, L. G., Koshnoodi, J., Zhang, J., E., M., Sandin, S., Ruotsalainen, V., Cheng, R. H., Jalanko, H., Skoglund, U., et al. 2004. Nephrin strands contribute to a porous slit diaphragm scaffold as revealed by electron tomography. J Clin Invest 114:1475-1483.
17. Shih, N. Y., Li, J., Karpitskii, V., Nguyen, A., Dustin, L. M., Kanagawa, O., Miner, J. H., and Shaw, A. S. 1999. Congenital nephrotic syndrome in mice lacking CD2-associated protein. Science 286:312-315.
18. Schnabel, E., Anderson, J. M., and Farquhar, M. G. 1990. The tight junction protein ZO-1 is concentrated along slit diaphragms of the glomerular epithelium. J Cell Biol 111:1255-1263.
19. Inoue, T., Yaoita, E., Kurihara, H., Shimizu, F., Sakai, T., Kobayashi, T., Ohsohiro, K., Kawachi, H., Okada, H., Suzuki, H., et al. 2001. FAT is a component of glomerular slit diaphragms. Kidney Int. 59:1003-1012.
20. Sellin, L., Huber, T. B., Gerke, P., Quack, I., Pavenstadt, H., and Walz, G. 2003. NEPH1 defines a novel family of podocin interacting proteins. FASEB J. 17:115-117.
21. Liu, G. Kaw, B., Kurfis, J., Rahmanuddin, S., Kanwar, Y. S., and Chugh, S. S. 2003. Neph1 and nephrin interaction in the slit diaphragm is an important determinant of glomerular permeability. J Clin Invest 112:209-221.
22. Barletta, G. M., Kovari, I. A., Verma, R. K., Kerjaschki, D., and Holzman, L. B. 2003. Nephrin and Neph1 co-localize at the podocyte foot process intercellular junction and form cis hetero-oligomers. J Biol Chem 278:19266-19271.
23. Reiser, J., Kriz, W., Kretzler, M., and Mundel, P. 2000. The glomerular slit diaphragm is a modified adherence junction. J Am Soc Nephrol 11:1-8.
24. Tsukaguchi, H., Sudhakar, A., Le, T. C., and al., e. 2002. NPHS2 mutations in late-onset focal segmental glomerulosclerosis. J Clin Invest 110: 1659-1666.
25. Kim, J. M., Wu, H., Green, G., and al., e. 2003. CD2-assoiciated protein haploinsufficiency is linked to glomerular disease susceptibility. Science 300:1298-1300.
26. Putaala, H., Soininen, R., Kilpelainen, P., Wartiovaara, J., and Tryggvason, K. 2001. The murine nephrin gene is specifically expressed in kidney, brain and pancreas: inactivation of the gene leads to massive protenuria and neonatal death. Hum Mol Genet 10:1-8.
27. Donoviel, D. B., Freed, D. D., Vogel, H., Potter, D. G., Hawkins, E., Barrish, J. P., Mathur, B. N., Turner, C. A., Geske, R., Montgomery, C. A., et al. 2001. Proteinuria and perinatal lethality in mice lacking NEPH1, a novel protein with homology to NEPHRIN. Mol Cell Biol 21:4829-4836.
28. Ciani, L., Patel, A., Allen, N. D., and Ffrench-Constant, C. 2003. Mice lacking the giant protocadherin mFAT1 exhibit renal slit junction abnormalities and a partially penetrant cyclopia and anophtalmia phenotype. Mol Cell Biol 23:3575-3582.
29. Shaw, A. S., and Miner, J. H. 2001. CD2-associated protein and the kidney. Curr Opin Nephrol Hypertens 10: 19-22.
30. Kaplan, J. M., Kim, S. H., North, K. N., Rennke, H., Correia, L. A., Tong, H. Q., Mathis, B. J., Rodriguez-Perez, J. C., Allen, P. G., Beggs, A. H., et al. 2000. Mutations in ACTN4, encoding alpha-actinin-4, cause familial focal segmental glomerulosclerosis. Nat. Genet. 35:408-417.
31. Kos, C. H., T. C., L., Sinha, S., and al., e. 2003. Mice deficient in alpha-actinin-4 have severe glomerular disease. J Clin Invest 111: 1683-1690.
32. Michaud, J. L., Lemieux, L. I., Dube, M., and al., e. 2003. Focal and segmental glomerulosclerosis in mice with podocyte-specific expression of mutant alpha-actinin-4. J Am Soc Nephrol 14:1200-1211.
33. Breier, G., Albrecht, U., Sterrer, S., and Risau, W. 1992. Expression of vascular endothelial growth factor during embryonic angiogenesis and endothelial cell differentiation. Development 114:521-532.
34. Maynard, S. E., Min, J. Y., Merchan, J., and al., e. 2003. Excess placental soluble fms-like tyrosine kinase 1 (sFlt1) may contribute to endothellial dysfunction, hypertension, and proteinuria in preeclamsia. J Clin Invest 111:649-658.
35. Sugimoto, H., Hamano, Y., Charytan, D., and al., e. 2003. Neutralization of circulating vascular endothelial growth factor (VEGF) by anti-VEGF antibodies and soluble VEGF receptor 1 (sFlt-1) induces proteinuria. J Biol Chem 278:12605-12608.
36. Eremina, V., Sood, M., Haigh, J., Nagy, A., Lajoie, G. Ferrara, N., Gerber, H. P., Kikkawa, Y., Miner, J. H., and Quaggin, S. E. 2003. Glomerular-specific alterations of VEGF-A expression lead to distinct congenital and acquired renal diseases. J Clin Invest 111:707-716.
37. Schiffer, M., Schiffer, L. E., Gupta, A., and al., e. 2002. Inhibitory smads and TGF-beta signaling in glomerular cells. J Am Soc Nephrol 13:2657-2666.
38. Satchell, S. C., Abnderson, K. L., and Mathieson, P. W. 2004. Angiopoietin 1 and vascular endothelial growth factor modulate human glomerular endothelial cell barrier properties. J Am Soc Nephrol 15:566-574.
39. Virlon, B., Cheval, L., Buhler, J. M., Billon, E., Doucet, A., and Elalouf, J. M. 1999. Serial microanalysis of renal transcriptomes. Proc Natl Acad Sci USA 96:15286-15291.
40. Elalouf, J. M., Aude, J. C., Billon, E., Cheval, L., Doucet, A., and Virlon, B. 2002. Renal transcriptomes: segmental analysis of differential expression. Exp Nephrol 10:75-81.
41. El-meanawy, M. A., Schelling, J. R., Pozuelo, F., Churpek, M. M., Ficker, E. K., Iyengar, S., and Sedor, J. R. 2000. Use of serial analysis of gene expression to generate kidney expression libraries. Am J Physiol Renal Physiol 279:F383-392.
42. Chabardes-Garonne, D., Mejean, A., Aude, J.-C., Cheval, L., Di Stefano, A., Gaillard, M.-C., Imbert-Teboul, M., Wittner, M., Balian, C., Anthouard, V., et al. 2003. A panoramic view of gene expression in the human kidney. Proc Natl Acad Sci USA 100:13710-13715.
43. Yano, N., Endoh, M., Fadden, K., Yamashita, H., Kane, A., Sakai, H., and Rifai, A. 2000. Comprehensive gene expression profile of the adult human renal cortex: Analysis by cDNA array hybridization. Kidney Int. 57:1452-1459.
44. Higgins, J. P. T., Wang, L., Kambham, N., Montgomery, K., Mason, V., Vogelmann, S. U., Lemley, K. V., Brown, P. O., Brooks, J. D., and van de Rijn, M. 2004. Gene expression in the normal adult human kidney assessed by comlementary DNA microarray. Mol Biol Cell 15:649-656.
45. Takemoto, M., Asker, N., Gerhardt, H., Lundkvist, A., Johansson, B. R., Saito, Y., and Betsholtz, C. 2002. A new method for large scale isolation of kidney glomeruli from mice. Am. J. Pathol. 161:799-805.
46. Consortium, I. H. G. S. 2004. Finishing the euchromatic sequence of the human genome. Nature 431:931-945.
47. Doyonnas, R., Kershaw, D. B., Duhme, C., Merkens, H., Chelliah, S., Graf, T., and McNagny, K. M. 2001. Anuria, omphalocele, and perinatal lethality in mice lacking the CD34-related protein podocalyxin. J Exp Med 194:13-27.
48. Mundel, P., Heid, H. W., Mundel, T. M., Kruger, M., Reiser, J., and LKriz, W. 1997. Synaptopodin: an actin-associated protein in telencephalic dendrites and renal podocytes. J Cell Biol 139:193-204.
49. Hiromura, K., Haseley, L. A., Zhang, P., Monkawa, T., Durvasula, R., Petermann, A. T., Alpers, C. E., Mundel, P., and Shankland, S. J. 2001. Podocyte expression of the CDK-inhibitor p57 during development and disease. Kidney Int. 60:2235-2246.
50. Wharram, B. L., Goyal, M., Gillespie, P. J., Wiggins, J. E., Kershaw, D. B., Holzman, L. B., Dysko, R. C., Saunders, T. L., Samuelson, L. C., and Wiggins, R. C. 2000. Altered podocyte structure in GLEPP1 (Ptpro)-deficient mice associated with hypertension and low glomerular filtration rate. J Clin Invest 106:1281-1290.
51. Heathcott, R. W., Morison, I. M., Gubler, M. C., Corbett, R., and Reeve, A. E. 2002. A review of the phenotypic variation due to the Denys-Drash syndrome-associated germline WT1 mutation R362X. Hum Mutat 19:462-???
52. Cui, S., Schwartz, L., and Quaggin, S. E. 2003. Pod1 is required in stromal cells for glomerulogenesis. Dev Dyn 226:512-522.
53. Kume, T., Deng, K., and Hogan, B. L. 2000. Murine forkhead/winged helix genes Foxc1 (Mf1) and Foxc2 (Mfh1) are required for the early organogenesis of the kidney and urinary tract. Development 127:1387-1395.
54. Enge, M., Bjarnegård, M., Gerhardt, H., Gustafsson, E., Kalén, M., Asker, N., Hammes, H.-P., Shani, M., Fässler, R., and Betsholtz, C. 2002. Endothelium-specific platelet-derived growth factor-B ablation mimics diabetic retinopathy. EMBO J. 21:4307-4316.
55. Belteki, G., Haigh, J., Kabacs, N., Haigh, K., Sison, K., Costantini, F., Whitsett, J. A., Quaggin, S. E., and Nagy, A. 2005. Conditional and inducible transgene expression in mice through the combinatorial use of Cre-mediated recombination and tetracycline induction. Nucl Acids Res In press.
56. Watanabe, G. Saito, Y., Madaule, P., Ishizaki, T., Fujisawa, K., Morii, N., Mukai, H., Ono, Y., Kakizuka, A., and Narumiya, S. 1996. Protein kinase N (PKN) and PKN-related protein rhophilin as targets of small GTPase Rho. Science 271:645-648.
57. Nakamura, K., Fujita, A., Murata, T., Watanabe, G. Mori, C., Fujita, J., Watanabe, N., Ishizaki, T., Yoshida, O., and Narumiya, S. 1999. Rhophilin, a small GTPase Rho-binding protein, is abundantly expressed in the mouse testis and localized in the prinipal piece of the sperm tail. FEBS Lett 445:9-13.
58. Tamagnone, L., and Comoglio, P. M. 2004. To move or not to move? Semaphorin signalling in cell migration. EMBO Reports 5:356-361.
59. Serini, G, Valdembri, D., Zanivan, S., Morterra, G, Burkhardt, C., Caccavari, F., Zammataro, L., Primo, L., Tamagnone, L., Logan, M., et al. 2003. Class 3 semaphorins control vascular morphogenesis by inhibiting integrin function. Nature 424:391-397.
60. Shoji, W., Isogai, S., Sato-Maeda, M., Obinata, M., and Kuwada, J. Y. 2003. Semaphorin3a1 regulates angioblast migration and vascular development in zebrafish embryos. Development 130:3227-3236.
61. Koshikawa, N., Nakamura, T., Tsuchiya, N., Isaji, M., Yasumitsu, H., Umeda, M., and Miyazaki, K. 1996. Purification ans identification of a novel and four known serine proteinase inhibitors secreted by human glioblastoma cells. J Biochem 119:334-339.
62. Shioda, T., Fenner, M. H., and Isselbacher, K. J. 1997. MSG1 and its related protein MRG1 share a transcription activating domain. Gene 204:235-241.
63. Yin, Z., Haynie, J., Yang, X., Han, B., Kiatchoosakun, S., Restivo, J., Yuan, S., Prabhakar, N. R., Herrup, K., Conlon, R. A., et al. 2002. The essential role of Cited2, a negative regulator for HIF-1alpha, in heart development and neurulation. Proc Natl Acad Sci USA 99:10488-10493.
64. Neuner-Jehle, M., Denizot, J. P., Borbely, A. A., and Mallet, J. 1996. Characterization and sleep deprivation-induced expression modulation of dendrin, a novel dendritic protein in rat brain neurons. J Neurosci Res 46:138-151.
65. Goutebroze, L., Brault, E., Muchardt, C., Camonis, J., and Thomas, G. 2000. Cloning and characterization of SCHIP-1, a novel protein interacting specifically with spliced isoforms and naturally occuring mutant NF2 proteins. Mol Cell Biol 20:1699-1712.
66. Pohl, U., Smith, J. S., Tachibana, I., Ueki, K., Lee, H. K., Ramaswamy, S., Wu, Q., Mohrenweiser, H. W., Jenkins, R. B., and Louis, D. N. 2000. EHD2, EHD3, and EHD4 encode novel members of a highly conserved family of EH domain-containing proteins. Genomics 63:255-262.
67. Galperin, E., Benjamin, S., Rapaport, D., Rotem-Yehudar, R., Tolchinsky, S., and Horowitz, M. 2002. EHD3: a protein that resides in recycling tubular and vesicular membrane structures and interacts with EHD1. Traffic 3:575-589.
68. Melkonyan, H. S., Chang, W. C., Shapiro, J. P., Mahadevappa, M., Fitzpatrick, P. A., Kiefer, M. C., Tomei, L. D., and Umansky, S. R. 1997. SARPs: a family of secreted apoptosis-related proteins. Proc Natl Acad Sci USA 94:13636-13641.
69. Lescher, B., Haenig, B., and Kispert, A. 1998. sFRP-2 is a target of the Wnt-4 signaling pathway in the developing kidney. Dev Dyn 213:440-451.
70. Morello, R., Zhou, G, Dreyer, S. D., Harvey, S. J., Ninomiya, Y., Thorner, P. S., Miner, J. H., Cole, W., Winterpacht, A., Zabel, B., et al. 2001. Regulation of glomerular basement membrane collagen expression by LMX1B contributes to renal disease in nail patella syndrome. Nat Genet 27:205-208.
71. Taragnat, C., Berger, M., and Jean, C. 1988. Preliminary characterization, androgen-dependence and ontogeny of an abundant protein from mouse vas deferens. J Reprod Fertil 83:835-842.
72. Putilina, T., Jaworski, C., Gentleman, S., McDonald, B., Kadiri, M., and Wong, P. 1998. Analysis of a human cDNA containing a tissue-specific alternatively spöliced LIM domain. Biochem Biophys Res Commun 252:433-439.
73. Ooshio, T., Irje, K., Morimoto, K., Fukuhara, A., Imai, T., and Takai, Y. 2004. Involvement of LMO7 in the asssociation of two cell-cell adhesion molecules, nectin and E-cadherin, through afadin and alpha-actinin in epithelial cells. J Biol Chem 279:31365-31373.
74. Kriz, W. 2003. Progression of chronic renal failure in focal segmental glomerulosclerosis: consequence of podocyte damage or of tuboluinterstitial fibrosis? Pediatr Nephrol 18:617-622.
75. Sadlier, D. M., Connolly, S. B., Kieran, N. E., Roxburgh, S., Brazil, D. P., Kairaitis, L., Wang, Y., Harris, D. C., Doran, P., and Brady, H. R. 2004. Sequential exctracellular matrix-focused and baited-global cluster analysis of serial transcriptomic profiles identifies candidate modulators of renal tubulointerstitial fibrosisin murine adriamycin-induced nephropathy. J Biol Chem 279:29670-29680.
76. Sarwal, M., Chua, M. S., Kambham, N., Hsieh, S. C., Satterwhite, T., Masek, M., and Salvatierra, O. J. 2003. Molecular heterogeneity in acute allograft rejection identified by DNA microarray profiling. N Engl J Med 349:125-138.
77. Wilson, K. H. S., Eckenrode, S. E., Li, Q.-Z., Ruan, Q.-G, Yang, P., Shi, J.-D., Davoodi-Semiromi, A., McIndoe, R. A., Crocker, B. P., and She, J.-X. 2003. Microarray analysis of gene expression in the kidneys of new- and post-onset diabetic NOD mice. Diabetes 52:2151-2159.
78. Wada, J., Zhang, H., Tsuchiyama, Y., Hiragushi, K., Hida, K., Shikata, K., Kanwar, Y. S., and Makino, H. 2001. Gene expression profile in streptozotocin-induced diabetic mice kidneys undergoing glomerulosclerosis. Kidney Int 59:1363-1373.
79. Susztak, K., Böttinger, E., Novetsky, A., Liang, D., Zhu, Y., Ciccone, E., Wu, D., Dunn, S., McCue, P., and Sharma, K. 2004. Molecular profiling of diabetic mouse kidney reveals novel genes linked to glomerular disease. Diabetes 53:784-794.
80. Peterson, K. S., Huang, J.-F., Zhu, J., D'Agati, V., Liu, X., Miller, N., Erlander, M. G., Jackson, M. R., and Winchester, R. J. 2004. Characterization of heterogeneity in the molecular pathogenesis of lupus nephritis from transcriptional profiles of laser captured glomeruli. J Clin Invest 113:1722-1733.
81. Baelde, H. J., Eikmans, M., Doran, P. P., Lappin, D. W. P., de Heer, E., and Bruijn, J. A. 2004. Gene expression profiling in glomeruli from human kidneys with diabetic nephropathy. Am J Kidney Diseases 43:636-650.
82. Eremina, V., Wong, M. A., Cui, S., Schwartz, L., and Quaggin, S. E. 2002. Glomerular-specific gene excision in vivo. J Am Soc Nephrol 13:788-793.
83. Soares, M. B., Bonaldo, M. F., Jelene, P., Su, L., Lawton, L., and Efstratiadis, A. 1994. Construction and characterization of a normalized cDNA library. Proc. Natl. Acad. Sci. USA 91:9228-9232.
84. Gargett, C. E., Bucak, K., and Rogers, P. A. 2000. Isolation, characterization and long-term culture of human myometrial microvascular endothelial cells. Hum. Reprod. 15:293-301.
85. Scheidl, S., Nilsson, S., Kalen, M., Hellstrom, M., Takemoto, M., Håkansson, J., and Lindahl, P. 2002. mRNA expression profiling of laser microbeam microdissected cells from slender embryonic structures. Am. J. Pathol. 160:801-813.
86. Smyth, G. K. 2004. Linear models and empirical Bayes methods for assessing differential expression in microarray experiments. Statistical Applications in Genetics and Molecular Biology 3: Article 3.
87. Benjamini, Y., and Hochberg, Y. 1995. Controlling the false discovery rate: a prectical and powerful approach to multiple testing. J Royal Statistical Society Series B 57:289-300.
88. Boström, H., Willetts, K., Pekny, M., Levéen, P., Lindahl, P., Hedstrand, H., Pekna, M., Hellström, M., Gebre-Medhin, S., Schalling, M., et al. 1996. PDGF-A signaling is a critical event in lung alveolar myofibroblast development and alveogenesis. Cell 85:863-873.
89. Herb, A., Wisden, W., Catania, M. V., Marechal, D., Dresse, A., and Seeburg, P. H. 1997. prominent dendritic localization in forebrain neurons of a novel mRNA and its product, dendrin. Mol Cell Neurosci 8:367-374.
90. Lahdenkari, A. T., Lounatmaa, K., Patrakka, J., Holmberg, C., Wartiovaara, J., Kestila, M., Koskimies, O., and Jalanko, H. 2004. Podocytes are firmly attached to glomerular basement membrane in kidneys with heavy proteinuria. J Am Soc Nephrol 15:2611-2618.

We claim:

1. A composition comprising a plurality of isolated probes that in total selectively bind to at least 2 the glomerular markers listed in Table 6 or Table 7, complements thereof, or their expression products, wherein at least 10% of the probes in total are selective for glomerular markers.

2. The composition of claim 1 wherein the plurality of isolated probes in total selectively binds to at least 5 the glomerular markers in Table 6 or Table 7, complements thereof, or their expression products.

3. The composition of claim 1 wherein at least 20% of the probes in total are selective for glomerular markers.

4. The composition of claim 1 wherein the plurality of isolated probes in total selectively binds to at least 2 of the glomerular markers disclosed herein in Table 11, complements thereof, human homologues thereof, or their expression products.

5. The composition of claim 1 wherein the plurality of isolated probes in total selectively binds to at least 10 of the glomerular markers disclosed herein in Table 11, complements thereof, human homologues thereof, or their expression products.

6. The composition of claim 1 wherein the plurality of isolated probes in total selectively binds to at least 2 of the glomerular markers disclosed herein in Table 3, complements thereof, human homologues thereof, or their expression products.

7. The composition of claim 1 wherein the plurality of isolated probes in total selectively binds to at least 10 of the glomerular markers disclosed herein in Table 3, complements thereof, human homologues thereof, or their expression products.

8. The composition of claim 1 wherein the plurality of isolated probes in total selectively binds to at least 2 of the glomerular markers disclosed herein in Table 4, complements thereof, human homologues thereof, or their expression products.

9. The composition of claim 1 wherein the plurality of isolated probes in total selectively binds to at least 10 of the glomerular markers disclosed herein in Table 4, complements thereof, human homologues thereof, or their expression products.

10. The composition of claim 1 wherein the plurality of isolated probes in total selectively binds to at least 2 of the glomerular markers disclosed herein in Table 5, complements thereof, human homologues thereof, or their expression products.

11. The composition of claim 1 wherein the plurality of isolated probes in total selectively binds to at least 10 of the glomerular markers disclosed herein in Table 5, complements thereof, human homologues thereof, or their expression products.

12. The composition of claim 1 wherein the plurality of isolated probes in total selectively binds to at least 2 of the glomerular markers disclosed herein in Table 13, complements thereof, human homologues thereof, or their expression products.

13. The composition of claim 1 wherein the plurality of isolated probes in total selectively binds to at least 10 of the glomerular markers disclosed herein in Table 13, complements thereof, human homologues thereof, or their expression products.

14. The composition of claim 1 wherein the plurality of isolated probes in total selectively binds to at least 2 of the glomerular markers disclosed herein in Table 9, complements thereof, human homologues thereof, or their expression products.

15. The composition of claim 1 wherein the plurality of isolated probes in total selectively binds to at least 10 of the glomerular markers disclosed herein in Table 9, complements thereof, human homologues thereof, or their expression products.

16. A composition comprising a plurality of isolated probes that in total selectively bind to at least 51 of the glomerular markers disclosed herein in Table 9, complements thereof, human homologues thereof, or their expression products, wherein at least 3.75% of the probes in total are selective for glomerular markers.

17. The composition of claim 16, wherein the plurality of isolated probes in total selectively binds to at least 100 of the glomerular markers disclosed herein in Table 9, complements thereof, human homologues thereof, or their expression products.

18. The composition of claim 1 wherein the plurality of isolated probes comprises polynucleotide probes.

19. The composition of claim 1 wherein the plurality of isolated probes comprises antibody probes.

20. The composition of claim 1 wherein the plurality of isolated probes are arrayed on a solid support.

21. A method to profile a glomerular expression pattern from a subject, comprising
a) providing one of more compositions according to claims 1;
b) contacting the one or more compositions with glomerular polynucleotides and/or polypeptides under conditions to promote selective binding of the probes to their glomerular marker target; and
c) detecting presence of the glomerular marker targets by binding of the probes to their glomerular marker target, wherein the glomerular marker targets detected comprise a glomerular expression pattern.

22. A method for identifying glomerular marker polynucleotides, comprising
a) perfusing a target kidney in an organism with a solution containing magnetic beads, wherein the magnetic bead diameter is approximately equivalent to the capillary diameter of glomerular capillaries;
b) removing glomerular-containing kidney tissue from the organism;
c) digesting the glomerular-containing kidney tissue to separate glomeruli from associated kidney tissue;
d) magnetically isolating glomeruli from the digested glomerular-containing kidney tissue;
e) isolating mRNA from the isolated glomeruli
f) normalizing the mRNA to at least partially suppress high copy number mRNA transcripts;
g) identifying mRNA that are expressed in the glomerulus, wherein such mRNA are glomerular marker polynucleotides.

22. The method of claim 21, further comprising identifying podocyte-specific glomerular polynucleotides, wherein such identifying comprises identifying those glomerular marker polynucleotides that are expressed in glomerular podocytes.

23. The method of claim 21, further comprising identifying non-podocyte-specific glomerular polynucleotides, wherein such identifying comprises identifying those glomerular marker polynucleotides that are expressed in glomerular endothelial and/or mesangial cells.
